SEQUENCE LISTING (151 sequences)

SEQ ID NO: 1 - protein, 508 aa, synthetic construct
ADTLCIGYHANNSTDTVDTVLEKNVTVTHSVNLLEDRHNGKLCKLGGIAPLHLGKCNIAGWLLGNPECELLLTVSSWSYIVETSNSDNGTCYPGDFINYEELREQLSSVSSFERFEIFPKTSSWPDHETNRGVTAACPYAGANSFYRNLIWLVEKGNSYPKLSKSYVNNKGKEVLVLWGIHHPPTSTDQQSLYQNADAYVFVGSSKYNRKFKPEIAARPKVRGQAGRMNYYWTLIEPGDTITFEATGNLVVPRYAFAMNRGSGSGIIISDAPVHDCNTKCQTPKGAINTSLPFQNIHPVTIGECPKYVKSTKLRMATGLRNIPSIQSRGLFGAIAGFIEGGWTGMIDGWYGYHHQNEQGSGYAADQRSTQNAIDGITNKVNSVIEKMNTQFTAVGKEFNHLEKRIENLNKKVDDGFLDIWTYNAELLVLLENERTLDFHDSNVKNLYEKVRSQLRNNAKEIGNGCFEFYHKCDDTCMESVKNGTYDYPKYSEESKLNREEIDGVKLES

SEQ ID NO: 2 - protein, 508 aa, synthetic construct
ADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHHQNEQGSGYAADRKSTQNAIDGITNKVNSVIEKMNTQFTSVGKEFNHLEKRIENLNRKVDDGFLDVWTYNAELLVLLENERTLDYHDSNVKNLYEKVRSQLKNNAKEIGNGCFEFYHKCDDSCMESVKNGTYDYPKYSEESKLNREEIDGVKLES

SEQ ID NO: 3 - protein, 508 aa, synthetic construct
ADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDS

SEQ ID NO: 4 - protein, 507 aa, synthetic construct
ADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLES

SEQ ID NO: 5 - protein, 508 aa, synthetic construct
ADTLCIGYHANNSTDTVDTVLEKNVTVTHSVNLLENRHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHDGTNSFYRNLIWLVKKGNSYPKINKSYINNKEKEILVLWAIHHPSTSADQQSLYQNADAYVFVGSSRYSRKFEPEVATRPKVRDQAGRMNYYWTLVEPGDKITFEATGNLVVPRYAFALKRNSGSGIIISDTSVHDCDTNCQTPNGAINTSLPFQNIHPVTIGECPKYVKSTKLRMATGLRNIPSIQSRGLFGAIAGFIEGGWTGMIDGWYGYHHQNEQGSGYAADLKSTQNAIDGITNKVNSVIEKMNTQFTAVGKEFSHLERRIENLNKKVDDGFLDIWTYNAELLVLLENERTLDYHDSNVKNLYEKVRSQLKNNAKEIGNGCFEFYHKCDDMCMESVKNGTYDYPKYSEEAKLNREEIDGVKLES

SEQ ID NO: 6 - protein, 507 aa, synthetic construct
ADTICIGYHANNSTDTVDTVLEKNVTVTHSVNLLEDSHNGKLCRLKGKAPLQLGNCNIAGWVLGNPECESLLSNRSWSYIAETPNSENGTCYPGDFADYEELREQLSSVSSFERFEIFPKERSWPNHTTRGVTAACPHARKSSFYKNLVWLTEANGSYPNLSRSYVNNQEKEVLVLWGVHHPSNIEEQRALYRKDNAYVSVVSSNYNRRFTPEIAKRPKVRDQSGRMNYYWTLLEPGDTIIFEATGNLIAPWYAFALSRGPGSGIITSNAPLDECDTKCQTPQGAINSSLPFQNIHPVTIGECPKYVRSTKLRMVTGLRNIPSVQSRGLFGAIAGFIEGGWTGMMDGWYGYHHQNEQGSGYAADQKSTQNAINGITNKVNSVIEKMNTQFTAVGKEFNKLEKRMENLNKKVDDGFMDIWTYNAELLVLLENERTLDFHDSNVKNLYEKVKNQLRNNAKELGNGCFEFYHKCDNECMESVKNGTYDYPKYSEESKLNREKIDGVKLES

SEQ ID NO: 7 - protein, 507 aa, synthetic construct
ADTICIGYHANNSTDTVDTVLEKNVTVTHSVNLLEDSHNGKLCLLKGIAPLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQGAINSSLPFQNVHPVTIGECPKYVRSTKLRMVTGLRNIPSIQSRGLFGAIAGFIEGGWTGMMDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLES

SEQ ID NO: 8 - protein, 508 aa, synthetic construct
ADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNKGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLES

SEQ ID NO: 9 - protein, 508 aa, synthetic construct
ADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLES

SEQ ID NO: 10 - protein, 508 aa, synthetic construct
ADTICVGYHANNSTDTVDTILEKNVTVTHSVNLLESNHNGKLCSLNGKAPLQLGNCNVAGWILGNPECDLLLTANSWSYIIETSNSKNGACYPGEFADYEELRELLSTVSSFERFEIFPKATSWPHHETTKGTTVACPHSGVNSFYRNLLWIVKKGNSYPKLSKSYTNSKGKEVLVIWGVHHPPTDSDQQTLYQNNHTYVSVGTSKYYRRFTPEIVARPKIREQAGRMNYYWTLLDQGDTITLEATGNLIVPWHAFALNKGSNSGIMISDADFHNCTTKCQTPHGALKSNLPFQNVHPITIGECPKYVKSTQLRMATGLRNVPSIQSRGLFGAIAGFIEGGWTGMIDGWYGYHHQNEQGSGYAADQKSTQIAIDGISNKVNSVIEKMNIQFASVGKEFNDLEKRIENLNKKVDDGFVDVWTYNAELLILLENERTLDFHDFNVKNLYEKVKSQLRNNAKEIGNGCFEFYHKCDNECMESVKNGTYNYPKYSEESKLNREEIDGVKLES

SEQ ID NO: 11 - protein, 508 aa, synthetic construct
ADTICIGYHANNSTDTVDTVLEKNVTVTHSVNLLENNHNGKLCNLNGIAPLQLGKCNVAGWLLGNPECGLLLNANAWSYIIETSDSKNGTCYPGDFIDYEELREQLGSVSSFEKFEIFPKASSWPDHETTKGTTAACPYSGVESFYRNLLWIIKKGNSYPKISKSYTNNRGKEVLVLWGVHHPPTTSDQQTLYQNIDAYVSVGSSKYNRRFTPEIATRPKVRGLAGRMNYYWTLLDQGDTIMFEATGNLIAPWYAFALNKGSDSGIITSDTPVHDCDTKCQTPYGALNSSLPFQNVHPITIGECPKYVKSTKLRMATGLRNVPSIQSKGLFGAIAGFIEGGWTGMIDGWYGYHHQNEQGSGYSADQKSTQNAIDGITNKVNSVIEKMNTQFTAVGKEFNNLERRIENLNKKVDDGFLDVWTYNAELLVLLENERTLDFHDSNVRDLYERVKSQLRNNAKEIGNGCFEFYHKCDDECMENVKNGTYDYPKYSEEAKLNREKIDGVKIES

SEQ ID NO: 12 - protein, 508 aa, synthetic construct
ADTLCIGYHANNSTDTVDTILEKNVTVTHSVNLLEDRHNGKLCNLRGEAPLHLGKCNIAGWLLGNPECELLFAVNSWSYIVETSNSDNGTCYPGDFTSYEELREQLSSVSSFERFEIFPKASSWPNHETNRGVTAACPYAGTNSFYRNLIWLVKKGNSYPKLSKSYVNNKKKEVLVLWGIHHPPTNADQQSLYQNADAYVFVGSSKYNKKFKPEIAKRPKVRGQAGRMNYYWTLVEPGDTITFEATGNLVAPRYAFAMNRDPGSGIITSDAPIHDCNATCQTPKGAINTSLPFQNIHPITIGECPKYVKSTRLRMATGLRNIPSIQSRGLFGAIAGFIEGGWTGMIDGWYGYHHQNGQGSGYAADQKSTQNAIDRITNKVNSVIEKMNIQFTAVGKEFNHLERRIENLNKKVDDGFLDVWTYNAELLVLLENERTLDFHDSNVKTLYEKVKTQLRNNAKEIGNGCFEFYHKCDDTCMESIKNGTYDYPKYSKESKLNREEIDGVQLES

SEQ ID NO: 13 - protein, 508 aa, synthetic construct
ADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLES

SEQ ID NO: 14 - protein, 508 aa, synthetic construct
ADTICIGYHANNSTDTVDTVLEKNVTVTHSVNLLEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALNRGFGSGIITSNASMDECDTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLES

SEQ ID NO: 15 - protein, 507 aa, synthetic construct
ADTICIGYHANNSTDTVDTVLEKNVTVTHSVNLLEDSHNGKLCLLKGIAPLQLGNCSVAGWILGNPECESLISKKSWSYIVETPNPENGACYPGEFADYEELREQLSSVSSFERFEIFPKESSWPNHTATGESASCSHNGKKSFYRNLIWLTVKNGLYPNLSKSYENDKEKEVLILWGVHHPPNIENQRTLYHTENAYVSVVSSHYSGRFTPEITKRPKVRDQEGRINYYWTLLEPGDTIIFEANGNLIAPWYAFALSRGLGSGIITSNAPMDECDSKCQTPQGAINSSLPFQNVHPVTIGECPKYVRSAKLRMVTGLRNIPSIQSRGLFGAIAGFIEGGWTGMVDGWYGYHHQNEQGSGYAADQESTQNAINGITNKVNSVIEKMNTQFTAVGKEFNKLERRMENLNKKVDDGFLDIWTYNAELLVLLENERTLDFHDSNVKSLYEKVKSQLKNNAKEIGNGCFEFYHKCNNECMESVKNGTYDYPKYYEESKLNREKIDGVKLDS

SEQ ID NO: 16 - protein, 500 aa, synthetic construct
TATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVKQNGTSSACIRKSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIQSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKS

SEQ ID NO: 17 - protein, 500 aa, synthetic construct
TATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSSCKRRSNNSFFSRLNWLTHLKFKYPALNVTMPNNEKFDKLYIWGVHHPGTNNDQISLYTQASGRITVSTKRSQQTVIPNIGSRPRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKS

SEQ ID NO: 18 - protein, 500 aa, synthetic construct
TATLCLGHHAVPNGTIVKTITNDRIEVTNATELVQNSSIGEICNSPHQILDGGNCTLIDALLGDPQCDGFQNKEWDLFVERSRANSSCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQFSLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKS

SEQ ID NO: 19 - protein, 500 aa, synthetic construct
TATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYTYPALNVTMPNNEQFDKLYIWGVHHPSTDKDQISLFAQPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKS

SEQ ID NO: 20 - protein, 500 aa, synthetic construct
TATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFTNEGFNWTGVAQDGTSYACKRGSVKSFFSRLNWLHKLEYKYPALNVTMPNNDKFDKLYIWGVHHPSTDSDQTSLYVQASGRVTVSTKRSQQTVIPNIGSRPWVRGISSRISIYWTIVKPGDILLIKSTGNLIAPRGYFKIRSGKSSIMRSDAPIGNCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKS

SEQ ID NO: 21 - protein, 500 aa, synthetic construct
TATLCLGHHAVPNGTLVKTITNDQIEVTNATELVQSSSTGRICDSPHRILDGKNCTLVDALLGDPHCDGFQNEKWDLFVERSKAFSNCYPYDVPDYASLRSLVASSGTLEFINESFNWTGVTQSGGSYACKRGSDNSFFSRLNWLYESESKYPVLNVTMPNNGNFDKLYIWGVHHPSTDKEQTNLYVRASGRVTVSTKRSQQTIIPNIGSRPWVRGLSSRISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAIAGFIENGWEGMVDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKS

SEQ ID NO: 22 - protein, 500 aa, synthetic construct
TATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKS

SEQ ID NO: 23 - protein, 499 aa, synthetic construct
ATLCLGHHAVPNGTLVKTITDDQIEVTNATELVQSSSTGGICNSPHQILDGKNCTLIDALLGDPHCDDFQNKEWDLFVERSTAYSNCYPYYVPDYATLRSLVASSGNLEFTQESFNWTGVAQGGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNKITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAEILVALENQHTIDLTDSEMSKLFERTRRQLRENAEDMGNGCFKIYHKCDNACIGSIRNGTYDHDIYRNEALNNRFQIKGVQLKS

SEQ ID NO: 24 - protein, 500 aa, synthetic construct
TATLCLGHHAVPNGTLVKTITNDQIEVTNATELVQSSSTGKICDNPHRILDGINCTLIDALLGDPHCDGFQNEKWDLFVERSKAFSNCYPYDVPDYASLRSLVASSGTLEFINEGFNWTGVTQNGGSSACKRGPDNGFFSRLNWLYKSGSTYPVQNVTMPNNDNSDKLYIWGVHHPSTDKEQTDLYVQASGKVTVSTKRSQQTVIPNVGSRPWVRGLSSRVSIYWTIVKPGDILVINSNGNLIAPRGYFKMRTGKSSIMRSDAPIGTCSSECITPNGSIPNDKPFQNVNKITYGACPKYVKQNTLKLATGMRNVPEKQTRGIFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKS

SEQ ID NO: 25 - protein, 500 aa, synthetic construct
TATLCLGHHAVPNGTIVKTITNDQIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKS

SEQ ID NO: 26 - protein, 500 aa, synthetic construct
TATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFLYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKS

SEQ ID NO: 27 - protein, 500 aa, synthetic construct
TATLCLGHHAVPNGTIVKTITNDRIEVTNATELVQNSSIGEICGSPHQILDGGNCTLIDALLGDPQCDGFQNKEWDLFVERSRANSNCYPYDVPGYASLRSLVASSGTLEFKNESFNWTGVKQNGTSSACIRGSSSSFFSRLNWLTSINNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRNGKSSIMRSDAPIGRCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKS

SEQ ID NO: 28 - protein, 500 aa, synthetic construct
TATLCLGHHAVPNGTLVKTITNDQIEVTNATELVQSSSTGRICDSPHRILDGENCTLIDALLGDPHCDGFQNKEWDLFVERSKAYSNCYPYDVPDYASLRSLVASSGTLEFNNESFNWTGVAQNGTSYACKRSSIKSFFSRLNWLHQLKYKYPALNVTMPNNDKFDKLYIWGVHHPSTDSDQTSIYAQASGRVTVSTKRSQQTVIPNIGSRPWVRGISSRISIHWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKS

SEQ ID NO: 29 - protein, 508 aa, synthetic construct
ADRICTGITSSNSPHVVKTATQGEVNVTGVIPLTTTPTKSHFANLKGTETRGKLCPKCLNCTDLDVALGRPKCTGKIPSAKVSILHEVRPVTSGCFPIMHDRTKIRQLPNLLRGYEHVRLSTNNVINAEDAPGGPYEIGTSGSCPNITNGKGFFATMAWAVPKNKTATNPLTIEVPYICTEEEDQITVWGFHSDDETQMAKLYGDSKPQKFTSSANGMTTHYVSQIGGFPNQTEDGGLQQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFS

SEQ ID NO: 30 - protein, 510 aa, synthetic construct
ADRICTGITSSNSPHVVKTATQGEVNVTGVIPLTTTPTKSHFANLKGTKTRGKLCPKCLNCTDLDVALGRPKCMGTIPSAKASILHEVKPVTSGCFPIMHDRTKIRQLPNLLRGYENIRLSTHNVINAEAAPGGPYIVGTSGSCPNVTNGNGFFATMAWAVPKNNNNKTATNPLTVEVPFICTEGEDQITVWGFHSDNEIQMVKLYGDSKPQKFTSSANGVTTHYVSQIGGFPKQAEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEF

SEQ ID NO: 31 - protein, 509 aa, synthetic construct
ADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEF

SEQ ID NO: 32 - protein, 508 aa, synthetic construct
ADRICTGITSSNSPHVVKTATQGEVNVTGVIPLTTTPTKSHFANLKGTKTRGKLCPNCLNCTDLDVALGRPMCMGTIPSAKASILHEVRPVTSGCFPIMHDRTKIRQLPNLLRGYENIRLSTHNVINAERAPGGPYRLGTSGSCPNVTSRNGFFATMAWAVPRDNKTATNPLTVEVPYICTKGEDQITVWGFHSDDKTQMKNLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEF

SEQ ID NO: 33 - protein, 508 aa, synthetic construct
ADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMVRLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFS

SEQ ID NO: 34 - protein, 509 aa, synthetic construct
ADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEGAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFS

SEQ ID NO: 35 - protein, 511 aa, synthetic construct
ADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFS

SEQ ID NO: 36 - protein, 508 aa, synthetic construct
ADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFS

SEQ ID NO: 37 - protein, 509 aa, synthetic construct
ADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSESCPSATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEF

SEQ ID NO: 38 - protein, 508 aa, synthetic construct
ADTICIGYHANNSTDTVDTVLEKNVTVTHSVNLLEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTAACSHKGKSSFYRNLLWLTEKDGSYPNLSKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGFGSGIITSNASMDECDTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLES

SEQ ID NO: 39 - protein, 508 aa, synthetic construct
ADTLCIGYHANNSTDTVDTVLEKNVTVTHSVNLLEDRHNGKLCKLRGVAPLHLGKCNIAGWLLGNPECELLFTASSWSYIVETSNSDNGTCYPGDFINYEELREQLSSVSSFERFEIFPKASSWPDHETNRGVTAACPYAGANSFYRNLIWLVKKGNSYPKLSKSYVNNKEKEVLVLWGIHHPPTSTDQQSLYQNADAYVFVGSSKYNKKFKPEIATRPKVRGQAGRMNYYWTLVEPGDTITFEATGNLVVPRYAFAMKRGSGSGIIISDTPVHDCNTTCQTPKGAINTSLPFQNIHPVTIGECPKYVKSTKLRMATGLRNIPSIQSRGLFGAIAGFIEGGWTGMIDGWYGYHHQNEQGSGYAADQKSTQNAIDGITNKVNSVIEKMNTQFTAVGKEFNHLEKRIENLNKKVDDGFLDVWTYNAELLVLLENERTLDYHDSNVKNLYEKVRSQLKNNAKEIGNGCFEFYHKCDDTCMESVKNGTYDYPKYSEESKLNREEIDGVKLES

SEQ ID NO: 40 - protein, 500 aa, synthetic construct
TATLCLGHHAVPNGTLVKTITNDQIEVTNATELVQSSSTGGICDSPHQILDGENCTLIDALLGDPHCDGFQNKEWDLFVERSKAYSNCYPYDVPDYASLRSLVASSGTLEFNNESFNWTGVAQNGTSSACKRRSNKSFFSRLNWLHQLKYKYPALNVTMPNNEKFDKLYIWGVHHPSTDSDQISLYAQAPGRVTVSTKRSQQTVIPNIGSRPWVRGVSSRISIYWTIVKPGDILLINCTGNLIAPRGYFKIRSGKSSIMRSDASIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKS

SEQ ID NO: 41 - protein, 500 aa, synthetic construct
TATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQDGTSSACIRGSKNSFFSRLNWLTHLNFKYPALNVTMPNNEQFDKLYIWGVHHPGTDKDQIFLYAQASGRITVSTKRSQQAAIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNKRFQIKGIELKS

SEQ ID NO: 42 - protein, 531 aa, synthetic construct
ADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHT

SEQ ID NO: 43 - protein, 42 aa, synthetic construct
MGVYQILAIYSTVASSLVLLVSLGAISFWMCSNGSLQCRICI

SEQ ID NO: 44 - protein, 42 aa, synthetic construct
TRIYQILAIYSTVASSLVLVVSLGAISFWMCSNGSLQCRICI

SEQ ID NO: 45 - protein, 42 aa, synthetic construct; identical to SEQ ID NO: 43

SEQ ID NO: 46 - protein, 42 aa, synthetic construct; identical to SEQ ID NO: 44

SEQ ID NO: 47 - protein, 42 aa, synthetic construct
MGVYRILAIYSTVASSLVLLVSLGAISFWMCSNGSLQCRICI

SEQ ID NO: 48 - protein, 42 aa, synthetic construct; identical to SEQ ID NO: 43

SEQ ID NO: 49 - protein, 42 aa, synthetic construct
MGIYQILAIYSTVASSLVLLVSLGAISFWMCSNGSLQCRICI

SEQ ID NO: 50 - protein, 42 aa, synthetic construct; identical to SEQ ID NO: 44

SEQ ID NO: 51 - protein, 41 aa, synthetic construct
GYKDWILWISFAISCFLLCVVLLGFIMWACQKGNIRCNICI

SEQ ID NO: 52 - protein, 41 aa, synthetic construct; identical to SEQ ID NO: 51

SEQ ID NO: 53 - protein, 41 aa, synthetic construct; identical to SEQ ID NO: 51

SEQ ID NO: 54 - protein, 41 aa, synthetic construct; identical to SEQ ID NO: 51

SEQ ID NO: 55 - protein, 41 aa, synthetic construct; identical to SEQ ID NO: 51

SEQ ID NO: 56 - protein, 41 aa, synthetic construct; identical to SEQ ID NO: 51

SEQ ID NO: 57 - protein, 41 aa, synthetic construct; identical to SEQ ID NO: 51

SEQ ID NO: 58 - protein, 41 aa, synthetic construct; identical to SEQ ID NO: 51

SEQ ID NO: 59 - protein, 42 aa, synthetic construct; identical to SEQ ID NO: 43

SEQ ID NO: 60 - protein, 42 aa, synthetic construct
TRIYQILAIYSTVASSLVLLVSLGAISFWMCSNGSLQCRICI

SEQ ID NO: 61 - protein, 41 aa, synthetic construct; identical to SEQ ID NO: 51

SEQ ID NO: 62 - protein, 41 aa, synthetic construct; identical to SEQ ID NO: 51

SEQ ID NO: 63 - protein, 37 aa, synthetic construct
ILLYYSTAASSLAVTLMIAIFVVYMVSRDNVSCSICL

SEQ ID NO: 64 - DNA, 1701 bp, synthetic construct
  source 1..1701: mol_type = other DNA
atgaaagtgaagttactggtcctcctttgcacctttaccgccacctacgctgacaccctgtgcattggctatcatgccaacaactccacagacaccgtggacacagtgctggagaagaacgtgacagtgacccactccgtgaacctgctggaagacaagcacaacggcaaactgtgcaagctgagaggcgtggcccctctgcacctgggcaagtgcaacatcgccggctggatcctgggcaacccagagtgcgagagcctgtccacagcccggtcctggtcttacattgtggagacatccaacagcgacaacggcacctgctaccctggcgacttcatcaattacgaggagctgagagagcagctgagctccgtgtccagcttcgagaggttcgagatctttcccaagacaagctcctggcccaaccacgacagcgacaacggggtgacagccgcctgccctcacgcaggcgccaagagcttctacaaaaacctgatctggctcgtgaagaaaggaaagagctaccccaagatcaaccagacctacatcaacgacaagggcaaggaggtgctggtgctttggggcatccaccaccctcccacaattgccgaccagcagtccctgtaccagaacgctgacgcctatgtcttcgtgggcacctctagatacagcaagaagttcaagcctgagatcgccacaagacccaaggtgcgggatcaggagggccggatgaattactactggaccctggtggagccaggcgacaagatcaccttcgaggccaccggcaacctggtggctccccgatacgccttcaccatggagcgggacgccggcagcggcatcatcatcagcgacacacctgtgcacgactgcaacaccacctgccagacccctgaaggagccatcaacaccagcctgcccttccagaatgtccaccccatcaccatcggcaaatgtccaaagtacgtgaagtccaccaaactgagactggccaccggcctgagaaacgtcccctcaatccagagcagaggcctgtttggggccattgccggcttcatcgagggcggatggaccggcatggtcgacggatggtacggatatcacttccagaacgaacagggaagcggctacgccgccgacctgaagtccacacagaacgccatcgacaagatcaccaacgccgtgaacagcgtgatcgagaaaatgaacacacagttcacagccgtgggcaaggaattcaatcacctggagaagaggatcgagaatctgaacaagaaggtggacgacggcttcctggacatctggacatacaacgcagagctcctggtgctgctggccaatgagcggacactggactaccacgactccaacgtgaagaacctgtacgagaaggtgcggaatcaactgaagaacaacgccaaggagattggcaacggctgctttgagttctaccacaagtgcgacaacacctgtatggagtccgtgaagaacggcacatatgactaccccaagtacagcgaagaagccaagctgaacagagagaagattgatggagtgaaactggacagcacccggatctaccaaatcctcgccatctactccacagtggcctctagcctggtgctcgtggtttcgctgggcgctatcagcttctggatgtgttccaacggctccctgcaatgccggatctgcatctga

SEQ ID NO: 65 - DNA, 1701 bp, synthetic construct
  source 1..1701: mol_type = other DNA
atgaaagtgaagttactggtcctcctttgcacctttaccgccacctacgccgacaccatctgcattggctaccatgccaacaacagcaccgacaccgtggacacagtgctggaaaagaacgtgaccgtgacacactctgtgaacctgctggaggacagccacaacggaaaactgtgcagactgaagggcatcgcccctctgcagctgggcaagtgcaacatcgccggctggatcctgggcaatcctgagtgcgagagcctgttctctaagaagtcctggagctacatcgccgagacacccaacagcgagaatggcacctgctaccctggctacttcgctgattacgaagaactgagagagcagctgagcagcgtcagctccttcgaaagattcgagatcttccccaaagaaagaagctggcccaaacacaacgtgaccaggggcgtgaccgccgcctgcagccacaagggcaagtcttccttttaccggaacctgctctggctgacagagaaggacggcagctaccccaacctgtccaagagctacgtgaacaacaaggagaaggaggtgctggtgctctggggggtgcaccacccttcaaacatcgaggatcagaagaccctgtaccggaaggagaatgcctatgtctccgtggtgtcttccaactacaatcggagattcacccctgagatcgccgagaggcccaaggtgcggggccaggccggcagaatcaactactactggaccctgctggaacctggcgacacaatcatctttgaggccaacggcaatctgatcgccccctggcatgcattcgccctgtccagaggcttcggcagcggcatcattaccagcaacgccagcatggatgagtgcgacaccaagtgccagacccctcagggcgccatcaattcaagcctgcctttccagaacatccatcctgtgaccattggcgagtgtcccaagtacgtgagaagcacaaagctgagaatggtgaccggactgagaaacatcccttccattcagagccggggcctgtttggcgccatcgccggcttcatcgagggcggctggaccggcatgatcgacggctggtacggctaccatttccagaacgaacagggcagcggctacgccgctgatcagaagtccacacagaacgccatcaacggcatcaccaacgccgtgaacagcgtgatcgagaagatgaacacacagttcaccgccgtgggcaaggagttcaacaaactggagaagagaatggagaatctgaacaagaaggtggacgacggcttcctggacatctggacctacaatgctgagctgctggtgctgctggccaacgaaagaaccctcgacttccacgactccaacgtgaagaacctgtacgagaaggtgaagtcacagctgaaaaacaacgccaaggagatcggcaacggctgcttcgagttctaccacaagtgcaacaacgagtgtatggagtccgtgaagaatggcacctacgattaccccaagtacagcgaggagagcaagctgaacagagaaaagatcgacggagtgaagctggagagcatgggcgtgtatcaaatcctcgccatctactccacagtggcctctagcctggtgctcctggtttcgctgggcgctatcagcttctggatgtgttccaacggctccctgcaatgccggatctgcatctga

SEQ ID NO: 66 - DNA, 1701 bp, synthetic construct
  source 1..1701: mol_type = other DNA
atgaaagtgaagttactggtcctcctttgcacctttaccgccacctacgccgacaccctgtgcattggctaccatgccaacaactcaacagacaccgtggacacagtgctggagaagaacgtgaccgtgacacacagcgtgaatctgctggaggaccggcacaacggcaaactgtgcaagctgagaggcgtggctcccctgcacctgggcaagtgcaacatcgccggatggctgctgggaaacccagagtgcgaactactgttcaccgccagctcctggagctacatcgtggaaacctccaacagcgacaacggcacatgctaccctggcgacttcatcaattacgaagaactgcgggaacagctgtcctcagtgagcagcttcgaaagatttgagatcttcccaaaggcctcctcttggcccgaccacgaaaccaaccggggcgtgaccgccgcctgtccctacgccggagccaacagcttctacagaaatctgatttggctggtgaagaaaggcaactcctaccccaaactgagcaagagctacgtgaacaacaaagaaaaggaggtgctggtgctctggggcatccaccaccctccgacctccacagaccagcaaagcctgtaccagaatgctgacgcctacgtcttcgtgggcagctccaagtacaacaagaagttcaagcccgagatcgccacccggccaaaggtgcggggccaggccggcagaatgaactactactggacactggtggagcctggcgacaccatcacattcgaggccaccggcaacctggtggtgcccagatacgccttcgccatgaagagaggctctggatccggcatcatcatcagcgacacccctgtgcacgactgcaacaccacatgccagacccccaagggcgccatcaacaccagcttacctttccagaacatccaccctgtgaccatcggagaatgtcccaagtacgtgaagtcaacaaagctgcggatggccacaggactgagaaacatccccagcatccagtccagaggactgtttggcgccatcgccggcttcatcgagggcggctggaccggaatgatcgatggatggtacggctaccacttccagaatgagcagggcagcggctacgccgctgatcagaagtccacccagaatgccatcgacggcatcaccaacgccgtgaactctgtgatcgagaagatgaacacccagtttaccgccgtgggaaaggagttcaatcacctggagaagagaatcgaaaacctgaacaagaaggtggacgacggcttcctggacgtgtggacctacaacgctgaactgctggtgctgctggccaatgagagaaccctggactaccacgactccaacgtgaagaacctgtacgagaaagtgcggagccagctgaaaaacaacgccaaggagattggaaatggctgcttcgaattctaccacaagtgcgacgacacatgtatggagtccgtgaaaaacggcacctacgactaccccaagtacagcgaggaaagcaagctgaacagagaggagatcgatggggtgaaactggagagcacccggatctaccaaatcctcgccatctactccacagtggcctctagcctggtgctcctggtttcgctgggcgctatcagcttctggatgtgttccaacggctccctgcaatgccgcatctgcatctga

SEQ ID NO: 67 - DNA, 1698 bp, synthetic construct
  source 1..1698: mol_type = other DNA
atgaaagtgaagttactggtcctcctttgcacctttaccgccacctacgccgacaccatctgcattggataccatgccaacaacagcaccgacaccgtggacacagtgctggaaaagaacgtgaccgtgacccacagcgtgaacctgctggaagacagccacaacggcaaactgtgcagactgaagggcaaggcccccctgcagctgggaaactgcaacatcgccggatgggtgctgggaaatcccgagtgcgagagcctgctgagcaacaggagctggagctacatcgctgaaacccccaacagcgagaacggcacctgctatcctggcgacttcgctgactacgaggaactgcgggagcagctgtcctccgtgtcctcctttgaaagatttgagatcttccccaaggagcggagctggcccaaccacacaaccaggggcgtgaccgccgcctgccctcatgccagaaagagcagcttctacaagaatctggtctggctgaccgaggccaatggcagctatcccaacctgtccagaagctacgtgaacaaccaggagaaggaggtgctggtcctgtggggggtgcaccaccccagcaacatcgaggagcagagagccctgtacagaaaggacaacgcctatgtcagcgtggtgtccagcaactacaacagacggttcacccccgaaattgccaagcggcccaaggtgcgggatcagagcggccggatgaactactactggaccctgctggagcctggagacaccatcatcttcgaggccaccggcaacctgatcgccccttggtacgccttcgccctgagcagaggccctggcagcggcatcatcaccagcaatgcccccctggacgagtgcgacacaaagtgtcagaccccccagggcgccatcaacagctccctgccctttcagaacatccaccctgtgacaatcggggagtgccccaagtacgtgagaagcaccaagctgcggatggtgaccggcctgcggaacattccctctgtgcagtcccggggcctgtttggggccatcgccggcttcatcgagggcggctggaccggcatgatggacggctggtacggctaccacttccagaacgaacaaggctccggctacgccgctgaccaaaagtccacccagaatgcaatcaacggcatcaccaacgccgtgaattccgtgatcgagaagatgaacacccagttcacagccgtgggcaaggagttcaacaagctggaaaagcggatggagaacctgaacaagaaagtggacgacggcttcatggacatctggacctacaacgctgaactgctggtgctgctggccaatgaaagaacactggacttccacgactccaacgtgaagaacctgtatgagaaggtgaagaatcagctcagaaacaacgccaaagagctgggcaacggctgcttcgagttctaccacaagtgcgacaacgagtgtatggagtccgtgaagaacggcacatacgactaccccaagtacagcgaggagagcaagctgaacagggagaagatcgatggcgtgaagctggagtccatgggcgtctacagaatcctcgccatctactccacagtggcctctagcctggtgctcctggtttcgctgggcgctatcagcttctggatgtgttccaacggctccctgcaatgccggatctgcatctga

SEQ ID NO: 68 - DNA, 1701 bp, synthetic construct
  source 1..1701: mol_type = other DNA
atgaaagtgaagttactggtcctcctttgcacctttaccgccacctacgctgacaccatctgcattggctaccatgccaacaactccaccgacaccgtggacacagtgctggagaagaacgtgaccgtgacacattccgtgaacctgctggaggattctcacaacggcaagctctgtcggctgaagggcatcgcccctctgcagctgggcaagtgcaacatcgccggctggctgctgggcaaccccgagtgcgaccctctgctgcccgtgagatcctggtcctacattgtggaaacccctaacagcgagaacggcatctgctaccctggcgacttcattgattacgaggagctgcgggagcagctgtcctcagtgagcagcttcgagaggtttgagatcttccccaaggagtcctcttggcccaaccacaacacaaacaagggcgtcaccgccgcctgcagccatgagggaaagagcagcttctacagaaacctgctgtggctgacagagaaggagggctcctaccccaagctgaagaattcctacgtgaacaagaaaggcaaagaggtgctggtgctgtgggggatccaccaccctcccaattccaaggaacagcagaacctgtaccagaacgaaaacgcctacgtctctgtggtgacctccaactacaacagaagattcacccctgagatcgcagagcggcccaaggtgcgggaccaagccggcagaatgaactactactggaccctgctgaagcctggcgacacaatcatcttcgaggccaatggaaacctgatcgccccaatgtacgccttcgccctgtcccggggcttcggaagcggcatcatcacaagcaacgcctccatgcacgagtgcaacaccaagtgccagaccccactgggcgccatcaattcctctctcccttaccagaacatccaccctgtgaccatcggggaatgccccaagtacgtgagatccgccaagctgcggatggtgaccggcctgagaaacatccccagcattcagagcaggggcctgtttggagccatcgctggcttcatcgagggcggatggaccggcatgatcgacggctggtacggctaccacttccagaatgaacagggctccggctacgcagccgatcagaaaagcacacagaatgccatcaacggcatcaccaacgccgtgaacaccgtgatcgaaaagatgaacatccagttcaccgccgtgggcaaggagttcaacaagctggagaagagaatggagaacctgaacaagaaggtggacgacggctttctggacatttggacatacaatgctgagctgctggtgctgctggccaatgagaggaccctggactttcacgactccaacgtgaaaaacctgtacgaaaaggtcaagagccagctgaagaacaatgccaaggagatcggcaacggctgcttcgagttctaccacaagtgcgacaacgagtgtatggagtccgtgagaaacggcacctatgactaccccaagtactctgaggagtccaaactgaacagagagaaggtggacggagtgaagctggagtctatgggcatctaccaaatcctcgccatctactccacagtggcctctagcctggtgctcctggtttcgctgggcgctatcagcttctggatgtgttccaacggctccctgcaatgccgcatctgcatctga

SEQ ID NO: 69 - DNA, 1698 bp, synthetic construct
  source 1..1698: mol_type = other DNA
atgaaagtgaagttactggtcctcctttgcacctttaccgccacctacgccgacaccatctgcattggctatcatgccaacaattccacagacaccgtggacacagtgctggagaagaacgtgaccgtgacccactctgtgaacctgctggagaactctcacaacggcaaactgtgcctgctgaagggcatcgcccctctgcagctcggcaactgcagcgtggccggctggatcctgggcaacccagagtgcgaactgctgatctccaaggaaagctggagctacatcgtggagaagcccaaccccgagaacggcacctgttaccctggccactttgccgactacgaggagctgcgggaacagctgtcttccgtgtcctccttcgaaagattcgagatctttcccaaggagagctcctggcccaaccacacagtgacaggcgtctccgccagctgctcccacaatggcgagtcctctttctacagaaacctgctgtggctgaccggcaagaacggcctgtatcccaacctgagcaagagctacgccaacaacaaggagaaggaagtgctggtgctgtggggggtgcaccaccctccgaacatcggcaatcagaaggccctgtatcacacagaaaacgcctacgtcagcgtggtgtcctcccactacagcagaaagttcacccctgagatcgccaagcggcccaaagtgcgggatcaggagggccggatcaattactactggaccctgctggaaccaggcgacacaatcatctttgaggccaacggcaacctgatcgccccaagatacgccttcgccctgagcagaggcttcggctctggcatcatcaacagcaatgccccgatggacaagtgcgatgccaagtgccagaccccacagggcgccatcaacagcagcctgcctttccagaatgtccaccccgtgacaattggcgagtgtcccaagtacgtgagaagcgccaagctgcggatggtgaccggcctgcggaacatcccttccattcagagcagaggcctgtttggcgccatcgccggattcattgaaggcggctggaccggcatggtggacggctggtacggctaccacttccagaacgaacagggctctggctacgccgctgaccaaaagtccacccagaatgccatcaacggcatcaccaacgccgtcaacagcgtgatcgagaagatgaacacccagttcaccgctgtgggcaaagagttcaacaagctggagcggcggatggaaaacctgaacaaaaaggtggacgacggcttcatcgacatttggacatacaacgcagagctgctggtgctgctggccaatgagagaaccctggatttccacgacagcaacgtgaagaacctgtacgagaaggtcaaatcccagctgaaaaacaacgccaaggagatcggcaatggctgcttcgagttctaccacaagtgcaacgatgagtgtatggagtccgtgaaaaacggcacctacgactacccgaagtacagcgaggaaagcaagctcaatcgggagaagatcgacggagtgaagctggagagcatgggcgtgtatcaaatcctcgccatctactccacagtggcctctagcctggtgctcctggtttcgctgggcgctatcagcttctggatgtgttccaacggctccctgcaatgccggatctgcatctga

SEQ ID NO: 70 - DNA, 1701 bp, synthetic construct
  source 1..1701: mol_type = other DNA
atgaaagtgaagttactggtcctcctttgcacctttaccgccacctacgctgacaccctgtgcattggctatcatgccaacaacagcaccgacacagtggacacagtgctggaaaagaacgtgaccgtgacacactccgtgaacctgctggaggacaagcacaacggcaaactgtgcaagctgagaggcgtggcccctctgcacctgggcaagtgcaacatcgccggctggatcctgggcaacccagagtgcgagagcctgagcaccgccagctcctggtcttacatcgtggagacacccagctccgacaacggcacatgctaccctggcgacttcatcgactacgaggaactgagagagcagctgagctccgtgtcctctttcgagaggttcgagatcttccccaagacctcatcttggcccaaccacgacagcaacaagggcgtgaccgccgcctgtcctcacgctggcgcaaagagcttctacaagaacctgatctggctcgtgaagaagggaaacagctaccccaagctgagcaagagctacatcaatgacaaaggcaaggaggtgctggtgctatggggcatccatcaccccagcacaagcgctgaccagcagagcctgtatcagaacgctgacgcctacgtcttcgtcggcagctcccggtacagcaagaaattcaagcccgagatcgccatccggcccaaggtgcgggaccaagagggcagaatgaactactactggaccctggtggagcctggcgacaagatcacctttgaggccacaggcaatctggtggtgcccagatacgcctttgccatggagagaaatgccggctccggcatcatcatcagcgacacacctgtgcacgactgcaacaccacctgtcagacacccaagggcgccatcaacacaagcctgccttttcagaacattcaccccatcaccattggcaagtgccccaagtacgtcaagagcaccaaactgcggctggccacaggactgcggaacatccccagcattcagagccggggcctgtttggagccatcgctggcttcatcgagggcggctggaccggcatggtggacggctggtacggctaccatttccagaatgaacagggcagcggctacgccgctgacctgaagtccacccagaacgccatcgacgaaatcaccaacctggtcaactctgtgatcgaaaagatgaacacccagttcaccgccgtgggaaaagagttcaaccacctggagaagaggatcgaaaatctgaacaaaaaggtggacgacggctttctggacatctggacctacaacgcagagctcctggtgctgctgctgaatgaaagaacactggactaccacgactccaacgtcaagaacctgtacgagaaggtgcggagccagctgaagaacaacgccaaggagatcggcaacggctgcttcgagttctaccacaagtgcgacaacacctgtatggagtccgtgaagaacggcacatacgactaccccaagtactctgaagaggccaagctgaacagagaggagattgatggagtgaagctggagtccacccggatctaccaaatcctcgccatctactccacagtggcctctagcctggtgctcgtggtttcgctgggcgctatcagcttctggatgtgttccaacggctccctgcaatgccggatctgcatctga

SEQ ID NO: 71 - DNA, 1698 bp, synthetic construct
  source 1..1698: mol_type = other DNA
atgaaagtgaagttactggtcctcctttgcacctttaccgccacctacgccgacaccatctgcattggctaccatgccaacaacagcaccgacaccgtggacacagtgctggagaagaacgtgacagtgacacactctgtgaacctgctggaggactcccacaacggaaagctgtgcctgctgaagggaatcgcccctctgcagctgggcaactgttccgtcgccggctggatcctgggcaatcctgagtgcgagagcctgatctccaaagaatcttggtcctacatcgtggagacacccaacccagagaacggcacctgctaccccggatactttgccgactacgaggagctgcgggagcaactgtcctcagtgagcagcttcgaaagattcgagatcttccccaaagagagctcttggcccaaccacacagtgacaggcgtgacagcctcctgcagccacaacggaaagagcagcttctacagaaatctgctgtggctcaccgagaagaatggcctgtatcccaacctctccaacagctacgtgaacaacaaggagaaggaagtgctggtgctatggggggtgcaccaccccagcaacatccgggaccagcgggccatctaccacacagagaacgcctacgtcagcgtggtgtcctcccactactctcggagattcactcccgagatcgccaagcggcccaaggtgcggggccaggagggccggatcaattactactggaccctgctggaaccaggcgacaccatcatcttcgaggccaacggcaatctcatcgccccctggtacgctttcgccctgtctcggggcttcggctccggcatcatcacaagcaacgcccccatgaatgagtgcgatgccaagtgccagacaccccagggcgccatcaacagctccctgcccttccagaacgtccaccctgtgaccatcggagagtgtcccaagtacgtgagatccaccaagctgagaatggtgaccggcctgagaaacattcccagcatccagagcagaggcctgttcggagccatcgccggattcattgagggaggatggaccggcatgatggacggctggtacggctaccacttccagaatgagcagggaagcggctacgccgctgaccagaaatccacacagaacgccatcaacggcatcaccaacctggtgaacagcgtgatcgaaaagatgaacacccagttcaccgccgtgggcaaagagttcaacaagctggaaagaagaatggagaacctgaacaagaaagtggacgacggcttcctggacatctggacatacaacgcagagctgctggtgctgctgctgaatgagagaacactggacttccacgacagcaacgtcaaaaacctgtacgagaaggtgaagtcacagctgaaaaacaacgccaaggagatcggcaatggctgcttcgaattctaccacaagtgcaacaacgagtgtatggaatccgtgaagaacggcacctacgactacccgaagtactctgaggagagcaaactgaacagagagaaaatcgacggggtgaagctggagagcatgggcgtgtatcaaatcctcgccatctactccacagtggcctctagcctggtgctcctggtttcgctgggcgctatcagcttctggatgtgttccaacggctccctgcaatgccggatctgcatctga

SEQ ID NO: 72 - DNA, 1701 bp, synthetic construct
  source 1..1701: mol_type = other DNA
atgaaagtgaagttactggtcctcctttgcacctttaccgccacctacgccgacacaatctgcattggctaccatgccaacaacagcacagacaccgtggacaccgtcctggagaaaaacgtgacagtgacccacagcgtgaacctgctggaagactcccacaatggaaaactgtgcaagctgaagggcatcgcccctctgcagctgggcaagtgcaacatcgccggctggctgctgggcaacccagagtgcgacctgctgctgacagccagtagctggagctacatcgtggaaaccagcaacagcgaaaatggcacatgctaccctggcgacttcatcgattacgaggagctgcgggagcagctgagcagcgtcagctctttcgagaagtttgagatcttccccaaaacctcaagctggcccaaccacgaaaccacaaagggcgtgaccgccgcctgcagctacgcaggcgccagctctttctacagaaacctgctgtggctgacaaagaaggggagcagctaccccaagctctccaaaagctacgtgaacaacaagggcaaggaggtgctggtgctatggggggtgcatcaccctccaaccggcaccgaccagcagagcctgtaccagaacgccgacgcctacgtctccgtgggctcctccaagtacaacagaagattcacccctgagatcgctgctcggcccaaggtgcgggaccaggccggaaggatgaactactactggaccctgctggagcctggcgacacaatcaccttcgaggccacaggcaacctgatcgccccttggtacgccttcgctctcaacagaggctccggcagcggcatcatcacctccgacgcccctgtgcacgactgcaacacaaagtgccagacccctcacggggccatcaactccagcctgcctttccagaacatccaccctgtgaccatcggagaatgccccaagtacgtgagaagcaccaagctgagaatggccacaggcctgagaaacatcccttccatccagagcaggggcctgtttggggccatcgccggcttcatcgagggcggctggaccggcatgatcgacggctggtacggctaccacttccagaacgaacagggctccggctacgccgccgacagaaagtccacccagaatgccattgacggcatcaccaacctggtgaattccgtgatcgagaagatgaacacccagttcaccagcgtgggaaaagagttcaaccacctggagaagagaatcgagaacctgaacaggaaggtggatgacggcttcctggacgtgtggacatacaacgctgaactgctggtgctgctgctgaacgaaagaaccctggactaccacgacagcaacgtgaagaacctgtacgagaaggtgcggagccagctgaagaacaacgccaaggaaatcggcaatggctgtttcgaattctaccacaagtgcgacgacagctgtatggaatccgtgaaaaacggcacatacgactaccccaagtacagcgaggagagcaagctgaacagagaggaaatcgatggcgtgaaactggagtccatgggcgtgtatcaaatcctcgccatctactccacagtggcctctagcctggtgctcctggtttcgctgggcgctatcagcttctggatgtgttccaacggctccctgcaatgccggatctgcatctga

SEQ ID NO: 73 - DNA, 1701 bp, synthetic construct
  source 1..1701: mol_type = other DNA
atgaaagtgaagttactggtcctcctttgcacctttaccgccacctacgctgacaccctgtgcattggctaccatgccaacaacagcaccgacaccgtcgacacagtcctggaaaagaacgtgacagtgacccactccgtgaatctgctggaaaacagacacaacggcaaactgtgcaagctgcggggcgtggcccctctgcacctgggcaaatgcaacatcgccggatggctgctgggcaacccagagtgcgaaagcctgagcaccgccagctcttggtcctacatcgtcgaaaccagcaacagcgacaatggcacatgctaccctggcgacttcatcaactacgaggaactgagagagcagctgagctctgtgtcctccttcgagaggtttgagatcttccccaagacctcaagctggcccaatcacgacacaaacagaggagtgaccgccgcctgtcctcacgatggaaccaacagcttctacagaaatctgatctggctcgtgaagaagggcaactcttaccccaagatcaacaagtcttacatcaacaacaaagagaaggaaattctggtgctgtgggccatccaccaccccagcacctccgctgaccagcaaagcctgtaccagaacgctgacgcctacgtcttcgtgggcagctcccggtacagcagaaagttcgaacctgaagtggccacccggcccaaggtgcgggaccaggccggcagaatgaactactactggaccctggtcgaacctggcgacaagatcaccttcgaagccaccggaaacctggtggtgcccagatacgccttcgccctgaagagaaattcgggcagcggcatcatcatcagcgacacatccgtccacgactgcgacaccaactgtcagaccccaaatggggccatcaacaccagcctgcctttccagaacatccaccctgtcaccatcggagagtgccccaagtacgtgaagtccacaaagctgagaatggccaccggcctgcggaacatccccagcatccagagccggggactgtttggcgccatcgccggcttcatcgagggcggctggaccggcatgatcgacggctggtacggctaccacttccagaatgagcagggcagcggctacgccgctgacctgaagtccacacagaacgccatcgacggcatcaccaacctggtgaacagcgtgatcgagaaaatgaacacccagttcaccgccgtgggcaaggaattctcccacctggagagacggattgagaacctgaacaagaaggtggacgacggctttctggacatctggacctacaacgctgaactgctggtgctgctgctgaatgagagaacactggactaccacgattccaatgtcaaaaacctgtatgagaaagtgcggagccagctgaagaacaatgccaaggaaattggcaacggctgtttcgagttctaccacaagtgcgatgatatgtgtatggagtccgtgaagaatggaacatacgactaccccaagtacagcgaagaggccaagctgaacagagaggagatcgacggggtgaagctggagtctacccggatctaccaaatcctcgccatctactccacagtggcctctagcctggtgctcgtggtttcgctgggcgctatcagcttctggatgtgttccaacggctccctgcaatgccggatctgcatctga

SEQ ID NO: 74 - DNA, 1701 bp, synthetic construct
  source 1..1701: mol_type = other DNA
atgaaaacgatcatcgccctatcctacatcctatgcctcgtcttcgcccaaaaactccccggcaacgacaacagcacagccacactgtgcctgggccatcacgctgtgcccaacggaaccatcgtgaagaccattaccaacgacagaatcgaagtgacaaatgccacagagctggtgcagaacagcagcattggcgagatctgcaacagccctcaccagatcctggacggaggcaactgcacactgatcgatgccctgctcggcgaccctcagtgcgatggcttccagaacaaggagtgggacctgttcgtggaaagaagcagagccaacagctcctgctacccctacgacgtccctgactacgccagcctgcggagcctggtggcctccagcggcaccctggagttcaaaaacgaaagcttcaattggaccggagtgaaacagaacggcacctccagcgcctgtatcagaggctccagcagctctttcttcagcagactgaactggctgacaagcctgaacaacatttaccccgcccagaacgtgacaatgcccaacaaggagcagtttgacaaactgtacatctggggagtgcaccaccctgataccgacaaaaaccagttctccctgttcgcccagtcttccggcagaatcaccgtgtctaccaagcggagccagcaggccgtgatccccaacatcggcagccggccccggattcgggacatcccttcccggatcagcatttactggaccatcgtgaagcctggagacattctcctgatcaacagcaccggcaacctgatcgcccctcggggctacttcaagatcagaagcggcaagtccagcatcatgagatctgacgcccctatcggcaagtgcaagtctgagtgtatcacacccaacggcagcatccccaacgacaagccctttcagaacgtgaacagaatcacctatggcgcctgtcccaggtacgtgaagcagagcaccctgaagctggccacagggatgagaaacgtccccgaaaagcagacccggggcatctttggcgctatcgccggcttcatcgagaacggctgggagggcatggtggacggctggtacggcttccggttccagaacagcgagggcagaggccaggccgctgacctgaagtccacccaggctgccatcgaccagatcagcggactgctgaacagactgatcggcaaaacaaacgaaaagttccatcagatcgagaaggagttctccgaggtggaaggccgggtgcaggacctggaaaagtacgtggaagacaccaagatcgacctgtggtcctacaacgctgaactgctggtcgccctgctgaatcagcacaccatcgacctgaccgacagcgagatgaacaaactgttcgagaagaccaagaagcagctgcgggaaaacgccgaggatatgggcaacggatgcttcaagatttaccacaagtgcgacaacgcctgcattggcagcatcagaaatgagacatacgaccacaatgtttaccgggatgaggccctgaacaacagattccagatcaagggagtggagctgaagtccggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 75 - DNA, 1701 bp, synthetic construct
  source 1..1701: mol_type = other DNA
atgaaaacgatcatcgccctatcctacatcctatgcctcgtcttcgcccaaaaactccccggcaacgacaacagcaccgccaccctgtgcctggggcaccacgctgtgccaaatggcaccattgtgaagaccattaccaatgacagaatcgaggtgaccaatgccaccgaactggtgcagaattccagcattggcgagatctgcgacagccctcaccagatcctggatggcgagaactgcaccctgatcgatgctctgctgggcgaccctcagtgcgatggcttccagaacaagaaatgggacctgttcgtggagcggagcaaggcctacagcaactgctacccctacgacgtccccgattacgccagcctgcggagcctggtggcctcctccggcacactggagttcaacaacgaaagcttcaactggacaggagtgaagcagaacggcacatccagcgcctgcatcagaaaaagctccagcagcttcttctctcgactgaactggctgacccacctgaactacacctaccccgccctgaacgtgaccatgccaaacaatgagcagtttgacaaactgtacatttggggagtgcaccaccccggcacagacaaggaccagatctttctgtacgcccagagcagcggcaggatcaccgtgtctaccaagagaagccagcaggccgtgatccccaacatcggcagcagacctcggatcagagacatcccctccagaatctccatctactggacaattgtgaagcctggagacattctgctgatcaacagcaccggcaacctgatcgcccctcgaggatacttcaagatccagagcggcaaaagcagcatcatgagatctgacgcccccatcggcaaatgcaaaagcgagtgtatcacccccaacggcagcatccccaacgacaagccctttcagaacgtgaacagaatcacctatggcgcctgtcccaggtacgtgaagcacagcaccctgaagctggccacaggaatgagaaacgtccccgagaagcaaacccggggcatctttggcgctattgccggcttcatcgagaacggctgggaagggatggtggacggctggtacggcttccggttccagaacagcgagggaagaggccaggccgctgacctgaagtccacccaggccgccattgatcagatcaacggcctgctgaacagactgatcggcaagaccaacgaaaaattccaccagatcgaaaaggaattcagcgaagtggagggcagaattcaggacctggaaaagtacgtggaggacacaaagatcgacctgtggtcctacaacgctgaactgctggtggccctgctgaatcagcacaccatcgacctgaccgacagcgagatgaacaaactgtttgaaaagaccaagaagcagctgagagaaaacgccgaggacatgggcaatggatgcttcaaaatctaccacaagtgcgacaacgcctgtattggcagcatcagaaatggcacctacgaccacaacgtctacagggacgaggccctgaacaacagatttcaaatcaagggcgtggagctgaagtctggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 76 - DNA, 1701 bp, synthetic construct
  source 1..1701: mol_type = other DNA
atgaaaacgatcatcgccctatcctacatcctatgcctcgtcttcgcccaaaaactccccggcaacgacaacagcacagccacactctgtctgggccatcacgctgtgccaaacggcacaatcgtgaagacaatcaccaacgaccagattgaggtgacaaatgccacagagctggtgcagagctccagcacaggcggcatctgcgacagccctcatcagatcctggatggcgagaactgcacactgatcgatgccctgctgggcgaccctcagtgcgacggcttccagaacaagaaatgggacctgttcgtggagcggagcaaggcttacagcaactgttacccctacgacgtccctgactacgcctccctgcggagcctggtggcctcctccggcaccctggagtttaacaatgaatcctttaactggacaggcgtcacccagaacggcacatcttccagctgcaagagaagaagcaacaactccttcttcagcagactgaattggctgacccacctgaagttcaagtaccctgccctgaacgtgaccatgccaaacaatgagaagttcgacaagctgtacatctggggggttcaccaccctggcacaaacaacgaccagatcagcctgtacacccaggccagcggccggatcaccgtgtctacaaagcggagccagcagacagtgatccccaacatcggctccagacctcgggtgagagacatcccctccagaatcagcatctactggaccatcgtgaagccaggcgacatcctgctcatcaacagcaccggaaacctgattgcacccagaggctacttcaagatcagaagcggcaagagcagcatcatgagaagcgatgcccccatcggcaagtgcaacagcgagtgtatcacccccaacggcagcatccccaatgacaagcccttccagaacgtgaacagaatcacctatggcgcctgccccaggtacgtcaagcagaacaccctgaagctggccaccggaatgagaaacgtccccgagaagcagaccagggggatctttggagccatcgccggcttcatcgagaacggctgggagggaatggtggacggatggtacggcttccggttccagaactctgagggcattggccaggccgcagatctgaagtccacccaggccgccatcaaccagatcaacggcctgctgaacagactgatcggcaagacaaatgaaaaattccaccagatcgagaaagaattcagcgaggtggaaggccggattcaggacctggagaagtacgtggaggacaccaagatcgacctgtggagctacaacgctgaactgctcgtggccctgctgaaccagcacacaatcgacctgacagacagcgagatgaacaagctgttcgaaagaacaaagaaacagctgagagaaaacgccgaggacatgggcaacggctgcttcaagatctaccacaagtgcgacaacgcctgcattggcagcatcagaaacggcacctacgaccatgacgtttaccgggacgaggccctgaacaaccggttccagatcaagggcgtggaactgaagtccggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 77 - DNA, 1701 bp, synthetic construct
  source 1..1701: mol_type = other DNA
atgaaaacgatcatcgccctatcctacatcctatgcctcgtcttcgcccaaaaactccccggcaacgacaacagcacagccaccctgtgcctgggacaccacgctgtgccaaatggcaccctggtgaagacaatcaccaacgaccagatcgaagtgacaaatgccacagagctggtgcagagctcctccaccggcagaatctgcgactctcctcacagaatcctggacggcaagaactgcaccctgatcgatgccctgctgggcgaccctcactgcgacggcttccagaacaaggagtgggacctgttcgtggagagaagcaaggcctactccaactgttacccttacgacgtccctgattacgcctccctgagaagcctggtggcctcctctggcaccctggagttcaccaatgagggcttcaactggaccggagtggcccaggatggcacctcctacgcctgcaagagaggctccgtgaaaagcttcttcagcagactgaactggctccacaagctggagtacaagtaccccgccctgaacgtgaccatgccaaacaacgacaagttcgacaagctctacatctggggcgtccaccaccccagcaccgacagcgaccagacatctctctatgtccaggccagcggcagagtgacagtcagcaccaagagaagccagcaaacagtgatccccaacatcggcagcagaccttgggtgcggggcatctcttccagaatcagcatctactggacaatcgtgaagcctggcgatatcctgctgatcaagagcaccggcaacctgatcgcccccagaggctacttcaagatcagaagcggaaagagctccatcatgagaagcgatgctcctatcggcaactgcaactctgagtgtatcacccccaacggctccattcccaacgacaagccttttcagaacgtgaacagaatcacctacggggcctgccccaggtacgtgaagcagaacaccctgaagctggccaccggaatgagaaacgtccccgagaagcagacacggggcatctttggcgccatcgccggcttcatcgagaacggctgggaggggatggtggatggctggtacggcttccggttccagaattcggagggcaccggccaggccgccgacttgaaatccacccaggccgccatcaaccagatcaacggcctgctgaacagactgatcgagaagacaaatgagaagttccaccagatcgagaaggagttcagcgaagtggagggaagaatccaggacctggagaagtacgtggaagacaccaagatcgacctgtggtcctacaacgcagaactgctggtggccctgctgaaccaacacacaatcgacctgacagacagcgaaatgaacaaactgttcgagagaaccagaaaacagctgagagagaacgccgaggacatgggcaacggctgtttcaagatctaccacaagtgcgacaacgcctgcattggctccatcagaaacggcacctacgatcacgatgtttaccgggacgaggccctgaacaatcgtttccagatcaagggagtggagctgaagtctggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 78 - DNA, 1701 bp, synthetic construct
  source 1..1701: mol_type = other DNA
atgaaaacgatcatcgccctatcctacatcctatgcctcgtcttcgcccaaaaactccccggcaacgacaacagcaccgccaccctgtgcctcgggcaccacgctgtgccaaacggcaccctggtgaagaccatcaccaacgatcagatcgaagtgaccaatgccaccgaactggtgcagagctcctccaccggcaagatctgcgacaaccctcacagaatcctggacggcatcaactgcaccctgatcgatgccctgctgggcgaccctcactgcgatggcttccagaatgagaagtgggacctgttcgtcgagagaagcaaggccttttccaactgttacccttacgacgtccccgattacgccagcctgagatccctggtggcctccagcggaacactggagttcatcaacgagggcttcaactggacaggcgtgacccagaacggcggctcctctgcctgcaagagaggccccgacaatggctttttcagcagactgaactggctgtacaagagcggcagcacctacccagtccagaacgtgaccatgccaaacaacgacaactccgacaagctgtacatctggggcgtccaccaccccagcacagacaaggagcagacagacctctacgtccaagcctccggcaaggtcaccgtgtccaccaagcggagccagcagaccgtgatccccaacgtgggcagccggccctgggtgagaggcctgagctcccgggtgtccatctactggaccatcgtgaaaccaggcgacatcctggtgatcaacagcaacggcaacctgatcgcccctcggggctacttcaagatgagaaccggcaagagcagcatcatgagaagcgatgccccaatcggaacatgctcctctgaatgcattacacccaacggaagcatccccaacgacaaacccttccagaacgtgaacaagattacatatggcgcctgccccaagtacgtgaagcagaacacactgaagctggccacaggaatgagaaacgtccctgagaagcagacaagaggcatctttggcgctatcgccggattcatcgaaaacggctgggagggaatgatcgacggctggtacggatttagattccaaaacagcgagggcaccggccaggccgctgacctgaagtcaacccaggccgccatcgaccagatcaacggcctgctgaacagagtgatcgaaaagaccaatgagaagttccatcagatcgagaaggagttcagcgaagtggagggcagaatccaggacctggagaagtacgtggaggataccaagatcgacctgtggagctacaacgcagagctgctggtggccctgctgaaccagcacaccatcgacctgacagacagcgagatgaacaagctgttcgagaagacaagaagacagctgagagagaacgccgaggatatgggcaacggctgtttcaagatctatcacaagtgcgacaacgcctgcattggcagcatcagaaacggcacctacgaccacgatgtctacagagacgaggctctgaacaacagattccagatcaagggcgtggagctgaagtccggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 79 - DNA, 1701 bp, synthetic construct
  source 1..1701: mol_type = other DNA
atgaaaacgatcatcgccctatcctacatcctatgcctcgtcttcgcccaaaaactccccggcaacgacaacagcaccgccacactgtgcctgggacaccacgctgtgccaaacggcacaatcgtgaagacaatcaccaatgacagaatcgaggtgaccaatgccacagagctggtgcagaacagctctatcggcgaaatctgcgacagcccccaccagatcctggacggcggcaattgcaccctgatcgatgccctgctgggcgaccctcagtgcgacggcttccagaacaaggaatgggacctgttcgtggagcggagcagagccaattcaaactgctacccctacgacgtccccgactacgccagcctgcggagcctggtggcctccagcggcacactggagttcaagaacgagtctttcaactggacaggcgtcaagcagaacggcacctccagcgcctgcatcaggggctccagcagcagcttcttcagcagactgaactggctgacccacctgaattacacctaccccgccctgaacgtgaccatgccaaacaacgaacagttcgacaagctgtacatctggggagtgcatcaccccagcacagacaaggaccagatcagcctgttcgctcagccctccggcagaattaccgtgtccaccaagagaagccagcaagccgtgatcccaaacatcggcagccggcctcggatccgggacatcccttccagaatctccatctactggaccatcgtgaagcctggcgacatcctgctgatcaacagcacaggcaatctgatcgcccccaggggctacttcaagatcagaagcggcaagagcagcatcatgagaagcgatgcccccatcggcaagtgcaagagcgagtgtatcacccctaacggcagcatcccaaacgacaagcctttccagaacgtgaacagaatcacatatggagcctgccccaggtacgtcaagcagagcacactgaagctggccacagggatgagaaacgtccctgagaagcagacccggggcatctttggcgccatcgccggctttatcgagaacggctgggagggaatggtggatggctggtatggcttccggttccagaacagcgagggaagaggccaggccgctgacctgaagtctacccaggccgccatcgaccagatcaatggcctgctcaatcgcctgattggcaagaccaatgaaaagtttcaccagatcgagaaggagttctccgaagtcgagggccgggtgcaggacctggagaagtacgtggaagacaccaagatcgacctgtggtcttacaacgctgaactgctggtggccctgctgaaccaacacacaatcgacctgaccgacagcgagatgaacaagctgtttgagaagaccaagaagcagctgagagagaacgccgaggacatgggcaacggctgcttcaagatctaccacaagtgcgacaatgcctgcattggcagcatccggaacgaaacctacgaccacaatgtctacagagacgaggccctgaacaacagattccagatcaaaggcgtggagctgaaatccggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 80 - DNA, 1701 bp, synthetic construct
  source 1..1701: mol_type = other DNA
atgaaaacgatcatcgccctatcctacatcctatgcctcgtcttcgcccaaaaactccccggcaacgacaacagcacagcaaccctgtgcctgggacaccacgcagtccccaacggcacaatcgtgaagacaatcaccaacgaccagattgaggtgacaaatgccacagagctggtgcagagctccagcacaggcgagatctgcgacagccctcaccagatcctggatggcaagaactgcaccctgatcgatgccctgctgggcgacccccagtgcgacggcttccagaacaaaaagtgggacctgttcgtggagagaagcaaggcctacagcaactgctacccttacgatgtccctgactacgccagcctgcggagcctggtggcctccagcggcaccctggagttcaacaacgagagcttcaattggacaggcgtgacccaggatggaacaagcagcgcctgcatccggggctccaagaattccttcttcagcagactgaactggctgacacacctgaacttcaagtaccctgccctgaacgtgacaatgccaaacaacgaacaattcgacaagctgtacatctggggagtgcaccaccccggcaccgacaaggatcagatctttctgtatgcccaggccagcggccggatcaccgtgtccaccaaaagaagccagcaggccgctatccccaatatcggcagccggcctcgggtgagaaacatcccttccagaatctcaatctactggacaatcgtgaagcctggcgacatcctgctgatcaatagcaccggcaacctgatcgcccccagaggctacttcaagatcagaagcggaaagagcagcatcatgagaagcgatgcccccatcggcaagtgcaattctgaatgcatcacccccaacggctccattcccaacgacaagcctttccagaacgtgaatcggatcacctatggcgcctgccccaggtacgtgaagcagaacacactgaagctggccaccgggatgagaaatgtccccgagaagcagaccaggggcatctttggcgccatcgccggcttcatcgagaacggctgggaaggcatggtggacggctggtatggcttcaggttccagaacagcgaaggcagaggccaggccgctgacctgaaatccacccaggctgccatcgaccagatcaatggactgctgaataggctgatcggcaagacaaacgagaaattccaccagatcgagaaggagttcagcgaagtggagggccggattcaggacctggagaagtacgtggaggacacaaagattgacctgtggagctacaacgcagagctgctggtggccctgctgaaccagcacaccatcgacctgacagacagcgagatgaacaagctgtttgagaagacaaaaaagcagctgagagagaatgccgaggacatgggaaacggctgtttcaagatctaccacaagtgcgacaacgcctgcattggcagcatccggaacggcacctacgatcacgatgtttaccgggacgaagccctgaacaaaagattccagatcaaggggatcgaactgaaatccggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 81 - DNA, 1701 bp, synthetic construct
  source 1..1701: mol_type = other DNA
atgaaaacgatcatcgccctatcctacatcctatgcctcgtcttcgcccaaaaactccccggcaacgacaacagcaccgccaccctgtgcctgggacaccacgctgtgccaaatggcaccctggtgaagaccatcacaaacgaccagatcgaggtcacaaatgccaccgaactggtccagtcttctagcacaggcggcatctgcgacagcccccaccaaattctggatggcgagaactgcaccctgatcgatgccctgctgggagatccccactgcgacggcttccagaacaaagaatgggacctgttcgtggagcggagcaaagcctactccaactgctacccctacgatgtccccgactacgcctccctgagaagcctggtggccagctctggcaccctggagttcaacaatgagtccttcaactggacaggcgtggcccagaatggcacatcctccgcctgcaagcggagatctaacaagagcttctttagcagactgaattggctgcaccagctgaagtacaagtaccctgccctgaacgtgaccatgccaaacaatgagaagttcgacaagctgtacatctggggcgtccatcacccctccaccgacagcgaccagatcagcctgtacgcccaagcccctggcagagtgacagtgagcacaaagcggagccagcagacagtgatcccgaacatcggcagccggccttgggtgagaggcgtgtccagcagaatcagcatctactggacaatcgtgaagcctggcgacatcctgctgatcaactgcacaggcaacctgatcgccccaagaggctacttcaagatccggtccggcaagagcagcatcatgagaagcgatgccagcatcggaaagtgcaacagcgagtgtatcacccccaacggcagcatccccaacgacaagcctttccagaacgtgaatcggatcacctatggcgcctgccccaggtacgtgaagcagaacaccctgaaactcgccaccgggatgagaaacgtccccgagaaacagacaagaggcatctttggagccatcgccggcttcatcgagaacggctgggaggggatggtggacggctggtacggattccggttccagaacagcgagggcaccggccaggccgctgacctgaaaagcacccaggccgccatcaatcaaatcaatggcctgctgaacagactgatcgaaaagacaaatgagaagttccaccagatcgaaaaggaattcagcgaggtggaaggcagaatccaggacctggagaagtacgtggaggataccaagatcgacctgtggagctacaacgctgaactgctggtggccctgctgaaccagcacacaatcgacctgaccgacagcgagatgaacaagctgttcgagagaaccaagaagcagctgagagagaacgccgaggacatgggcaatggttgcttcaagatctaccacaagtgcgacaacgcctgcattggaagcatcagaaacggcacatacgaccacgatgtctacagagacgaggctctgaacaatcggtttcagatcaagggcgtggagctgaagtctggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 82 - DNA, 1701 bp, synthetic construct
  source 1..1701: mol_type = other DNA
atgaaaacgatcatcgccctatcctacatcctatgcctcgtcttcgcccaaaaactccccggcaacgacaacagcaccgccaccctgtgcctgggccaccacgctgtgccaaacggcaccctggtgaagaccatcaccaacgatcagattgaagtgaccaatgccacagagctggtgcagagctcctccactggcagaatctgcgactcccctcacagaatcctggatggcaaaaactgcacactcgtggatgccctgctgggcgacccccactgcgacggcttccagaatgagaagtgggacctgttcgtggagagaagcaaggccttcagcaactgctacccttacgacgtccctgactacgccagcctgagaagcctggtggcctccagcggcacactggagttcatcaatgagtccttcaactggacaggcgtgacacagtctggcggctcttacgcctgcaagcggggcagcgacaacagcttcttctcccggctgaattggctgtacgagagcgagagcaagtaccctgtgctgaacgtgaccatgccaaacaatggcaatttcgacaaactgtacatctggggagtgcaccaccccagcaccgacaaggagcagaccaacctctacgtgagagccagcggcagagtgaccgtgtccacaaagcggagccagcagaccatcatccccaacatcggcagccggccctgggtgagaggcctgagctcccggatcagcatctactggacaatcgtgaaacccggagacatcctgctgatcaacagcaacggcaacctgatcgcccccagaggctactttaagatcagaacaggcaagagctccatcatgagaagcgatgcccccatcggcacatgcagcagcgagtgtatcacccccaacggctcaatccccaacgacaagcctttccagaacgtgaacaagattacctatggcgcctgccccaagtacgtgaagcagaacacactgaagctggccacagggatgaggaatgtccccgaaaagcagacaagaggcatctttggggccatcgccggcttcatcgagaacggctgggaaggaatggtggacggctggtacggcttcaggttccagaacagcgagggcaccggccaggccgctgacctgaagtccacccaggccgccatcgaccagatcaatggactgctgaacagagtgatcgagaagacaaacgaaaagttccaccagatcgaaaaggaattctccgaggtggaaggacggattcaggacctggaaaagtacgtggaagacaccaagatcgacctgtggagctacaacgcagagctgctggtggctctgctgaaccagcacacaatcgacctgacagacagcgagatgaacaaactgttcgaaaagacacggagacagctgagggagaacgctgaagacatgggaaacggctgctttaagatttaccacaagtgcgacaatgcctgcattggcagcattcggaacggcacatacgaccatgatgtctacagagacgaggctctgaacaaccggttccagatcaagggcgtggagctgaagtctggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 83 - DNA, 1701 bp, synthetic construct
  source 1..1701: mol_type = other DNA
atgaaaacgatcatcgccctatcctacatcctatgcctcgtcttcgcccaaaaactccccggcaacgacaacagcacagccaccctgtgcctgggccaccacgctgtgccaaacggcacactggtgaagacaatcaccgacgaccagatcgaggtgaccaatgcaacagagctggtgcagagttccagcaccggaaagatctgcaacaaccctcaccggatcctggacggcatcgactgcaccctgattgatgccctgctcggcgaccctcactgcgacgtgttccagaacgaaacatgggacctgttcgtggagcggtccaaggcctttagcaactgctacccctacgacgtccccgattacgccagcctgagatctctcgtggcctccagcggcaccctggagttcattacagagggcttcacatggacaggcgtgacacagaacggcggcagcaacgcctgcaagagaggccctggctctggcttcttcagcaggctgaattggctgacaaagagcggaagcacctaccctgtgctgaacgtgaccatgccaaacaacgacaatttcgacaagctgtacatctggggcgtccaccaccccagcaccaaccaggagcagaccagcctctatgtccaggccagcggcagagtgacagtgagcaccagacggtcccagcagaccatcatccctaacatcggatccagaccttgggtgcggggcctgagcagcagaatctccatctactggaccatcgtgaaacctggcgatgtcctggtgatcaactccaacggcaacctgatcgcccctcggggctacttcaagatgcggacaggcaagagctcaatcatgagaagcgatgctcctatcgacacctgcatcagcgagtgtatcacacccaacggctctatccccaacgacaagcccttccagaacgtgaacaagatcacatatggagcctgccccaagtacgtgaaacagaacaccctgaagctggccaccgggatgagaaacgtccctgagaagcagacccggggcctgtttggagccatcgccggcttcatcgagaacggctgggaaggcatgatcgacggctggtacggattccggttccagaacagcgaaggcaccggccaggctgctgacctgaagtccacacaggccgccatcgaccagatcaatggcctgctgaacagagtgatcgaaaagacaaacgaaaagttccaccagatcgaaaaggagttcagcgaagtggagggcagaatccaggacctggagaagtacgtggaggacaccaagatcgacctgtggagctacaacgcagaactgctggtcgccctgctgaaccagcacaccatcgacctgacagattcggagatgaacaagctgttcgagaaaacccggagacagctgagagagaacgccgaggacatgggaaacggctgcttcaagatttaccacaagtgcgacaacgcctgcattgaatccattcggaacggcacctacgaccacgatgtctacagagacgaggccctgaacaaccggtttcagatcaagggcgtggagctgaagtccggctacaaggactggatcttgtggatcagcttcgccatctcctgcttcctgctctgtgtggtcctgctcggcttcattatgtgggcctgtcagaagggcaacatccgatgcaacatctgcatttga

SEQ ID NO: 84 - DNA, 1749 bp, synthetic construct
  source 1..1749: mol_type = other DNA
atgaaggcgatcatcgtcctcctgatggtagtgacaagcaatgccgacagaatttgcaccggcattaccagcagcaacagccctcacgtggtcaagaccgccacccagggcgaggtgaacgtgacaggcgtgattcccctgacaaccacccccaccaagagccacttcgccaacctgaaaggcaccgagacacggggcaaactgtgccccaagtgcctcaactgtaccgacctggacgtggccctgggcagacccaagtgcaccggcaaaatcccaagcgccagagtgagcatcctgcacgaggtgcggcccgtgaccagcggctgcttccccattatgcacgatagaaccaagatcagacagctgccaaatctgctgagaggctacgaacacgtcagactgtccacccacaacgtgatcaacacagaggacgcccctggcggcccttacgagatcggcacaagcggctcctgcctgaacattacaaacggcaaaggcttcttcgccaccatggcttgggccgtccccaagaacaagacagccaccaatcctctgaccatcgaggtcccttacatctgcaccgaagaggaggatcagatcacagtgtggggcttccacagcgatgacgagacacagatggccagactgtacggcgacagcaagcctcagaagttcaccagcagcgccaacggcgtgaccacccactacgtcagccagattggcggctttcctaaccagaccgaggacggcggcctgcctcagagcggcagaattgtggtggactacatggttcagaaaagcggcaagacaggcacaatcacctaccagcggggcatcctgctgcctcagaaggtgtggtgcgcctccggcaaatctaaggtgatcaagggctccctgcctctgatcggagaggccgactgcctgcacgagaagtacggaggcctgaacaagagcaaaccctactacacaggcgaacatgccaaggccatcggcaactgccccatctgggtcaagacccctctgaagctggccaatggaacaaagtacagacctcctgccaagctgctgaaggagagaggcttctttggcgccatcgccggcttcctggaaggcggctgggagggcatgatcgccggctggtacggatacacctctcatggagcacacggcgtggccgtggccgctgacctcaagagcacccaggaggccattaacaagatcacaaagaacctgaactccctgagcgaactggaggtgaagaatctgcagagactgtcaggcgccatggacgaactgcacaatgagatcctggagctggacgagaaggtggacgacctgcgggccgacaccatctcctcccagatcgaactggcagtcctgctgagcaacgagggcatcatcaactccgaggacgagcacctgctggccctggagagaaagctgaagaagatgctgggcccctctgccgtggagatcggcaacggctgcttcgaaaccaagcacaaatgcaaccagacctgtctggaccggattgcagccggcacctttgacgccggggaattcagcctgcctaccttcgactccctgaacatcaccgccgcttctctgaacgacgacggcctcgacaatcacaccatcctgctctactactccaccgccgcctccagcctggccgtgaccctgatgatcgccatcttcgtggtgtacatggtttccagagacaacgtctcctgcagcatctgcctgtga

SEQ ID NO: 85 - protein, 566 aa, synthetic construct
MKVKLLVLLCTFTATYADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHFQNEQGSGYAADRKSTQNAIDGITNLVNSVIEKMNTQFTSVGKEFNHLEKRIENLNRKVDDGFLDVWTYNAELLVLLLNERTLDYHDSNVKNLYEKVRSQLKNNAKEIGNGCFEFYHKCDDSCMESVKNGTYDYPKYSEESKLNREEIDGVKLESMGVYQILAIYSTVASSLVLLVSLGAISFWMCSNGSLQCRICI

SEQ ID NO: 86 - protein, 566 aa, synthetic construct
MKVKLLVLLCTFTATYADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHFQNEQGSGYAADLKSTQNAIDKITNAVNSVIEKMNTQFTAVGKEFNHLEKRIENLNKKVDDGFLDIWTYNAELLVLLANERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 87 - protein, 565 aa, synthetic construct
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHFQNEQGSGYAADQKSTQNAINGITNAVNSVIEKMNTQFTAVGKEFNKLERRMENLNKKVDDGFIDIWTYNAELLVLLANERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 88 - protein, 566 aa, synthetic construct
MKVKLLVLLCTFTATYADTLCIGYHANNSTDTVDTVLEKNVTVTHSVNLLENRHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHDGTNSFYRNLIWLVKKGNSYPKINKSYINNKEKEILVLWAIHHPSTSADQQSLYQNADAYVFVGSSRYSRKFEPEVATRPKVRDQAGRMNYYWTLVEPGDKITFEATGNLVVPRYAFALKRNSGSGIIISDTSVHDCDTNCQTPNGAINTSLPFQNIHPVTIGECPKYVKSTKLRMATGLRNIPSIQSRGLFGAIAGFIEGGWTGMIDGWYGYHFQNEQGSGYAADLKSTQNAIDGITNLVNSVIEKMNTQFTAVGKEFSHLERRIENLNKKVDDGFLDIWTYNAELLVLLLNERTLDYHDSNVKNLYEKVRSQLKNNAKEIGNGCFEFYHKCDDMCMESVKNGTYDYPKYSEEAKLNREEIDGVKLESTRIYQILAIYSTVASSLVLVVSLGAISFWMCSNGSLQCRICI

SEQ ID NO: 89 - protein, 565 aa, synthetic construct
MKVKLLVLLCTFTATYADTICIGYHANNSTDTVDTVLEKNVTVTHSVNLLEDSHNGKLCRLKGKAPLQLGNCNIAGWVLGNPECESLLSNRSWSYIAETPNSENGTCYPGDFADYEELREQLSSVSSFERFEIFPKERSWPNHTTRGVTAACPHARKSSFYKNLVWLTEANGSYPNLSRSYVNNQEKEVLVLWGVHHPSNIEEQRALYRKDNAYVSVVSSNYNRRFTPEIAKRPKVRDQSGRMNYYWTLLEPGDTIIFEATGNLIAPWYAFALSRGPGSGIITSNAPLDECDTKCQTPQGAINSSLPFQNIHPVTIGECPKYVRSTKLRMVTGLRNIPSVQSRGLFGAIAGFIEGGWTGMMDGWYGYHFQNEQGSGYAADQKSTQNAINGITNAVNSVIEKMNTQFTAVGKEFNKLEKRMENLNKKVDDGFMDIWTYNAELLVLLANERTLDFHDSNVKNLYEKVKNQLRNNAKELGNGCFEFYHKCDNECMESVKNGTYDYPKYSEESKLNREKIDGVKLESMGVYRILAIYSTVASSLVLLVSLGAISFWMCSNGSLQCRICI

SEQ ID NO: 90 - protein, 565 aa, synthetic construct
MKVKLLVLLCTFTATYADTICIGYHANNSTDTVDTVLEKNVTVTHSVNLLEDSHNGKLCLLKGIAPLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQGAINSSLPFQNVHPVTIGECPKYVRSTKLRMVTGLRNIPSIQSRGLFGAIAGFIEGGWTGMMDGWYGYHFQNEQGSGYAADQKSTQNAINGITNLVNSVIEKMNTQFTAVGKEFNKLERRMENLNKKVDDGFLDIWTYNAELLVLLLNERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 91 - protein, 566 aa, synthetic construct
MKVKLLVLLCTFTATYADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNKGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHFQNEQGSGYAADQKSTQNAINGITNAVNTVIEKMNIQFTAVGKEFNKLEKRMENLNKKVDDGFLDIWTYNAELLVLLANERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 92 - protein, 566 aa, synthetic construct
MKVKLLVLLCTFTATYADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHFQNEQGSGYAADLKSTQNAIDEITNLVNSVIEKMNTQFTAVGKEFNHLEKRIENLNKKVDDGFLDIWTYNAELLVLLLNERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 93 - protein, 566 aa, synthetic construct
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVKQNGTSSACIRKSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIQSGKSSIMRSDAPIGKCKSECITPNGSIPNDKPFQNVNRITYGACPRYVKHSTLKLATGMRNVPEKQTRGIFGAIAGFIENGWEGMVDGWYGFRFQNSEGRGQAADLKSTQAAIDQINGLLNRLIGKTNEKFHQIEKEFSEVEGRIQDLEKYVEDTKIDLWSYNAELLVALLNQHTIDLTDSEMNKLFEKTKKQLRENAEDMGNGCFKIYHKCDNACIGSIRNGTYDHNVYRDEALNNRFQIKGVELKSGYKDWILWISFAISCFLLCVVLLGFIMWACQKGNIRCNICI

SEQ ID NO: 94 - protein, 566 aa, synthetic construct
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSSCKRRSNNSFFSRLNWLTHLKFKYPALNVTMPNNEKFDKLYIWGVHHPGTNNDQISLYTQASGRITVSTKRSQQTVIPNIGSRPRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRFQNSEGIGQAADLKSTQAAINQINGLLNRLIGKTNEKFHQIEKEFSEVEGRIQDLEKYVEDTKIDLWSYNAELLVALLNQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 95 - protein, 566 aa, synthetic construct
MKTIIALSYILCLVFAQKLPGNDNSTATLCLGHHAVPNGTIVKTITNDRIEVTNATELVQNSSIGEICNSPHQILDGGNCTLIDALLGDPQCDGFQNKEWDLFVERSRANSSCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQFSLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRFQNSEGRGQAADLKSTQAAIDQISGLLNRLIGKTNEKFHQIEKEFSEVEGRVQDLEKYVEDTKIDLWSYNAELLVALLNQHTIDLTDSEMNKLFEKTKKQLRENAEDMGNGCFKIYHKCDNACIGSIRNETYDHNVYRDEALNNRFQIKGVELKSGYKDWILWISFAISCFLLCVVLLGFIMWACQKGNIRCNICI

SEQ ID NO: 96 - protein, 566 aa, synthetic construct
MKTIIALSYILCLVFAQKLPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYTYPALNVTMPNNEQFDKLYIWGVHHPSTDKDQISLFAQPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRFQNSEGRGQAADLKSTQAAIDQINGLLNRLIGKTNEKFHQIEKEFSEVEGRVQDLEKYVEDTKIDLWSYNAELLVALLNQHTIDLTDSEMNKLFEKTKKQLRENAEDMGNGCFKIYHKCDNACIGSIRNETYDHNVYRDEALNNRFQIKGVELKSGYKDWILWISFAISCFLLCVVLLGFIMWACQKGNIRCNICI

SEQ ID NO: 97 - protein, 566 aa, synthetic construct
MKTIIALSYILCLVFAQKLPGNDNSTATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFTNEGFNWTGVAQDGTSYACKRGSVKSFFSRLNWLHKLEYKYPALNVTMPNNDKFDKLYIWGVHHPSTDSDQTSLYVQASGRVTVSTKRSQQTVIPNIGSRPWVRGISSRISIYWTIVKPGDILLIKSTGNLIAPRGYFKIRSGKSSIMRSDAPIGNCNSECITPNGSIPNDKPFQNVNRITYGACPRYVKQNTLKLATGMRNVPEKQTRGIFGAIAGFIENGWEGMVDGWYGFRFQNSEGTGQAADLKSTQAAINQINGLLNRLIEKTNEKFHQIEKEFSEVEGRIQDLEKYVEDTKIDLWSYNAELLVALLNQHTIDLTDSEMNKLFERTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 98 - protein, 566 aa, synthetic construct
MKTIIALSYILCLVFAQKLPGNDNSTATLCLGHHAVPNGTLVKTITNDQIEVTNATELVQSSSTGRICDSPHRILDGKNCTLVDALLGDPHCDGFQNEKWDLFVERSKAFSNCYPYDVPDYASLRSLVASSGTLEFINESFNWTGVTQSGGSYACKRGSDNSFFSRLNWLYESESKYPVLNVTMPNNGNFDKLYIWGVHHPSTDKEQTNLYVRASGRVTVSTKRSQQTIIPNIGSRPWVRGLSSRISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAIAGFIENGWEGMVDGWYGFRFQNSEGTGQAADLKSTQAAIDQINGLLNRVIEKTNEKFHQIEKEFSEVEGRIQDLEKYVEDTKIDLWSYNAELLVALLNQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 99 - protein, 566 aa, synthetic construct
MKTIIALSYILCLVFAQKLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRFQNSEGTGQAADLKSTQAAIDQINGLLNRVIEKTNEKFHQIEKEFSEVEGRIQDLEKYVEDTKIDLWSYNAELLVALLNQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQKGNIRCNICI

SEQ ID NO: 100 - protein, 566 aa, synthetic construct
MKTIIALSYILCLVFAQKLPGNDNSTATLCLGHHAVPNGTLVKTITNDQIEVTNATELVQSSSTGKICDNPHRILDGINCTLIDALLGDPHCDGFQNEKWDLFVERSKAFSNCYPYDVPDYASLRSLVASSGTLEFINEGFNWTGVTQNGGSSACKRGPDNGFFSRLNWLYKSGSTYPVQNVTMPNNDNSDKLYIWGVHHPSTDKEQTDLYVQASGKVTVSTKRSQQTVIPNVGSRPWVRGLSSRVSIYWTIVKPGDILVINSNGNLIAPRGYFKMRTGKSSIMRSDAPIGTCSSECITPNGSIPNDKPFQNVNKITYGACPKYVKQNTLKLATGMRNVPEKQTRGIFGAIAGFIENGWEGMIDGWYGFRFQNSEGTGQAADLKSTQAAIDQINGLLNRVIEKTNEKFHQIEKEFSEVEGRIQDLEKYVEDTKIDLWSYNAELLVALLNQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 101 - protein, 566 aa, synthetic construct
MKVKLLVLLCTFTATYADTICIGYHANNSTDTVDTVLEKNVTVTHSVNLLEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTAACSHKGKSSFYRNLLWLTEKDGSYPNLSKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGFGSGIITSNASMDECDTKCQTPQGAINSSLPFQNIHPVTIGECPKYVRSTKLRMVTGLRNIPSIQSRGLFGAIAGFIEGGWTGMIDGWYGYHFQNEQGSGYAADQKSTQNAINGITNAVNSVIEKMNTQFTAVGKEFNKLEKRMENLNKKVDDGFLDIWTYNAELLVLLANERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 102 - protein, 566 aa, synthetic construct
MKVKLLVLLCTFTATYADTLCIGYHANNSTDTVDTVLEKNVTVTHSVNLLEDRHNGKLCKLRGVAPLHLGKCNIAGWLLGNPECELLFTASSWSYIVETSNSDNGTCYPGDFINYEELREQLSSVSSFERFEIFPKASSWPDHETNRGVTAACPYAGANSFYRNLIWLVKKGNSYPKLSKSYVNNKEKEVLVLWGIHHPPTSTDQQSLYQNADAYVFVGSSKYNKKFKPEIATRPKVRGQAGRMNYYWTLVEPGDTITFEATGNLVVPRYAFAMKRGSGSGIIISDTPVHDCNTTCQTPKGAINTSLPFQNIHPVTIGECPKYVKSTKLRMATGLRNIPSIQSRGLFGAIAGFIEGGWTGMIDGWYGYHFQNEQGSGYAADQKSTQNAIDGITNAVNSVIEKMNTQFTAVGKEFNHLEKRIENLNKKVDDGFLDVWTYNAELLVLLANERTLDYHDSNVKNLYEKVRSQLKNNAKEIGNGCFEFYHKCDDTCMESVKNGTYDYPKYSEESKLNREEIDGVKLESTRIYQILAIYSTVASSLVLLVSLGAISFWMCSNGSLQCRICI

SEQ ID NO: 103 - protein, 566 aa, synthetic construct
MKTIIALSYILCLVFAQKLPGNDNSTATLCLGHHAVPNGTLVKTITNDQIEVTNATELVQSSSTGGICDSPHQILDGENCTLIDALLGDPHCDGFQNKEWDLFVERSKAYSNCYPYDVPDYASLRSLVASSGTLEFNNESFNWTGVAQNGTSSACKRRSNKSFFSRLNWLHQLKYKYPALNVTMPNNEKFDKLYIWGVHHPSTDSDQISLYAQAPGRVTVSTKRSQQTVIPNIGSRPWVRGVSSRISIYWTIVKPGDILLINCTGNLIAPRGYFKIRSGKSSIMRSDASIGKCNSECITPNGSIPNDKPFQNVNRITYGACPRYVKQNTLKLATGMRNVPEKQTRGIFGAIAGFIENGWEGMVDGWYGFRFQNSEGTGQAADLKSTQAAINQINGLLNRLIEKTNEKFHQIEKEFSEVEGRIQDLEKYVEDTKIDLWSYNAELLVALLNQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 104 - protein, 566 aa, synthetic construct
MKTIIALSYILCLVFAQKLPGNDNSTATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQDGTSSACIRGSKNSFFSRLNWLTHLNFKYPALNVTMPNNEQFDKLYIWGVHHPGTDKDQIFLYAQASGRITVSTKRSQQAAIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRFQNSEGRGQAADLKSTQAAIDQINGLLNRLIGKTNEKFHQIEKEFSEVEGRIQDLEKYVEDTKIDLWSYNAELLVALLNQHTIDLTDSEMNKLFEKTKKQLRENAEDMGNGCFKIYHKCDNACIGSIRNGTYDHDVYRDEALNKRFQIKGIELKSGYKDWILWISFAISCFLLCVVLLGFIMWACQKGNIRCNICI

SEQ ID NO: 105 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 106 - DNA, 54 bp, synthetic construct
  source 1..54: mol_type = other DNA
gagaataaactagtattcttctggtccccacagactcagagagaacccgccacc

SEQ ID NO: 107 - DNA, 302 bp, synthetic construct
  source 1..302: mol_type = other DNA
tgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagca

SEQ ID NO: 108 - protein, 508 aa, synthetic construct
ADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHFQNEQGSGYAADRKSTQNAIDGITNLVNSVIEKMNTQFTSVGKEFNHLEKRIENLNRKVDDGFLDVWTYNAELLVLLLNERTLDYHDSNVKNLYEKVRSQLKNNAKEIGNGCFEFYHKCDDSCMESVKNGTYDYPKYSEESKLNREEIDGVKLES

SEQ ID NO: 109 - protein, 508 aa, synthetic construct
ADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHFQNEQGSGYAADLKSTQNAIDKITNAVNSVIEKMNTQFTAVGKEFNHLEKRIENLNKKVDDGFLDIWTYNAELLVLLANERTLDYHDSNVKNLYEKVRNQLKNNAKEIGNGCFEFYHKCDNTCMESVKNGTYDYPKYSEEAKLNREKIDGVKLDS

SEQ ID NO: 110 - protein, 507 aa, synthetic construct
ADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHFQNEQGSGYAADQKSTQNAINGITNAVNSVIEKMNTQFTAVGKEFNKLERRMENLNKKVDDGFIDIWTYNAELLVLLANERTLDFHDSNVKNLYEKVKSQLKNNAKEIGNGCFEFYHKCNDECMESVKNGTYDYPKYSEESKLNREKIDGVKLES

SEQ ID NO: 111 - protein, 508 aa, synthetic construct
ADTLCIGYHANNSTDTVDTVLEKNVTVTHSVNLLENRHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHDGTNSFYRNLIWLVKKGNSYPKINKSYINNKEKEILVLWAIHHPSTSADQQSLYQNADAYVFVGSSRYSRKFEPEVATRPKVRDQAGRMNYYWTLVEPGDKITFEATGNLVVPRYAFALKRNSGSGIIISDTSVHDCDTNCQTPNGAINTSLPFQNIHPVTIGECPKYVKSTKLRMATGLRNIPSIQSRGLFGAIAGFIEGGWTGMIDGWYGYHFQNEQGSGYAADLKSTQNAIDGITNLVNSVIEKMNTQFTAVGKEFSHLERRIENLNKKVDDGFLDIWTYNAELLVLLLNERTLDYHDSNVKNLYEKVRSQLKNNAKEIGNGCFEFYHKCDDMCMESVKNGTYDYPKYSEEAKLNREEIDGVKLES

SEQ ID NO: 112 - protein, 507 aa, synthetic construct
ADTICIGYHANNSTDTVDTVLEKNVTVTHSVNLLEDSHNGKLCRLKGKAPLQLGNCNIAGWVLGNPECESLLSNRSWSYIAETPNSENGTCYPGDFADYEELREQLSSVSSFERFEIFPKERSWPNHTTRGVTAACPHARKSSFYKNLVWLTEANGSYPNLSRSYVNNQEKEVLVLWGVHHPSNIEEQRALYRKDNAYVSVVSSNYNRRFTPEIAKRPKVRDQSGRMNYYWTLLEPGDTIIFEATGNLIAPWYAFALSRGPGSGIITSNAPLDECDTKCQTPQGAINSSLPFQNIHPVTIGECPKYVRSTKLRMVTGLRNIPSVQSRGLFGAIAGFIEGGWTGMMDGWYGYHFQNEQGSGYAADQKSTQNAINGITNAVNSVIEKMNTQFTAVGKEFNKLEKRMENLNKKVDDGFMDIWTYNAELLVLLANERTLDFHDSNVKNLYEKVKNQLRNNAKELGNGCFEFYHKCDNECMESVKNGTYDYPKYSEESKLNREKIDGVKLES

SEQ ID NO: 113 - protein, 507 aa, synthetic construct
ADTICIGYHANNSTDTVDTVLEKNVTVTHSVNLLEDSHNGKLCLLKGIAPLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQGAINSSLPFQNVHPVTIGECPKYVRSTKLRMVTGLRNIPSIQSRGLFGAIAGFIEGGWTGMMDGWYGYHFQNEQGSGYAADQKSTQNAINGITNLVNSVIEKMNTQFTAVGKEFNKLERRMENLNKKVDDGFLDIWTYNAELLVLLLNERTLDFHDSNVKNLYEKVKSQLKNNAKEIGNGCFEFYHKCNNECMESVKNGTYDYPKYSEESKLNREKIDGVKLES

SEQ ID NO: 114 - protein, 508 aa, synthetic construct
ADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNKGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHFQNEQGSGYAADQKSTQNAINGITNAVNTVIEKMNIQFTAVGKEFNKLEKRMENLNKKVDDGFLDIWTYNAELLVLLANERTLDFHDSNVKNLYEKVKSQLKNNAKEIGNGCFEFYHKCDNECMESVRNGTYDYPKYSEESKLNREKVDGVKLES

SEQ ID NO: 115 - protein, 508 aa, synthetic construct
ADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHFQNEQGSGYAADLKSTQNAIDEITNLVNSVIEKMNTQFTAVGKEFNHLEKRIENLNKKVDDGFLDIWTYNAELLVLLLNERTLDYHDSNVKNLYEKVRSQLKNNAKEIGNGCFEFYHKCDNTCMESVKNGTYDYPKYSEEAKLNREEIDGVKLES

SEQ ID NO: 116 - protein, 500 aa, synthetic construct
TATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVKQNGTSSACIRKSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIQSGKSSIMRSDAPIGKCKSECITPNGSIPNDKPFQNVNRITYGACPRYVKHSTLKLATGMRNVPEKQTRGIFGAIAGFIENGWEGMVDGWYGFRFQNSEGRGQAADLKSTQAAIDQINGLLNRLIGKTNEKFHQIEKEFSEVEGRIQDLEKYVEDTKIDLWSYNAELLVALLNQHTIDLTDSEMNKLFEKTKKQLRENAEDMGNGCFKIYHKCDNACIGSIRNGTYDHNVYRDEALNNRFQIKGVELKS

SEQ ID NO: 117 - protein, 500 aa, synthetic construct
TATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSSCKRRSNNSFFSRLNWLTHLKFKYPALNVTMPNNEKFDKLYIWGVHHPGTNNDQISLYTQASGRITVSTKRSQQTVIPNIGSRPRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRFQNSEGIGQAADLKSTQAAINQINGLLNRLIGKTNEKFHQIEKEFSEVEGRIQDLEKYVEDTKIDLWSYNAELLVALLNQHTIDLTDSEMNKLFERTKKQLRENAEDMGNGCFKIYHKCDNACIGSIRNGTYDHDVYRDEALNNRFQIKGVELKS

SEQ ID NO: 118 - protein, 500 aa, synthetic construct
TATLCLGHHAVPNGTIVKTITNDRIEVTNATELVQNSSIGEICNSPHQILDGGNCTLIDALLGDPQCDGFQNKEWDLFVERSRANSSCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQFSLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRFQNSEGRGQAADLKSTQAAIDQISGLLNRLIGKTNEKFHQIEKEFSEVEGRVQDLEKYVEDTKIDLWSYNAELLVALLNQHTIDLTDSEMNKLFEKTKKQLRENAEDMGNGCFKIYHKCDNACIGSIRNETYDHNVYRDEALNNRFQIKGVELKS

SEQ ID NO: 119 - protein, 500 aa, synthetic construct
TATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYTYPALNVTMPNNEQFDKLYIWGVHHPSTDKDQISLFAQPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRFQNSEGRGQAADLKSTQAAIDQINGLLNRLIGKTNEKFHQIEKEFSEVEGRVQDLEKYVEDTKIDLWSYNAELLVALLNQHTIDLTDSEMNKLFEKTKKQLRENAEDMGNGCFKIYHKCDNACIGSIRNETYDHNVYRDEALNNRFQIKGVELKS

SEQ ID NO: 120 - protein, 500 aa, synthetic construct
TATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFTNEGFNWTGVAQDGTSYACKRGSVKSFFSRLNWLHKLEYKYPALNVTMPNNDKFDKLYIWGVHHPSTDSDQTSLYVQASGRVTVSTKRSQQTVIPNIGSRPWVRGISSRISIYWTIVKPGDILLIKSTGNLIAPRGYFKIRSGKSSIMRSDAPIGNCNSECITPNGSIPNDKPFQNVNRITYGACPRYVKQNTLKLATGMRNVPEKQTRGIFGAIAGFIENGWEGMVDGWYGFRFQNSEGTGQAADLKSTQAAINQINGLLNRLIEKTNEKFHQIEKEFSEVEGRIQDLEKYVEDTKIDLWSYNAELLVALLNQHTIDLTDSEMNKLFERTRKQLRENAEDMGNGCFKIYHKCDNACIGSIRNGTYDHDVYRDEALNNRFQIKGVELKS

SEQ ID NO: 121 - protein, 500 aa, synthetic construct
TATLCLGHHAVPNGTLVKTITNDQIEVTNATELVQSSSTGRICDSPHRILDGKNCTLVDALLGDPHCDGFQNEKWDLFVERSKAFSNCYPYDVPDYASLRSLVASSGTLEFINESFNWTGVTQSGGSYACKRGSDNSFFSRLNWLYESESKYPVLNVTMPNNGNFDKLYIWGVHHPSTDKEQTNLYVRASGRVTVSTKRSQQTIIPNIGSRPWVRGLSSRISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAIAGFIENGWEGMVDGWYGFRFQNSEGTGQAADLKSTQAAIDQINGLLNRVIEKTNEKFHQIEKEFSEVEGRIQDLEKYVEDTKIDLWSYNAELLVALLNQHTIDLTDSEMNKLFEKTRRQLRENAEDMGNGCFKIYHKCDNACIGSIRNGTYDHDVYRDEALNNRFQIKGVELKS

SEQ ID NO: 122 - protein, 500 aa, synthetic construct
TATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRFQNSEGTGQAADLKSTQAAIDQINGLLNRVIEKTNEKFHQIEKEFSEVEGRIQDLEKYVEDTKIDLWSYNAELLVALLNQHTIDLTDSEMNKLFEKTRRQLRENAEDMGNGCFKIYHKCDNACIESIRNGTYDHDVYRDEALNNRFQIKGVELKS

SEQ ID NO: 123 - protein, 500 aa, synthetic construct
TATLCLGHHAVPNGTLVKTITNDQIEVTNATELVQSSSTGKICDNPHRILDGINCTLIDALLGDPHCDGFQNEKWDLFVERSKAFSNCYPYDVPDYASLRSLVASSGTLEFINEGFNWTGVTQNGGSSACKRGPDNGFFSRLNWLYKSGSTYPVQNVTMPNNDNSDKLYIWGVHHPSTDKEQTDLYVQASGKVTVSTKRSQQTVIPNVGSRPWVRGLSSRVSIYWTIVKPGDILVINSNGNLIAPRGYFKMRTGKSSIMRSDAPIGTCSSECITPNGSIPNDKPFQNVNKITYGACPKYVKQNTLKLATGMRNVPEKQTRGIFGAIAGFIENGWEGMIDGWYGFRFQNSEGTGQAADLKSTQAAIDQINGLLNRVIEKTNEKFHQIEKEFSEVEGRIQDLEKYVEDTKIDLWSYNAELLVALLNQHTIDLTDSEMNKLFEKTRRQLRENAEDMGNGCFKIYHKCDNACIGSIRNGTYDHDVYRDEALNNRFQIKGVELKS

SEQ ID NO: 124 - protein, 508 aa, synthetic construct
ADTICIGYHANNSTDTVDTVLEKNVTVTHSVNLLEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTAACSHKGKSSFYRNLLWLTEKDGSYPNLSKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGFGSGIITSNASMDECDTKCQTPQGAINSSLPFQNIHPVTIGECPKYVRSTKLRMVTGLRNIPSIQSRGLFGAIAGFIEGGWTGMIDGWYGYHFQNEQGSGYAADQKSTQNAINGITNAVNSVIEKMNTQFTAVGKEFNKLEKRMENLNKKVDDGFLDIWTYNAELLVLLANERTLDFHDSNVKNLYEKVKSQLKNNAKEIGNGCFEFYHKCNNECMESVKNGTYDYPKYSEESKLNREKIDGVKLES

SEQ ID NO: 125 - protein, 508 aa, synthetic construct
ADTLCIGYHANNSTDTVDTVLEKNVTVTHSVNLLEDRHNGKLCKLRGVAPLHLGKCNIAGWLLGNPECELLFTASSWSYIVETSNSDNGTCYPGDFINYEELREQLSSVSSFERFEIFPKASSWPDHETNRGVTAACPYAGANSFYRNLIWLVKKGNSYPKLSKSYVNNKEKEVLVLWGIHHPPTSTDQQSLYQNADAYVFVGSSKYNKKFKPEIATRPKVRGQAGRMNYYWTLVEPGDTITFEATGNLVVPRYAFAMKRGSGSGIIISDTPVHDCNTTCQTPKGAINTSLPFQNIHPVTIGECPKYVKSTKLRMATGLRNIPSIQSRGLFGAIAGFIEGGWTGMIDGWYGYHFQNEQGSGYAADQKSTQNAIDGITNAVNSVIEKMNTQFTAVGKEFNHLEKRIENLNKKVDDGFLDVWTYNAELLVLLANERTLDYHDSNVKNLYEKVRSQLKNNAKEIGNGCFEFYHKCDDTCMESVKNGTYDYPKYSEESKLNREEIDGVKLES

SEQ ID NO: 126 - protein, 500 aa, synthetic construct
TATLCLGHHAVPNGTLVKTITNDQIEVTNATELVQSSSTGGICDSPHQILDGENCTLIDALLGDPHCDGFQNKEWDLFVERSKAYSNCYPYDVPDYASLRSLVASSGTLEFNNESFNWTGVAQNGTSSACKRRSNKSFFSRLNWLHQLKYKYPALNVTMPNNEKFDKLYIWGVHHPSTDSDQISLYAQAPGRVTVSTKRSQQTVIPNIGSRPWVRGVSSRISIYWTIVKPGDILLINCTGNLIAPRGYFKIRSGKSSIMRSDASIGKCNSECITPNGSIPNDKPFQNVNRITYGACPRYVKQNTLKLATGMRNVPEKQTRGIFGAIAGFIENGWEGMVDGWYGFRFQNSEGTGQAADLKSTQAAINQINGLLNRLIEKTNEKFHQIEKEFSEVEGRIQDLEKYVEDTKIDLWSYNAELLVALLNQHTIDLTDSEMNKLFERTKKQLRENAEDMGNGCFKIYHKCDNACIGSIRNGTYDHDVYRDEALNNRFQIKGVELKS

SEQ ID NO: 127 - protein, 500 aa, synthetic construct
TATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQDGTSSACIRGSKNSFFSRLNWLTHLNFKYPALNVTMPNNEQFDKLYIWGVHHPGTDKDQIFLYAQASGRITVSTKRSQQAAIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRFQNSEGRGQAADLKSTQAAIDQINGLLNRLIGKTNEKFHQIEKEFSEVEGRIQDLEKYVEDTKIDLWSYNAELLVALLNQHTIDLTDSEMNKLFEKTKKQLRENAEDMGNGCFKIYHKCDNACIGSIRNGTYDHDVYRDEALNKRFQIKGIELKS

SEQ ID NO: 128 - protein, 530 aa, synthetic construct
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHT

SEQ ID NO: 129 - DNA, 111 bp, synthetic construct
  source 1..111: mol_type = other DNA
gctggagcctcggtggccatgcttcttgccccttgggcctccccccagcccctcctccccttcctgcacccgtacccccgtggtctttgaataaagtctgagtgggcggca

SEQ ID NO: 130 - protein, 42 aa, synthetic construct; identical to SEQ ID NO: 60

SEQ ID NO: 131 - protein, 42 aa, synthetic construct
MGVHQILAIYSTVASSLVLLVSLGAISFWMCSNGSLQCRVCI

SEQ ID NO: 132 - protein, 42 aa, synthetic construct; identical to SEQ ID NO: 43

SEQ ID NO: 133 - protein, 42 aa, synthetic construct
TRLYQILAIYSTAASSLVLLVSLGAIGFWMCSNGSLQCRICI

SEQ ID NO: 134 - protein, 42 aa, synthetic construct; identical to SEQ ID NO: 44

SEQ ID NO: 135 - protein, 42 aa, synthetic construct; identical to SEQ ID NO: 43

SEQ ID NO: 136 - protein, 42 aa, synthetic construct; identical to SEQ ID NO: 47

SEQ ID NO: 137 - protein, 41 aa, synthetic construct; identical to SEQ ID NO: 51

SEQ ID NO: 138 - protein, 41 aa, synthetic construct
GYKDWILWISFAISCFLLCVALLGFIMWACQKGNIRCNICI

SEQ ID NO: 139 - protein, 41 aa, synthetic construct; identical to SEQ ID NO: 138

SEQ ID NO: 140 - protein, 41 aa, synthetic construct
GYKDWILWISFAMSCFLLCIALLGFIMWACQKGNIRCNICI

SEQ ID NO: 141 - protein, 41 aa, synthetic construct; identical to SEQ ID NO: 51

SEQ ID NO: 142 - protein, 60 aa, synthetic construct
LPTFDSLNITAASLNDDGLDNHTILLYYSTAASSLAVTLMIAIFVVYMVSRDNVSCSICL

SEQ ID NO: 143 - protein, 60 aa, synthetic construct
LPTFDSLNITAASLNDDGLDNHTILLYYSTAASSLAVTLMIAIFIVYMVSRDNVSCSICL

SEQ ID NO: 144 - protein, 60 aa, synthetic construct
LPTFDSLNITAASLNDDGLDNHTILLYYSTAASSLAVTLMLAIFIVYMVSRDNVSCSICL

SEQ ID NO: 145 - protein, 60 aa, synthetic construct; identical to SEQ ID NO: 143

SEQ ID NO: 146 - protein, 60 aa, synthetic construct; identical to SEQ ID NO: 142

SEQ ID NO: 147 - protein, 60 aa, synthetic construct; identical to SEQ ID NO: 142

SEQ ID NO: 148 - protein, 60 aa, synthetic construct; identical to SEQ ID NO: 142

SEQ ID NO: 149 - protein, 60 aa, synthetic construct; identical to SEQ ID NO: 142

SEQ ID NO: 150 - protein, 60 aa, synthetic construct; identical to SEQ ID NO: 144

SEQ ID NO: 151 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0097112 A1
(43) Pub. Date: Apr. 9, 2026

(54) VACCINE COMPOSITIONS AND USES THEREOF

(71) Applicant: Centivax, Inc., South San Francisco, CA (US)

(72) Inventors: Jacob Glanville, San Francisco, CA (US); Rishi Bedi, Menlo Park, CA (US); Nicholas Bayless, San Francisco, CA (US); Sangil Kim, Belmont, CA (US); Gusti Zeiner, Pacifica, CA (US); Sawsan Youssef, Menlo Park, CA (US)

(21) Appl. No.: 19/361,440

(22) Filed: Oct. 17, 2025

Related U.S. Application Data
(63) Continuation of application No. PCT/US2024/025583, filed on Apr. 19, 2024.
Application No. PCT/US2024/025583 is a continuation-in-part of application No. PCT/US2023/079255, filed on Nov. 9, 2023.
(60) Provisional application No. 63/497,404, filed on Apr. 20, 2023.
(60) Provisional application No. 63/597,625, filed on Nov. 9, 2023.

(51) Int. Cl. A61K 39/145; A61K 39/00; A61P 37/04; C07K 14/005

(52) U.S. Cl. CPC A61K 39/145; A61P 37/04; C07K 14/005; A61K 2039/53; A61K 2039/6093; C12N 2760/16022; C12N 2760/16034; C12N 2760/16071

(57) ABSTRACT

The present disclosure provides vaccine compositions comprising a plurality of distinct antigens. Also provided are nucleic acid vaccine composition comprising one or more nucleic acids encoding for a plurality of distinct antigens. The plurality of distinct antigens comprises a combination of influenza antigens. The vaccine composition can be formulated for delivery as a mRNA/LNP, a recombinant protein, a virus-like particle (VLP), or DNA. Methods of preventing an influenza infection and methods of inducing an immune response are also disclosed.

### CROSS-REFERENCE

This application is a continuation application of International Patent Application No. PCT/US2024/025583, filed Apr. 19, 2024, which is a continuation-in-part of International Patent Application No. PCT/US2023/079255, filed Nov. 9, 2023, which is incorporated herein by reference in its entirety; PCT/US2024/025583 also claims the benefit of U.S. Provisional Application No. 63/497,404, filed Apr. 20, 2023, and U.S. Provisional Application No. 63/597,625, filed Nov. 9, 2023, the entirety of each is incorporated herein by reference.

### SEQUENCE LISTING

The instant application contains a Sequence Listing which has been submitted electronically in XML format and is hereby incorporated by reference in its entirety. Said XML copy, created on Jun. 18, 2024, is named 60333-729_601_SL.xml and is 212,719 bytes in size.

### BACKGROUND OF THE INVENTION

Pathogenic agents, such as viruses, may evolve to evade detection and neutralization by a host's immune system. Thus, most flu vaccines are effective against a limited subset of viruses that cause influenza. Vaccines that are effective against a wider variety of strains for flu are needed.

### SUMMARY OF THE INVENTION

In some aspects, the present disclosure provides a vaccine composition comprising a plurality of distinct antigens, the vaccine composition comprising at least one of: an antigen comprising an amino acid sequence corresponding to SEQ ID NO: 3 or a sequence at least 70% identical to SEQ ID NO: 3; an antigen comprising an amino acid sequence corresponding to SEQ ID NO: 16 or a sequence at least 70% identical to SEQ ID NO: 16; an antigen comprising an amino acid sequence corresponding to SEQ ID NO: 18 or a sequence at least 70% identical to SEQ ID NO: 18; an antigen comprising an amino acid sequence corresponding to SEQ ID NO: 19 or a sequence at least 70% identical to SEQ ID NO: 19; an antigen comprising an amino acid sequence corresponding to SEQ ID NO: 21 or a sequence at least 70% identical to SEQ ID NO: 21; an antigen comprising an amino acid sequence corresponding to SEQ ID NO: 24 or a sequence at least 70% identical to SEQ ID NO: 24; an antigen comprising an amino acid sequence corresponding to SEQ ID NO: 38 or a sequence at least 70% identical to SEQ ID NO: 38; an antigen comprising an amino acid sequence corresponding to SEQ ID NO: 39 or a sequence at least 70% identical to SEQ ID NO: 39; an antigen comprising an amino acid sequence corresponding to SEQ ID NO: 40 or a sequence at least 70% identical to SEQ ID NO: 40; an antigen comprising an amino acid sequence corresponding to SEQ ID NO: 41 or a sequence at least 70% identical to SEQ ID NO: 41; or an antigen comprising an amino acid sequence corresponding to SEQ ID NO: 42 or a sequence at least 70% identical to SEQ ID NO: 42. In some embodiments, the vaccine composition comprises at least one of: an antigen comprising an amino acid sequence corresponding to SEQ ID NO: 3; an antigen comprising an amino acid sequence corresponding to SEQ ID NO: 16; an antigen comprising an amino acid sequence corresponding to SEQ ID NO: 18; an antigen comprising an amino acid sequence corresponding to SEQ ID NO: 19; an antigen comprising an amino acid sequence corresponding to SEQ ID NO: 21; an antigen comprising an amino acid sequence corresponding to SEQ ID NO: 24; an antigen comprising an amino acid sequence corresponding to SEQ ID NO: 38; an antigen comprising an amino acid sequence corresponding to SEQ ID NO: 39; an antigen comprising an amino acid sequence corresponding to SEQ ID NO: 40; an antigen comprising an amino acid sequence corresponding to SEQ ID NO: 41; or an antigen comprising an amino acid sequence corresponding to SEQ ID NO: 42. In some embodiments, the plurality of distinct antigens comprises eight distinct antigens, each of which comprises amino acid sequences selected from the group consisting of SEQ ID NOS: 2-9, 16-22, 24 and 38-42 or sequences at least 90% identical thereto. In some embodiments, the plurality of distinct antigens comprises twelve distinct antigens, each of which comprises amino acid sequences selected from the group consisting of SEQ ID NOS: 2-9, 16-22, 24 and 38-42 or sequences at least 90% identical thereto. In some embodiments, the plurality of distinct antigens comprises eighteen distinct antigens, each of which comprises amino acid sequences selected from the group consisting of SEQ ID NOS: 2-9, 16-22, 24 and 38-42 or sequences at least 90% identical thereto. In some embodiments, the vaccine composition comprises an antigen comprising an amino acid sequence corresponding to SEQ ID NO: 3. In some embodiments, the vaccine composition comprises an antigen comprising an amino acid sequence corresponding to SEQ ID NO: 18. In some embodiments, the vaccine composition comprises an antigen comprising an amino acid sequence corresponding to SEQ ID NO: 19. In some embodiments, the vaccine composition comprises an antigen comprising an amino acid sequence corresponding to SEQ ID NO: 21. In some embodiments, the vaccine composition comprises an antigen comprising an amino acid sequence corresponding to SEQ ID NO: 24. In some embodiments, the vaccine composition comprises an antigen comprising an amino acid sequence corresponding to SEQ ID NO: 38. In some embodiments, the vaccine composition comprises an antigen comprising an amino acid sequence corresponding to SEQ ID NO: 39. In some embodiments, the vaccine composition comprises an antigen comprising an amino acid sequence corresponding to SEQ ID NO: 40. In some embodiments, the vaccine composition comprises an antigen comprising an amino acid sequence corresponding to SEQ ID NO: 41. In some embodiments, the vaccine composition comprises an antigen comprising an amino acid sequence corresponding to SEQ ID NO: 42. In some embodiments, the vaccine composition comprises an antigen comprising an amino acid sequence corresponding to SEQ ID NO: 2. In some embodiments, the vaccine composition comprises an antigen comprising an amino acid sequence corresponding to SEQ ID NO: 4. In some embodiments, the vaccine composition comprises an antigen comprising an amino acid sequence corresponding to SEQ ID NO: 5. In some embodiments, the vaccine composition comprises an antigen comprising an amino acid sequence corresponding to SEQ ID NO: 6. In some embodiments, the vaccine composition comprises an antigen comprising an amino acid sequence corresponding to SEQ ID NO: 7. In some embodiments, the vaccine composition comprises an antigen comprising an amino acid sequence corresponding to SEQ ID NO: 8. In some embodiments, the vaccine composition comprises an antigen comprising an amino acid sequence corresponding to SEQ ID NO: 16. In some embodiments, the vaccine composition comprises an antigen comprising an amino acid sequence corresponding to SEQ ID NO: 17. In some embodiments, the vaccine composition comprises an antigen comprising an amino acid sequence corresponding to SEQ ID NO: 20. In some embodiments, the vaccine composition comprises an antigen comprising an amino acid sequence corresponding to SEQ ID NO: 22. In some embodiments, the plurality of distinct antigens comprises eighteen distinct antigens selected from the group consisting of SEQ ID NOS: 2-9, 16-22, 24, 38-42, or sequences at least 70% identical thereto. In some embodiments, the plurality of distinct antigens comprises at least 6, at least 8, at least 10, at least 12, at least 14, or at least 16 distinct antigens comprising amino acid sequences selected from the group consisting of SEQ ID NO: 2-9, 16-22, 24, 38-42, or sequences at least 70% identical thereto. In some embodiments, the vaccine composition further comprises a pharmaceutically acceptable carrier. In some embodiments, each antigen of the plurality of distinct antigens is present in the vaccine composition in an amount of less than about 5 micrograms (μg), such as between 1 ng and 5 μg. In some embodiments, each antigen of the plurality of distinct antigens is present in the vaccine composition in an amount of less than about 3 μg, such as between 1 ng and 3 μg. In some embodiments, the plurality of antigens comprises at 6, at least 8, at least 10, at least 12, at least 14, at least 16 or at least 17 distinct antigens. In some embodiments, the composition has no more than 30, no more than 25, or no more than 20 distinct antigens. In some embodiments, the plurality of antigens are collectively sufficient to elicit an immune response in a subject yet each individual antigen of the plurality of antigens in present in the composition in an amount that is insufficient to elicit in an immune response in a subject. In some embodiments, the vaccine composition comprises SEQ ID NOS: 2-9, 16-22, 24, and 38-42. In some embodiments, the vaccine composition comprises twenty-one different sequences selected from the group consisting of SEQ ID NOS: 108-128, or sequences at least 90% identical thereto. In some embodiments, the vaccine composition comprises twenty-one different sequences selected from the group consisting of SEQ ID NOS: 108-128, or sequences at least 95% identical thereto. In some embodiments, the vaccine composition comprises twenty-one different sequences selected from the group consisting of SEQ ID NOS: 108-128, or sequences at least 98% identical thereto. In some embodiments, the vaccine composition comprises twenty-one different sequences selected from the group consisting of SEQ ID NOS: 108-128, or sequences at least 99% identical thereto. In some embodiments, the vaccine composition comprises twenty-one different sequences selected from the group consisting of SEQ ID NOS: 108-128.

In some aspects, the present disclosure provides a nucleic acid vaccine composition comprising one or more nucleic acids encoding for a plurality of distinct antigens, wherein the plurality of distinct antigens comprises at least one of: an antigen comprising an amino acid sequence corresponding to SEQ ID NO: 3 or a sequence at least 70% identical to SEQ ID NO: 3; an antigen comprising an amino acid sequence corresponding to SEQ ID NO: 16 or a sequence at least 70% identical to SEQ ID NO: 16; an antigen comprising an amino acid sequence corresponding to SEQ ID NO: 18 or a sequence at least 70% identical to SEQ ID NO: 18; an antigen comprising an amino acid sequence corresponding to SEQ ID NO: 19 or a sequence at least 70% identical to SEQ ID NO: 19; an antigen comprising an amino acid sequence corresponding to SEQ ID NO: 21 or a sequence at least 70% identical to SEQ ID NO: 21; an antigen comprising an amino acid sequence corresponding to SEQ ID NO: 24 or a sequence at least 70% identical to SEQ ID NO: 24; an antigen comprising an amino acid sequence corresponding to SEQ ID NO: 38 or a sequence at least 70% identical to SEQ ID NO: 38; an antigen comprising an amino acid sequence corresponding to SEQ ID NO: 39 or a sequence at least 70% identical to SEQ ID NO: 39; an antigen comprising an amino acid sequence corresponding to SEQ ID NO: 40 or a sequence at least 70% identical to SEQ ID NO: 40; an antigen comprising an amino acid sequence corresponding to SEQ ID NO: 41 or a sequence at least 70% identical to SEQ ID NO: 41; or an antigen comprising an amino acid sequence corresponding to SEQ ID NO: 42 or a sequence at least 70% identical to SEQ ID NO: 42. In some embodiments, the plurality of distinct antigens comprises at least one of: an antigen comprising an amino acid sequence corresponding to SEQ ID NO: 3; an antigen comprising an amino acid sequence corresponding to SEQ ID NO: 16; an antigen comprising an amino acid sequence corresponding to SEQ ID NO: 18; an antigen comprising an amino acid sequence corresponding to SEQ ID NO: 19; an antigen comprising an amino acid sequence corresponding to SEQ ID NO: 21; an antigen comprising an amino acid sequence corresponding to SEQ ID NO: 24; an antigen comprising an amino acid sequence corresponding to SEQ ID NO: 38; an antigen comprising an amino acid sequence corresponding to SEQ ID NO: 39; an antigen comprising an amino acid sequence corresponding to SEQ ID NO: 40; an antigen comprising an amino acid sequence corresponding to SEQ ID NO: 41; or an antigen comprising an amino acid sequence corresponding to SEQ ID NO: 42. In some embodiments, the one or more nucleic acids comprise messenger RNA (mRNA). In some embodiments, the mRNA is encapsulated in lipid nanoparticles (LNPs). In some embodiments, each nucleic acid encoding for each distinct antigen is present in the nucleic acid vaccine composition in an amount of less than about 5 μg. In some embodiments, each nucleic acid encoding for each distinct antigen is present in the nucleic acid vaccine composition in an amount of less than about 3 μg. In some embodiments, the one or more nucleic acids encode eight distinct antigens, each of which is selected from a group consisting of SEQ ID NOS: 2-9, 16-22, 24 and 38-42. In some embodiments, the one or more nucleic acids encode twelve distinct antigens, each of which is selected from a group consisting of SEQ ID NOS: 2-9, 16-22, 24 and 38-42. In some embodiments, the one or more nucleic acids encode eighteen distinct antigens, each of which is selected from a group consisting of SEQ ID NOS: 2-9, 16-22, 24 and 38-42. In some embodiments, the one or more nucleic acids encode for at least five distinct antigens comprising amino acid sequences selected from the group consisting of SEQ ID NOS: 38-42. In some embodiments, the one or more nucleic acids encode for an antigen comprising an amino acid sequence corresponding to SEQ ID NO: 3. In some embodiments, the one or more nucleic acids encode for an antigen comprising an amino acid sequence corresponding to SEQ ID NO: 18. In some embodiments, the one or more nucleic acids encode for an antigen comprising an amino acid sequence corresponding to SEQ ID NO: 19. In some embodiments, the one or more nucleic acids encode for an antigen comprising an amino acid sequence corresponding to SEQ ID NO: 21. In some embodiments, the one or more nucleic acids encode for an antigen comprising an amino acid sequence corresponding to SEQ ID NO: 24. In some embodiments, the one or more nucleic acids encode for an antigen comprising an amino acid sequence corresponding to SEQ ID NO: 38. In some embodiments, the one or more nucleic acids encode for an antigen comprising an amino acid sequence corresponding to SEQ ID NO: 39. In some embodiments, the one or more nucleic acids encode for an antigen comprising an amino acid sequence corresponding to SEQ ID NO: 40. In some embodiments, the one or more nucleic acids encode for an antigen comprising an amino acid sequence corresponding to SEQ ID NO: 41. In some embodiments, the one or more nucleic acids encode for an antigen comprising an amino acid sequence corresponding to SEQ ID NO: 42. In some embodiments, the one or more nucleic acids encode for an antigen comprising an amino acid sequence corresponding to SEQ ID NO: 2. In some embodiments, the one or more nucleic acids encode for an antigen comprising an amino acid sequence corresponding to SEQ ID NO: 4. In some embodiments, the one or more nucleic acids encode for an antigen comprising an amino acid sequence corresponding to SEQ ID NO: 5. In some embodiments, the one or more nucleic acids encode for an antigen comprising an amino acid sequence corresponding to SEQ ID NO: 6. In some embodiments, the one or more nucleic acids encode for an antigen comprising an amino acid sequence corresponding to SEQ ID NO: 7. In some embodiments, the one or more nucleic acids encode for an antigen comprising an amino acid sequence corresponding to SEQ ID NO: 8. In some embodiments, the one or more nucleic acids encode for an antigen comprising an amino acid sequence corresponding to SEQ ID NO: 16. In some embodiments, the one or more nucleic acids encode for an antigen comprising an amino acid sequence corresponding to SEQ ID NO: 17. In some embodiments, the one or more nucleic acids encode for an antigen comprising an amino acid sequence corresponding to SEQ ID NO: 20. In some embodiments, the one or more nucleic acids encode for an antigen comprising an amino acid sequence corresponding to SEQ ID NO: 22. In some embodiments, the one or more nucleic acids encode for an antigen comprising an amino acid sequence corresponding to SEQ ID NO: 38. In some embodiments, the one or more nucleic acids encode for an antigen comprising an amino acid sequence corresponding to SEQ ID NO: 39. In some embodiments, the one or more nucleic acids encode for an antigen comprising an amino acid sequence corresponding to SEQ ID NO: 40. In some embodiments, the one or more nucleic acids encode for an antigen comprising an amino acid sequence corresponding to SEQ ID NO: 41. In some embodiments, the one or more nucleic acids encode for an antigen comprising an amino acid sequence corresponding to SEQ ID NO: 42. In some embodiments, the one or more nucleic acids encode for at least eight distinct antigens comprising amino acid sequences selected from the group consisting of SEQ ID NOS: 2-9, 16-22, 24, 38-42. In some embodiments, the plurality of distinct antigens comprises at least 6, at least 8, at least 10, at least 12, at least 14, or at least 16 distinct antigens comprising amino acid sequences selected from the group consisting of SEQ ID NOS: 2-9, 16-22, 24, 38-42. In some embodiments, the nucleic acid vaccine composition further comprises a pharmaceutically acceptable carrier. In some embodiments, each nucleic acid encoding a distinct antigen is present in the nucleic acid vaccine composition in an amount of less than about 5 micrograms (μg), such as from about 1 ng to about 5 μg. In some embodiments, each nucleic acid encoding a distinct antigen is present in the nucleic acid vaccine composition in an amount of less than about 3 μg, such as from about 1 ng to about 3 μg. In some embodiments, the plurality of antigens comprises at least 6, at least 7, at least 8, at least 9, at least 10, at least 11, at least 12, at least 13, at least 14, at least 15, at least 16, or at least 17 distinct antigens. In some embodiments, the one or more nucleic acids encode for no more than 30, no more than 25, or no more than 20 distinct antigens. In some embodiments, upon administration of the vaccine to a subject, the vaccine elicits an immune response in the subject yet none of the plurality of distinct antigens is individually expressed at a level that is sufficient to elicit an immune response in a subject by itself. In some embodiments, the nucleic acid vaccine composition is formulated as a LNP-encapsulated mRNA, DNA, or viral vector. In some embodiments, the one or more nucleic acids encode for SEQ ID NOS: 2-9, 16-22, 24, and 38-42. In some embodiments, the one or more nucleic acids further encode a transmembrane domain. In some embodiments, the transmembrane domain comprises any one of SEQ ID NOS: 43-63. In some embodiments, the one or more nucleic acids encode each of SEQ ID NOS: 43-63. In some embodiments, the one or more nucleic acids further encode a signaling peptide. In some embodiments, the one or more nucleic acids comprise any one of SEQ ID NOS: 64-84. In some embodiments, the one or more nucleic acids comprise each of SEQ ID NOS: 64-84. In some embodiments, the one or more nucleic acids encode any one of SEQ ID NOS: 85-105. In some embodiments, the one or more nucleic acids encode each of SEQ ID NOS: 85-105. In some embodiments, the one or more nucleic acids comprise any one of SEQ ID NOS: 64-83. In some embodiments, the one or more nucleic acids comprise each of SEQ ID NOS: 64-83. In some embodiments, the one or more nucleic acids comprise any one of SEQ ID NOS: 64, 67-79, and 82-83. In some embodiments, the one or more nucleic acids comprise each of SEQ ID NOS: 64, 67-79, and 82-83. In some embodiments, the one or more nucleic acids encode any one of SEQ ID NOS: 85-104. In some embodiments, the one or more nucleic acids encode each of SEQ ID NOS: 85-104. In some embodiments, the one or more nucleic acids encode any one of SEQ ID NOS: 85-100. In some embodiments, the one or more nucleic acids encode each of SEQ ID NOS: 85-100. In some embodiments, the vaccine composition comprises twenty-one nucleic acid sequences encoding SEQ ID NOS: 108-128, or sequences at least 90% identical thereto. In some embodiments, the vaccine composition comprises twenty-one nucleic acid sequences encoding SEQ ID NOS: 108-128, or sequences at least 95% identical thereto. In some embodiments, the vaccine composition comprises twenty-one nucleic acid sequences encoding SEQ ID NOS: 108-128, or sequences at least 98% identical thereto. In some embodiments, the vaccine composition comprises twenty-one nucleic acid sequences encoding SEQ ID NOS: 108-128, or sequences at least 99% identical thereto.

In some aspects, the present disclosure provides a vaccine composition comprising a plurality of distinct antigens, the vaccine composition comprising at least one of: an antigen comprising an amino acid sequence corresponding to SEQ ID NO: 3 or a sequence at least 70% identical to SEQ ID NO: 3; an antigen comprising an amino acid sequence corresponding to SEQ ID NO: 16 or a sequence at least 70% identical to SEQ ID NO: 16; an antigen comprising an amino acid sequence corresponding to SEQ ID NO: 18 or a sequence at least 70% identical to SEQ ID NO: 18; an antigen comprising an amino acid sequence corresponding to SEQ ID NO: 19 or a sequence at least 70% identical to SEQ ID NO: 19; an antigen comprising an amino acid sequence corresponding to SEQ ID NO: 21 or a sequence at least 70% identical to SEQ ID NO: 21; an antigen comprising an amino acid sequence corresponding to SEQ ID NO: 23 or a sequence at least 70% identical to SEQ ID NO: 23; an antigen comprising an amino acid sequence corresponding to SEQ ID NO: 24 or a sequence at least 70% identical to SEQ ID NO: 24; an antigen comprising an amino acid sequence corresponding to SEQ ID NO: 25 or a sequence at least 70% identical to SEQ ID NO: 25; an antigen comprising an amino acid sequence corresponding to SEQ ID NO: 27 or a sequence at least 70% identical to SEQ ID NO: 27; an antigen comprising an amino acid sequence corresponding to SEQ ID NO: 29 or a sequence at least 70% identical to SEQ ID NO: 29; an antigen comprising an amino acid sequence corresponding to SEQ ID NO: 33 or a sequence at least 70% identical to SEQ ID NO: 33; an antigen comprising an amino acid sequence corresponding to SEQ ID NO: 34 or a sequence at least 70% identical to SEQ ID NO: 34; an antigen comprising an amino acid sequence corresponding to SEQ ID NO: 36 or a sequence at least 70% identical to SEQ ID NO: 36; and an antigen comprising an amino acid sequence corresponding to SEQ ID NO: 37 or a sequence at least 70% identical to SEQ ID NO: 37. In some embodiments, the vaccine composition comprises at least one of: an antigen comprising an amino acid sequence corresponding to SEQ ID NO: 3; an antigen comprising an amino acid sequence corresponding to SEQ ID NO: 13; an antigen comprising an amino acid sequence corresponding to SEQ ID NO: 18; an antigen comprising an amino acid sequence corresponding to SEQ ID NO: 19; an antigen comprising an amino acid sequence corresponding to SEQ ID NO: 21; an antigen comprising an amino acid sequence corresponding to SEQ ID NO: 23; an antigen comprising an amino acid sequence corresponding to SEQ ID NO: 24; an antigen comprising an amino acid sequence corresponding to SEQ ID NO: 25; an antigen comprising an amino acid sequence corresponding to SEQ ID NO: 27; an antigen comprising an amino acid sequence corresponding to SEQ ID NO: 29; an antigen comprising an amino acid sequence corresponding to SEQ ID NO: 33; an antigen comprising an amino acid sequence corresponding to SEQ ID NO: 34; an antigen comprising an amino acid sequence corresponding to SEQ ID NO: 36; and an antigen comprising an amino acid sequence corresponding to SEQ ID NO: 37. In some embodiments, the plurality of distinct antigens comprises eight distinct antigens comprising amino acid sequences selected from the group consisting of SEQ ID NOS: 1-8 or sequences at least 70% identical thereto. In some embodiments, the plurality of distinct antigens comprises eight distinct antigens, wherein the eight distinct antigens correspond to amino acid sequences selected from the group consisting of SEQ ID NOS: 16-23 or sequences at least 70% identical thereto. In some embodiments, the plurality of distinct antigens comprises eight distinct antigens, wherein the eight distinct antigens correspond to amino acid sequences selected from the group consisting of SEQ ID NOS: 1-15 or sequences at least 70% identical thereto. In some embodiments, the plurality of distinct antigens comprises six distinct antigens, wherein the six distinct antigens correspond to amino acid sequences selected from the group consisting of SEQ ID NOS: 16-28 or sequences at least 70% identical thereto. In some embodiments, the vaccine composition comprises an antigen comprising an amino acid sequence corresponding to SEQ ID NO: 3. In some embodiments, the vaccine composition comprises an antigen comprising an amino acid sequence corresponding to SEQ ID NO: 13. In some embodiments, the vaccine composition comprises an antigen comprising an amino acid sequence corresponding to SEQ ID NO: 18. In some embodiments, the vaccine composition comprises an antigen comprising an amino acid sequence corresponding to SEQ ID NO: 19. In some embodiments, the vaccine composition comprises an antigen comprising an amino acid sequence corresponding to SEQ ID NO: 21. In some embodiments, the vaccine composition comprises an antigen comprising an amino acid sequence corresponding to SEQ ID NO: 23. In some embodiments, the vaccine composition comprises an antigen comprising an amino acid sequence corresponding to SEQ ID NO: 24. In some embodiments, the vaccine composition comprises an antigen comprising an amino acid sequence corresponding to SEQ ID NO: 25. In some embodiments, the vaccine composition comprises an antigen comprising an amino acid sequence corresponding to SEQ ID NO: 27. In some embodiments, the vaccine composition comprises an antigen comprising an amino acid sequence corresponding to SEQ ID NO: 29. In some embodiments, the vaccine composition comprises an antigen comprising an amino acid sequence corresponding to SEQ ID NO: 33. In some embodiments, the vaccine composition comprises an antigen comprising an amino acid sequence corresponding to SEQ ID NO: 34. In some embodiments, the vaccine composition comprises an antigen comprising an amino acid sequence corresponding to SEQ ID NO: 36. In some embodiments, the vaccine composition comprises an antigen comprising an amino acid sequence corresponding to SEQ ID NO: 37. In some embodiments, the vaccine composition comprises an antigen comprising an amino acid sequence corresponding to SEQ ID NO: 1. In some embodiments, the vaccine composition comprises an antigen comprising an amino acid sequence corresponding to SEQ ID NO: 2. In some embodiments, the vaccine composition comprises an antigen comprising an amino acid sequence corresponding to SEQ ID NO: 4. In some embodiments, the vaccine composition comprises an antigen comprising an amino acid sequence corresponding to SEQ ID NO: 5. In some embodiments, the vaccine composition comprises an antigen comprising an amino acid sequence corresponding to SEQ ID NO: 6. In some embodiments, the vaccine composition comprises an antigen comprising an amino acid sequence corresponding to SEQ ID NO: 7. In some embodiments, the vaccine composition comprises an antigen comprising an amino acid sequence corresponding to SEQ ID NO: 8. In some embodiments, the vaccine composition comprises an antigen comprising an amino acid sequence corresponding to SEQ ID NO: 16. In some embodiments, the vaccine composition comprises an antigen comprising an amino acid sequence corresponding to SEQ ID NO: 17. In some embodiments, the vaccine composition comprises an antigen comprising an amino acid sequence corresponding to SEQ ID NO: 20. In some embodiments, the vaccine composition comprises an antigen comprising an amino acid sequence corresponding to SEQ ID NO: 22. In some embodiments, the vaccine composition comprises an antigen comprising an amino acid sequence corresponding to SEQ ID NO: 26. In some embodiments, the vaccine composition comprises an antigen comprising an amino acid sequence corresponding to SEQ ID NO: 28. In some embodiments, the plurality of distinct antigens comprises eighteen distinct antigens selected from the group consisting of SEQ ID NOS: 1-8, 16-23, and 29, or sequences at least 70% identical thereto. In some embodiments, the plurality of distinct antigens comprises at least 6, at least 8, at least 10, at least 12, at least 14, or at least 16 distinct antigens comprising amino acid sequences selected from the group consisting of SEQ ID NO: 1-8, 16-23, and 29, or sequences at least 70% identical thereto. In some embodiments, the vaccine composition further comprises a pharmaceutically acceptable carrier. In some embodiments, each antigen of the plurality of distinct antigens is present in the vaccine composition in an amount of less than about 5 micrograms (μg), such as between 1 ng and 5 μg. In some embodiments, each antigen of the plurality of distinct antigens is present in the vaccine composition in an amount of less than about 3 μg, such as between 1 ng and 3 μg. In some embodiments, the plurality of antigens comprises at 6, at least 8, at least 10, at least 12, at least 14, at least 16 or at least 17 distinct antigens. In some embodiments, the composition has no more than 30, no more than 25, or no more than 20 distinct antigens. In some embodiments, the plurality of antigens are collectively sufficient to elicit an immune response in a subject yet each individual antigen of the plurality of antigens in present in the composition in an amount that is insufficient to elicit in an immune response in a subject.

In some aspects, the present disclosure provides a nucleic acid vaccine composition comprising one or more nucleic acids encoding for a plurality of distinct antigens, wherein the plurality of distinct antigens comprises at least one of: an antigen comprising an amino acid sequence corresponding to SEQ ID NO: 3 or a sequence at least 70% identical to SEQ ID NO: 3; an antigen comprising an amino acid sequence corresponding to SEQ ID NO: 13 or a sequence at least 70% identical to SEQ ID NO: 13; an antigen comprising an amino acid sequence corresponding to SEQ ID NO: 18 or a sequence at least 70% identical to SEQ ID NO: 18; an antigen comprising an amino acid sequence corresponding to SEQ ID NO: 19 or a sequence at least 70% identical to SEQ ID NO: 19; an antigen comprising an amino acid sequence corresponding to SEQ ID NO: 21 or a sequence at least 70% identical to SEQ ID NO: 21; an antigen comprising an amino acid sequence corresponding to SEQ ID NO: 23 or a sequence at least 70% identical to SEQ ID NO: 23; an antigen comprising an amino acid sequence corresponding to SEQ ID NO: 24 or a sequence at least 70% identical to SEQ ID NO: 24; an antigen comprising an amino acid sequence corresponding to SEQ ID NO: 25 or a sequence at least 70% identical to SEQ ID NO: 25; an antigen comprising an amino acid sequence corresponding to SEQ ID NO: 27 or a sequence at least 70% identical to SEQ ID NO: 27; an antigen comprising an amino acid sequence corresponding to SEQ ID NO: 29 or a sequence at least 70% identical to SEQ ID NO: 29; an antigen comprising an amino acid sequence corresponding to SEQ ID NO: 33 or a sequence at least 70% identical to SEQ ID NO: 33; an antigen comprising an amino acid sequence corresponding to SEQ ID NO: 34 or a sequence at least 70% identical to SEQ ID NO: 34; an antigen comprising an amino acid sequence corresponding to SEQ ID NO: 36 or a sequence at least 70% identical to SEQ ID NO: 36; and an antigen comprising an amino acid sequence corresponding to SEQ ID NO: 37 or a sequence at least 70% identical to SEQ ID NO: 37. In some embodiments, the plurality of distinct antigens comprises at least one of: an antigen comprising an amino acid sequence corresponding to SEQ ID NO: 3; an antigen comprising an amino acid sequence corresponding to SEQ ID NO: 13; an antigen comprising an amino acid sequence corresponding to SEQ ID NO: 18; an antigen comprising an amino acid sequence corresponding to SEQ ID NO: 19; an antigen comprising an amino acid sequence corresponding to SEQ ID NO: 21; an antigen comprising an amino acid sequence corresponding to SEQ ID NO: 23; an antigen comprising an amino acid sequence corresponding to SEQ ID NO: 24; an antigen comprising an amino acid sequence corresponding to SEQ ID NO: 25; an antigen comprising an amino acid sequence corresponding to SEQ ID NO: 27; an antigen comprising an amino acid sequence corresponding to SEQ ID NO: 29; an antigen comprising an amino acid sequence corresponding to SEQ ID NO: 33; an antigen comprising an amino acid sequence corresponding to SEQ ID NO: 34; an antigen comprising an amino acid sequence corresponding to SEQ ID NO: 36; and an antigen comprising an amino acid sequence corresponding to SEQ ID NO: 37. In some embodiments, the one or more nucleic acids comprise messenger RNA (mRNA). In some embodiments, the mRNA is encapsulated in lipid nanoparticles (LNPs). In some embodiments, each nucleic acid encoding for each distinct antigen is present in the nucleic acid vaccine composition in an amount of less than about 5 μg. In some embodiments, each nucleic acid encoding for each distinct antigen is present in the nucleic acid vaccine composition in an amount of less than about 3 μg. In some embodiments, the one or more nucleic acids encode for at least eight distinct antigens comprising amino acid sequences selected from the group consisting of SEQ ID NOS: 1-8. In some embodiments, the one or more nucleic acids encode for at least eight distinct antigens comprising amino acid sequences selected from the group consisting of SEQ ID NOS: 16-23. In some embodiments, the one or more nucleic acids encode for at least eight distinct antigens comprising amino acid sequences selected from the group consisting of SEQ ID NOS: 1-15. In some embodiments, the one or more nucleic acids encode for at least six distinct antigens comprising amino acid sequences selected from the group consisting of SEQ ID NOS: 16-28. In some embodiments, the one or more nucleic acids encode for an antigen comprising an amino acid sequence corresponding to SEQ ID NO: 3. In some embodiments, the one or more nucleic acids encode for an antigen comprising an amino acid sequence corresponding to SEQ ID NO: 13. In some embodiments, the one or more nucleic acids encode for an antigen comprising an amino acid sequence corresponding to SEQ ID NO: 18. In some embodiments, the one or more nucleic acids encode for an antigen comprising an amino acid sequence corresponding to SEQ ID NO: 19. In some embodiments, the one or more nucleic acids encode for an antigen comprising an amino acid sequence corresponding to SEQ ID NO: 21. In some embodiments, the one or more nucleic acids encode for an antigen comprising an amino acid sequence corresponding to SEQ ID NO: 23. In some embodiments, the one or more nucleic acids encode for an antigen comprising an amino acid sequence corresponding to SEQ ID NO: 24. In some embodiments, the one or more nucleic acids encode for an antigen comprising an amino acid sequence corresponding to SEQ ID NO: 25. In some embodiments, the one or more nucleic acids encode for an antigen comprising an amino acid sequence corresponding to SEQ ID NO: 27. In some embodiments, the one or more nucleic acids encode for an antigen comprising an amino acid sequence corresponding to SEQ ID NO: 29. In some embodiments, the one or more nucleic acids encode for an antigen comprising an amino acid sequence corresponding to SEQ ID NO: 33. In some embodiments, the one or more nucleic acids encode for an antigen comprising an amino acid sequence corresponding to SEQ ID NO: 34. In some embodiments, the one or more nucleic acids encode for an antigen comprising an amino acid sequence corresponding to SEQ ID NO: 36. In some embodiments, the one or more nucleic acids encode for an antigen comprising an amino acid sequence corresponding to SEQ ID NO: 37. In some embodiments, the one or more nucleic acids encode for an antigen comprising an amino acid sequence corresponding to SEQ ID NO: 1. In some embodiments, the one or more nucleic acids encode for an antigen comprising an amino acid sequence corresponding to SEQ ID NO: 2. In some embodiments, the one or more nucleic acids encode for an antigen comprising an amino acid sequence corresponding to SEQ ID NO: 4. In some embodiments, the one or more nucleic acids encode for an antigen comprising an amino acid sequence corresponding to SEQ ID NO: 5. In some embodiments, the one or more nucleic acids encode for an antigen comprising an amino acid sequence corresponding to SEQ ID NO: 6. In some embodiments, the one or more nucleic acids encode for an antigen comprising an amino acid sequence corresponding to SEQ ID NO: 7. In some embodiments, the one or more nucleic acids encode for an antigen comprising an amino acid sequence corresponding to SEQ ID NO: 8. In some embodiments, the one or more nucleic acids encode for an antigen comprising an amino acid sequence corresponding to SEQ ID NO: 16. In some embodiments, the one or more nucleic acids encode for an antigen comprising an amino acid sequence corresponding to SEQ ID NO: 17. In some embodiments, the one or more nucleic acids encode for an antigen comprising an amino acid sequence corresponding to SEQ ID NO: 20. In some embodiments, the one or more nucleic acids encode for an antigen comprising an amino acid sequence corresponding to SEQ ID NO: 22. In some embodiments, the one or more nucleic acids encode for an antigen comprising an amino acid sequence corresponding to SEQ ID NO: 26. In some embodiments, the one or more nucleic acids encode for an antigen comprising an amino acid sequence corresponding to SEQ ID NO: 28. In some embodiments, the one or more nucleic acids encode for at least eight distinct antigens comprising amino acid sequences selected from the group consisting of SEQ ID NOS: 1-8, 16-23, or 29. In some embodiments, the plurality of distinct antigens comprises at least 6, at least 8, at least 10, at least 12, at least 14, or at least 16 distinct antigens comprising amino acid sequences selected from the group consisting of SEQ ID NOS: 1-8, 16-23, or 29. In some embodiments, the nucleic acid vaccine composition further comprises a pharmaceutically acceptable carrier. In some embodiments, each nucleic acid encoding a distinct antigen is present in the nucleic acid vaccine composition in an amount of less than about 5 micrograms (μg), such as from about 1 ng to about 5 μg. In some embodiments, each nucleic acid encoding a distinct antigen is present in the nucleic acid vaccine composition in an amount of less than about 3 μg, such as from about 1 ng to about 3 μg. In some embodiments, the plurality of antigens comprises at least 6, at least 7, at least 8, at least 9, at least 10, at least 11, at least 12, at least 13, at least 14, at least 15, at least 16, or at least 17 distinct antigens. In some embodiments, the one or more nucleic acids encode for no more than 30, no more than 25, or no more than 20 distinct antigens. In some embodiments, upon administration of the vaccine to a subject, the vaccine elicits an immune response in the subject yet none of the plurality of distinct antigens is individually expressed at a level that is sufficient to elicit an immune response in a subject by itself. In some embodiments, the nucleic acid vaccine composition is formulated as an LNP-encapsulated mRNA or DNA or viral vector. In some embodiments, the one or more nucleic acids further encode a transmembrane domain. In some embodiments, the transmembrane domain comprises any one of SEQ ID NOS: 43-63. In some embodiments, the one or more nucleic acids encode any one of SEQ ID NOS: 43-63. In some embodiments, the one or more nucleic acids further encode a signaling peptide. In some embodiments, the one or more nucleic acids comprise any one of SEQ ID NOS: 64-84. In some embodiments, the one or more nucleic acids comprise each of SEQ ID NOS: 64-84. In some embodiments, the one or more nucleic acids encode any one of SEQ ID NOS: 85-105. In some embodiments, the one or more nucleic acids encode each of SEQ ID NOS: 85-105. In some embodiments, the one or more nucleic acids comprise any one of SEQ ID NOS: 64-83. In some embodiments, the one or more nucleic acids comprise each of SEQ ID NOS: 64-83. In some embodiments, the one or more nucleic acids comprise any one of SEQ ID NOS: 64, 67-79, and 82-83. In some embodiments, the one or more nucleic acids comprise each of SEQ ID NOS: 64, 67-79, and 82-83. In some embodiments, the one or more nucleic acids encode any one of SEQ ID NOS: 85-104. In some embodiments, the one or more nucleic acids encode each of SEQ ID NOS: 85-104. In some embodiments, the one or more nucleic acids encode any one of SEQ ID NOS: 85-100. In some embodiments, the one or more nucleic acids encode each of SEQ ID NOS: 85-100. A vaccine composition comprising twenty-one distinct antigens, wherein each of the twenty-one distinct antigens comprises a sequence selected from SEQ ID NOS: 108-128, or a sequence having at least 90%, at least 95%, at least 98%, at least 99% sequence identity thereto. In some embodiments, each of the twenty-one distinct antigens comprises a sequence selected from SEQ ID NOS: 108-128. In some embodiments, each of the twenty-one distinct antigens comprises or consists of a sequence selected from SEQ ID NOS: 85-105 A nucleic acid vaccine composition comprising one or more nucleic acids encoding for at least twenty-one distinct antigens, wherein each the twenty-one distinct antigens comprises a sequence selected from SEQ ID NOS: 108-128, or a sequence having at least 90%, at least 95%, at least 98%, or at least 99% sequence identity thereto. In some embodiments, each of the at least twenty-one distinct antigens comprises a sequence selected from SEQ ID NOS: 108-128. In some embodiments, each of the twenty-one distinct antigens comprises or consists of a sequence selected from SEQ ID NOS: 85-105. In some embodiments, the vaccine composition comprises each of SEQ ID NOS: 64-84.

In some aspects, the present disclosure provides a method for preventing an influenza infection in a subject, comprising administering to the subject any vaccine composition as disclosed herein. In some aspects, the present disclosure provides a method of inducing an immune response in a subject, the method comprising administering to the subject any vaccine composition as disclosed herein. In some embodiments, the immune response is a protective immune response. In some embodiments, the vaccine composition is administered to the subject intramuscularly, subcutaneously, intramuscularly, intranasally, or orally. In some embodiments, the nucleic acid vaccine composition is formulated for delivery as a LNP-encapsulated mRNA or DNA or viral vector. An infection rate in subjects receiving the vaccine may be reduced by 2% (or more) relative or more relative to a placebo control or relative to a subject who did not receive the vaccine composition.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts pairwise sequence identity for antigens in (or antigens expressed by nucleic acid sequences in) an exemplary influenza vaccine composition.

FIG. 2A depicts the amount of induced antibodies measured by ELISA reactive to a particular recombinant influenza hemagglutinin antigen in the serum of mice in response to vaccination with either Centi-Flu (0.031 ug of mRNA/antigen), Flu-Biv (0.25 ug/antigen), or Flu-Biv (0.031 ug/antigen). Reactivity is shown to antigens present in Centi-Flu (H3N2 Hong Kong/1/1968, H3N2 Alaska/01/2021), present in Centi-Flu and Flu-Biv (California/07/2004), or heterologous antigens present in neither (H3N2 A/Victoria/361/2011, A/Maryland/02/2021). FIG. 2B depicts the amount of induced antibodies measured by ELISA reactive to a particular recombinant influenza hemagglutinin antigen in the serum of mice in response to vaccination with either Centi-Flu (0.25 ug of mRNA/antigen), Flu-Biv (2 ug/antigen), or Flu-Biv (0.25 ug/antigen). Reactivity is shown to antigens present in Centi-Flu (H3N2 Hong Kong/1/1968, H3N2 Alaska/01/2021), present in Centi-Flu and Flu-Biv (California/07/2004), or heterologous antigens present in neither (H3N2 A/Victoria/361/2011, A/Maryland/02/2021).

FIG. 3A depicts serum reactivity measured by ELISA in ferrets immunized with 1 μg of mRNA encoding influenza hemagglutinin antigens of H3N2 Alaska/01/2021, H3N2 California/07/2004, H3N2 Cambodia/2020, H3N2 Indiana/11/2018, H3N2 Bilthoven/1761/1976, H3N2 Indiana/08/2011, H3N2 Nanchang/933/1995, or H3N2 Memphis/1/1980, to the same antigen that the animal was immunized with; FIG. 3B depicts serum reactivity measured by ELISA in ferrets immunized with approximately 0.5 ug of mRNA per antigen encoding 8 influenza hemagglutinin antigens of H3N2 Alaska/01/2021, H3N2 California/07/2004, H3N2 Cambodia/2020, H3N2 Indiana/11/2018, H3N2 Bilthoven/1761/1976, H3N2 Nanchang/933/1995, H3N2 Memphis/1/1980, H3N2 Hong Kong/1/1968, to the indicated recombinant protein antigens.

FIG. 4 depicts pairwise sequence identity between a set of H3N2 hemagglutinin homologous distinct antigen components.

FIG. 5 depicts the results of hemagglutinin inhibition (HAI) studies in mice immunized with a vaccine composition having twenty mRNAs encoding hemagglutinin antigens (LNP-20) at different doses.

FIG. 6 depicts the results of HAI studies in mice immunized with a vaccine composition having eighteen mRNAs encoding hemagglutinin antigens (LNP-18).

FIG. 7 depicts the results of HAI studies in rats immunized with a vaccine composition having eighteen mRNAs encoding hemagglutinin antigens (LNP-18).

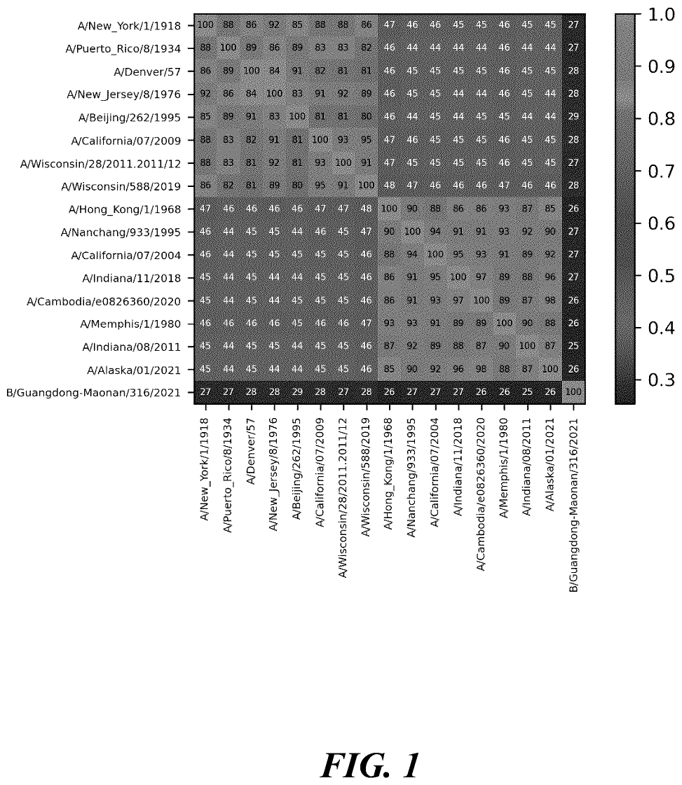

### DETAILED DESCRIPTION OF THE INVENTION

The present disclosure provides vaccine compositions and methods for treating influenza. In some embodiments, the vaccine composition comprises one or more antigens. In some other embodiments, the vaccine composition comprises nucleic acids that encode for the plurality of antigens. In some embodiments, the plurality of antigens includes one or more influenza proteins, or derivatives thereof. In some embodiments, the one or more antigens comprise hemagglutinin proteins, or derivatives thereof. In some embodiments, the hemagglutinin proteins or derivatives thereof comprises one or more hemagglutinin subtypes selected from H1, H2, H3, H4, H5, H6, H7, H8, H9, H10, H11, H12, H13, H14, H15, H16, H17, or H18. In some embodiments, the one or more antigens include hemagglutinin proteins from multiple subtypes, such as from H1 and H3. In some embodiments, the antigens include hemagglutinin proteins from the influenza B subtype (HAB). In some embodiments, the plurality of antigens comprises the amino acid sequences of any one of SEQ ID NOS: 1-42, as disclosed in Table 1. In some embodiments, the plurality of antigens comprises the amino acid sequences of any one of SEQ ID NOS: 108-128, as disclosed in Table 4. In some embodiments, an antigen of the plurality of antigens comprises the extracellular domain of the antigen. In some embodiments, an antigen of the plurality of antigens further comprises a transmembrane domain. In some embodiments, the transmembrane domain comprises the sequence of any one of SEQ ID NOS: 43-63 or 130-150. In some embodiments, the transmembrane domain comprises the sequence of any one of SEQ ID NOS: 43-63. In some embodiments, the transmembrane domain may be coupled to an extracellular domain of the antigen. In some embodiments, the vaccine composition comprises nucleic acid sequences encoding any one of SEQ ID NOS: 85-105. In some embodiments, the vaccine composition comprises one or more nucleic acids. In some embodiments, each nucleic acid of the nucleic acid vaccine may encode a signal peptide of an influenza hemagglutinin. In some embodiments, each nucleic acid may encode an extracellular domain of an influenza hemagglutinin. In some embodiments, each nucleic acid may encode a transmembrane domain of an influenza hemagglutinin. In some embodiments, each nucleic acid may encode a signal peptide, an extracellular domain, and a transmembrane domain, wherein the signal peptide, the extracellular domain, and the transmembrane domain are each operably linked with one another. In some embodiments, each nucleic acid may encode an open reading frame coding for a signal peptide, extracellular domain, and transmembrane domain. In some embodiments, each nucleic acid encodes only a signal peptide and an extracellular domain of an antigen. In some embodiments, the nucleic acid encodes an antigen extracellular domain that is not connected to a signal peptide. In some embodiments, the nucleic acid encodes an antigen extracellular domain that is not connected to a transmembrane domain. In some embodiments, the nucleic acid encodes an antigen extracellular domain that is not connected to a signal peptide or a transmembrane domain. In some embodiments, a nucleic acid of the vaccine composition may comprise any of SEQ ID NOs 64-84. In some embodiments, the antigen extracellular domain may have 1-5 amino acid mutations relative to the wild type sequence to increase the stability of the antigen. In some embodiments, the antigen extracellular domain may have 3 amino acid mutations relative to the wild type sequence to increase the stability of the antigen.

TABLE1
Amino acid sequences of antigens and corresponding transmembrane domains for
a vaccine composition

| | | | Amino Acid | |
|---|---|---|---|---|
| | | | Sequence of | |
| | | SEQ | Antigen | SEQ |
| | Amino Acid Sequence of Antigen | ID | Transmembrane | ID |
| Strain | Extracellular Domain | NO: | Domain | NO: |
| A/New_Jersey/ | ADTLCIGYHANNSTDTVDTVLEKNVTVTHSVN | 1 | TRIYQILAIYSTV | 130 |
| 8/1976 | LLEDRHNGKLCKLGGIAPLHLGKCNIAGWLLG | | ASSLVLLVSLGAI | |
| A_/_H1N1 | NPECELLLTVSSWSYIVETSNSDNGTCYPGDF | | SFWMCSNGSLQCR | |
| | INYEELREQLSSVSSFERFEIFPKTSSWPDHE | | ICI | |
| | TNRGVTAACPYAGANSFYRNLIWLVEKGNSYP | | | |
| | KLSKSYVNNKGKEVLVLWGIHHPPTSTDQQSL | | | |
| | YQNADAYVFVGSSKYNRKFKPEIAARPKVRGQ | | | |
| | AGRMNYYWTLIEPGDTITFEATGNLVVPRYAF | | | |
| | AMNRGSGSGIIISDAPVHDCNTKCQTPKGAIN | | | |
| | TSLPFQNIHPVTIGECPKYVKSTKLRMATGLR | | | |
| | NIPSIQSRGLFGAIAGFIEGGWTGMIDGWYGY | | | |
| | HHQNEQGSGYAADQRSTQNAIDGITNKVNSVI | | | |
| | EKMNTQFTAVGKEFNHLEKRIENLNKKVDDGF | | | |
| | LDIWTYNAELLVLLENERTLDFHDSNVKNLYE | | | |
| | KVRSQLRNNAKEIGNGCFEFYHKCDDTCMESV | | | |
| | KNGTYDYPKYSEESKLNREEIDGVKLES | | | |
| A/New York/1/ | ADTICIGYHANNSTDTVDTVLEKNVTVTHSVN | 2 | MGVYQILAIYSTV | 43 |
| 1918 A_/ | LLEDSHNGKLCKLKGIAPLQLGKCNIAGWLLG | | ASSLVLLVSLGAI | |
| _H1N1 | NPECDLLLTASSWSYIVETSNSFNGTCYPGDF | | SFWMCSNGSLQCR | |
| | IDYEELREQLSSVSSFEKFEIFPKTSSWPNHE | | ICI | |
| | TTKGVTAACSYAGASSFYRNLLWLTKKGSSYP | | | |
| | KLSKSYVNNKGKEVLVLWGVHHPPTGTDQQSL | | | |
| | YQNADAYVSVGSSKYNRRFTPEIAARPKVRDQ | | | |
| | AGRMNYYWTLLEPGDTITFEATGNLIAPWYAF | | | |
| | ALNRGSGSGIITSDAPVHDCNTKCQTPHGAIN | | | |
| | SSLPFQNIHPVTIGECPKYVRSTKLRMATGLR | | | |
| | NIPSIQSRGLFGAIAGFIEGGWTGMIDGWYGY | | | |
| | HHQNEQGSGYAADRKSTQNAIDGITNKVNSVI | | | |
| | EKMNTQFTSVGKEFNHLEKRIENLNRKVDDGF | | | |
| | LDVWTYNAELLVLLENERTLDYHDSNVKNLYE | | | |
| | KVRSQLKNNAKEIGNGCFEFYHKCDDSCMESV | | | |
| | KNGTYDYPKYSEESKLNREEIDGVKLES | | | |
| A/Wisconsin/ | ADTLCIGYHANNSTDTVDTVLEKNVTVTHSVN | 3 | TRIYQILATYSTV | 44 |
| 588/2019 | LLEDKHNGKLCKLRGVAPLHLGKCNIAGWILG | | ASSLVLVVSLGAI | |
| A_/_H1N1 | NPECESLSTARSWSYIVETSNSDNGTCYPGDF | | SFWMCSNGSLQCR | |
| | INYEELREQLSSVSSFERFEIFPKTSSWPNHD | | ICI | |
| | SDNGVTAACPHAGAKSFYKNLIWLVKKGKSYP | | | |
| | KINQTYINDKGKEVLVLWGIHHPPTIADQQSL | | | |
| | YQNADAYVFVGTSRYSKKFKPEIATRPKVRDQ | | | |
| | EGRMNYYWTLVEPGDKITFEATGNLVAPRYAF | | | |
| | TMERDAGSGIIISDTPVHDCNTTCQTPEGAIN | | | |
| | TSLPFQNVHPITIGKCPKYVKSTKLRLATGLR | | | |
| | NVPSIQSRGLFGAIAGFIEGGWTGMVDGWYGY | | | |
| | HHQNEQGSGYAADLKSTQNAIDKITNKVNSVI | | | |
| | EKMNTQFTAVGKEFNHLEKRIENLNKKVDDGF | | | |
| | LDIWTYNAELLVLLENERTLDYHDSNVKNLYE | | | |
| | KVRNQLKNNAKEIGNGCFEFYHKCDNTCMESV | | | |
| | KNGTYDYPKYSEEAKLNREKIDGVKLDS | | | |
| A/Brisbane/ | ADTICIGYHANNSTDTVDTVLEKNVTVTHSVN | 4 | MGVYQILAIYSTV | 45 |
| 59/2007 | LLENSHNGKLCLLKGIAPLQLGNCSVAGWILG | | ASSLVLLVSLGAI | |
| A_/_H1N1 | NPECELLISKESWSYIVEKPNPENGTCYPGHF | | SFWMCSNGSLQCR | |
| | ADYEELREQLSSVSSFERFEIFPKESSWPNHT | | ICI | |
| | VTGVSASCSHNGESSFYRNLLWLTGKNGLYPN | | | |
| | LSKSYANNKEKEVLVLWGVHHPPNIGNQKALY | | | |
| | HTENAYVSVVSSHYSRKFTPEIAKRPKVRDQE | | | |
| | GRINYYWTLLEPGDTIIFEANGNLIAPRYAFA | | | |
| | LSRGFGSGIINSNAPMDKCDAKCQTPQGAINS | | | |
| | SLPFQNVHPVTIGECPKYVRSAKLRMVTGLRN | | | |
| | IPSIQSRGLFGAIAGFIEGGWTGMVDGWYGYH | | | |
| | HQNEQGSGYAADQKSTQNAINGITNKVNSVIE | | | |
| | KMNTQFTAVGKEFNKLERRMENLNKKVDDGFI | | | |
| | DIWTYNAELLVLLENERTLDFHDSNVKNLYEK | | | |
| | VKSQLKNNAKEIGNGCFEFYHKCNDECMESVK | | | |
| | NGTYDYPKYSEESKLNREKIDGVKLES | | | |
| A/Wisconsin/ | ADTLCIGYHANNSTDTVDTVLEKNVTVTHSVN | 5 | TRIYQILAIYSTV | 46 |
| 28/2011.2011/ | LLENRHNGKLCKLRGVAPLHLGKCNIAGWLLG | | ASSLVLVVSLGAI | |
| 12 A_/_H1N1 | NPECESLSTASSWSYIVETSNSDNGTCYPGDF | | SFWMCSNGSLQCR | |
| | INYEELREQLSSVSSFERFEIFPKTSSWPNHD | | ICI | |
| | TNRGVTAACPHDGTNSFYRNLIWLVKKGNSYP | | | |
| | KINKSYINNKEKEILVLWAIHHPSTSADQQSL | | | |
| | YQNADAYVFVGSSRYSRKFEPEVATRPKVRDQ | | | |
| | AGRMNYYWTLVEPGDKITFEATGNLVVPRYAF | | | |
| | ALKRNSGSGIIISDTSVHDCDTNCQTPNGAIN | | | |
| | TSLPFQNIHPVTIGECPKYVKSTKLRMATGLR | | | |
| | NIPSIQSRGLFGAIAGFIEGGWTGMIDGWYGY | | | |
| | HHQNEQGSGYAADLKSTQNAIDGITNKVNSVI | | | |
| | EKMNTQFTAVGKEFSHLERRIENLNKKVDDGF | | | |
| | LDIWTYNAELLVLLENERTLDYHDSNVKNLYE | | | |
| | KVRSQLKNNAKEIGNGCFEFYHKCDDMCMESV | | | |
| | KNGTYDYPKYSEEAKLNREEIDGVKLES | | | |
| A/Denver/57 | ADTICIGYHANNSTDTVDTVLEKNVTVTHSVN | 6 | MGVYRILAIYSTV | 47 |
| A_/_H1N1 | LLEDSHNGKLCRLKGKAPLQLGNCNIAGWVLG | | ASSLVLLVSLGAI | |
| | NPECESLLSNRSWSYIAETPNSFNGTCYPGDF | | SFWMCSNGSLQCR | |
| | ADYEELREQLSSVSSFERFEIFPKERSWPNHT | | ICI | |
| | TRGVTAACPHARKSSFYKNLVWLTEANGSYPN | | | |
| | LSRSYVNNQEKEVLVLWGVHHPSNIEEQRALY | | | |
| | RKDNAYVSVVSSNYNRRFTPEIAKRPKVRDQS | | | |
| | GRMNYYWTLLEPGDTIIFEATGNLIAPWYAFA | | | |
| | LSRGPGSGIITSNAPLDECDTKCQTPQGAINS | | | |
| | SLPFQNIHPVTIGECPKYVRSTKLRMVTGLRN | | | |
| | IPSVQSRGLFGAIAGFIEGGWTGMMDGWYGYH | | | |
| | HQNEQGSGYAADQKSTQNAINGITNKVNSVIE | | | |
| | KMNTQFTAVGKEFNKLEKRMENLNKKVDDGFM | | | |
| | DIWTYNAELLVLLENERTLDFHDSNVKNLYEK | | | |
| | VKNQLRNNAKELGNGCFEFYHKCDNECMESVK | | | |
| | NGTYDYPKYSEESKLNREKIDGVKLES | | | |
| A/Beijing/ | ADTICIGYHANNSTDTVDTVLEKNVTVTHSVN | 7 | MGVYQILAIYSTV | 48 |
| 262/1995 | LLEDSHNGKLCLLKGIAPLQLGNCSVAGWILG | | ASSLVLLVSLGAI | |
| A_/_H1N1 | NPECESLISKESWSYIVETPNPENGTCYPGYF | | SFWMCSNGSLQCR | |
| | ADYEELREQLSSVSSFERFEIFPKESSWPNHT | | ICI | |
| | VTGVTASCSHNGKSSFYRNLLWLTEKNGLYPN | | | |
| | LSNSYVNNKEKEVLVLWGVHHPSNIRDQRAIY | | | |
| | HTENAYVSVVSSHYSRRFTPEIAKRPKVRGQE | | | |
| | GRINYYWTLLEPGDTIIFEANGNLIAPWYAFA | | | |
| | LSRGFGSGIITSNAPMNECDAKCQTPQGAINS | | | |
| | SLPFQNVHPVTIGECPKYVRSTKLRMVTGLRN | | | |
| | IPSIQSRGLFGAIAGFIEGGWTGMMDGWYGYH | | | |
| | HQNEQGSGYAADQKSTQNAINGITNKVNSVIE | | | |
| | KMNTQFTAVGKEFNKLERRMENLNKKVDDGFL | | | |
| | DIWTYNAELLVLLENERTLDFHDSNVKNLYEK | | | |
| | VKSQLKNNAKEIGNGCFEFYHKCNNECMESVK | | | |
| | NGTYDYPKYSEESKLNREKIDGVKLES | | | |
| A/Puerto Rico/ | ADTICIGYHANNSTDTVDTVLEKNVTVTHSVN | 8 | MGIYQILAIYSTV | 49 |
| 8/1934 | LLEDSHNGKLCRLKGIAPLQLGKCNIAGWLLG | | ASSLVLLVSLGAI | |
| A_/_H1N1 | NPECDPLLPVRSWSYIVETPNSFNGICYPGDF | | SFWMCSNGSLQCR | |
| | IDYEELREQLSSVSSFERFEIFPKESSWPNHN | | ICI | |
| | TNKGVTAACSHEGKSSFYRNLLWLTEKEGSYP | | | |
| | KLKNSYVNKKGKEVLVLWGIHHPPNSKEQQNL | | | |
| | YQNENAYVSVVTSNYNRRFTPEIAERPKVRDQ | | | |
| | AGRMNYYWTLLKPGDTIIFEANGNLIAPMYAF | | | |
| | ALSRGFGSGIITSNASMHECNTKCQTPLGAIN | | | |
| | SSLPYQNIHPVTIGECPKYVRSAKLRMVTGLR | | | |
| | NIPSIQSRGLFGAIAGFIEGGWTGMIDGWYGY | | | |
| | HHQNEQGSGYAADQKSTQNAINGITNKVNTVI | | | |
| | EKMNIQFTAVGKEFNKLEKRMENLNKKVDDGF | | | |
| | LDIWTYNAELLVLLENERTLDFHDSNVKNLYE | | | |
| | KVKSQLKNNAKEIGNGCFEFYHKCDNECMESV | | | |
| | RNGTYDYPKYSEESKLNREKVDGVKLES | | | |
| A/California/ | ADTLCIGYHANNSTDTVDTVLEKNVTVTHSVN | 9 | TRIYQILAIYSTV | 50 |
| 07/2009 | LLEDKHNGKLCKLRGVAPLHLGKCNIAGWILG | | ASSLVLVVSLGAI | |
| A_/_H1N1 | NPECESLSTASSWSYIVETPSSDNGTCYPGDF | | SFWMCSNGSLQCR | |
| | IDYEELREQLSSVSSFERFEIFPKTSSWPNHD | | ICI | |
| | SNKGVTAACPHAGAKSFYKNLIWLVKKGNSYP | | | |
| | KLSKSYINDKGKEVLVLWGIHHPSTSADQQSL | | | |
| | YQNADAYVFVGSSRYSKKFKPEIAIRPKVRDQ | | | |
| | EGRMNYYWTLVEPGDKITFEATGNLVVPRYAF | | | |
| | AMERNAGSGIIISDTPVHDCNTTCQTPKGAIN | | | |
| | TSLPFQNIHPITIGKCPKYVKSTKLRLATGLR | | | |
| | NIPSIQSRGLFGAIAGFIEGGWTGMVDGWYGY | | | |
| | HHQNEQGSGYAADLKSTQNAIDEITNKVNSVI | | | |
| | EKMNTQFTAVGKEFNHLEKRIENLNKKVDDGF | | | |
| | LDIWTYNAELLVLLENERTLDYHDSNVKNLYE | | | |
| | KVRSQLKNNAKEIGNGCFEFYHKCDNTCMESV | | | |
| | KNGTYDYPKYSEEAKLNREEIDGVKLES | | | |
| swine/Denmark/ | ADTICVGYHANNSTDTVDTILEKNVTVTHSVN | 10 | MGVHQILAIYSTV | 131 |
| 12813-1/2004 | LLESNHNGKLCSLNGKAPLQLGNCNVAGWILG | | ASSLVLLVSLGAI | |
| A_/_H1N1 | NPECDLLLTANSWSYIIETSNSKNGACYPGEF | | SFWMCSNGSLQCR | |
| | ADYEELRELLSTVSSFERFEIFPKATSWPHHE | | VCI | |
| | TTKGTTVACPHSGVNSFYRNLLWIVKKGNSYP | | | |
| | KLSKSYTNSKGKEVLVIWGVHHPPTDSDQQTL | | | |
| | YQNNHTYVSVGTSKYYRRFTPEIVARPKIREQ | | | |
| | AGRMNYYWTLLDQGDTITLEATGNLIVPWHAF | | | |
| | ALNKGSNSGIMISDADFHNCTTKCQTPHGALK | | | |
| | SNLPFQNVHPITIGECPKYVKSTQLRMATGLR | | | |
| | NVPSIQSRGLFGAIAGFIEGGWTGMIDGWYGY | | | |
| | HHQNEQGSGYAADQKSTQIAIDGISNKVNSVI | | | |
| | EKMNIQFASVGKEFNDLEKRIENLNKKVDDGF | | | |
| | VDVWTYNAELLILLENERTLDFHDENVKNLYE | | | |
| | KVKSQLRNNAKEIGNGCFEFYHKCDNECMESV | | | |
| | KNGTYNYPKYSEESKLNREEIDGVKLES | | | |
| A/swine/Eire/ | ADTICIGYHANNSTDTVDTVLEKNVTVTHSVN | 11 | MGVYQILAIYSTV | 132 |
| 89/1996.1996 | LLENNHNGKLCNLNGIAPLQLGKCNVAGWLLG | | ASSLVLLVSLGAI | |
| A_/_H1N1 | NPECGLLLNANAWSYIIETSDSKNGTCYPGDF | | SFWMCSNGSLQCR | |
| | IDYEELREQLGSVSSFEKFEIFPKASSWPDHE | | ICI | |
| | TTKGTTAACPYSGVESFYRNLLWIIKKGNSYP | | | |
| | KISKSYTNNRGKEVLVLWGVHHPPTTSDQQTL | | | |
| | YQNIDAYVSVGSSKYNRRFTPEIATRPKVRGL | | | |
| | AGRMNYYWTLLDQGDTIMFEATGNLIAPWYAF | | | |
| | ALNKGSDSGIITSDTPVHDCDTKCQTPYGALN | | | |
| | SSLPFQNVHPITIGECPKYVKSTKLRMATGLR | | | |
| | NVPSIQSKGLFGAIAGFIEGGWTGMIDGWYGY | | | |
| | HHQNEQGSGYSADQKSTQNAIDGITNKVNSVI | | | |
| | EKMNTQFTAVGKEFNNLERRIENLNKKVDDGF | | | |
| | LDVWTYNAELLVLLENERTLDFHDSNVRDLYE | | | |
| | RVKSQLRNNAKEIGNGCFEFYHKCDDECMENV | | | |
| | KNGTYDYPKYSEEAKLNREKIDGVKIES | | | |
| A/Thailand/ | ADTLCIGYHANNSTDTVDTILEKNVTVTHSVN | 12 | TRLYQILAIYSTA | 133 |
| 271/2005.2005/ | LLEDRHNGKLCNLRGEAPLHLGKCNIAGWLLG | | ASSLVLLVSLGAI | |
| 07 A_/_H1N1 | NPECELLFAVNSWSYIVETSNSDNGTCYPGDF | | GFWMCSNGSLQCR | |
| | TSYEELREQLSSVSSFERFEIFPKASSWPNHE | | ICI | |
| | TNRGVTAACPYAGTNSFYRNLIWLVKKGNSYP | | | |
| | KLSKSYVNNKKKEVLVLWGIHHPPTNADQQSL | | | |
| | YQNADAYVFVGSSKYNKKFKPEIAKRPKVRGQ | | | |
| | AGRMNYYWTLVEPGDTITFEATGNLVAPRYAF | | | |
| | AMNRDPGSGIITSDAPIHDCNATCQTPKGAIN | | | |
| | TSLPFQNIHPITIGECPKYVKSTRLRMATGLR | | | |
| | NIPSIQSRGLFGAIAGFIEGGWTGMIDGWYGY | | | |
| | HHQNGQGSGYAADQKSTQNAIDRITNKVNSVI | | | |
| | EKMNIQFTAVGKEFNHLERRIENLNKKVDDGF | | | |
| | LDVWTYNAELLVLLENERTLDFHDSNVKTLYE | | | |
| | KVKTQLRNNAKEIGNGCFEFYHKCDDTCMESI | | | |
| | KNGTYDYPKYSKESKLNREEIDGVQLES | | | |
| A/Hawaii/70/ | ADTLCIGYHANNSTDTVDTVLEKNVTVTHSVN | 13 | TRIYQILAIYSTV | 134 |
| 2019 A_/_H1N1 | LLEDKHNGKLCKLRGVAPLHLGKCNIAGWILG | | ASSLVLVVSLGAI | |
| | NPECESLSTARSWSYIVETSNSDNGTCYPGDF | | SFWMCSNGSLQCR | |
| | INYEELREQLSSVSSFERFEIFPKTSSWPNHD | | ICI | |
| | SDKGVTAACPHAGAKSFYKNLIWLVKKGNSYP | | | |
| | KLNQTYINDKGKEVLVLWGIHHPPTIAAQESL | | | |
| | YQNADAYVFVGTSRYSKKFKPEIATRPKVRDQ | | | |
| | EGRMNYYWTLVEPGDKITFEATGNLVVPRYAF | | | |
| | TMERDAGSGIIISDTPVHDCNTTCQTPEGAIN | | | |
| | TSLPFQNVHPITIGKCPKYVKSTKLRLATGLR | | | |
| | NVPSIQSRGLFGAIAGFIEGGWTGMVDGWYGY | | | |
| | HHQNEQGSGYAADLKSTQNAIDKITNKVNSVI | | | |
| | EKMNTQFTAVGKEFNHLEKRIENLNKKVDDGF | | | |
| | LDIWTYNAELLVLLENERTLDYHDSNVKNLYE | | | |
| | KVRNQLKNNAKEIGNGCFEFYHKCDNTCMESV | | | |
| | KNGTYDYPKYSEEAKLNREKIDGVKLES | | | |
| A/USSR/90/ | ADTICIGYHANNSTDTVDTVLEKNVTVTHSVN | 14 | MGVYQILAIYSTV | 135 |
| 1977 A_/_H1N1 | LLEDSHNGKLCRLKGIAPLQLGKCNIAGWILG | | ASSLVLLVSLGAI | |
| | NPECESLFSKKSWSYIAETPNSFNGTCYPGYE | | SFWMCSNGSLQCR | |
| | ADYEELREQLSSVSSFERFEIFPKERSWPKHN | | ICI | |
| | VTRGVTASCSHKGKSSFYRNLLWLTEKNGSYP | | | |
| | NLSKSYVNNKEKEVLVLWGVHHPSNIEDQKTI | | | |
| | YRKENAYVSVVSSNYNRRFTPEIAERPKVRGQ | | | |
| | AGRINYYWTLLEPGDTIIFEANGNLIAPWHAF | | | |
| | ALNRGFGSGIITSNASMDECDTKCQTPQGAIN | | | |
| | SSLPFQNIHPVTIGECPKYVRSTKLRMVTGLR | | | |
| | NIPSIQSRGLFGAIAGFIEGGWTGMIDGWYGY | | | |
| | HHQNEQGSGYAADQKSTQNAINGITNKVNSVI | | | |
| | EKMNTQFTAVGKEFNKLEKRMENLNKKVDDGF | | | |
| | LDIWTYNAELLVLLENERTLDFHDSNVKNLYE | | | |
| | KVKSQLKNNAKEIGNGCFEFYHKCNNECMESV | | | |
| | KNGTYDYPKYSEESKLNREKIDGVKLES | | | |
| A/Minnesota/ | ADTICIGYHANNSTDTVDTVLEKNVTVTHSVN | 15 | MGVYRILAIYSTV | 136 |
| 14/2012.2012/ | LLEDSHNGKLCLLKGIAPLQLGNCSVAGWILG | | ASSLVLLVSLGAI | |
| 12 A_/_H1N1 | NPECESLISKKSWSYIVETPNPENGACYPGEF | | SFWMCSNGSLQ | |
| | ADYEELREQLSSVSSFERFEIFPKESSWPNHT | | CRICI | |
| | ATGESASCSHNGKKSFYRNLIWLTVKNGLYPN | | | |
| | LSKSYENDKEKEVLILWGVHHPPNIENQRTLY | | | |
| | HTENAYVSVVSSHYSGRETPEITKRPKVRDQE | | | |
| | GRINYYWTLLEPGDTIIFEANGNLIAPWYAFA | | | |
| | LSRGLGSGIITSNAPMDECDSKCQTPQGAINS | | | |
| | SLPFQNVHPVTIGECPKYVRSAKLRMVTGLRN | | | |
| | IPSIQSRGLFGAIAGFIEGGWTGMVDGWYGYH | | | |
| | HQNEQGSGYAADQESTQNAINGITNKVNSVIE | | | |
| | KMNTQFTAVGKEFNKLERRMENLNKKVDDGFL | | | |
| | DIWTYNAELLVLLENERTLDFHDSNVKSLYEK | | | |
| | VKSQLKNNAKEIGNGCFEFYHKCNNECMESVK | | | |
| | NGTYDYPKYYEESKLNREKIDGVKLDS | | | |
| A/Indiana/11/ | TATLCLGHHAVPNGTIVKTITNDRIEVTNATE | 16 | GYKDWILWISFAI | 51 |
| 2018 A_/_H3N2 | LVQNSSIGEICDSPHQILDGENCTLIDALLGD | | SCFLLCVVLLGFI | |
| | PQCDGFQNKKWDLFVERSKAYSNCYPYDVPDY | | MWACQKGNIRCNI | |
| | ASLRSLVASSGTLEFNNESFNWTGVKQNGTSS | | CI | |
| | ACIRKSSSSFFSRLNWLTHLNYTYPALNVTMP | | | |
| | NNEQFDKLYIWGVHHPGTDKDQIFLYAQSSGR | | | |
| | ITVSTKRSQQAVIPNIGSRPRIRDIPSRISIY | | | |
| | WTIVKPGDILLINSTGNLIAPRGYFKIQSGKS | | | |
| | SIMRSDAPIGKCKSECITPNGSIPNDKPFQNV | | | |
| | NRITYGACPRYVKHSTLKLATGMRNVPEKQTR | | | |
| | GIFGAIAGFIENGWEGMVDGWYGFRHQNSEGR | | | |
| | GQAADLKSTQAAIDQINGKLNRLIGKTNEKFH | | | |
| | QIEKEFSEVEGRIQDLEKYVEDTKIDLWSYNA | | | |
| | ELLVALENQHTIDLTDSEMNKLFEKTKKQLRE | | | |
| | NAEDMGNGCFKIYHKCDNACIGSIRNGTYDHN | | | |
| | VYRDEALNNRFQIKGVELKS | | | |
| A/California/ | TATLCLGHHAVPNGTIVKTITNDQIEVTNATE | 17 | GYKDWILWISFAI | 52 |
| 07/2004 | LVQSSSTGGICDSPHQILDGENCTLIDALLGD | | SCFLLCVVLLGFI | |
| A_/_H3N2 | PQCDGFQNKKWDLFVERSKAYSNCYPYDVPDY | | MWACQKGNIRCNI | |
| | ASLRSLVASSGTLEFNNESFNWTGVTQNGTSS | | CI | |
| | SCKRRSNNSFFSRLNWLTHLKFKYPALNVTMP | | | |
| | NNEKFDKLYIWGVHHPGTNNDQISLYTQASGR | | | |
| | ITVSTKRSQQTVIPNIGSRPRVRDIPSRISIY | | | |
| | WTIVKPGDILLINSTGNLIAPRGYFKIRSGKS | | | |
| | SIMRSDAPIGKCNSECITPNGSIPNDKPFQNV | | | |
| | NRITYGACPRYVKQNTLKLATGMRNVPEKQTR | | | |
| | GIFGAIAGFIENGWEGMVDGWYGFRHQNSEGI | | | |
| | GQAADLKSTQAAINQINGKLNRLIGKTNEKFH | | | |
| | QIEKEFSEVEGRIQDLEKYVEDTKIDLWSYNA | | | |
| | ELLVALENQHTIDLTDSEMNKLFERTKKQLRE | | | |
| | NAEDMGNGCFKIYHKCDNACIGSIRNGTYDHD | | | |
| | VYRDEALNNRFQIKGVELKS | | | |
| A/Alaska/01/ | TATLCLGHHAVPNGTIVKTITNDRIEVTNATE | 18 | GYKDWILWISFAI | 53 |
| 2021 A_/_H3N2 | LVQNSSIGEICNSPHQILDGGNCTLIDALLGD | | SCFLLCVVLLGFI | |
| | PQCDGFQNKEWDLFVERSRANSSCYPYDVPDY | | MWACQKGNIRCNI | |
| | ASLRSLVASSGTLEFKNESFNWTGVKQNGTSS | | CI | |
| | ACIRGSSSSFFSRLNWLTSLNNIYPAQNVTMP | | | |
| | NKEQFDKLYIWGVHHPDTDKNQFSLFAQSSGR | | | |
| | ITVSTKRSQQAVIPNIGSRPRIRDIPSRISIY | | | |
| | WTIVKPGDILLINSTGNLIAPRGYFKIRSGKS | | | |
| | SIMRSDAPIGKCKSECITPNGSIPNDKPFQNV | | | |
| | NRITYGACPRYVKQSTLKLATGMRNVPEKQTR | | | |
| | GIFGAIAGFIENGWEGMVDGWYGFRHQNSEGR | | | |
| | GQAADLKSTQAAIDQISGKLNRLIGKTNEKFH | | | |
| | QIEKEFSEVEGRVQDLEKYVEDTKIDLWSYNA | | | |
| | ELLVALENQHTIDLTDSEMNKLFEKTKKQLRE | | | |
| | NAEDMGNGCFKIYHKCDNACIGSIRNETYDHN | | | |
| | VYRDEALNNRFQIKGVELKS | | | |
| A/Cambodia/ | TATLCLGHHAVPNGTIVKTITNDRIEVTNATE | 19 | GYKDWILWISFAI | 54 |
| e0826360/2020 | LVQNSSIGEICDSPHQILDGGNCTLIDALLGD | | SCFLLCVVLLGFI | |
| A_/_H3N2 | PQCDGFQNKEWDLEVERSRANSNCYPYDVPDY | | MWACQKGNIRCNI | |
| | ASLRSLVASSGTLEFKNESFNWTGVKQNGTSS | | CI | |
| | ACIRGSSSSFFSRLNWLTHLNYTYPALNVTMP | | | |
| | NNEQFDKLYIWGVHHPSTDKDQISLFAQPSGR | | | |
| | ITVSTKRSQQAVIPNIGSRPRIRDIPSRISIY | | | |
| | WTIVKPGDILLINSTGNLIAPRGYFKIRSGKS | | | |
| | SIMRSDAPIGKCKSECITPNGSIPNDKPFQNV | | | |
| | NRITYGACPRYVKQSTLKLATGMRNVPEKQTR | | | |
| | GIFGAIAGFIENGWEGMVDGWYGFRHQNSEGR | | | |
| | GQAADLKSTQAAIDQINGKLNRLIGKTNEKFH | | | |
| | QIEKEFSEVEGRVQDLEKYVEDTKIDLWSYNA | | | |
| | ELLVALENQHTIDLTDSEMNKLFEKTKKQLRE | | | |
| | NAEDMGNGCFKIYHKCDNACIGSIRNETYDHN | | | |
| | VYRDEALNNRFQIKGVELKS | | | |
| A/Nanchang/ | TATLCLGHHAVPNGTLVKTITNDQIEVTNATE | 20 | GYKDWILWISFAI | 55 |
| 933/1995 | LVQSSSTGRICDSPHRILDGKNCTLIDALLGD | | SCFLLCVVLLGFI | |
| A_/_H3N2 | PHCDGFQNKEWDLFVERSKAYSNCYPYDVPDY | | MWACQKGNIRCNI | |
| | ASLRSLVASSGTLEFTNEGFNWTGVAQDGTSY | | CI | |
| | ACKRGSVKSFFSRLNWLHKLEYKYPALNVTMP | | | |
| | NNDKFDKLYIWGVHHPSTDSDQTSLYVQASGR | | | |
| | VTVSTKRSQQTVIPNIGSRPWVRGISSRISIY | | | |
| | WTIVKPGDILLIKSTGNLIAPRGYFKIRSGKS | | | |
| | SIMRSDAPIGNCNSECITPNGSIPNDKPFQNV | | | |
| | NRITYGACPRYVKQNTLKLATGMRNVPEKQTR | | | |
| | GIFGAIAGFIENGWEGMVDGWYGFRHQNSEGT | | | |
| | GQAADLKSTQAAINQINGKLNRLIEKTNEKFH | | | |
| | QIEKEFSEVEGRIQDLEKYVEDTKIDLWSYNA | | | |
| | ELLVALENQHTIDLTDSEMNKLFERTRKQLRE | | | |
| | NAEDMGNGCFKIYHKCDNACIGSIRNGTYDHD | | | |
| | VYRDEALNNRFQIKGVELKS | | | |
| A/Memphis/1/ | TATLCLGHHAVPNGTLVKTITNDQIEVTNATE | 21 | GYKDWILWISFAI | 56 |
| 1980 A_/_H3N2 | LVQSSSTGRICDSPHRILDGKNCTLVDALLGD | | SCFLLCVVLLGFI | |
| | PHCDGFQNEKWDLFVERSKAFSNCYPYDVPDY | | MWACQKGNIRCNI | |
| | ASLRSLVASSGTLEFINESFNWTGVTQSGGSY | | CI | |
| | ACKRGSDNSFFSRLNWLYESESKYPVLNVTMP | | | |
| | NNGNEDKLYIWGVHHPSTDKEQTNLYVRASGR | | | |
| | VTVSTKRSQQTIIPNIGSRPWVRGLSSRISIY | | | |
| | WTIVKPGDILLINSNGNLIAPRGYFKIRTGKS | | | |
| | SIMRSDAPIGTCSSECITPNGSIPNDKPFQNV | | | |
| | NKITYGACPKYVKQNTLKLATGMRNVPEKQTR | | | |
| | GIFGAIAGFIENGWEGMVDGWYGFRHQNSEGT | | | |
| | GQAADLKSTQAAIDQINGKLNRVIEKTNEKFH | | | |
| | QIEKEFSEVEGRIQDLEKYVEDTKIDLWSYNA | | | |
| | ELLVALENQHTIDLTDSEMNKLFEKTRRQLRE | | | |
| | NAEDMGNGCFKIYHKCDNACIGSIRNGTYDHD | | | |
| | VYRDEALNNRFQIKGVELKS | | | |
| A/Hong Kong/ | TATLCLGHHAVPNGTLVKTITDDQIEVTNATE | 22 | GYKDWILWISFAI | 57 |
| 1/1968 | LVQSSSTGKICNNPHRILDGIDCTLIDALLGD | | SCFLLCVVLLGFI | |
| A_/_H3N2 | PHCDVFQNETWDLEVERSKAFSNCYPYDVPDY | | MWACQKGNIRCNI | |
| | ASLRSLVASSGTLEFITEGFTWTGVTQNGGSN | | CI | |
| | ACKRGPGSGFFSRLNWLTKSGSTYPVLNVTMP | | | |
| | NNDNEDKLYIWGVHHPSTNQEQTSLYVQASGR | | | |
| | VTVSTRRSQQTIIPNIGSRPWVRGLSSRISIY | | | |
| | WTIVKPGDVLVINSNGNLIAPRGYFKMRTGKS | | | |
| | SIMRSDAPIDTCISECITPNGSIPNDKPFQNV | | | |
| | NKITYGACPKYVKQNTLKLATGMRNVPEKQTR | | | |
| | GLFGAIAGFIENGWEGMIDGWYGFRHQNSEGT | | | |
| | GQAADLKSTQAAIDQINGKLNRVIEKTNEKFH | | | |
| | QIEKEFSEVEGRIQDLEKYVEDTKIDLWSYNA | | | |
| | ELLVALENQHTIDLTDSEMNKLFEKTRRQLRE | | | |
| | NAEDMGNGCFKIYHKCDNACIESIRNGTYDHD | | | |
| | VYRDEALNNRFQIKGVELKS | | | |
| A/Indiana/08/ | ATLCLGHHAVPNGTLVKTITDDQIEVTNATEL | 23 | GYKDWILWISFAI | 137 |
| 2011 A_/_H3N2 | VQSSSTGGICNSPHQILDGKNCTLIDALLGDP | | SCFLLCVVLLGFI | |
| | HCDDFQNKEWDLEVERSTAYSNCYPYYVPDYA | | MWACQKGNIRCNI | |
| | TLRSLVASSGNLEFTQESFNWTGVAQGGSSYA | | CI | |
| | CRRGSVNSFFSRLNWLYNLNYKYPEQNVTMPN | | | |
| | NDKFDKLYIWGVHHPGTDKDQTNLYVQASGRV | | | |
| | IVSTKRSQQTVIPNIGSRPWVRGVSSIISIYW | | | |
| | TIVKPGDILLINSTGNLIAPRGYFKIQSGKSS | | | |
| | IMRSDAHIDECNSECITPNGSIPNDKPFQNVN | | | |
| | KITYGACPRYVKQNTLKLATGMRNVPEKQTRG | | | |
| | IFGAIAGFIENGWEGMVDGWYGFRHQNSEGTG | | | |
| | QAADLKSTQAAINQITGKLNRVIKKTNEKFHQ | | | |
| | IEKEFSEVEGRIQDLEKYVEDTKIDLWSYNAE | | | |
| | ILVALENQHTIDLTDSEMSKLFERTRRQLREN | | | |
| | AEDMGNGCFKIYHKCDNACIGSIRNGTYDHDI | | | |
| | YRNEALNNRFQIKGVQLKS | | | |
| A/Bilthoven/ | TATLCLGHHAVPNGTLVKTITNDQIEVTNATE | 24 | GYKDWILWISFAI | 58 |
| 1761/1976 | LVQSSSTGKICDNPHRILDGINCTLIDALLGD | | SCFLLCVVLLGFI | |
| A_/_H3N2 | PHCDGFQNEKWDLFVERSKAFSNCYPYDVPDY | | MWACQKGNIRCNI | |
| | ASLRSLVASSGTLEFINEGFNWTGVTQNGGSS | | CI | |
| | ACKRGPDNGFFSRLNWLYKSGSTYPVQNVTMP | | | |
| | NNDNSDKLYIWGVHHPSTDKEQTDLYVQASGK | | | |
| | VTVSTKRSQQTVIPNVGSRPWVRGLSSRVSIY | | | |
| | WTIVKPGDILVINSNGNLIAPRGYFKMRTGKS | | | |
| | SIMRSDAPIGTCSSECITPNGSIPNDKPFQNV | | | |
| | NKITYGACPKYVKQNTLKLATGMRNVPEKQTR | | | |
| | GIFGAIAGFIENGWEGMIDGWYGFRHQNSEGT | | | |
| | GQAADLKSTQAAIDQINGKLNRVIEKTNEKFH | | | |
| | QIEKEFSEVEGRIQDLEKYVEDTKIDLWSYNA | | | |
| | ELLVALENQHTIDLTDSEMNKLFEKTRRQLRE | | | |
| | NAEDMGNGCFKIYHKCDNACIGSIRNGTYDHD | | | |
| | VYRDEALNNRFQIKGVELKS | | | |
| A/Victoria/ | TATLCLGHHAVPNGTIVKTITNDQIEVTNATE | 25 | GYKDWILWISFAI | 138 |
| 361/2011 | LVQNSSIGEICDSPHQILDGENCTLIDALLGD | | SCFLLCVALLGFI | |
| A_/_H3N2 | PQCDGFQNKKWDLEVERSKAYSNCYPYDVPDY | | MWACQKGNI | |
| | ASLRSLVASSGTLEFNNESFNWTGVTQNGTSS | | RCNICI | |
| | ACIRRSNNSFFSRLNWLTHLNFKYPALNVTMP | | | |
| | NNEQFDKLYIWGVHHPGTDKDQIFLYAQSSGR | | | |
| | ITVSTKRSQQAVIPNIGSRPRIRNIPSRISIY | | | |
| | WTIVKPGDILLINSTGNLIAPRGYFKIRSGKS | | | |
| | SIMRSDAPIGKCNSECITPNGSIPNDKPFQNV | | | |
| | NRITYGACPRYVKQSTLKLATGMRNVPEKQTR | | | |
| | GIFGAIAGFIENGWEGMVDGWYGFRHQNSEGR | | | |
| | GQAADLKSTQAAIDQINGKLNRLIGKTNEKFH | | | |
| | QIEKEFSEVEGRIQDLEKYVEDTKIDLWSYNA | | | |
| | ELLVALENQHTIDLTDSEMNKLFEKTKKQLRE | | | |
| | NAEDMGNGCFKIYHKCDNACIGSIRNGTYDHD | | | |
| | VYRDEALNNRFQIKGVELKS | | | |
| A/Brisbane/ | TATLCLGHHAVPNGTIVKTITNDQIEVTNATE | 26 | GYKDWILWISFAI | 139 |
| 10/2007 | LVQSSSTGEICDSPHQILDGENCTLIDALLGD | | SCFLLCVALLGFI | |
| A_/_H3N2 | PQCDGFQNKKWDLFVERSKAYSNCYPYDVPDY | | MWACQKGNIRCNI | |
| | ASLRSLVASSGTLEFNNESFNWTGVTQNGTSS | | CI | |
| | ACIRRSNNSFFSRLNWLTHLKFKYPALNVTMP | | | |
| | NNEKFDKLYIWGVHHPGTDNDQIFLYAQASGR | | | |
| | ITVSTKRSQQTVIPNIGSRPRVRNIPSRISIY | | | |
| | WTIVKPGDILLINSTGNLIAPRGYFKIRSGKS | | | |
| | SIMRSDAPIGKCNSECITPNGSIPNDKPFQNV | | | |
| | NRITYGACPRYVKQNTLKLATGMRNVPEKQTR | | | |
| | GIFGAIAGFIENGWEGMVDGWYGFRHQNSEGI | | | |
| | GQAADLKSTQAAIDQINGKLNRLIGKTNEKFH | | | |
| | QIEKEFSEVEGRIQDLEKYVEDTKIDLWSYNA | | | |
| | ELLVALENQHTIDLTDSEMNKLFEKTKKQLRE | | | |
| | NAEDMGNGCFKIYHKCDNACIGSIRNGTYDHD | | | |
| | VYRDEALNNRFQIKGVELKS | | | |
| A/Maryland/ | TATLCLGHHAVPNGTIVKTITNDRIEVTNATE | 27 | GYKDWILWISFAM | 140 |
| 02/2021 | LVQNSSIGEICGSPHQILDGGNCTLIDALLGD | | SCELLCIALLGFI | |
| A_/_H3N2 | PQCDGFQNKEWDLFVERSRANSNCYPYDVPGY | | MWACQKGNIRCNI | |
| | ASLRSLVASSGTLEFKNESFNWTGVKQNGTSS | | CI | |
| | ACIRGSSSSFFSRLNWLTSINNIYPAQNVTMP | | | |
| | NKEQFDKLYIWGVHHPDTDKNQISLFAQSSGR | | | |
| | ITVSTKRSQQAVIPNIGSRPRIRDIPSRISIY | | | |
| | WTIVKPGDILLINSTGNLIAPRGYFKIRNGKS | | | |
| | SIMRSDAPIGRCKSECITPNGSIPNDKPFQNV | | | |
| | NRITYGACPRYVKQSTLKLATGMRNVPEKQTR | | | |
| | GIFGAIAGFIENGWEGMVDGWYGFRHQNSEGR | | | |
| | GQAADLKSTQAAIDQINGKLNRLIGKTNEKFH | | | |
| | QIEKEFSEVEGRVQDLEKYVEDTKIDLWSYNA | | | |
| | ELLVALENQHTIDLTDSEMNKLFEKTKKQLRE | | | |
| | NAEDMGNGCFKIYHKCDNACIGSIRNETYDHN | | | |
| | VYRDEALNNRFQIKGVELKS | | | |
| A/Sydney/5/ | TATLCLGHHAVPNGTLVKTITNDQIEVTNATE | 28 | GYKDWILWISFAI | 141 |
| 1997 A_/_H3N2 | LVQSSSTGRICDSPHRILDGENCTLIDALLGD | | SCFLLCVVLLGFI | |
| | PHCDGFQNKEWDLFVERSKAYSNCYPYDVPDY | | MWACQKGNIRCNI | |
| | ASLRSLVASSGTLEFNNESFNWTGVAQNGTSY | | CI | |
| | ACKRSSIKSFFSRLNWLHQLKYKYPALNVTMP | | | |
| | NNDKEDKLYIWGVHHPSTDSDQTSIYAQASGR | | | |
| | VTVSTKRSQQTVIPNIGSRPWVRGISSRISIH | | | |
| | WTIVKPGDILLINSTGNLIAPRGYFKIRSGKS | | | |
| | SIMRSDAPIGKCNSECITPNGSIPNDKPFQNV | | | |
| | NRITYGACPRYVKQNTLKLATGMRNVPEKQTR | | | |
| | GIFGAIAGFIENGWEGMVDGWYGFRHQNSEGT | | | |
| | GQAADLKSTQAAINQINGKLNRLIEKTNEKFH | | | |
| | QIEKEFSEVEGRIQDLEKYVEDTKIDLWSYNA | | | |
| | ELLVALENQHTIDLTDSEMNKLFERTRKQLRE | | | |
| | NAEDMGNGCFKIYHKCDNACIGSIRNGTYDHD | | | |
| | VYRDEALNNRFQIKGVELKS | | | |
| B_/_HABB/ | ADRICTGITSSNSPHVVKTATQGEVNVTGVIP | 29 | LPTFDSLNITAAS | 142 |
| Guangdong- | LTTTPTKSHFANLKGTETRGKLCPKCLNCTDL | | LNDDGLDNHTILL | |
| Maonan/316/ | DVALGRPKCTGKIPSAKVSILHEVRPVTSGCF | | YYSTAASSLAVTL | |
| 2021 | PIMHDRTKIRQLPNLLRGYEHVRLSTNNVINA | | MIAIFVVYMVSRD | |
| | EDAPGGPYEIGTSGSCPNITNGKGFFATMAWA | | NVSCSICL | |
| | VPKNKTATNPLTIEVPYICTEEEDQITVWGFH | | | |
| | SDDETQMAKLYGDSKPQKFTSSANGMTTHYVS | | | |
| | QIGGFPNQTEDGGLQQSGRIVVDYMVQKSGKT | | | |
| | GTITYQRGILLPQKVWCASGKSKVIKGSLPLI | | | |
| | GEADCLHEKYGGLNKSKPYYTGEHAKAIGNCP | | | |
| | IWVKTPLKLANGTKYRPPAKLLKERGFFGAIA | | | |
| | GFLEGGWEGMIAGWHGYTSHGAHGVAVAADLK | | | |
| | STQEAINKITKNLNSLSELEVKNLQRLSGAMD | | | |
| | ELHNEILELDEKVDDLRADTISSQIELAVLLS | | | |
| | NEGIINSEDEHLLALERKLKKMLGPSAVEIGN | | | |
| | GCFETKHKCNQTCLDRIAAGTEDAGEFS | | | |
| B_/_HABB/ | ADRICTGITSSNSPHVVKTATQGEVNVTGVIP | 30 | LPTEDSLNITAAS | 143 |
| Ann_Arbor/ | LTTTPTKSHFANLKGTKTRGKLCPKCLNCTDL | | LNDDGLDNHTILL | |
| 1/1986 | DVALGRPKCMGTIPSAKASILHEVKPVTSGCF | | YYSTAASSLAVTL | |
| | PIMHDRTKIRQLPNLLRGYENIRLSTHNVINA | | MIAIFIVYMVSRD | |
| | EAAPGGPYIVGTSGSCPNVTNGNGEFATMAWA | | NVSCSICL | |
| | VPKNNNNKTATNPLTVEVPFICTEGEDQITVW | | | |
| | GFHSDNEIQMVKLYGDSKPQKFTSSANGVTTH | | | |
| | YVSQIGGFPKQAEDGGLPQSGRIVVDYMVQKS | | | |
| | GKTGTITYQRGILLPQKVWCASGRSKVIKGSL | | | |
| | PLIGEADCLHEKYGGLNKSKPYYTGEHAKAIG | | | |
| | NCPIWVKTPLKLANGTKYRPPAKLLKERGEFG | | | |
| | AIAGFLEGGWEGMIAGWHGYTSHGAHGVAVAA | | | |
| | DLKSTQEAINKITKNLNSLSELEVKNLQRLSG | | | |
| | AMDELHNEILELDEKVDDLRADTISSQIELAV | | | |
| | LLSNEGIINSEDEHLLALERKLKKMLGPSAVD | | | |
| | IGNGCFETKHKCNQTCLDRIAAGTENAGEF | | | |
| B_/_HABB/ | ADRICTGITSSNSPHVVKTATQGEVNVTGVIP | 31 | LPTFDSLNITAAS | 144 |
| Utah/02/ | LTTTPTKSYFANLKGTRTRGKLCPDCLNCTDL | | LNDDGLDNHTILL | |
| 2012 | DVALGRPMCVGTTPSAKASILHEVRPVTSGCF | | YYSTAASSLAVTL | |
| | PIMHDRTKIRQLPNLLRGYEKIRLSTQNVIDA | | MLAIFIVYMVSRD | |
| | EKAPGGPYRLGTSGSCPNATSKIGFFATMAWA | | NVSCSICL | |
| | VPKDNYKNATNPLTVEVPYICTEGEDQITVWG | | | |
| | FHSDNKTQMKSLYGDSNPQKFTSSANGVTTHY | | | |
| | VSQIGDFPDQTEDGGLPQSGRIVVDYMMQKPG | | | |
| | KTGTIVYQRGVLLPQKVWCASGRSKVIKGSLP | | | |
| | LIGEADCLHEEYGGLNKSKPYYTGKHAKAIGN | | | |
| | CPIWVKTPLKLANGTKYRPPAKLLKERGFFGA | | | |
| | IAGFLEGGWEGMIAGWHGYTSHGAHGVAVAAD | | | |
| | LKSTQEAINKITKNLNSLSELEVKNLQRLSGA | | | |
| | MDELHNEILELDEKVDDLRADTISSQIELAVL | | | |
| | LSNEGIINSEDEHLLALERKLKKMLGPSAVDI | | | |
| | GNGCFETKHKCNQTCLDRIAAGTENAGEF | | | |
| B_/HABB/ | ADRICTGITSSNSPHVVKTATQGEVNVTGVIP | 32 | LPTEDSLNITAAS | 145 |
| Yamagata/ | LTTTPTKSHFANLKGTKTRGKLCPNCLNCTDL | | LNDDGLDNHTILL | |
| 16/1988 | DVALGRPMCMGTIPSAKASILHEVRPVTSGCF | | YYSTAASSLAVTL | |
| | PIMHDRTKIRQLPNLLRGYENIRLSTHNVINA | | MIAIFIVYMVSRD | |
| | ERAPGGPYRLGTSGSCPNVTSRNGFFATMAWA | | NVSCSICL | |
| | VPRDNKTATNPLTVEVPYICTKGEDQITVWGE | | | |
| | HSDDKTQMKNLYGDSNPQKFTSSANGVTTHYV | | | |
| | SQIGDEPNQTEDGGLPQSGRIVVDYMVQKPGK | | | |
| | TGTIVYQRGVLLPQKVWCASGRSKVIKGSLPL | | | |
| | IGEADCLHEKYGGLNKSKPYYTGEHAKAIGNC | | | |
| | PIWVKTPLKLANGTKYRPPAKLLKERGFFGAI | | | |
| | AGFLEGGWEGMIAGWHGYTSHGAHGVAVAADL | | | |
| | KSTQEAINKITKNLNSLSELEVKNLQRLSGAM | | | |
| | DELHNEILELDEKVDDLRADTISSQIELAVLL | | | |
| | SNEGIINSEDEHLLALERKLKKMLGPSAVDIG | | | |
| | NGCFETKHKCNQTCLDRIAAGTENAGEF | | | |
| B_/_HABB/ | ADRICTGITSSNSPHVVKTATQGEVNVTGVIP | 33 | LPTEDSLNITAAS | 146 |
| Singapore/ | LTTTPTKSHFANLKGTETRGKLCPKCLNCTDL | | LNDDGLDNHTILL | |
| WUH0784/2021 | DVALGRPKCTGKIPSARVSILHEVRPVTSGCF | | YYSTAASSLAVTL | |
| | PIMHDRTKIRQLPNLLRGYEHVRLSTHNVINT | | MIAIFVVYMVSRD | |
| | EDAPGGPYEIGTSGSCLNITNGKGFFATMAWA | | NVSCSICL | |
| | VPKNKTATNPLTIEVPYICTEEEDQITVWGFH | | | |
| | SDDETQMVRLYGDSKPQKFTSSANGVTTHYVS | | | |
| | QIGGFPNQTEDGGLPQSGRIVVDYMVQKSGKT | | | |
| | GTITYQRGILLPQKVWCASGKSKVIKGSLPLI | | | |
| | GEADCLHEKYGGLNKSKPYYTGEHAKAIGNCP | | | |
| | IWVKTPLKLANGTKYRPPAKLLKERGFFGAIA | | | |
| | GFLEGGWEGMIAGWHGYTSHGAHGVAVAADLK | | | |
| | STQEAINKITKNLNSLSELEVKNLQRLSGAMD | | | |
| | ELHNEILELDEKVDDLRADTISSQIELAVLLS | | | |
| | NEGIINSEDEHLLALERKLKKMLGPSAVEIGN | | | |
| | GCFETKHKCNQTCLDRIAAGTEDAGEFS | | | |
| B_/_HABB/ | ADRICTGITSSNSPHVVKTATQGEVNVTGVIP | 34 | LPTFDSLNITAAS | 147 |
| Colorado/ | LTTTPTKSHFANLKGTETRGKLCPKCLNCTDL | | LNDDGLDNHTILL | |
| 06/2017 | DVALGRPKCTGKIPSARVSILHEVRPVTSGCF | | YYSTAASSLAVTL | |
| | PIMHDRTKIRQLPNLLRGYEHVRLSTHNVINA | | MIAIFVVYMVSRD | |
| | EGAPGGPYKIGTSGSCPNITNGNGFFATMAWA | | NVSCSICL | |
| | VPDKNKTATNPLTIEVPYVCTEGEDQITVWGF | | | |
| | HSDNETQMAKLYGDSKPQKFTSSANGVTTHYV | | | |
| | SQIGGFPNQTEDGGLPQSGRIVVDYMVQKSGK | | | |
| | TGTITYQRGILLPQKVWCASGRSKVIKGSLPL | | | |
| | IGEADCLHEKYGGLNKSKPYYTGEHAKAIGNC | | | |
| | PIWVKTPLKLANGTKYRPPAKLLKERGFFGAI | | | |
| | AGFLEGGWEGMIAGWHGYTSHGAHGVAVAADL | | | |
| | KSTQEAINKITKNLNSLSELEVKNLQRLSGAM | | | |
| | DELHNEILELDEKVDDLRADTISSQIELAVLL | | | |
| | SNEGIINSEDEHLLALERKLKKMLGPSAVEIG | | | |
| | NGCFETKHKCNQTCLDKIAAGTEDAGEFS | | | |
| B_/_HABB/ | ADRICTGITSSNSPHVVKTATQGEVNVTGVIP | 35 | LPTEDSLNITAAS | 148 |
| Brisbane/ | LTTTPTKSHFANLKGTETRGKLCPKCLNCTDL | | LNDDGLDNHTILL | |
| 60/2008 | DVALGRPKCTGKIPSARVSILHEVRPVTSGCF | | YYSTAASSLAVTL | |
| | PIMHDRTKIRQLPNLLRGYEHIRLSTHNVINA | | MIAIFVVYMVSRD | |
| | ENAPGGPYKIGTSGSCPNITNGNGFFATMAWA | | NVSCSICL | |
| | VPKNDKNKTATNPLTIEVPYICTEGEDQITVW | | | |
| | GFHSDNETQMAKLYGDSKPQKFTSSANGVTTH | | | |
| | YVSQIGGFPNQTEDGGLPQSGRIVVDYMVQKS | | | |
| | GKTGTITYQRGILLPQKVWCASGRSKVIKGSL | | | |
| | PLIGEADCLHEKYGGLNKSKPYYTGEHAKAIG | | | |
| | NCPIWVKTPLKLANGTKYRPPAKLLKERGFFG | | | |
| | AIAGFLEGGWEGMIAGWHGYTSHGAHGVAVAA | | | |
| | DLKSTQEAINKITKNLNSLSELEVKNLQRLSG | | | |
| | AMDELHNEILELDEKVDDLRADTISSQIELAV | | | |
| | LLSNEGIINSEDEHLLALERKLKKMLGPSAVE | | | |
| | IGNGCFETKHKCNQTCLDRIAAGTEDAGEFS | | | |
| B_/_HABB/ | ADRICTGITSSNSPHVVKTATQGEVNVTGVIP | 36 | LPTFDSLNITAAS | 149 |
| Washington/ | LTTTPTKSHFANLKGTETRGKLCPKCLNCTDL | | LNDDGLDNHTILL | |
| 02/2019 | DVALGRPKCTGKIPSARVSILHEVRPVTSGCF | | YYSTAASSLAVTL | |
| | PIMHDRTKIRQLPNLLRGYEHVRLSTHNVINA | | MIAIFVVYMVSRD | |
| | EDAPGRPYEIGTSGSCPNITNGNGFFATMAWA | | NVSCSICL | |
| | VPKNKTATNPLTIEVPYICTEGEDQITVWGFH | | | |
| | SDNETQMAKLYGDSKPQKFTSSANGVTTHYVS | | | |
| | QIGGFPNQTEDGGLPQSGRIVVDYMVQKSGKT | | | |
| | GTITYQRGILLPQKVWCASGRSKVIKGSLPLI | | | |
| | GEADCLHEKYGGLNKSKPYYTGEHAKAIGNCP | | | |
| | IWVKTPLKLANGTKYRPPAKLLKERGFFGAIA | | | |
| | GFLEGGWEGMIAGWHGYTSHGAHGVAVAADLK | | | |
| | STQEAINKITKNLNSLSELEVKNLQRLSGAMD | | | |
| | ELHNEILELDEKVDDLRADTISSQIELAVLLS | | | |
| | NEGIINSEDEHLLALERKLKKMLGPSAVEIGN | | | |
| | GCFETKHKCNQTCLDRIAAGTEDAGEFS | | | |
| B_/_HABB/ | ADRICTGITSSNSPHVVKTATQGEVNVTGVIP | 37 | LPTEDSLNITAAS | 150 |
| Florida/4/ | LTTTPTKSYFANLKGTRTRGKLCPDCLNCTDL | | LNDDGLDNHTILL | |
| 2006 | DVALGRPMCVGTTPSAKASILHEVKPVTSGCF | | YYSTAASSLAVTL | |
| | PIMHDRTKIRQLPNLLRGYENIRLSTQNVIDA | | MLAIFIVYMVSRD | |
| | EKAPGGPYRLGTSESCPSATSKSGFFATMAWA | | NVSCSICL | |
| | VPKDNNKNATNPLTVEVPYICTEGEDQITVWG | | | |
| | FHSDDKTQMKNLYGDSNPQKFTSSANGVTTHY | | | |
| | VSQIGSFPDQTEDGGLPQSGRIVVDYMMQKPG | | | |
| | KTGTIVYQRGVLLPQKVWCASGRSKVIKGSLP | | | |
| | LIGEADCLHEKYGGLNKSKPYYTGEHAKAIGN | | | |
| | CPIWVKTPLKLANGTKYRPPAKLLKERGFFGA | | | |
| | IAGFLEGGWEGMIAGWHGYTSHGAHGVAVAAD | | | |
| | LKSTQEAINKITKNLNSLSELEVKNLQRLSGA | | | |
| | MDELHNEILELDEKVDDLRADTISSQIELAVL | | | |
| | LSNEGIINSEDEHLLALERKLKKMLGPSAVEI | | | |
| | GNGCFETKHKCNQTCLDRIAAGTENAGEF | | | |
| A/Roma/1949 | ADTICIGYHANNSTDTVDTVLEKNVTVTHSVN | 38 | MGVYQILAIYSTV | 59 |
| | LLEDSHNGKLCRLKGIAPLQLGKCNIAGWILG | | ASSLVLLVSLGAI | |
| | NPECESLESKKSWSYIAETPNSFNGTCYPGYF | | SFWMCSNGSLQCR | |
| | ADYEELREQLSSVSSFERFEIFPKERSWPKHN | | ICI | |
| | VTRGVTAACSHKGKSSFYRNLLWLTEKDGSYP | | | |
| | NLSKSYVNNKEKEVLVLWGVHHPSNIEDQKTL | | | |
| | YRKENAYVSVVSSNYNRRFTPEIAERPKVRGQ | | | |
| | AGRINYYWTLLEPGDTIIFEANGNLIAPWHAF | | | |
| | ALSRGFGSGIITSNASMDECDTKCQTPQGAIN | | | |
| | SSLPFQNIHPVTIGECPKYVRSTKLRMVTGLR | | | |
| | NIPSIQSRGLFGAIAGFIEGGWTGMIDGWYGY | | | |
| | HHQNEQGSGYAADQKSTQNAINGITNKVNSVI | | | |
| | EKMNTQFTAVGKEFNKLEKRMENLNKKVDDGF | | | |
| | LDIWTYNAELLVLLENERTLDFHDSNVKNLYE | | | |
| | KVKSQLKNNAKEIGNGCFEFYHKCNNECMESV | | | |
| | KNGTYDYPKYSEESKLNREKIDGVKLES | | | |
| A/MD/12/1991 | ADTLCIGYHANNSTDTVDTVLEKNVTVTHSVN | 39 | TRIYQILAIYSTV | 60 |
| | LLEDRHNGKLCKLRGVAPLHLGKCNIAGWLLG | | ASSLVLLVSLGAI | |
| | NPECELLFTASSWSYIVETSNSDNGTCYPGDF | | SFWMCSNGSLQCR | |
| | INYEELREQLSSVSSFERFEIFPKASSWPDHE | | ICI | |
| | TNRGVTAACPYAGANSFYRNLIWLVKKGNSYP | | | |
| | KLSKSYVNNKEKEVLVLWGIHHPPTSTDQQSL | | | |
| | YQNADAYVFVGSSKYNKKFKPEIATRPKVRGQ | | | |
| | AGRMNYYWTLVEPGDTITFEATGNLVVPRYAF | | | |
| | AMKRGSGSGIIISDTPVHDCNTTCQTPKGAIN | | | |
| | TSLPFQNIHPVTIGECPKYVKSTKLRMATGLR | | | |
| | NIPSIQSRGLFGAIAGFIEGGWTGMIDGWYGY | | | |
| | HHQNEQGSGYAADQKSTQNAIDGITNKVNSVI | | | |
| | EKMNTQFTAVGKEFNHLEKRIENLNKKVDDGF | | | |
| | LDVWTYNAELLVLLENERTLDYHDSNVKNLYE | | | |
| | KVRSQLKNNAKEIGNGCFEFYHKCDDTCMESV | | | |
| | KNGTYDYPKYSEESKLNREEIDGVKLES | | | |
| A/Auckland/ | TATLCLGHHAVPNGTLVKTITNDQIEVTNATE | 40 | GYKDWILWISFAI | 61 |
| 588/2000 | LVQSSSTGGICDSPHQILDGENCTLIDALLGD | | SCFLLCVVLLGFI | |
| | PHCDGFQNKEWDLFVERSKAYSNCYPYDVPDY | | MWACQKGNIRCNI | |
| | ASLRSLVASSGTLEFNNESFNWTGVAQNGTSS | | CI | |
| | ACKRRSNKSFFSRLNWLHQLKYKYPALNVTMP | | | |
| | NNEKFDKLYIWGVHHPSTDSDQISLYAQAPGR | | | |
| | VTVSTKRSQQTVIPNIGSRPWVRGVSSRISIY | | | |
| | WTIVKPGDILLINCTGNLIAPRGYFKIRSGKS | | | |
| | SIMRSDASIGKCNSECITPNGSIPNDKPFQNV | | | |
| | NRITYGACPRYVKQNTLKLATGMRNVPEKQTR | | | |
| | GIFGAIAGFIENGWEGMVDGWYGFRHQNSEGT | | | |
| | GQAADLKSTQAAINQINGKLNRLIEKTNEKFH | | | |
| | QIEKEFSEVEGRIQDLEKYVEDTKIDLWSYNA | | | |
| | ELLVALENQHTIDLTDSEMNKLFERTKKQLRE | | | |
| | NAEDMGNGCFKIYHKCDNACIGSIRNGTYDHD | | | |
| | VYRDEALNNRFQIKGVELKS | | | |
| A/Argentina/ | TATLCLGHHAVPNGTIVKTITNDQIEVTNATE | 41 | GYKDWILWISFAI | 62 |
| 28302/2010 | LVQSSSTGEICDSPHQILDGKNCTLIDALLGD | | SCFLLCVVLLGFI | |
| | PQCDGFQNKKWDLFVERSKAYSNCYPYDVPDY | | MWACQKGNIRCNI | |
| | ASLRSLVASSGTLEFNNESFNWTGVTQDGTSS | | CI | |
| | ACIRGSKNSFFSRLNWLTHLNFKYPALNVTMP | | | |
| | NNEQFDKLYIWGVHHPGTDKDQIFLYAQASGR | | | |
| | ITVSTKRSQQAAIPNIGSRPRVRNIPSRISIY | | | |
| | WTIVKPGDILLINSTGNLIAPRGYFKIRSGKS | | | |
| | SIMRSDAPIGKCNSECITPNGSIPNDKPFQNV | | | |
| | NRITYGACPRYVKQNTLKLATGMRNVPEKQTR | | | |
| | GIFGAIAGFIENGWEGMVDGWYGFRHQNSEGR | | | |
| | GQAADLKSTQAAIDQINGKLNRLIGKTNEKFH | | | |
| | QIEKEFSEVEGRIQDLEKYVEDTKIDLWSYNA | | | |
| | ELLVALENQHTIDLTDSEMNKLFEKTKKQLRE | | | |
| | NAEDMGNGCFKIYHKCDNACIGSIRNGTYDHD | | | |
| | VYRDEALNKRFQIKGIELKS | | | |
| B/Austria/ | ADRICTGITSSNSPHVVKTATQGEVNVTGVIPLTTTPTKSHFANLKG | 42 | ILLYYSTAASSLAVTLMIAIF | 63 |
| 1359417/2021 | TETRGKLCPKCLNCTDLDVALGRPKCTGKIPSARVSILHEVRPVTSG | | VVYMVSRDNVSCSICL | |
| | CFPIMHDRTKIRQLPNLLRGYEHVRLSTHNVINTEDAPGGPYEIGTS | | | |
| | GSCLNITNGKGFFATMAWAVPKNKTATNPLTIEVPYICTEEEDQITV | | | |
| | WGFHSDDETQMARLYGDSKPQKFTSSANGVTTHYVSQIGGFPNQT | | | |
| | EDGGLPQSGRIVVDYMVQKSGKTGTITYQRGILLPQKVWCASGKS | | | |
| | KVIKGSLPLIGEADCLHEKYGGLNKSKPYYTGEHAKAIGNCPIWVK | | | |
| | TPLKLANGTKYRPPAKLLKERGFFGAIAGFLEGGWEGMIAGWHGY | | | |
| | TSHGAHGVAVAADLKSTQEAINKITKNLNSLSELEVKNLQRLSGA | | | |
| | MDELHNEILELDEKVDDLRADTISSQIELAVLLSNEGIINSEDEHLLA | | | |
| | LERKLKKMLGPSAVEIGNGCFETKHKCNQTCLDRIAAGTFDAGEFS | | | |
| | LPTFDSLNITAASLNDDGLDNHT | | | |

TABLE 2
Nucleic acid sequences for a vaccine composition

| SEQ | | |
|---|---|---|
| ID | | |
| NO: | Nucleic Acid Sequence | Strain |
| 64 | ATGAAAGTGAAGTTACTGGTCCTCCTTTGCACCTTTACCGCCACCTACGCTGACACCCTGTGCA | A/ |
| | TTGGCTATCATGCCAACAACTCCACAGACACCGTGGACACAGTGCTGGAGAAGAACGTGACAGT | Wisconsin/ |
| | GACCCACTCCGTGAACCTGCTGGAAGACAAGCACAACGGCAAACTGTGCAAGCTGAGAGGCGTG | 588/ |
| | GCCCCTCTGCACCTGGGCAAGTGCAACATCGCCGGCTGGATCCTGGGCAACCCAGAGTGCGAGA | 2019 |
| | GCCTGTCCACAGCCCGGTCCTGGTCTTACATTGTGGAGACATCCAACAGCGACAACGGCACCTG | |
| | CTACCCTGGCGACTTCATCAATTACGAGGAGCTGAGAGAGCAGCTGAGCTCCGTGTCCAGCTTC | |
| | GAGAGGTTCGAGATCTTTCCCAAGACAAGCTCCTGGCCCAACCACGACAGCGACAACGGGGTGA | |
| | CAGCCGCCTGCCCTCACGCAGGCGCCAAGAGCTTCTACAAAAACCTGATCTGGCTCGTGAAGAA | |
| | AGGAAAGAGCTACCCCAAGATCAACCAGACCTACATCAACGACAAGGGCAAGGAGGTGCTGGTG | |
| | CTTTGGGGCATCCACCACCCTCCCACAATTGCCGACCAGCAGTCCCTGTACCAGAACGCTGACG | |
| | CCTATGTCTTCGTGGGCACCTCTAGATACAGCAAGAAGTTCAAGCCTGAGATCGCCACAAGACC | |
| | CAAGGTGCGGGATCAGGAGGGCCGGATGAATTACTACTGGACCCTGGTGGAGCCAGGCGACAAG | |
| | ATCACCTTCGAGGCCACCGGCAACCTGGTGGCTCCCCGATACGCCTTCACCATGGAGCGGGACG | |
| | CCGGCAGCGGCATCATCATCAGCGACACACCTGTGCACGACTGCAACACCACCTGCCAGACCCC | |
| | TGAAGGAGCCATCAACACCAGCCTGCCCTTCCAGAATGTCCACCCCATCACCATCGGCAAATGT | |
| | CCAAAGTACGTGAAGTCCACCAAACTGAGACTGGCCACCGGCCTGAGAAACGTCCCCTCAATCC | |
| | AGAGCAGAGGCCTGTTTGGGGCCATTGCCGGCTTCATCGAGGGCGGATGGACCGGCATGGTCGA | |
| | CGGATGGTACGGATATCACttCCAGAACGAACAGGGAAGCGGCTACGCCGCCGACCTGAAGTCC | |
| | ACACAGAACGCCATCGACAAGATCACCAACgccGTGAACAGCGTGATCGAGAAAATGAACACAC | |
| | AGTTCACAGCCGTGGGCAAGGAATTCAATCACCTGGAGAAGAGGATCGAGAATCTGAACAAGAA | |
| | GGTGGACGACGGCTTCCTGGACATCTGGACATACAACGCAGAGCTCCTGGTGCTGCTGgCCAAT | |
| | GAGCGGACACTGGACTACCACGACTCCAACGTGAAGAACCTGTACGAGAAGGTGCGGAATCAAC | |
| | TGAAGAACAACGCCAAGGAGATTGGCAACGGCTGCTTTGAGTTCTACCACAAGTGCGACAACAC | |
| | CTGTATGGAGTCCGTGAAGAACGGCACATATGACTACCCCAAGTACAGCGAAGAAGCCAAGCTG | |
| | AACAGAGAGAAGATTGATGGAGTGAAACTGGACAGCACCCGGATCTACCAAATCCTCGCCATCT | |
| | ACTCCACAGTGGCCTCTAGCCTGGTGCTCGTGGTTTCGCTGGGCGCTATCAGCTTCTGGATGTG | |
| | TTCCAACGGCTCCCTGCAATGCCGGATCTGCATCtga | |
| 65 | ATGAAAGTGAAGTTACTGGTCCTCCTTTGCACCTTTACCGCCACCTACGCCGACACCATCTGCA | A/ |
| | TTGGCTACCATGCCAACAACAGCACCGACACCGTGGACACAGTGCTGGAAAAGAACGTGACCGT | Roma/ |
| | GACACACTCTGTGAACCTGCTGGAGGACAGCCACAACGGAAAACTGTGCAGACTGAAGGGCATC | 1949 |
| | GCCCCTCTGCAGCTGGGCAAGTGCAACATCGCCGGCTGGATCCTGGGCAATCCTGAGTGCGAGA | |
| | GCCTGTTCTCTAAGAAGTCCTGGAGCTACATCGCCGAGACACCCAACAGCGAGAATGGCACCTG | |
| | CTACCCTGGCTACTTCGCTGATTACGAAGAACTGAGAGAGCAGCTGAGCAGCGTCAGCTCCTTC | |
| | GAAAGATTCGAGATCTTCCCCAAAGAAAGAAGCTGGCCCAAACACAACGTGACCAGGGGCGTGA | |
| | CCGCCGCCTGCAGCCACAAGGGCAAGTCTTCCTTTTACCGGAACCTGCTCTGGCTGACAGAGAA | |
| | GGACGGCAGCTACCCCAACCTGTCCAAGAGCTACGTGAACAACAAGGAGAAGGAGGTGCTGGTG | |
| | CTCTGGGGGGTGCACCACCCTTCAAACATCGAGGATCAGAAGACCCTGTACCGGAAGGAGAATG | |
| | CCTATGTCTCCGTGGTGTCTTCCAACTACAATCGGAGATTCACCCCTGAGATCGCCGAGAGGCC | |
| | CAAGGTGCGGGGCCAGGCCGGCAGAATCAACTACTACTGGACCCTGCTGGAACCTGGCGACACA | |
| | ATCATCTTTGAGGCCAACGGCAATCTGATCGCCCCCTGGCATGCATTCGCCCTGTCCAGAGGCT | |
| | TCGGCAGCGGCATCATTACCAGCAACGCCAGCATGGATGAGTGCGACACCAAGTGCCAGACCCC | |
| | TCAGGGCGCCATCAATTCAAGCCTGCCTTTCCAGAACATCCATCCTGTGACCATTGGCGAGTGT | |
| | CCCAAGTACGTGAGAAGCACAAAGCTGAGAATGGTGACCGGACTGAGAAACATCCCTTCCATTC | |
| | AGAGCCGGGGCCTGTTTGGCGCCATCGCCGGCTTCATCGAGGGCGGCTGGACCGGCATGATCGA | |
| | CGGCTGGTACGGCTACCATttCCAGAACGAACAGGGCAGCGGCTACGCCGCTGATCAGAAGTCC | |
| | ACACAGAACGCCATCAACGGCATCACCAACgccGTGAACAGCGTGATCGAGAAGATGAACACAC | |
| | AGTTCACCGCCGTGGGCAAGGAGTTCAACAAACTGGAGAAGAGAATGGAGAATCTGAACAAGAA | |
| | GGTGGACGACGGCTTCCTGGACATCTGGACCTACAATGCTGAGCTGCTGGTGCTGCTGgccAAC | |
| | GAAAGAACCCTCGACTTCCACGACTCCAACGTGAAGAACCTGTACGAGAAGGTGAAGTCACAGC | |
| | TGAAAAACAACGCCAAGGAGATCGGCAACGGCTGCTTCGAGTTCTACCACAAGTGCAACAACGA | |
| | GTGTATGGAGTCCGTGAAGAATGGCACCTACGATTACCCCAAGTACAGCGAGGAGAGCAAGCTG | |
| | AACAGAGAAAAGATCGACGGAGTGAAGCTGGAGAGCATGGGCGTGTATCAAATCCTCGCCATCT | |
| | ACTCCACAGTGGCCTCTAGCCTGGTGCTCCTGGTTTCGCTGGGCGCTATCAGCTTCTGGATGTG | |
| | TTCCAACGGCTCCCTGCAATGCCGGATCTGCATCtga | |
| 66 | ATGAAAGTGAAGTTACTGGTCCTCCTTTGCACCTTTACCGCCACCTACGCCGACACCCTGTGCA | A/ |
| | TTGGCTACCATGCCAACAACTCAACAGACACCGTGGACACAGTGCTGGAGAAGAACGTGACCGT | MD/ |
| | GACACACAGCGTGAATCTGCTGGAGGACCGGCACAACGGCAAACTGTGCAAGCTGAGAGGCGTG | 12/ |
| | GCTCCCCTGCACCTGGGCAAGTGCAACATCGCCGGATGGCTGCTGGGAAACCCAGAGTGCGAAC | 1991 |
| | TACTGTTCACCGCCAGCTCCTGGAGCTACATCGTGGAAACCTCCAACAGCGACAACGGCACATG | |
| | CTACCCTGGCGACTTCATCAATTACGAAGAACTGCGGGAACAGCTGTCCTCAGTGAGCAGCTTC | |
| | GAAAGATTTGAGATCTTCCCAAAGGCCTCCTCTTGGCCCGACCACGAAACCAACCGGGGCGTGA | |
| | CCGCCGCCTGTCCCTACGCCGGAGCCAACAGCTTCTACAGAAATCTGATTTGGCTGGTGAAGAA | |
| | AGGCAACTCCTACCCCAAACTGAGCAAGAGCTACGTGAACAACAAAGAAAAGGAGGTGCTGGTG | |
| | CTCTGGGGCATCCACCACCCTCCGACCTCCACAGACCAGCAAAGCCTGTACCAGAATGCTGACG | |
| | CCTACGTCTTCGTGGGCAGCTCCAAGTACAACAAGAAGTTCAAGCCCGAGATCGCCACCCGGCC | |
| | AAAGGTGCGGGGCCAGGCCGGCAGAATGAACTACTACTGGACACTGGTGGAGCCTGGCGACACC | |
| | ATCACATTCGAGGCCACCGGCAACCTGGTGGTGCCCAGATACGCCTTCGCCATGAAGAGAGGCT | |
| | CTGGATCCGGCATCATCATCAGCGACACCCCTGTGCACGACTGCAACACCACATGCCAGACCCC | |
| | CAAGGGCGCCATCAACACCAGCTTACCTTTCCAGAACATCCACCCTGTGACCATCGGAGAATGT | |
| | CCCAAGTACGTGAAGTCAACAAAGCTGCGGATGGCCACAGGACTGAGAAACATCCCCAGCATCC | |
| | AGTCCAGAGGACTGTTTGGCGCCATCGCCGGCTTCATCGAGGGCGGCTGGACCGGAATGATCGA | |
| | TGGATGGTACGGCTACCACttCCAGAATGAGCAGGGCAGCGGCTACGCCGCTGATCAGAAGTCC | |
| | ACCCAGAATGCCATCGACGGCATCACCAACgccGTGAACTCTGTGATCGAGAAGATGAACACCC | |
| | AGTTTACCGCCGTGGGAAAGGAGTTCAATCACCTGGAGAAGAGAATCGAAAACCTGAACAAGAA | |
| | GGTGGACGACGGCTTCCTGGACGTGTGGACCTACAACGCTGAACTGCTGGTGCTGCTGgCCAAT | |
| | GAGAGAACCCTGGACTACCACGACTCCAACGTGAAGAACCTGTACGAGAAAGTGCGGAGCCAGC | |
| | TGAAAAACAACGCCAAGGAGATTGGAAATGGCTGCTTCGAATTCTACCACAAGTGCGACGACAC | |
| | ATGTATGGAGTCCGTGAAAAACGGCACCTACGACTACCCCAAGTACAGCGAGGAAAGCAAGCTG | |
| | AACAGAGAGGAGATCGATGGGGTGAAACTGGAGAGCACCCGGATCTACCAAATCCTCGCCATCT | |
| | ACTCCACAGTGGCCTCTAGCCTGGTGCTCCTGGTTTCGCTGGGCGCTATCAGCTTCTGGATGTG | |
| | TTCCAACGGCTCCCTGCAATGCCGCATCTGCATCtga | |
| 67 | ATGAAAGTGAAGTTACTGGTCCTCCTTTGCACCTTTACCGCCACCTACGCCGACACCATCTGCA | A/ |
| | TTGGATACCATGCCAACAACAGCACCGACACCGTGGACACAGTGCTGGAAAAGAACGTGACCGT | Denver/ |
| | GACCCACAGCGTGAACCTGCTGGAAGACAGCCACAACGGCAAACTGTGCAGACTGAAGGGCAAG | 57 |
| | GCCCCCCTGCAGCTGGGAAACTGCAACATCGCCGGATGGGTGCTGGGAAATCCCGAGTGCGAGA | |
| | GCCTGCTGAGCAACAGGAGCTGGAGCTACATCGCTGAAACCCCCAACAGCGAGAACGGCACCTG | |
| | CTATCCTGGCGACTTCGCTGACTACGAGGAACTGCGGGAGCAGCTGTCCTCCGTGTCCTCCTTT | |
| | GAAAGATTTGAGATCTTCCCCAAGGAGCGGAGCTGGCCCAACCACACAACCAGGGGCGTGACCG | |
| | CCGCCTGCCCTCATGCCAGAAAGAGCAGCTTCTACAAGAATCTGGTCTGGCTGACCGAGGCCAA | |
| | TGGCAGCTATCCCAACCTGTCCAGAAGCTACGTGAACAACCAGGAGAAGGAGGTGCTGGTCCTG | |
| | TGGGGGGTGCACCACCCCAGCAACATCGAGGAGCAGAGAGCCCTGTACAGAAAGGACAACGCCT | |
| | ATGTCAGCGTGGTGTCCAGCAACTACAACAGACGGTTCACCCCCGAAATTGCCAAGCGGCCCAA | |
| | GGTGCGGGATCAGAGCGGCCGGATGAACTACTACTGGACCCTGCTGGAGCCTGGAGACACCATC | |
| | ATCTTCGAGGCCACCGGCAACCTGATCGCCCCTTGGTACGCCTTCGCCCTGAGCAGAGGCCCTG | |
| | GCAGCGGCATCATCACCAGCAATGCCCCCCTGGACGAGTGCGACACAAAGTGTCAGACCCCCCA | |
| | GGGCGCCATCAACAGCTCCCTGCCCTTTCAGAACATCCACCCTGTGACAATCGGGGAGTGCCCC | |
| | AAGTACGTGAGAAGCACCAAGCTGCGGATGGTGACCGGCCTGCGGAACATTCCCTCTGTGCAGT | |
| | CCCGGGGCCTGTTTGGGGCCATCGCCGGCTTCATCGAGGGCGGCTGGACCGGCATGATGGACGG | |
| | CTGGTACGGCTACCACttCCAGAACGAACAAGGCTCCGGCTACGCCGCTGACCAAAAGTCCACC | |
| | CAGAATGCAATCAACGGCATCACCAACgccGTGAATTCCGTGATCGAGAAGATGAACACCCAGT | |
| | TCACAGCCGTGGGCAAGGAGTTCAACAAGCTGGAAAAGCGGATGGAGAACCTGAACAAGAAAGT | |
| | GGACGACGGCTTCATGGACATCTGGACCTACAACGCTGAACTGCTGGTGCTGCTGgccAATGAA | |
| | AGAACACTGGACTTCCACGACTCCAACGTGAAGAACCTGTATGAGAAGGTGAAGAATCAGCTCA | |
| | GAAACAACGCCAAAGAGCTGGGCAACGGCTGCTTCGAGTTCTACCACAAGTGCGACAACGAGTG | |
| | TATGGAGTCCGTGAAGAACGGCACATACGACTACCCCAAGTACAGCGAGGAGAGCAAGCTGAAC | |
| | AGGGAGAAGATCGATGGCGTGAAGCTGGAGTCCATGGGCGTCTACAGAATCCTCGCCATCTACT | |
| | CCACAGTGGCCTCTAGCCTGGTGCTCCTGGTTTCGCTGGGCGCTATCAGCTTCTGGATGTGTTC | |
| | CAACGGCTCCCTGCAATGCCGGATCTGCATCtga | |
| 68 | ATGAAAGTGAAGTTACTGGTCCTCCTTTGCACCTTTACCGCCACCTACGCTGACACCATCTGCA | A/ |
| | TTGGCTACCATGCCAACAACTCCACCGACACCGTGGACACAGTGCTGGAGAAGAACGTGACCGT | Puerto Rico/ |
| | GACACATTCCGTGAACCTGCTGGAGGATTCTCACAACGGCAAGCTCTGTCGGCTGAAGGGCATC | 8/ |
| | GCCCCTCTGCAGCTGGGCAAGTGCAACATCGCCGGCTGGCTGCTGGGCAACCCCGAGTGCGACC | 1934 |
| | CTCTGCTGCCCGTGAGATCCTGGTCCTACATTGTGGAAACCCCTAACAGCGAGAACGGCATCTG | |
| | CTACCCTGGCGACTTCATTGATTACGAGGAGCTGCGGGAGCAGCTGTCCTCAGTGAGCAGCTTC | |
| | GAGAGGTTTGAGATCTTCCCCAAGGAGTCCTCTTGGCCCAACCACAACACAAACAAGGGCGTCA | |
| | CCGCCGCCTGCAGCCATGAGGGAAAGAGCAGCTTCTACAGAAACCTGCTGTGGCTGACAGAGAA | |
| | GGAGGGCTCCTACCCCAAGCTGAAGAATTCCTACGTGAACAAGAAAGGCAAAGAGGTGCTGGTG | |
| | CTGTGGGGGATCCACCACCCTCCCAATTCCAAGGAACAGCAGAACCTGTACCAGAACGAAAACG | |
| | CCTACGTCTCTGTGGTGACCTCCAACTACAACAGAAGATTCACCCCTGAGATCGCAGAGCGGCC | |
| | CAAGGTGCGGGACCAAGCCGGCAGAATGAACTACTACTGGACCCTGCTGAAGCCTGGCGACACA | |
| | ATCATCTTCGAGGCCAATGGAAACCTGATCGCCCCAATGTACGCCTTCGCCCTGTCCCGGGGCT | |
| | TCGGAAGCGGCATCATCACAAGCAACGCCTCCATGCACGAGTGCAACACCAAGTGCCAGACCCC | |
| | ACTGGGCGCCATCAATTCCTCTCTCCCTTACCAGAACATCCACCCTGTGACCATCGGGGAATGC | |
| | CCCAAGTACGTGAGATCCGCCAAGCTGCGGATGGTGACCGGCCTGAGAAACATCCCCAGCATTC | |
| | AGAGCAGGGGCCTGTTTGGAGCCATCGCTGGCTTCATCGAGGGCGGATGGACCGGCATGATCGA | |
| | CGGCTGGTACGGCTACCACttCCAGAATGAACAGGGCTCCGGCTACGCAGCCGATCAGAAAAGC | |
| | ACACAGAATGCCATCAACGGCATCACCAACgccGTGAACACCGTGATCGAAAAGATGAACATCC | |
| | AGTTCACCGCCGTGGGCAAGGAGTTCAACAAGCTGGAGAAGAGAATGGAGAACCTGAACAAGAA | |
| | GGTGGACGACGGCTTTCTGGACATTTGGACATACAATGCTGAGCTGCTGGTGCTGCTGgccAAT | |
| | GAGAGGACCCTGGACTTTCACGACTCCAACGTGAAAAACCTGTACGAAAAGGTCAAGAGCCAGC | |
| | TGAAGAACAATGCCAAGGAGATCGGCAACGGCTGCTTCGAGTTCTACCACAAGTGCGACAACGA | |
| | GTGTATGGAGTCCGTGAGAAACGGCACCTATGACTACCCCAAGTACTCTGAGGAGTCCAAACTG | |
| | AACAGAGAGAAGGTGGACGGAGTGAAGCTGGAGTCTATGGGCATCTACCAAATCCTCGCCATCT | |
| | ACTCCACAGTGGCCTCTAGCCTGGTGCTCCTGGTTTCGCTGGGCGCTATCAGCTTCTGGATGTG | |
| | TTCCAACGGCTCCCTGCAATGCCGCATCTGCATCtga | |
| 69 | ATGAAAGTGAAGTTACTGGTCCTCCTTTGCACCTTTACCGCCACCTACGCCGACACCATCTGCA | A/ |
| | TTGGCTATCATGCCAACAATTCCACAGACACCGTGGACACAGTGCTGGAGAAGAACGTGACCGT | Brisbane/ |
| | GACCCACTCTGTGAACCTGCTGGAGAACTCTCACAACGGCAAACTGTGCCTGCTGAAGGGCATC | 59/ |
| | GCCCCTCTGCAGCTCGGCAACTGCAGCGTGGCCGGCTGGATCCTGGGCAACCCAGAGTGCGAAC | 2007 |
| | TGCTGATCTCCAAGGAAAGCTGGAGCTACATCGTGGAGAAGCCCAACCCCGAGAACGGCACCTG | |
| | TTACCCTGGCCACTTTGCCGACTACGAGGAGCTGCGGGAACAGCTGTCTTCCGTGTCCTCCTTC | |
| | GAAAGATTCGAGATCTTTCCCAAGGAGAGCTCCTGGCCCAACCACACAGTGACAGGCGTCTCCG | |
| | CCAGCTGCTCCCACAATGGCGAGTCCTCTTTCTACAGAAACCTGCTGTGGCTGACCGGCAAGAA | |
| | CGGCCTGTATCCCAACCTGAGCAAGAGCTACGCCAACAACAAGGAGAAGGAAGTGCTGGTGCTG | |
| | TGGGGGGTGCACCACCCTCCGAACATCGGCAATCAGAAGGCCCTGTATCACACAGAAAACGCCT | |
| | ACGTCAGCGTGGTGTCCTCCCACTACAGCAGAAAGTTCACCCCTGAGATCGCCAAGCGGCCCAA | |
| | AGTGCGGGATCAGGAGGGCCGGATCAATTACTACTGGACCCTGCTGGAACCAGGCGACACAATC | |
| | ATCTTTGAGGCCAACGGCAACCTGATCGCCCCAAGATACGCCTTCGCCCTGAGCAGAGGCTTCG | |
| | GCTCTGGCATCATCAACAGCAATGCCCCGATGGACAAGTGCGATGCCAAGTGCCAGACCCCACA | |
| | GGGCGCCATCAACAGCAGCCTGCCTTTCCAGAATGTCCACCCCGTGACAATTGGCGAGTGTCCC | |
| | AAGTACGTGAGAAGCGCCAAGCTGCGGATGGTGACCGGCCTGCGGAACATCCCTTCCATTCAGA | |
| | GCAGAGGCCTGTTTGGCGCCATCGCCGGATTCATTGAAGGCGGCTGGACCGGCATGGTGGACGG | |
| | CTGGTACGGCTACCACttCCAGAACGAACAGGGCTCTGGCTACGCCGCTGACCAAAAGTCCACC | |
| | CAGAATGCCATCAACGGCATCACCAACgccGTCAACAGCGTGATCGAGAAGATGAACACCCAGT | |
| | TCACCGCTGTGGGCAAAGAGTTCAACAAGCTGGAGCGGCGGATGGAAAACCTGAACAAAAAGGT | |
| | GGACGACGGCTTCATCGACATTTGGACATACAACGCAGAGCTGCTGGTGCTGCTGgccAATGAG | |
| | AGAACCCTGGATTTCCACGACAGCAACGTGAAGAACCTGTACGAGAAGGTCAAATCCCAGCTGA | |
| | AAAACAACGCCAAGGAGATCGGCAATGGCTGCTTCGAGTTCTACCACAAGTGCAACGATGAGTG | |
| | TATGGAGTCCGTGAAAAACGGCACCTACGACTACCCGAAGTACAGCGAGGAAAGCAAGCTCAAT | |
| | CGGGAGAAGATCGACGGAGTGAAGCTGGAGAGCATGGGCGTGTATCAAATCCTCGCCATCTACT | |
| | CCACAGTGGCCTCTAGCCTGGTGCTCCTGGTTTCGCTGGGCGCTATCAGCTTCTGGATGTGTTC | |
| | CAACGGCTCCCTGCAATGCCGGATCTGCATCtga | |
| 70 | ATGAAAGTGAAGTTACTGGTCCTCCTTTGCACCTTTACCGCCACCTACGCTGACACCCTGTGCA | A/ |
| | TTGGCTATCATGCCAACAACAGCACCGACACAGTGGACACAGTGCTGGAAAAGAACGTGACCGT | California/ |
| | GACACACTCCGTGAACCTGCTGGAGGACAAGCACAACGGCAAACTGTGCAAGCTGAGAGGCGTG | 07/ |
| | GCCCCTCTGCACCTGGGCAAGTGCAACATCGCCGGCTGGATCCTGGGCAACCCAGAGTGCGAGA | 2009 |
| | GCCTGAGCACCGCCAGCTCCTGGTCTTACATCGTGGAGACACCCAGCTCCGACAACGGCACATG | |
| | CTACCCTGGCGACTTCATCGACTACGAGGAACTGAGAGAGCAGCTGAGCTCCGTGTCCTCTTTC | |
| | GAGAGGTTCGAGATCTTCCCCAAGACCTCATCTTGGCCCAACCACGACAGCAACAAGGGCGTGA | |
| | CCGCCGCCTGTCCTCACGCTGGCGCAAAGAGCTTCTACAAGAACCTGATCTGGCTCGTGAAGAA | |
| | GGGAAACAGCTACCCCAAGCTGAGCAAGAGCTACATCAATGACAAAGGCAAGGAGGTGCTGGTG | |
| | CTATGGGGCATCCATCACCCCAGCACAAGCGCTGACCAGCAGAGCCTGTATCAGAACGCTGACG | |
| | CCTACGTCTTCGTCGGCAGCTCCCGGTACAGCAAGAAATTCAAGCCCGAGATCGCCATCCGGCC | |
| | CAAGGTGCGGGACCAAGAGGGCAGAATGAACTACTACTGGACCCTGGTGGAGCCTGGCGACAAG | |
| | ATCACCTTTGAGGCCACAGGCAATCTGGTGGTGCCCAGATACGCCTTTGCCATGGAGAGAAATG | |
| | CCGGCTCCGGCATCATCATCAGCGACACACCTGTGCACGACTGCAACACCACCTGTCAGACACC | |
| | CAAGGGCGCCATCAACACAAGCCTGCCTTTTCAGAACATTCACCCCATCACCATTGGCAAGTGC | |
| | CCCAAGTACGTCAAGAGCACCAAACTGCGGCTGGCCACAGGACTGCGGAACATCCCCAGCATTC | |
| | AGAGCCGGGGCCTGTTTGGAGCCATCGCTGGCTTCATCGAGGGCGGCTGGACCGGCATGGTGGA | |
| | CGGCTGGTACGGCTACCATttCCAGAATGAACAGGGCAGCGGCTACGCCGCTGACCTGAAGTCC | |
| | ACCCAGAACGCCATCGACGAAATCACCAACctgGTCAACTCTGTGATCGAAAAGATGAACACCC | |
| | AGTTCACCGCCGTGGGAAAAGAGTTCAACCACCTGGAGAAGAGGATCGAAAATCTGAACAAAAA | |
| | GGTGGACGACGGCTTTCTGGACATCTGGACCTACAACGCAGAGCTCCTGGTGCTGCTGctgAAT | |
| | GAAAGAACACTGGACTACCACGACTCCAACGTCAAGAACCTGTACGAGAAGGTGCGGAGCCAGC | |
| | TGAAGAACAACGCCAAGGAGATCGGCAACGGCTGCTTCGAGTTCTACCACAAGTGCGACAACAC | |
| | CTGTATGGAGTCCGTGAAGAACGGCACATACGACTACCCCAAGTACTCTGAAGAGGCCAAGCTG | |
| | AACAGAGAGGAGATTGATGGAGTGAAGCTGGAGTCCACCCGGATCTACCAAATCCTCGCCATCT | |
| | ACTCCACAGTGGCCTCTAGCCTGGTGCTCGTGGTTTCGCTGGGCGCTATCAGCTTCTGGATGTG | |
| | TTCCAACGGCTCCCTGCAATGCCGGATCTGCATCtga | |
| 71 | ATGAAAGTGAAGTTACTGGTCCTCCTTTGCACCTTTACCGCCACCTACGCCGACACCATCTGCA | A/ |
| | TTGGCTACCATGCCAACAACAGCACCGACACCGTGGACACAGTGCTGGAGAAGAACGTGACAGT | Beijing/ |
| | GACACACTCTGTGAACCTGCTGGAGGACTCCCACAACGGAAAGCTGTGCCTGCTGAAGGGAATC | 262/ |
| | GCCCCTCTGCAGCTGGGCAACTGTTCCGTCGCCGGCTGGATCCTGGGCAATCCTGAGTGCGAGA | 1995 |
| | GCCTGATCTCCAAAGAATCTTGGTCCTACATCGTGGAGACACCCAACCCAGAGAACGGCACCTG | |
| | CTACCCCGGATACTTTGCCGACTACGAGGAGCTGCGGGAGCAACTGTCCTCAGTGAGCAGCTTC | |
| | GAAAGATTCGAGATCTTCCCCAAAGAGAGCTCTTGGCCCAACCACACAGTGACAGGCGTGACAG | |
| | CCTCCTGCAGCCACAACGGAAAGAGCAGCTTCTACAGAAATCTGCTGTGGCTCACCGAGAAGAA | |
| | TGGCCTGTATCCCAACCTCTCCAACAGCTACGTGAACAACAAGGAGAAGGAAGTGCTGGTGCTA | |
| | TGGGGGGTGCACCACCCCAGCAACATCCGGGACCAGCGGGCCATCTACCACACAGAGAACGCCT | |
| | ACGTCAGCGTGGTGTCCTCCCACTACTCTCGGAGATTCACTCCCGAGATCGCCAAGCGGCCCAA | |
| | GGTGCGGGGCCAGGAGGGCCGGATCAATTACTACTGGACCCTGCTGGAACCAGGCGACACCATC | |
| | ATCTTCGAGGCCAACGGCAATCTCATCGCCCCCTGGTACGCTTTCGCCCTGTCTCGGGGCTTCG | |
| | GCTCCGGCATCATCACAAGCAACGCCCCCATGAATGAGTGCGATGCCAAGTGCCAGACACCCCA | |
| | GGGCGCCATCAACAGCTCCCTGCCCTTCCAGAACGTCCACCCTGTGACCATCGGAGAGTGTCCC | |
| | AAGTACGTGAGATCCACCAAGCTGAGAATGGTGACCGGCCTGAGAAACATTCCCAGCATCCAGA | |
| | GCAGAGGCCTGTTCGGAGCCATCGCCGGATTCATTGAGGGAGGATGGACCGGCATGATGGACGG | |
| | CTGGTACGGCTACCACttCCAGAATGAGCAGGGAAGCGGCTACGCCGCTGACCAGAAATCCACA | |
| | CAGAACGCCATCAACGGCATCACCAACctgGTGAACAGCGTGATCGAAAAGATGAACACCCAGT | |
| | TCACCGCCGTGGGCAAAGAGTTCAACAAGCTGGAAAGAAGAATGGAGAACCTGAACAAGAAAGT | |
| | GGACGACGGCTTCCTGGACATCTGGACATACAACGCAGAGCTGCTGGTGCTGCTGctgAATGAG | |
| | AGAACACTGGACTTCCACGACAGCAACGTCAAAAACCTGTACGAGAAGGTGAAGTCACAGCTGA | |
| | AAAACAACGCCAAGGAGATCGGCAATGGCTGCTTCGAATTCTACCACAAGTGCAACAACGAGTG | |
| | TATGGAATCCGTGAAGAACGGCACCTACGACTACCCGAAGTACTCTGAGGAGAGCAAACTGAAC | |
| | AGAGAGAAAATCGACGGGGTGAAGCTGGAGAGCATGGGCGTGTATCAAATCCTCGCCATCTACT | |
| | CCACAGTGGCCTCTAGCCTGGTGCTCCTGGTTTCGCTGGGCGCTATCAGCTTCTGGATGTGTTC | |
| | CAACGGCTCCCTGCAATGCCGGATCTGCATCtga | |
| 72 | ATGAAAGTGAAGTTACTGGTCCTCCTTTGCACCTTTACCGCCACCTACGCCGACACAATCTGCA | A/ |
| | TTGGCTACCATGCCAACAACAGCACAGACACCGTGGACACCGTCCTGGAGAAAAACGTGACAGT | NewYork/ |
| | GACCCACAGCGTGAACCTGCTGGAAGACTCCCACAATGGAAAACTGTGCAAGCTGAAGGGCATC | 1/ |
| | GCCCCTCTGCAGCTGGGCAAGTGCAACATCGCCGGCTGGCTGCTGGGCAACCCAGAGTGCGACC | 1918 |
| | TGCTGCTGACAGCCAGTAGCTGGAGCTACATCGTGGAAACCAGCAACAGCGAAAATGGCACATG | |
| | CTACCCTGGCGACTTCATCGATTACGAGGAGCTGCGGGAGCAGCTGAGCAGCGTCAGCTCTTTC | |
| | GAGAAGTTTGAGATCTTCCCCAAAACCTCAAGCTGGCCCAACCACGAAACCACAAAGGGCGTGA | |
| | CCGCCGCCTGCAGCTACGCAGGCGCCAGCTCTTTCTACAGAAACCTGCTGTGGCTGACAAAGAA | |
| | GGGGAGCAGCTACCCCAAGCTCTCCAAAAGCTACGTGAACAACAAGGGCAAGGAGGTGCTGGTG | |
| | CTATGGGGGGTGCATCACCCTCCAACCGGCACCGACCAGCAGAGCCTGTACCAGAACGCCGACG | |
| | CCTACGTCTCCGTGGGCTCCTCCAAGTACAACAGAAGATTCACCCCTGAGATCGCTGCTCGGCC | |
| | CAAGGTGCGGGACCAGGCCGGAAGGATGAACTACTACTGGACCCTGCTGGAGCCTGGCGACACA | |
| | ATCACCTTCGAGGCCACAGGCAACCTGATCGCCCCTTGGTACGCCTTCGCTCTCAACAGAGGCT | |
| | CCGGCAGCGGCATCATCACCTCCGACGCCCCTGTGCACGACTGCAACACAAAGTGCCAGACCCC | |
| | TCACGGGGCCATCAACTCCAGCCTGCCTTTCCAGAACATCCACCCTGTGACCATCGGAGAATGC | |
| | CCCAAGTACGTGAGAAGCACCAAGCTGAGAATGGCCACAGGCCTGAGAAACATCCCTTCCATCC | |
| | AGAGCAGGGGCCTGTTTGGGGCCATCGCCGGCTTCATCGAGGGCGGCTGGACCGGCATGATCGA | |
| | CGGCTGGTACGGCTACCACttCCAGAACGAACAGGGCTCCGGCTACGCCGCCGACAGAAAGTCC | |
| | ACCCAGAATGCCATTGACGGCATCACCAACctgGTGAATTCCGTGATCGAGAAGATGAACACCC | |
| | AGTTCACCAGCGTGGGAAAAGAGTTCAACCACCTGGAGAAGAGAATCGAGAACCTGAACAGGAA | |
| | GGTGGATGACGGCTTCCTGGACGTGTGGACATACAACGCTGAACTGCTGGTGCTGCTGctgAAC | |
| | GAAAGAACCCTGGACTACCACGACAGCAACGTGAAGAACCTGTACGAGAAGGTGCGGAGCCAGC | |
| | TGAAGAACAACGCCAAGGAAATCGGCAATGGCTGTTTCGAATTCTACCACAAGTGCGACGACAG | |
| | CTGTATGGAATCCGTGAAAAACGGCACATACGACTACCCCAAGTACAGCGAGGAGAGCAAGCTG | |
| | AACAGAGAGGAAATCGATGGCGTGAAACTGGAGTCCATGGGCGTGTATCAAATCCTCGCCATCT | |
| | ACTCCACAGTGGCCTCTAGCCTGGTGCTCCTGGTTTCGCTGGGCGCTATCAGCTTCTGGATGTG | |
| | TTCCAACGGCTCCCTGCAATGCCGGATCTGCATCtga | |
| 73 | ATGAAAGTGAAGTTACTGGTCCTCCTTTGCACCTTTACCGCCACCTACGCTGACACCCTGTGCA | A/ |
| | TTGGCTACCATGCCAACAACAGCACCGACACCGTCGACACAGTCCTGGAAAAGAACGTGACAGT | Wisconsin/ |
| | GACCCACTCCGTGAATCTGCTGGAAAACAGACACAACGGCAAACTGTGCAAGCTGCGGGGCGTG | 28/ |
| | GCCCCTCTGCACCTGGGCAAATGCAACATCGCCGGATGGCTGCTGGGCAACCCAGAGTGCGAAA | 2011. |
| | GCCTGAGCACCGCCAGCTCTTGGTCCTACATCGTCGAAACCAGCAACAGCGACAATGGCACATG | 2011/ |
| | CTACCCTGGCGACTTCATCAACTACGAGGAACTGAGAGAGCAGCTGAGCTCTGTGTCCTCCTTC | 12 |
| | GAGAGGTTTGAGATCTTCCCCAAGACCTCAAGCTGGCCCAATCACGACACAAACAGAGGAGTGA | |
| | CCGCCGCCTGTCCTCACGATGGAACCAACAGCTTCTACAGAAATCTGATCTGGCTCGTGAAGAA | |
| | GGGCAACTCTTACCCCAAGATCAACAAGTCTTACATCAACAACAAAGAGAAGGAAATTCTGGTG | |
| | CTGTGGGCCATCCACCACCCCAGCACCTCCGCTGACCAGCAAAGCCTGTACCAGAACGCTGACG | |
| | CCTACGTCTTCGTGGGCAGCTCCCGGTACAGCAGAAAGTTCGAACCTGAAGTGGCCACCCGGCC | |
| | CAAGGTGCGGGACCAGGCCGGCAGAATGAACTACTACTGGACCCTGGTCGAACCTGGCGACAAG | |
| | ATCACCTTCGAAGCCACCGGAAACCTGGTGGTGCCCAGATACGCCTTCGCCCTGAAGAGAAATT | |
| | CGGGCAGCGGCATCATCATCAGCGACACATCCGTCCACGACTGCGACACCAACTGTCAGACCCC | |
| | AAATGGGGCCATCAACACCAGCCTGCCTTTCCAGAACATCCACCCTGTCACCATCGGAGAGTGC | |
| | CCCAAGTACGTGAAGTCCACAAAGCTGAGAATGGCCACCGGCCTGCGGAACATCCCCAGCATCC | |
| | AGAGCCGGGGACTGTTTGGCGCCATCGCCGGCTTCATCGAGGGCGGCTGGACCGGCATGATCGA | |
| | CGGCTGGTACGGCTACCACttCCAGAATGAGCAGGGCAGCGGCTACGCCGCTGACCTGAAGTCC | |
| | ACACAGAACGCCATCGACGGCATCACCAACctgGTGAACAGCGTGATCGAGAAAATGAACACCC | |
| | AGTTCACCGCCGTGGGCAAGGAATTCTCCCACCTGGAGAGACGGATTGAGAACCTGAACAAGAA | |
| | GGTGGACGACGGCTTTCTGGACATCTGGACCTACAACGCTGAACTGCTGGTGCTGCTGctgAAT | |
| | GAGAGAACACTGGACTACCACGATTCCAATGTCAAAAACCTGTATGAGAAAGTGCGGAGCCAGC | |
| | TGAAGAACAATGCCAAGGAAATTGGCAACGGCTGTTTCGAGTTCTACCACAAGTGCGATGATAT | |
| | GTGTATGGAGTCCGTGAAGAATGGAACATACGACTACCCCAAGTACAGCGAAGAGGCCAAGCTG | |
| | AACAGAGAGGAGATCGACGGGGTGAAGCTGGAGTCTACCCGGATCTACCAAATCCTCGCCATCT | |
| | ACTCCACAGTGGCCTCTAGCCTGGTGCTCGTGGTTTCGCTGGGCGCTATCAGCTTCTGGATGTG | |
| | TTCCAACGGCTCCCTGCAATGCCGGATCTGCATCtga | |
| 74 | ATGAAAACGATCATCGCCCTATCCTACATCCTATGCCTCGTCTTCGCCCAAAAACTCCCCGGCA | A/ |
| | ACGACAACAGCACAGCCACACTGTGCCTGGGCCATCACGCTGTGCCCAACGGAACCATCGTGAA | Alaska/ |
| | GACCATTACCAACGACAGAATCGAAGTGACAAATGCCACAGAGCTGGTGCAGAACAGCAGCATT | 01/ |
| | GGCGAGATCTGCAACAGCCCTCACCAGATCCTGGACGGAGGCAACTGCACACTGATCGATGCCC | 2021 |
| | TGCTCGGCGACCCTCAGTGCGATGGCTTCCAGAACAAGGAGTGGGACCTGTTCGTGGAAAGAAG | |
| | CAGAGCCAACAGCTCCTGCTACCCCTACGACGTCCCTGACTACGCCAGCCTGCGGAGCCTGGTG | |
| | GCCTCCAGCGGCACCCTGGAGTTCAAAAACGAAAGCTTCAATTGGACCGGAGTGAAACAGAACG | |
| | GCACCTCCAGCGCCTGTATCAGAGGCTCCAGCAGCTCTTTCTTCAGCAGACTGAACTGGCTGAC | |
| | AAGCCTGAACAACATTTACCCCGCCCAGAACGTGACAATGCCCAACAAGGAGCAGTTTGACAAA | |
| | CTGTACATCTGGGGAGTGCACCACCCTGATACCGACAAAAACCAGTTCTCCCTGTTCGCCCAGT | |
| | CTTCCGGCAGAATCACCGTGTCTACCAAGCGGAGCCAGCAGGCCGTGATCCCCAACATCGGCAG | |
| | CCGGCCCCGGATTCGGGACATCCCTTCCCGGATCAGCATTTACTGGACCATCGTGAAGCCTGGA | |
| | GACATTCTCCTGATCAACAGCACCGGCAACCTGATCGCCCCTCGGGGCTACTTCAAGATCAGAA | |
| | GCGGCAAGTCCAGCATCATGAGATCTGACGCCCCTATCGGCAAGTGCAAGTCTGAGTGTATCAC | |
| | ACCCAACGGCAGCATCCCCAACGACAAGCCCTTTCAGAACGTGAACAGAATCACCTATGGCGCC | |
| | TGTCCCAGGTACGTGAAGCAGAGCACCCTGAAGCTGGCCACAGGGATGAGAAACGTCCCCGAAA | |
| | AGCAGACCCGGGGCATCTTTGGCGCTATCGCCGGCTTCATCGAGAACGGCTGGGAGGGCATGGT | |
| | GGACGGCTGGTACGGCTTCCGGttCCAGAACAGCGAGGGCAGAGGCCAGGCCGCTGACCTGAAG | |
| | TCCACCCAGGCTGCCATCGACCAGATCAGCGGActgCTGAACAGACTGATCGGCAAAACAAACG | |
| | AAAAGTTCCATCAGATCGAGAAGGAGTTCTCCGAGGTGGAAGGCCGGGTGCAGGACCTGGAAAA | |
| | GTACGTGGAAGACACCAAGATCGACCTGTGGTCCTACAACGCTGAACTGCTGGTCGCCCTGctg | |
| | AATCAGCACACCATCGACCTGACCGACAGCGAGATGAACAAACTGTTCGAGAAGACCAAGAAGC | |
| | AGCTGCGGGAAAACGCCGAGGATATGGGCAACGGATGCTTCAAGATTTACCACAAGTGCGACAA | |
| | CGCCTGCATTGGCAGCATCAGAAATGAGACATACGACCACAATGTTTACCGGGATGAGGCCCTG | |
| | AACAACAGATTCCAGATCAAGGGAGTGGAGCTGAAGTCCGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 75 | ATGAAAACGATCATCGCCCTATCCTACATCCTATGCCTCGTCTTCGCCCAAAAACTCCCCGGCA | A/ |
| | ACGACAACAGCACCGCCACCCTGTGCCTGGGGCACCACGCTGTGCCAAATGGCACCATTGTGAA | Indiana/ |
| | GACCATTACCAATGACAGAATCGAGGTGACCAATGCCACCGAACTGGTGCAGAATTCCAGCATT | 11/ |
| | GGCGAGATCTGCGACAGCCCTCACCAGATCCTGGATGGCGAGAACTGCACCCTGATCGATGCTC | 2018 |
| | TGCTGGGCGACCCTCAGTGCGATGGCTTCCAGAACAAGAAATGGGACCTGTTCGTGGAGCGGAG | |
| | CAAGGCCTACAGCAACTGCTACCCCTACGACGTCCCCGATTACGCCAGCCTGCGGAGCCTGGTG | |
| | GCCTCCTCCGGCACACTGGAGTTCAACAACGAAAGCTTCAACTGGACAGGAGTGAAGCAGAACG | |
| | GCACATCCAGCGCCTGCATCAGAAAAAGCTCCAGCAGCTTCTTCTCTCGACTGAACTGGCTGAC | |
| | CCACCTGAACTACACCTACCCCGCCCTGAACGTGACCATGCCAAACAATGAGCAGTTTGACAAA | |
| | CTGTACATTTGGGGAGTGCACCACCCCGGCACAGACAAGGACCAGATCTTTCTGTACGCCCAGA | |
| | GCAGCGGCAGGATCACCGTGTCTACCAAGAGAAGCCAGCAGGCCGTGATCCCCAACATCGGCAG | |
| | CAGACCTCGGATCAGAGACATCCCCTCCAGAATCTCCATCTACTGGACAATTGTGAAGCCTGGA | |
| | GACATTCTGCTGATCAACAGCACCGGCAACCTGATCGCCCCTCGAGGATACTTCAAGATCCAGA | |
| | GCGGCAAAAGCAGCATCATGAGATCTGACGCCCCCATCGGCAAATGCAAAAGCGAGTGTATCAC | |
| | CCCCAACGGCAGCATCCCCAACGACAAGCCCTTTCAGAACGTGAACAGAATCACCTATGGCGCC | |
| | TGTCCCAGGTACGTGAAGCACAGCACCCTGAAGCTGGCCACAGGAATGAGAAACGTCCCCGAGA | |
| | AGCAAACCCGGGGCATCTTTGGCGCTATTGCCGGCTTCATCGAGAACGGCTGGGAAGGGATGGT | |
| | GGACGGCTGGTACGGCTTCCGGttCCAGAACAGCGAGGGAAGAGGCCAGGCCGCTGACCTGAAG | |
| | TCCACCCAGGCCGCCATTGATCAGATCAACGGCctgCTGAACAGACTGATCGGCAAGACCAACG | |
| | AAAAATTCCACCAGATCGAAAAGGAATTCAGCGAAGTGGAGGGCAGAATTCAGGACCTGGAAAA | |
| | GTACGTGGAGGACACAAAGATCGACCTGTGGTCCTACAACGCTGAACTGCTGGTGGCCCTGctg | |
| | AATCAGCACACCATCGACCTGACCGACAGCGAGATGAACAAACTGTTTGAAAAGACCAAGAAGC | |
| | AGCTGAGAGAAAACGCCGAGGACATGGGCAATGGATGCTTCAAAATCTACCACAAGTGCGACAA | |
| | CGCCTGTATTGGCAGCATCAGAAATGGCACCTACGACCACAACGTCTACAGGGACGAGGCCCTG | |
| | AACAACAGATTTCAAATCAAGGGCGTGGAGCTGAAGTCTGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 76 | ATGAAAACGATCATCGCCCTATCCTACATCCTATGCCTCGTCTTCGCCCAAAAACTCCCCGGCA | A/ |
| | ACGACAACAGCACAGCCACACTCTGTCTGGGCCATCACGCTGTGCCAAACGGCACAATCGTGAA | California/ |
| | GACAATCACCAACGACCAGATTGAGGTGACAAATGCCACAGAGCTGGTGCAGAGCTCCAGCACA | 07/ |
| | GGCGGCATCTGCGACAGCCCTCATCAGATCCTGGATGGCGAGAACTGCACACTGATCGATGCCC | 2004 |
| | TGCTGGGCGACCCTCAGTGCGACGGCTTCCAGAACAAGAAATGGGACCTGTTCGTGGAGCGGAG | |
| | CAAGGCTTACAGCAACTGTTACCCCTACGACGTCCCTGACTACGCCTCCCTGCGGAGCCTGGTG | |
| | GCCTCCTCCGGCACCCTGGAGTTTAACAATGAATCCTTTAACTGGACAGGCGTCACCCAGAACG | |
| | GCACATCTTCCAGCTGCAAGAGAAGAAGCAACAACTCCTTCTTCAGCAGACTGAATTGGCTGAC | |
| | CCACCTGAAGTTCAAGTACCCTGCCCTGAACGTGACCATGCCAAACAATGAGAAGTTCGACAAG | |
| | CTGTACATCTGGGGGGTTCACCACCCTGGCACAAACAACGACCAGATCAGCCTGTACACCCAGG | |
| | CCAGCGGCCGGATCACCGTGTCTACAAAGCGGAGCCAGCAGACAGTGATCCCCAACATCGGCTC | |
| | CAGACCTCGGGTGAGAGACATCCCCTCCAGAATCAGCATCTACTGGACCATCGTGAAGCCAGGC | |
| | GACATCCTGCTCATCAACAGCACCGGAAACCTGATTGCACCCAGAGGCTACTTCAAGATCAGAA | |
| | GCGGCAAGAGCAGCATCATGAGAAGCGATGCCCCCATCGGCAAGTGCAACAGCGAGTGTATCAC | |
| | CCCCAACGGCAGCATCCCCAATGACAAGCCCTTCCAGAACGTGAACAGAATCACCTATGGCGCC | |
| | TGCCCCAGGTACGTCAAGCAGAACACCCTGAAGCTGGCCACCGGAATGAGAAACGTCCCCGAGA | |
| | AGCAGACCAGGGGGATCTTTGGAGCCATCGCCGGCTTCATCGAGAACGGCTGGGAGGGAATGGT | |
| | GGACGGATGGTACGGCTTCCGGttCCAGAACTCTGAGGGCATTGGCCAGGCCGCAGATCTGAAG | |
| | TCCACCCAGGCCGCCATCAACCAGATCAACGGCctgCTGAACAGACTGATCGGCAAGACAAATG | |
| | AAAAATTCCACCAGATCGAGAAAGAATTCAGCGAGGTGGAAGGCCGGATTCAGGACCTGGAGAA | |
| | GTACGTGGAGGACACCAAGATCGACCTGTGGAGCTACAACGCTGAACTGCTCGTGGCCCTGctg | |
| | AACCAGCACACAATCGACCTGACAGACAGCGAGATGAACAAGCTGTTCGAAAGAACAAAGAAAC | |
| | AGCTGAGAGAAAACGCCGAGGACATGGGCAACGGCTGCTTCAAGATCTACCACAAGTGCGACAA | |
| | CGCCTGCATTGGCAGCATCAGAAACGGCACCTACGACCATGACGTTTACCGGGACGAGGCCCTG | |
| | AACAACCGGTTCCAGATCAAGGGCGTGGAACTGAAGTCCGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 77 | ATGAAAACGATCATCGCCCTATCCTACATCCTATGCCTCGTCTTCGCCCAAAAACTCCCCGGCA | A/ |
| | ACGACAACAGCACAGCCACCCTGTGCCTGGGACACCACGCTGTGCCAAATGGCACCCTGGTGAA | Nanchang / |
| | GACAATCACCAACGACCAGATCGAAGTGACAAATGCCACAGAGCTGGTGCAGAGCTCCTCCACC | 933/ |
| | GGCAGAATCTGCGACTCTCCTCACAGAATCCTGGACGGCAAGAACTGCACCCTGATCGATGCCC | 1995 |
| | TGCTGGGCGACCCTCACTGCGACGGCTTCCAGAACAAGGAGTGGGACCTGTTCGTGGAGAGAAG | |
| | CAAGGCCTACTCCAACTGTTACCCTTACGACGTCCCTGATTACGCCTCCCTGAGAAGCCTGGTG | |
| | GCCTCCTCTGGCACCCTGGAGTTCACCAATGAGGGCTTCAACTGGACCGGAGTGGCCCAGGATG | |
| | GCACCTCCTACGCCTGCAAGAGAGGCTCCGTGAAAAGCTTCTTCAGCAGACTGAACTGGCTCCA | |
| | CAAGCTGGAGTACAAGTACCCCGCCCTGAACGTGACCATGCCAAACAACGACAAGTTCGACAAG | |
| | CTCTACATCTGGGGCGTCCACCACCCCAGCACCGACAGCGACCAGACATCTCTCTATGTCCAGG | |
| | CCAGCGGCAGAGTGACAGTCAGCACCAAGAGAAGCCAGCAAACAGTGATCCCCAACATCGGCAG | |
| | CAGACCTTGGGTGCGGGGCATCTCTTCCAGAATCAGCATCTACTGGACAATCGTGAAGCCTGGC | |
| | GATATCCTGCTGATCAAGAGCACCGGCAACCTGATCGCCCCCAGAGGCTACTTCAAGATCAGAA | |
| | GCGGAAAGAGCTCCATCATGAGAAGCGATGCTCCTATCGGCAACTGCAACTCTGAGTGTATCAC | |
| | CCCCAACGGCTCCATTCCCAACGACAAGCCTTTTCAGAACGTGAACAGAATCACCTACGGGGCC | |
| | TGCCCCAGGTACGTGAAGCAGAACACCCTGAAGCTGGCCACCGGAATGAGAAACGTCCCCGAGA | |
| | AGCAGACACGGGGCATCTTTGGCGCCATCGCCGGCTTCATCGAGAACGGCTGGGAGGGGATGGT | |
| | GGATGGCTGGTACGGCTTCCGGttCCAGAATTCGGAGGGCACCGGCCAGGCCGCCGACTTGAAA | |
| | TCCACCCAGGCCGCCATCAACCAGATCAACGGCctgCTGAACAGACTGATCGAGAAGACAAATG | |
| | AGAAGTTCCACCAGATCGAGAAGGAGTTCAGCGAAGTGGAGGGAAGAATCCAGGACCTGGAGAA | |
| | GTACGTGGAAGACACCAAGATCGACCTGTGGTCCTACAACGCAGAACTGCTGGTGGCCCTGctg | |
| | AACCAACACACAATCGACCTGACAGACAGCGAAATGAACAAACTGTTCGAGAGAACCAGAAAAC | |
| | AGCTGAGAGAGAACGCCGAGGACATGGGCAACGGCTGTTTCAAGATCTACCACAAGTGCGACAA | |
| | CGCCTGCATTGGCTCCATCAGAAACGGCACCTACGATCACGATGTTTACCGGGACGAGGCCCTG | |
| | AACAATCGTTTCCAGATCAAGGGAGTGGAGCTGAAGTCTGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 78 | ATGAAAACGATCATCGCCCTATCCTACATCCTATGCCTCGTCTTCGCCCAAAAACTCCCCGGCA | A/ |
| | ACGACAACAGCACCGCCACCCTGTGCCTCGGGCACCACGCTGTGCCAAACGGCACCCTGGTGAA | Bilthoven/ |
| | GACCATCACCAACGATCAGATCGAAGTGACCAATGCCACCGAACTGGTGCAGAGCTCCTCCACC | 1761/ |
| | GGCAAGATCTGCGACAACCCTCACAGAATCCTGGACGGCATCAACTGCACCCTGATCGATGCCC | 1976 |
| | TGCTGGGCGACCCTCACTGCGATGGCTTCCAGAATGAGAAGTGGGACCTGTTCGTCGAGAGAAG | |
| | CAAGGCCTTTTCCAACTGTTACCCTTACGACGTCCCCGATTACGCCAGCCTGAGATCCCTGGTG | |
| | GCCTCCAGCGGAACACTGGAGTTCATCAACGAGGGCTTCAACTGGACAGGCGTGACCCAGAACG | |
| | GCGGCTCCTCTGCCTGCAAGAGAGGCCCCGACAATGGCTTTTTCAGCAGACTGAACTGGCTGTA | |
| | CAAGAGCGGCAGCACCTACCCAGTCCAGAACGTGACCATGCCAAACAACGACAACTCCGACAAG | |
| | CTGTACATCTGGGGCGTCCACCACCCCAGCACAGACAAGGAGCAGACAGACCTCTACGTCCAAG | |
| | CCTCCGGCAAGGTCACCGTGTCCACCAAGCGGAGCCAGCAGACCGTGATCCCCAACGTGGGCAG | |
| | CCGGCCCTGGGTGAGAGGCCTGAGCTCCCGGGTGTCCATCTACTGGACCATCGTGAAACCAGGC | |
| | GACATCCTGGTGATCAACAGCAACGGCAACCTGATCGCCCCTCGGGGCTACTTCAAGATGAGAA | |
| | CCGGCAAGAGCAGCATCATGAGAAGCGATGCCCCAATCGGAACATGCTCCTCTGAATGCATTAC | |
| | ACCCAACGGAAGCATCCCCAACGACAAACCCTTCCAGAACGTGAACAAGATTACATATGGCGCC | |
| | TGCCCCAAGTACGTGAAGCAGAACACACTGAAGCTGGCCACAGGAATGAGAAACGTCCCTGAGA | |
| | AGCAGACAAGAGGCATCTTTGGCGCTATCGCCGGATTCATCGAAAACGGCTGGGAGGGAATGAT | |
| | CGACGGCTGGTACGGATTTAGAttCCAAAACAGCGAGGGCACCGGCCAGGCCGCTGACCTGAAG | |
| | TCAACCCAGGCCGCCATCGACCAGATCAACGGCctgCTGAACAGAGTGATCGAAAAGACCAATG | |
| | AGAAGTTCCATCAGATCGAGAAGGAGTTCAGCGAAGTGGAGGGCAGAATCCAGGACCTGGAGAA | |
| | GTACGTGGAGGATACCAAGATCGACCTGTGGAGCTACAACGCAGAGCTGCTGGTGGCCCTGctg | |
| | AACCAGCACACCATCGACCTGACAGACAGCGAGATGAACAAGCTGTTCGAGAAGACAAGAAGAC | |
| | AGCTGAGAGAGAACGCCGAGGATATGGGCAACGGCTGTTTCAAGATCTATCACAAGTGCGACAA | |
| | CGCCTGCATTGGCAGCATCAGAAACGGCACCTACGACCACGATGTCTACAGAGACGAGGCTCTG | |
| | AACAACAGATTCCAGATCAAGGGCGTGGAGCTGAAGTCCGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 79 | ATGAAAACGATCATCGCCCTATCCTACATCCTATGCCTCGTCTTCGCCCAAAAACTCCCCGGCA | A/ |
| | ACGACAACAGCACCGCCACACTGTGCCTGGGACACCACGCTGTGCCAAACGGCACAATCGTGAA | Cambodia/ |
| | GACAATCACCAATGACAGAATCGAGGTGACCAATGCCACAGAGCTGGTGCAGAACAGCTCTATC | e0826360/ |
| | GGCGAAATCTGCGACAGCCCCCACCAGATCCTGGACGGCGGCAATTGCACCCTGATCGATGCCC | 2020 |
| | TGCTGGGCGACCCTCAGTGCGACGGCTTCCAGAACAAGGAATGGGACCTGTTCGTGGAGCGGAG | |
| | CAGAGCCAATTCAAACTGCTACCCCTACGACGTCCCCGACTACGCCAGCCTGCGGAGCCTGGTG | |
| | GCCTCCAGCGGCACACTGGAGTTCAAGAACGAGTCTTTCAACTGGACAGGCGTCAAGCAGAACG | |
| | GCACCTCCAGCGCCTGCATCAGGGGCTCCAGCAGCAGCTTCTTCAGCAGACTGAACTGGCTGAC | |
| | CCACCTGAATTACACCTACCCCGCCCTGAACGTGACCATGCCAAACAACGAACAGTTCGACAAG | |
| | CTGTACATCTGGGGAGTGCATCACCCCAGCACAGACAAGGACCAGATCAGCCTGTTCGCTCAGC | |
| | CCTCCGGCAGAATTACCGTGTCCACCAAGAGAAGCCAGCAAGCCGTGATCCCAAACATCGGCAG | |
| | CCGGCCTCGGATCCGGGACATCCCTTCCAGAATCTCCATCTACTGGACCATCGTGAAGCCTGGC | |
| | GACATCCTGCTGATCAACAGCACAGGCAATCTGATCGCCCCCAGGGGCTACTTCAAGATCAGAA | |
| | GCGGCAAGAGCAGCATCATGAGAAGCGATGCCCCCATCGGCAAGTGCAAGAGCGAGTGTATCAC | |
| | CCCTAACGGCAGCATCCCAAACGACAAGCCTTTCCAGAACGTGAACAGAATCACATATGGAGCC | |
| | TGCCCCAGGTACGTCAAGCAGAGCACACTGAAGCTGGCCACAGGGATGAGAAACGTCCCTGAGA | |
| | AGCAGACCCGGGGCATCTTTGGCGCCATCGCCGGCTTTATCGAGAACGGCTGGGAGGGAATGGT | |
| | GGATGGCTGGTATGGCTTCCGGTTCCAGAACAGCGAGGGAAGAGGCCAGGCCGCTGACCTGAAG | |
| | TCTACCCAGGCCGCCATCGACCAGATCAATGGCCTGCTCAATCGCCTGATTGGCAAGACCAATG | |
| | AAAAGTTTCACCAGATCGAGAAGGAGTTCTCCGAAGTCGAGGGCCGGGTGCAGGACCTGGAGAA | |
| | GTACGTGGAAGACACCAAGATCGACCTGTGGTCTTACAACGCTGAACTGCTGGTGGCCCTGCTG | |
| | AACCAACACACAATCGACCTGACCGACAGCGAGATGAACAAGCTGTTTGAGAAGACCAAGAAGC | |
| | AGCTGAGAGAGAACGCCGAGGACATGGGCAACGGCTGCTTCAAGATCTACCACAAGTGCGACAA | |
| | TGCCTGCATTGGCAGCATCCGGAACGAAACCTACGACCACAATGTCTACAGAGACGAGGCCCTG | |
| | AACAACAGATTCCAGATCAAAGGCGTGGAGCTGAAATCCGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 80 | ATGAAAACGATCATCGCCCTATCCTACATCCTATGCCTCGTCTTCGCCCAAAAACTCCCCGGCA | A/ |
| | ACGACAACAGCACAGCAACCCTGTGCCTGGGACACCACGCAGTCCCCAACGGCACAATCGTGAA | Argentina/ |
| | GACAATCACCAACGACCAGATTGAGGTGACAAATGCCACAGAGCTGGTGCAGAGCTCCAGCACA | 28302/ |
| | GGCGAGATCTGCGACAGCCCTCACCAGATCCTGGATGGCAAGAACTGCACCCTGATCGATGCCC | 2010 |
| | TGCTGGGCGACCCCCAGTGCGACGGCTTCCAGAACAAAAAGTGGGACCTGTTCGTGGAGAGAAG | |
| | CAAGGCCTACAGCAACTGCTACCCTTACGATGTCCCTGACTACGCCAGCCTGCGGAGCCTGGTG | |
| | GCCTCCAGCGGCACCCTGGAGTTCAACAACGAGAGCTTCAATTGGACAGGCGTGACCCAGGATG | |
| | GAACAAGCAGCGCCTGCATCCGGGGCTCCAAGAATTCCTTCTTCAGCAGACTGAACTGGCTGAC | |
| | ACACCTGAACTTCAAGTACCCTGCCCTGAACGTGACAATGCCAAACAACGAACAATTCGACAAG | |
| | CTGTACATCTGGGGAGTGCACCACCCCGGCACCGACAAGGATCAGATCTTTCTGTATGCCCAGG | |
| | CCAGCGGCCGGATCACCGTGTCCACCAAAAGAAGCCAGCAGGCCGCTATCCCCAATATCGGCAG | |
| | CCGGCCTCGGGTGAGAAACATCCCTTCCAGAATCTCAATCTACTGGACAATCGTGAAGCCTGGC | |
| | GACATCCTGCTGATCAATAGCACCGGCAACCTGATCGCCCCCAGAGGCTACTTCAAGATCAGAA | |
| | GCGGAAAGAGCAGCATCATGAGAAGCGATGCCCCCATCGGCAAGTGCAATTCTGAATGCATCAC | |
| | CCCCAACGGCTCCATTCCCAACGACAAGCCTTTCCAGAACGTGAATCGGATCACCTATGGCGCC | |
| | TGCCCCAGGTACGTGAAGCAGAACACACTGAAGCTGGCCACCGGGATGAGAAATGTCCCCGAGA | |
| | AGCAGACCAGGGGCATCTTTGGCGCCATCGCCGGCTTCATCGAGAACGGCTGGGAAGGCATGGT | |
| | GGACGGCTGGTATGGCTTCAGGttCCAGAACAGCGAAGGCAGAGGCCAGGCCGCTGACCTGAAA | |
| | TCCACCCAGGCTGCCATCGACCAGATCAATGGActgCTGAATAGGCTGATCGGCAAGACAAACG | |
| | AGAAATTCCACCAGATCGAGAAGGAGTTCAGCGAAGTGGAGGGCCGGATTCAGGACCTGGAGAA | |
| | GTACGTGGAGGACACAAAGATTGACCTGTGGAGCTACAACGCAGAGCTGCTGGTGGCCCTGctg | |
| | AACCAGCACACCATCGACCTGACAGACAGCGAGATGAACAAGCTGTTTGAGAAGACAAAAAAGC | |
| | AGCTGAGAGAGAATGCCGAGGACATGGGAAACGGCTGTTTCAAGATCTACCACAAGTGCGACAA | |
| | CGCCTGCATTGGCAGCATCCGGAACGGCACCTACGATCACGATGTTTACCGGGACGAAGCCCTG | |
| | AACAAAAGATTCCAGATCAAGGGGATCGAACTGAAATCCGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 81 | ATGAAAACGATCATCGCCCTATCCTACATCCTATGCCTCGTCTTCGCCCAAAAACTCCCCGGCA | A/ |
| | ACGACAACAGCACCGCCACCCTGTGCCTGGGACACCACGCTGTGCCAAATGGCACCCTGGTGAA | Auckland/ |
| | GACCATCACAAACGACCAGATCGAGGTCACAAATGCCACCGAACTGGTCCAGTCTTCTAGCACA | 588/ |
| | GGCGGCATCTGCGACAGCCCCCACCAAATTCTGGATGGCGAGAACTGCACCCTGATCGATGCCC | 2000 |
| | TGCTGGGAGATCCCCACTGCGACGGCTTCCAGAACAAAGAATGGGACCTGTTCGTGGAGCGGAG | |
| | CAAAGCCTACTCCAACTGCTACCCCTACGATGTCCCCGACTACGCCTCCCTGAGAAGCCTGGTG | |
| | GCCAGCTCTGGCACCCTGGAGTTCAACAATGAGTCCTTCAACTGGACAGGCGTGGCCCAGAATG | |
| | GCACATCCTCCGCCTGCAAGCGGAGATCTAACAAGAGCTTCTTTAGCAGACTGAATTGGCTGCA | |
| | CCAGCTGAAGTACAAGTACCCTGCCCTGAACGTGACCATGCCAAACAATGAGAAGTTCGACAAG | |
| | CTGTACATCTGGGGCGTCCATCACCCCTCCACCGACAGCGACCAGATCAGCCTGTACGCCCAAG | |
| | CCCCTGGCAGAGTGACAGTGAGCACAAAGCGGAGCCAGCAGACAGTGATCCCGAACATCGGCAG | |
| | CCGGCCTTGGGTGAGAGGCGTGTCCAGCAGAATCAGCATCTACTGGACAATCGTGAAGCCTGGC | |
| | GACATCCTGCTGATCAACTGCACAGGCAACCTGATCGCCCCAAGAGGCTACTTCAAGATCCGGT | |
| | CCGGCAAGAGCAGCATCATGAGAAGCGATGCCAGCATCGGAAAGTGCAACAGCGAGTGTATCAC | |
| | CCCCAACGGCAGCATCCCCAACGACAAGCCTTTCCAGAACGTGAATCGGATCACCTATGGCGCC | |
| | TGCCCCAGGTACGTGAAGCAGAACACCCTGAAACTCGCCACCGGGATGAGAAACGTCCCCGAGA | |
| | AACAGACAAGAGGCATCTTTGGAGCCATCGCCGGCTTCATCGAGAACGGCTGGGAGGGGATGGT | |
| | GGACGGCTGGTACGGATTCCGGttCCAGAACAGCGAGGGCACCGGCCAGGCCGCTGACCTGAAA | |
| | AGCACCCAGGCCGCCATCAATCAAATCAATGGCctgCTGAACAGACTGATCGAAAAGACAAATG | |
| | AGAAGTTCCACCAGATCGAAAAGGAATTCAGCGAGGTGGAAGGCAGAATCCAGGACCTGGAGAA | |
| | GTACGTGGAGGATACCAAGATCGACCTGTGGAGCTACAACGCTGAACTGCTGGTGGCCCTGctg | |
| | AACCAGCACACAATCGACCTGACCGACAGCGAGATGAACAAGCTGTTCGAGAGAACCAAGAAGC | |
| | AGCTGAGAGAGAACGCCGAGGACATGGGCAATGGTTGCTTCAAGATCTACCACAAGTGCGACAA | |
| | CGCCTGCATTGGAAGCATCAGAAACGGCACATACGACCACGATGTCTACAGAGACGAGGCTCTG | |
| | AACAATCGGTTTCAGATCAAGGGCGTGGAGCTGAAGTCTGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 82 | ATGAAAACGATCATCGCCCTATCCTACATCCTATGCCTCGTCTTCGCCCAAAAACTCCCCGGCA | A/ |
| | ACGACAACAGCACCGCCACCCTGTGCCTGGGCCACCACGCTGTGCCAAACGGCACCCTGGTGAA | Memphis/ |
| | GACCATCACCAACGATCAGATTGAAGTGACCAATGCCACAGAGCTGGTGCAGAGCTCCTCCACT | 1/ |
| | GGCAGAATCTGCGACTCCCCTCACAGAATCCTGGATGGCAAAAACTGCACACTCGTGGATGCCC | 1980 |
| | TGCTGGGCGACCCCCACTGCGACGGCTTCCAGAATGAGAAGTGGGACCTGTTCGTGGAGAGAAG | |
| | CAAGGCCTTCAGCAACTGCTACCCTTACGACGTCCCTGACTACGCCAGCCTGAGAAGCCTGGTG | |
| | GCCTCCAGCGGCACACTGGAGTTCATCAATGAGTCCTTCAACTGGACAGGCGTGACACAGTCTG | |
| | GCGGCTCTTACGCCTGCAAGCGGGGCAGCGACAACAGCTTCTTCTCCCGGCTGAATTGGCTGTA | |
| | CGAGAGCGAGAGCAAGTACCCTGTGCTGAACGTGACCATGCCAAACAATGGCAATTTCGACAAA | |
| | CTGTACATCTGGGGAGTGCACCACCCCAGCACCGACAAGGAGCAGACCAACCTCTACGTGAGAG | |
| | CCAGCGGCAGAGTGACCGTGTCCACAAAGCGGAGCCAGCAGACCATCATCCCCAACATCGGCAG | |
| | CCGGCCCTGGGTGAGAGGCCTGAGCTCCCGGATCAGCATCTACTGGACAATCGTGAAACCCGGA | |
| | GACATCCTGCTGATCAACAGCAACGGCAACCTGATCGCCCCCAGAGGCTACTTTAAGATCAGAA | |
| | CAGGCAAGAGCTCCATCATGAGAAGCGATGCCCCCATCGGCACATGCAGCAGCGAGTGTATCAC | |
| | CCCCAACGGCTCAATCCCCAACGACAAGCCTTTCCAGAACGTGAACAAGATTACCTATGGCGCC | |
| | TGCCCCAAGTACGTGAAGCAGAACACACTGAAGCTGGCCACAGGGATGAGGAATGTCCCCGAAA | |
| | AGCAGACAAGAGGCATCTTTGGGGCCATCGCCGGCTTCATCGAGAACGGCTGGGAAGGAATGGT | |
| | GGACGGCTGGTACGGCTTCAGGttCCAGAACAGCGAGGGCACCGGCCAGGCCGCTGACCTGAAG | |
| | TCCACCCAGGCCGCCATCGACCAGATCAATGGActgCTGAACAGAGTGATCGAGAAGACAAACG | |
| | AAAAGTTCCACCAGATCGAAAAGGAATTCTCCGAGGTGGAAGGACGGATTCAGGACCTGGAAAA | |
| | GTACGTGGAAGACACCAAGATCGACCTGTGGAGCTACAACGCAGAGCTGCTGGTGGCTCTGctg | |
| | AACCAGCACACAATCGACCTGACAGACAGCGAGATGAACAAACTGTTCGAAAAGACACGGAGAC | |
| | AGCTGAGGGAGAACGCTGAAGACATGGGAAACGGCTGCTTTAAGATTTACCACAAGTGCGACAA | |
| | TGCCTGCATTGGCAGCATTCGGAACGGCACATACGACCATGATGTCTACAGAGACGAGGCTCTG | |
| | AACAACCGGTTCCAGATCAAGGGCGTGGAGCTGAAGTCTGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 83 | ATGAAAACGATCATCGCCCTATCCTACATCCTATGCCTCGTCTTCGCCCAAAAACTCCCCGGCA | A/ |
| | ACGACAACAGCACAGCCACCCTGTGCCTGGGCCACCACGCTGTGCCAAACGGCACACTGGTGAA | HongKong/ |
| | GACAATCACCGACGACCAGATCGAGGTGACCAATGCAACAGAGCTGGTGCAGAGTTCCAGCACC | 1/ |
| | GGAAAGATCTGCAACAACCCTCACCGGATCCTGGACGGCATCGACTGCACCCTGATTGATGCCC | 1968 |
| | TGCTCGGCGACCCTCACTGCGACGTGTTCCAGAACGAAACATGGGACCTGTTCGTGGAGCGGTC | |
| | CAAGGCCTTTAGCAACTGCTACCCCTACGACGTCCCCGATTACGCCAGCCTGAGATCTCTCGTG | |
| | GCCTCCAGCGGCACCCTGGAGTTCATTACAGAGGGCTTCACATGGACAGGCGTGACACAGAACG | |
| | GCGGCAGCAACGCCTGCAAGAGAGGCCCTGGCTCTGGCTTCTTCAGCAGGCTGAATTGGCTGAC | |
| | AAAGAGCGGAAGCACCTACCCTGTGCTGAACGTGACCATGCCAAACAACGACAATTTCGACAAG | |
| | CTGTACATCTGGGGCGTCCACCACCCCAGCACCAACCAGGAGCAGACCAGCCTCTATGTCCAGG | |
| | CCAGCGGCAGAGTGACAGTGAGCACCAGACGGTCCCAGCAGACCATCATCCCTAACATCGGATC | |
| | CAGACCTTGGGTGCGGGGCCTGAGCAGCAGAATCTCCATCTACTGGACCATCGTGAAACCTGGC | |
| | GATGTCCTGGTGATCAACTCCAACGGCAACCTGATCGCCCCTCGGGGCTACTTCAAGATGCGGA | |
| | CAGGCAAGAGCTCAATCATGAGAAGCGATGCTCCTATCGACACCTGCATCAGCGAGTGTATCAC | |
| | ACCCAACGGCTCTATCCCCAACGACAAGCCCTTCCAGAACGTGAACAAGATCACATATGGAGCC | |
| | TGCCCCAAGTACGTGAAACAGAACACCCTGAAGCTGGCCACCGGGATGAGAAACGTCCCTGAGA | |
| | AGCAGACCCGGGGCCTGTTTGGAGCCATCGCCGGCTTCATCGAGAACGGCTGGGAAGGCATGAT | |
| | CGACGGCTGGTACGGATTCCGGttCCAGAACAGCGAAGGCACCGGCCAGGCTGCTGACCTGAAG | |
| | TCCACACAGGCCGCCATCGACCAGATCAATGGCctgCTGAACAGAGTGATCGAAAAGACAAACG | |
| | AAAAGTTCCACCAGATCGAAAAGGAGTTCAGCGAAGTGGAGGGCAGAATCCAGGACCTGGAGAA | |
| | GTACGTGGAGGACACCAAGATCGACCTGTGGAGCTACAACGCAGAACTGCTGGTCGCCCTGctg | |
| | AACCAGCACACCATCGACCTGACAGATTCGGAGATGAACAAGCTGTTCGAGAAAACCCGGAGAC | |
| | AGCTGAGAGAGAACGCCGAGGACATGGGAAACGGCTGCTTCAAGATTTACCACAAGTGCGACAA | |
| | CGCCTGCATTGAATCCATTCGGAACGGCACCTACGACCACGATGTCTACAGAGACGAGGCCCTG | |
| | AACAACCGGTTTCAGATCAAGGGCGTGGAGCTGAAGTCCGGCTACAAGGACTGGATCTTGTGGA | |
| | TCAGCTTCGCCATCTCCTGCTTCCTGCTCTGTGTGGTCCTGCTCGGCTTCATTATGTGGGCCTG | |
| | TCAGAAGGGCAACATCCGATGCAACATCTGCATTtga | |
| 84 | ATGAAGGCGATCATCGTCCTCCTGATGGTAGTGACAAGCAATGCCGACAGAATTTGCACCGGCA | B/ |
| | TTACCAGCAGCAACAGCCCTCACGTGGTCAAGACCGCCACCCAGGGCGAGGTGAACGTGACAGG | Austria/ |
| | CGTGATTCCCCTGACAACCACCCCCACCAAGAGCCACTTCGCCAACCTGAAAGGCACCGAGACA | 1359417/ |
| | CGGGGCAAACTGTGCCCCAAGTGCCTCAACTGTACCGACCTGGACGTGGCCCTGGGCAGACCCA | 2021 |
| | AGTGCACCGGCAAAATCCCAAGCGCCAGAGTGAGCATCCTGCACGAGGTGCGGCCCGTGACCAG | |
| | CGGCTGCTTCCCCATTATGCACGATAGAACCAAGATCAGACAGCTGCCAAATCTGCTGAGAGGC | |
| | TACGAACACGTCAGACTGTCCACCCACAACGTGATCAACACAGAGGACGCCCCTGGCGGCCCTT | |
| | ACGAGATCGGCACAAGCGGCTCCTGCCTGAACATTACAAACGGCAAAGGCTTCTTCGCCACCAT | |
| | GGCTTGGGCCGTCCCCAAGAACAAGACAGCCACCAATCCTCTGACCATCGAGGTCCCTTACATC | |
| | TGCACCGAAGAGGAGGATCAGATCACAGTGTGGGGCTTCCACAGCGATGACGAGACACAGATGG | |
| | CCAGACTGTACGGCGACAGCAAGCCTCAGAAGTTCACCAGCAGCGCCAACGGCGTGACCACCCA | |
| | CTACGTCAGCCAGATTGGCGGCTTTCCTAACCAGACCGAGGACGGCGGCCTGCCTCAGAGCGGC | |
| | AGAATTGTGGTGGACTACATGGTTCAGAAAAGCGGCAAGACAGGCACAATCACCTACCAGCGGG | |
| | GCATCCTGCTGCCTCAGAAGGTGTGGTGCGCCTCCGGCAAATCTAAGGTGATCAAGGGCTCCCT | |
| | GCCTCTGATCGGAGAGGCCGACTGCCTGCACGAGAAGTACGGAGGCCTGAACAAGAGCAAACCC | |
| | TACTACACAGGCGAACATGCCAAGGCCATCGGCAACTGCCCCATCTGGGTCAAGACCCCTCTGA | |
| | AGCTGGCCAATGGAACAAAGTACAGACCTCCTGCCAAGCTGCTGAAGGAGAGAGGCTTCTTTGG | |
| | CGCCATCGCCGGCTTCCTGGAAGGCGGCTGGGAGGGCATGATCGCCGGCTGGTACGGATACACC | |
| | TCTCATGGAGCACACGGCGTGGCCGTGGCCGCTGACCTCAAGAGCACCCAGGAGGCCATTAACA | |
| | AGATCACAAAGAACCTGAACTCCCTGAGCGAACTGGAGGTGAAGAATCTGCAGAGACTGTCAGG | |
| | CGCCATGGACGAACTGCACAATGAGATCCTGGAGCTGGACGAGAAGGTGGACGACCTGCGGGCC | |
| | GACACCATCTCCTCCCAGATCGAACTGGCAGTCCTGCTGAGCAACGAGGGCATCATCAACTCCG | |
| | AGGACGAGCACCTGCTGGCCCTGGAGAGAAAGCTGAAGAAGATGCTGGGCCCCTCTGCCGTGGA | |
| | GATCGGCAACGGCTGCTTCGAAACCAAGCACAAATGCAACCAGACCTGTCTGGACCGGATTGCA | |
| | GCCGGCACCTTTGACGCCGGGGAATTCAGCCTGCCTACCTTCGACTCCCTGAACATCACCGCCG | |
| | CTTCTCTGAACGACGACGGCCTCGACAATCACACCATCCTGCTCTACTACTCCACCGCCGCCTC | |
| | CAGCCTGGCCGTGACCCTGATGATCGCCATCTTCGTGGTGTACATGGTTTCCAGAGACAACGTC | |
| | TCCTGCAGCATCTGCCTGtga | |

Table 2 depicts nucleic acid sequences of the invention. Each nucleic acid sequence encodes for a signaling peptide sequence (denoted with single underlining), a stabilized extracellular sequence, and a transmembrane domain sequence (denoted with double underlining). In some embodiments, a vaccine composition comprises nucleic acids comprising any one of (or all of) the sequences in Table 2.

TABLE 3
Example Composition A with amino acid sequences for antigens comprising signal
peptide, stabilized extracellular domain, and transmembrane domain

| | | SEQ ID |
|---|---|---|
| Strain | Amino Acid Sequence of Antigen | NO: |
| A/New_York/1/191 | MKVKLLVLLCTFTATYADTICIGYHANNSTDTVDTVLEKNVTVTHSVNLLE | 85 |
| 8 A_/_H1N1 | DSHNGKLCKLKGIAPLQLGKCNIAGWLLGNPECDLLLTASSWSYIVETSNS | |
| | ENGTCYPGDFIDYEELREQLSSVSSFEKFEIFPKTSSWPNHETTKGVTAAC | |
| | SYAGASSFYRNLLWLTKKGSSYPKLSKSYVNNKGKEVLVLWGVHHPPTGTD | |
| | QQSLYQNADAYVSVGSSKYNRRFTPEIAARPKVRDQAGRMNYYWTLLEPGD | |
| | TITFEATGNLIAPWYAFALNRGSGSGIITSDAPVHDCNTKCQTPHGAINSS | |
| | LPFQNIHPVTIGECPKYVRSTKLRMATGLRNIPSIQSRGLFGAIAGFIEGG | |
| | WTGMIDGWYGYHFQNEQGSGYAADRKSTQNAIDGITNLVNSVIEKMNTQFT | |
| | SVGKEFNHLEKRIENLNRKVDDGELDVWTYNAELLVLLLNERTLDYHDSNV | |
| | KNLYEKVRSQLKNNAKEIGNGCFEFYHKCDDSCMESVKNGTYDYPKYSEES | |
| | KLNREEIDGVKLESMGVYQILAIYSTVASSLVLLVSLGAISFWMCSNGSLQ | |
| | CRICI | |
| A/Wisconsin/588/ | MKVKLLVLLCTFTATYADTLCIGYHANNSTDTVDTVLEKNVTVTHSVNLLE | 86 |
| 2019 A_/_H1N1 | DKHNGKLCKLRGVAPLHLGKCNIAGWILGNPECESLSTARSWSYIVETSNS | |
| | DNGTCYPGDFINYEELREQLSSVSSFERFEIFPKTSSWPNHDSDNGVTAAC | |
| | PHAGAKSFYKNLIWLVKKGKSYPKINQTYINDKGKEVLVLWGIHHPPTIAD | |
| | QQSLYQNADAYVFVGTSRYSKKEKPEIATRPKVRDQEGRMNYYWTLVEPGD | |
| | KITFEATGNLVAPRYAFTMERDAGSGIIISDTPVHDCNTTCQTPEGAINTS | |
| | LPFQNVHPITIGKCPKYVKSTKLRLATGLRNVPSIQSRGLFGAIAGFIEGG | |
| | WTGMVDGWYGYHFQNEQGSGYAADLKSTQNAIDKITNAVNSVIEKMNTQFT | |
| | AVGKEFNHLEKRIENLNKKVDDGELDIWTYNAELLVLLANERTLDYHDSNV | |
| | KNLYEKVRNQLKNNAKEIGNGCFEFYHKCDNTCMESVKNGTYDYPKYSEEA | |
| | KLNREKIDGVKLDSTRIYQILAIYSTVASSLVLVVSLGAISFWMCSNGSLQ | |
| | CRICI | |
| A/Brisbane/59/20 | MKVKLLVLLCTFTATYADTICIGYHANNSTDTVDTVLEKNVTVTHSVNLLE | 87 |
| 07 A_/_H1N1 | NSHNGKLCLLKGIAPLQLGNCSVAGWILGNPECELLISKESWSYIVEKPNP | |
| | ENGTCYPGHEADYEELREQLSSVSSFERFEIFPKESSWPNHTVTGVSASCS | |
| | HNGESSFYRNLLWLTGKNGLYPNLSKSYANNKEKEVLVLWGVHHPPNIGNQ | |
| | KALYHTENAYVSVVSSHYSRKFTPEIAKRPKVRDQEGRINYYWTLLEPGDT | |
| | IIFEANGNLIAPRYAFALSRGFGSGIINSNAPMDKCDAKCQTPQGAINSSL | |
| | PFQNVHPVTIGECPKYVRSAKLRMVTGLRNIPSIQSRGLFGAIAGFIEGGW | |
| | TGMVDGWYGYHFQNEQGSGYAADQKSTQNAINGITNAVNSVIEKMNTQFTA | |
| | VGKEFNKLERRMENLNKKVDDGFIDIWTYNAELLVLLANERTLDFHDSNVK | |
| | NLYEKVKSQLKNNAKEIGNGCFEFYHKCNDECMESVKNGTYDYPKYSEESK | |
| | LNREKIDGVKLESMGVYQILAIYSTVASSLVLLVSLGAISFWMCSNGSLQC | |
| | RICI | |
| A/Wisconsin/28/2 | MKVKLLVLLCTFTATYADTLCIGYHANNSTDTVDTVLEKNVTVTHSVNLLE | 88 |
| 011.2011/12 | NRHNGKLCKLRGVAPLHLGKCNIAGWLLGNPECESLSTASSWSYIVETSNS | |
| A_/_H1N1 | DNGTCYPGDFINYEELREQLSSVSSFERFEIFPKTSSWPNHDTNRGVTAAC | |
| | PHDGTNSFYRNLIWLVKKGNSYPKINKSYINNKEKEILVLWAIHHPSTSAD | |
| | QQSLYQNADAYVFVGSSRYSRKFEPEVATRPKVRDQAGRMNYYWTLVEPGD | |
| | KITFEATGNLVVPRYAFALKRNSGSGIIISDTSVHDCDTNCQTPNGAINTS | |
| | LPFQNIHPVTIGECPKYVKSTKLRMATGLRNIPSIQSRGLFGAIAGFIEGG | |
| | WTGMIDGWYGYHFQNEQGSGYAADLKSTQNAIDGITNLVNSVIEKMNTQFT | |
| | AVGKEFSHLERRIENLNKKVDDGELDIWTYNAELLVLLLNERTLDYHDSNV | |
| | KNLYEKVRSQLKNNAKEIGNGCFEFYHKCDDMCMESVKNGTYDYPKYSEEA | |
| | KLNREEIDGVKLESTRIYQILAIYSTVASSLVLVVSLGAISFWMCSNGSLQ | |
| | CRICI | |
| A/Denver/57 | MKVKLLVLLCTFTATYADTICIGYHANNSTDTVDTVLEKNVTVTHSVNLLE | 89 |
| A_/_H1N1 | DSHNGKLCRLKGKAPLQLGNCNIAGWVLGNPECESLLSNRSWSYIAETPNS | |
| | ENGTCYPGDFADYEELREQLSSVSSFERFEIFPKERSWPNHTTRGVTAACP | |
| | HARKSSFYKNLVWLTEANGSYPNLSRSYVNNQEKEVLVLWGVHHPSNIEEQ | |
| | RALYRKDNAYVSVVSSNYNRRFTPEIAKRPKVRDQSGRMNYYWTLLEPGDT | |
| | IIFEATGNLIAPWYAFALSRGPGSGIITSNAPLDECDTKCQTPQGAINSSL | |
| | PFQNIHPVTIGECPKYVRSTKLRMVTGLRNIPSVQSRGLFGAIAGFIEGGW | |
| | TGMMDGWYGYHFQNEQGSGYAADQKSTQNAINGITNAVNSVIEKMNTQFTA | |
| | VGKEENKLEKRMENLNKKVDDGEMDIWTYNAELLVLLANERTLDFHDSNVK | |
| | NLYEKVKNQLRNNAKELGNGCFEFYHKCDNECMESVKNGTYDYPKYSEESK | |
| | LNREKIDGVKLESMGVYRILAIYSTVASSLVLLVSLGAISFWMCSNGSLQC | |
| | RICI | |
| A/Beijing/262/19 | MKVKLLVLLCTFTATYADTICIGYHANNSTDTVDTVLEKNVTVTHSVNLLE | 90 |
| 95 A_/_H1N1 | DSHNGKLCLLKGIAPLQLGNCSVAGWILGNPECESLISKESWSYIVETPNP | |
| | ENGTCYPGYFADYEELREQLSSVSSFERFEIFPKESSWPNHTVTGVTASCS | |
| | HNGKSSFYRNLLWLTEKNGLYPNLSNSYVNNKEKEVLVLWGVHHPSNIRDQ | |
| | RAIYHTENAYVSVVSSHYSRRFTPEIAKRPKVRGQEGRINYYWTLLEPGDT | |
| | IIFEANGNLIAPWYAFALSRGFGSGIITSNAPMNECDAKCQTPQGAINSSL | |
| | PFQNVHPVTIGECPKYVRSTKLRMVTGLRNIPSIQSRGLFGAIAGFIEGGW | |
| | TGMMDGWYGYHFQNEQGSGYAADQKSTQNAINGITNLVNSVIEKMNTQFTA | |
| | VGKEFNKLERRMENLNKKVDDGELDIWTYNAELLVLLLNERTLDFHDSNVK | |
| | NLYEKVKSQLKNNAKEIGNGCFEFYHKCNNECMESVKNGTYDYPKYSEESK | |
| | LNREKIDGVKLESMGVYQILAIYSTVASSLVLLVSLGAISFWMCSNGSLQC | |
| | RICI | |
| A/Puerto_Rico/8/ | MKVKLLVLLCTFTATYADTICIGYHANNSTDTVDTVLEKNVTVTHSVNLLE | 91 |
| 1934 A_/_H1N1 | DSHNGKLCRLKGIAPLQLGKCNIAGWLLGNPECDPLLPVRSWSYIVETPNS | |
| | ENGICYPGDFIDYEELREQLSSVSSFERFEIFPKESSWPNHNTNKGVTAAC | |
| | SHEGKSSFYRNLLWLTEKEGSYPKLKNSYVNKKGKEVLVLWGIHHPPNSKE | |
| | QQNLYQNENAYVSVVTSNYNRRFTPEIAERPKVRDQAGRMNYYWTLLKPGD | |
| | TIIFEANGNLIAPMYAFALSRGFGSGIITSNASMHECNTKCQTPLGAINSS | |
| | LPYQNIHPVTIGECPKYVRSAKLRMVTGLRNIPSIQSRGLFGAIAGFIEGG | |
| | WTGMIDGWYGYHFQNEQGSGYAADQKSTQNAINGITNAVNTVIEKMNIQFT | |
| | AVGKEENKLEKRMENLNKKVDDGELDIWTYNAELLVLLANERTLDFHDSNV | |
| | KNLYEKVKSQLKNNAKEIGNGCFEFYHKCDNECMESVRNGTYDYPKYSEES | |
| | KLNREKVDGVKLESMGIYQILAIYSTVASSLVLLVSLGAISFWMCSNGSLQ | |
| | CRICI | |
| A/California/07/ | MKVKLLVLLCTFTATYADTLCIGYHANNSTDTVDTVLEKNVTVTHSVNLLE | 92 |
| 2009 A_/_H1N1 | DKHNGKLCKLRGVAPLHLGKCNIAGWILGNPECESLSTASSWSYIVETPSS | |
| | DNGTCYPGDFIDYEELREQLSSVSSFERFEIFPKTSSWPNHDSNKGVTAAC | |
| | PHAGAKSFYKNLIWLVKKGNSYPKLSKSYINDKGKEVLVLWGIHHPSTSAD | |
| | QQSLYQNADAYVFVGSSRYSKKFKPEIAIRPKVRDQEGRMNYYWTLVEPGD | |
| | KITFEATGNLVVPRYAFAMERNAGSGIIISDTPVHDCNTTCQTPKGAINTS | |
| | LPFQNIHPITIGKCPKYVKSTKLRLATGLRNIPSIQSRGLFGAIAGFIEGG | |
| | WTGMVDGWYGYHFQNEQGSGYAADLKSTQNAIDEITNLVNSVIEKMNTQFT | |
| | AVGKEFNHLEKRIENLNKKVDDGELDIWTYNAELLVLLLNERTLDYHDSNV | |
| | KNLYEKVRSQLKNNAKEIGNGCFEFYHKCDNTCMESVKNGTYDYPKYSEEA | |
| | KLNREEIDGVKLESTRIYQILAIYSTVASSLVLVVSLGAISFWMCSNGSLQ | |
| | CRICI | |
| A/Indiana/11/201 | MKTIIALSYILCLVFAQKLPGNDNSTATLCLGHHAVPNGTIVKTITNDRIE | 93 |
| 8 A_/_H3N2 | VTNATELVQNSSIGEICDSPHQILDGENCTLIDALLGDPQCDGFQNKKWDL | |
| | FVERSKAYSNCYPYDVPDYASLRSLVASSGTLEENNESENWTGVKQNGTSS | |
| | ACIRKSSSSFFSRLNWLTHLNYTYPALNVTMPNNEQFDKLYIWGVHHPGTD | |
| | KDQIFLYAQSSGRITVSTKRSQQAVIPNIGSRPRIRDIPSRISIYWTIVKP | |
| | GDILLINSTGNLIAPRGYFKIQSGKSSIMRSDAPIGKCKSECITPNGSIPN | |
| | DKPFQNVNRITYGACPRYVKHSTLKLATGMRNVPEKQTRGIFGAIAGFIEN | |
| | GWEGMVDGWYGFRFQNSEGRGQAADLKSTQAAIDQINGLLNRLIGKTNEKF | |
| | HQIEKEFSEVEGRIQDLEKYVEDTKIDLWSYNAELLVALLNQHTIDLTDSE | |
| | MNKLFEKTKKQLRENAEDMGNGCFKIYHKCDNACIGSIRNGTYDHNVYRDE | |
| | ALNNRFQIKGVELKSGYKDWILWISFAISCELLCVVLLGFIMWACQKGNIR | |
| | CNICI | |
| A/California/07/ | MKTIIALSYILCLVFAQKLPGNDNSTATLCLGHHAVPNGTIVKTITNDQIE | 94 |
| 2004 A_/_H3N2 | VTNATELVQSSSTGGICDSPHQILDGENCTLIDALLGDPQCDGFQNKKWDL | |
| | FVERSKAYSNCYPYDVPDYASLRSLVASSGTLEENNESENWTGVTQNGTSS | |
| | SCKRRSNNSFFSRLNWLTHLKFKYPALNVTMPNNEKEDKLYIWGVHHPGTN | |
| | NDQISLYTQASGRITVSTKRSQQTVIPNIGSRPRVRDIPSRISIYWTIVKP | |
| | GDILLINSTGNLIAPRGYFKIRSGKSSIMRSDAPIGKCNSECITPNGSIPN | |
| | DKPFQNVNRITYGACPRYVKQNTLKLATGMRNVPEKQTRGIFGAIAGFIEN | |
| | GWEGMVDGWYGFRFQNSEGIGQAADLKSTQAAINQINGLLNRLIGKTNEKF | |
| | HQIEKEFSEVEGRIQDLEKYVEDTKIDLWSYNAELLVALLNQHTIDLTDSE | |
| | MNKLFERTKKQLRENAEDMGNGCFKIYHKCDNACIGSIRNGTYDHDVYRDE | |
| | ALNNRFQIKGVELKSGYKDWILWISFAISCELLCVVLLGFIMWACQKGNIR | |
| | CNICI | |
| A/Alaska/01/2021 | MKTIIALSYILCLVFAQKLPGNDNSTATLCLGHHAVPNGTIVKTITNDRIE | 95 |
| A_/_H3N2 | VTNATELVQNSSIGEICNSPHQILDGGNCTLIDALLGDPQCDGFQNKEWDL | |
| | FVERSRANSSCYPYDVPDYASLRSLVASSGTLEFKNESENWTGVKQNGTSS | |
| | ACIRGSSSSFFSRLNWLTSLNNIYPAQNVTMPNKEQFDKLYIWGVHHPDTD | |
| | KNQFSLFAQSSGRITVSTKRSQQAVIPNIGSRPRIRDIPSRISIYWTIVKP | |
| | GDILLINSTGNLIAPRGYFKIRSGKSSIMRSDAPIGKCKSECITPNGSIPN | |
| | DKPFQNVNRITYGACPRYVKQSTLKLATGMRNVPEKQTRGIFGAIAGFIEN | |
| | GWEGMVDGWYGFRFQNSEGRGQAADLKSTQAAIDQISGLLNRLIGKTNEKE | |
| | HQIEKEFSEVEGRVQDLEKYVEDTKIDLWSYNAELLVALLNQHTIDLTDSE | |
| | MNKLFEKTKKQLRENAEDMGNGCFKIYHKCDNACIGSIRNETYDHNVYRDE | |
| | ALNNRFQIKGVELKSGYKDWILWISFAISCELLCVVLLGFIMWACQKGNIR | |
| | CNICI | |
| A/Cambodia/e0826 | MKTIIALSYILCLVFAQKLPGNDNSTATLCLGHHAVPNGTIVKTITNDRIE | 96 |
| 360/2020 | VTNATELVQNSSIGEICDSPHQILDGGNCTLIDALLGDPQCDGFQNKEWDL | |
| A_/_H3N2 | FVERSRANSNCYPYDVPDYASLRSLVASSGTLEFKNESENWTGVKQNGTSS | |
| | ACIRGSSSSFFSRLNWLTHLNYTYPALNVTMPNNEQFDKLYIWGVHHPSTD | |
| | KDQISLFAQPSGRITVSTKRSQQAVIPNIGSRPRIRDIPSRISIYWTIVKP | |
| | GDILLINSTGNLIAPRGYFKIRSGKSSIMRSDAPIGKCKSECITPNGSIPN | |
| | DKPFQNVNRITYGACPRYVKQSTLKLATGMRNVPEKQTRGIFGAIAGFIEN | |
| | GWEGMVDGWYGFRFQNSEGRGQAADLKSTQAAIDQINGLLNRLIGKTNEKF | |
| | HQIEKEFSEVEGRVQDLEKYVEDTKIDLWSYNAELLVALLNQHTIDLTDSE | |
| | MNKLFEKTKKQLRENAEDMGNGCFKIYHKCDNACIGSIRNETYDHNVYRDE | |
| | ALNNRFQIKGVELKSGYKDWILWISFAISCELLCVVLLGFIMWACQKGNIR | |
| | CNICI | |
| A/Nanchang/933/1 | MKTIIALSYILCLVFAQKLPGNDNSTATLCLGHHAVPNGTLVKTITNDQIE | 97 |
| 995 A_/_H3N2 | VTNATELVQSSSTGRICDSPHRILDGKNCTLIDALLGDPHCDGFQNKEWDL | |
| | FVERSKAYSNCYPYDVPDYASLRSLVASSGTLEFTNEGENWTGVAQDGTSY | |
| | ACKRGSVKSFFSRLNWLHKLEYKYPALNVTMPNNDKFDKLYIWGVHHPSTD | |
| | SDQTSLYVQASGRVTVSTKRSQQTVIPNIGSRPWVRGISSRISIYWTIVKP | |
| | GDILLIKSTGNLIAPRGYFKIRSGKSSIMRSDAPIGNCNSECITPNGSIPN | |
| | DKPFQNVNRITYGACPRYVKQNTLKLATGMRNVPEKQTRGIFGAIAGFIEN | |
| | GWEGMVDGWYGFRFQNSEGTGQAADLKSTQAAINQINGLLNRLIEKTNEKE | |
| | HQIEKEFSEVEGRIQDLEKYVEDTKIDLWSYNAELLVALLNQHTIDLTDSE | |
| | MNKLFERTRKQLRENAEDMGNGCFKIYHKCDNACIGSIRNGTYDHDVYRDE | |
| | ALNNRFQIKGVELKSGYKDWILWISFAISCELLCVVLLGFIMWACQKGNIR | |
| | CNICI | |
| A/Memphis/1/1980 | MKTIIALSYILCLVFAQKLPGNDNSTATLCLGHHAVPNGTLVKTITNDQIE | 98 |
| A_/_H3N2 | VTNATELVQSSSTGRICDSPHRILDGKNCTLVDALLGDPHCDGFQNEKWDL | |
| | FVERSKAFSNCYPYDVPDYASLRSLVASSGTLEFINESENWTGVTQSGGSY | |
| | ACKRGSDNSFFSRLNWLYESESKYPVLNVTMPNNGNEDKLYIWGVHHPSTD | |
| | KEQTNLYVRASGRVTVSTKRSQQTIIPNIGSRPWVRGLSSRISIYWTIVKP | |
| | GDILLINSNGNLIAPRGYFKIRTGKSSIMRSDAPIGTCSSECITPNGSIPN | |
| | DKPFQNVNKITYGACPKYVKQNTLKLATGMRNVPEKQTRGIFGAIAGFIEN | |
| | GWEGMVDGWYGFRFQNSEGTGQAADLKSTQAAIDQINGLLNRVIEKTNEKF | |
| | HQIEKEFSEVEGRIQDLEKYVEDTKIDLWSYNAELLVALLNQHTIDLTDSE | |
| | MNKLFEKTRRQLRENAEDMGNGCFKIYHKCDNACIGSIRNGTYDHDVYRDE | |
| | ALNNRFQIKGVELKSGYKDWILWISFAISCELLCVVLLGFIMWACQKGNIR | |
| | CNICI | |
| A/Hong_Kong/1/19 | MKTIIALSYILCLVFAQKLPGNDNSTATLCLGHHAVPNGTLVKTITDDQIE | 99 |
| 68 A_/_H3N2 | VTNATELVQSSSTGKICNNPHRILDGIDCTLIDALLGDPHCDVEQNETWDL | |
| | FVERSKAFSNCYPYDVPDYASLRSLVASSGTLEFITEGFTWTGVTQNGGSN | |
| | ACKRGPGSGFFSRLNWLTKSGSTYPVLNVTMPNNDNEDKLYIWGVHHPSTN | |
| | QEQTSLYVQASGRVTVSTRRSQQTIIPNIGSRPWVRGLSSRISIYWTIVKP | |
| | GDVLVINSNGNLIAPRGYFKMRTGKSSIMRSDAPIDTCISECITPNGSIPN | |
| | DKPFQNVNKITYGACPKYVKQNTLKLATGMRNVPEKQTRGLFGAIAGFIEN | |
| | GWEGMIDGWYGFREQNSEGTGQAADLKSTQAAIDQINGLLNRVIEKTNEKF | |
| | HQIEKEFSEVEGRIQDLEKYVEDTKIDLWSYNAELLVALLNQHTIDLTDSE | |
| | MNKLFEKTRRQLRENAEDMGNGCFKIYHKCDNACIESIRNGTYDHDVYRDE | |
| | ALNNRFQIKGVELKSGYKDWILWISFAISCELLCVVLLGFIMWACQKGNIR | |
| | CNICI | |
| A/Bilthoven/1761 | MKTIIALSYILCLVFAQKLPGNDNSTATLCLGHHAVPNGTLVKTITNDQIE | 100 |
| /1976 A_/_H3N2 | VTNATELVQSSSTGKICDNPHRILDGINCTLIDALLGDPHCDGFQNEKWDL | |
| | FVERSKAFSNCYPYDVPDYASLRSLVASSGTLEFINEGENWTGVTQNGGSS | |
| | ACKRGPDNGFFSRLNWLYKSGSTYPVQNVTMPNNDNSDKLYIWGVHHPSTD | |
| | KEQTDLYVQASGKVTVSTKRSQQTVIPNVGSRPWVRGLSSRVSIYWTIVKP | |
| | GDILVINSNGNLIAPRGYFKMRTGKSSIMRSDAPIGTCSSECITPNGSIPN | |
| | DKPFQNVNKITYGACPKYVKQNTLKLATGMRNVPEKQTRGIFGAIAGFIEN | |
| | GWEGMIDGWYGFRFQNSEGTGQAADLKSTQAAIDQINGLLNRVIEKTNEKE | |
| | HQIEKEFSEVEGRIQDLEKYVEDTKIDLWSYNAELLVALLNQHTIDLTDSE | |
| | MNKLFEKTRRQLRENAEDMGNGCFKIYHKCDNACIGSIRNGTYDHDVYRDE | |
| | ALNNRFQIKGVELKSGYKDWILWISFAISCELLCVVLLGFIMWACQKGNIR | |
| | CNICI | |
| A/Roma/1949 | MKVKLLVLLCTFTATYADTICIGYHANNSTDTVDTVLEKNVTVTHSVNLLE | 101 |
| | DSHNGKLCRLKGIAPLQLGKCNIAGWILGNPECESLESKKSWSYIAETPNS | |
| | ENGTCYPGYFADYEELREQLSSVSSFERFEIFPKERSWPKHNVTRGVTAAC | |
| | SHKGKSSFYRNLLWLTEKDGSYPNLSKSYVNNKEKEVLVLWGVHHPSNIED | |
| | QKTLYRKENAYVSVVSSNYNRRFTPEIAERPKVRGQAGRINYYWTLLEPGD | |
| | TIIFEANGNLIAPWHAFALSRGFGSGIITSNASMDECDTKCQTPQGAINSS | |
| | LPFQNIHPVTIGECPKYVRSTKLRMVTGLRNIPSIQSRGLFGAIAGFIEGG | |
| | WTGMIDGWYGYHFQNEQGSGYAADQKSTQNAINGITNAVNSVIEKMNTQFT | |
| | AVGKEFNKLEKRMENLNKKVDDGELDIWTYNAELLVLLANERTLDFHDSNV | |
| | KNLYEKVKSQLKNNAKEIGNGCFEFYHKCNNECMESVKNGTYDYPKYSEES | |
| | KLNREKIDGVKLESMGVYQILAIYSTVASSLVLLVSLGAISFWMCSNGSLQ | |
| | CRICI | |
| A/MD/12/1991 | MKVKLLVLLCTFTATYADTLCIGYHANNSTDTVDTVLEKNVTVTHSVNLLE | 102 |
| | DRHNGKLCKLRGVAPLHLGKCNIAGWLLGNPECELLFTASSWSYIVETSNS | |
| | DNGTCYPGDFINYEELREQLSSVSSFERFEIFPKASSWPDHETNRGVTAAC | |
| | PYAGANSFYRNLIWLVKKGNSYPKLSKSYVNNKEKEVLVLWGIHHPPTSTD | |
| | QQSLYQNADAYVFVGSSKYNKKEKPEIATRPKVRGQAGRMNYYWTLVEPGD | |
| | TITFEATGNLVVPRYAFAMKRGSGSGIIISDTPVHDCNTTCQTPKGAINTS | |
| | LPFQNIHPVTIGECPKYVKSTKLRMATGLRNIPSIQSRGLFGAIAGFIEGG | |
| | WTGMIDGWYGYHFQNEQGSGYAADQKSTQNAIDGITNAVNSVIEKMNTQFT | |
| | AVGKEFNHLEKRIENLNKKVDDGELDVWTYNAELLVLLANERTLDYHDSNV | |
| | KNLYEKVRSQLKNNAKEIGNGCFEFYHKCDDTCMESVKNGTYDYPKYSEES | |
| | KLNREEIDGVKLESTRIYQILAIYSTVASSLVLLVSLGAISFWMCSNGSLQ | |
| | CRICI | |
| A/Auckland/588/2 | MKTIIALSYILCLVFAQKLPGNDNSTATLCLGHHAVPNGTLVKTITNDQIE | 103 |
| 000 | VTNATELVQSSSTGGICDSPHQILDGENCTLIDALLGDPHCDGFQNKEWDL | |
| | FVERSKAYSNCYPYDVPDYASLRSLVASSGTLEENNESENWTGVAQNGTSS | |
| | ACKRRSNKSFFSRLNWLHQLKYKYPALNVTMPNNEKEDKLYIWGVHHPSTD | |
| | SDQISLYAQAPGRVTVSTKRSQQTVIPNIGSRPWVRGVSSRISIYWTIVKP | |
| | GDILLINCTGNLIAPRGYFKIRSGKSSIMRSDASIGKCNSECITPNGSIPN | |
| | DKPFQNVNRITYGACPRYVKQNTLKLATGMRNVPEKQTRGIFGAIAGFIEN | |
| | GWEGMVDGWYGFRFQNSEGTGQAADLKSTQAAINQINGLLNRLIEKTNEKF | |
| | HQIEKEFSEVEGRIQDLEKYVEDTKIDLWSYNAELLVALLNQHTIDLTDSE | |
| | MNKLFERTKKQLRENAEDMGNGCFKIYHKCDNACIGSIRNGTYDHDVYRDE | |
| | ALNNRFQIKGVELKSGYKDWILWISFAISCELLCVVLLGFIMWACQKGNIR | |
| | CNICI | |
| A/Argentina/2830 | MKTIIALSYILCLVFAQKLPGNDNSTATLCLGHHAVPNGTIVKTITNDQIE | 104 |
| 2/2010 | VTNATELVQSSSTGEICDSPHQILDGKNCTLIDALLGDPQCDGFQNKKWDL | |
| | FVERSKAYSNCYPYDVPDYASLRSLVASSGTLEENNESENWTGVTQDGTSS | |
| | ACIRGSKNSFFSRLNWLTHLNFKYPALNVTMPNNEQFDKLYIWGVHHPGTD | |
| | KDQIFLYAQASGRITVSTKRSQQAAIPNIGSRPRVRNIPSRISIYWTIVKP | |
| | GDILLINSTGNLIAPRGYFKIRSGKSSIMRSDAPIGKCNSECITPNGSIPN | |
| | DKPFQNVNRITYGACPRYVKQNTLKLATGMRNVPEKQTRGIFGAIAGFIEN | |
| | GWEGMVDGWYGFREQNSEGRGQAADLKSTQAAIDQINGLLNRLIGKTNEKF | |
| | HQIEKEFSEVEGRIQDLEKYVEDTKIDLWSYNAELLVALLNQHTIDLTDSE | |
| | MNKLFEKTKKQLRENAEDMGNGCFKIYHKCDNACIGSIRNGTYDHDVYRDE | |
| | ALNKRFQIKGIELKSGYKDWILWISFAISCELLCVVLLGFIMWACQKGNIR | |
| | CNICI | |
| B/Austria/135941 | MKAIIVLLMVVTSNADRICTGITSSNSPHVVKTATQGEVNVTGVIPLTTTP | 105 |
| 7/2021 | TKSHFANLKGTETRGKLCPKCLNCTDLDVALGRPKCTGKIPSARVSILHEV | |
| | RPVTSGCFPIMHDRTKIRQLPNLLRGYEHVRLSTHNVINTEDAPGGPYEIG | |
| | TSGSCLNITNGKGFFATMAWAVPKNKTATNPLTIEVPYICTEEEDQITVWG | |
| | FHSDDETQMARLYGDSKPQKFTSSANGVTTHYVSQIGGFPNQTEDGGLPQS | |
| | GRIVVDYMVQKSGKTGTITYQRGILLPQKVWCASGKSKVIKGSLPLIGEAD | |
| | CLHEKYGGLNKSKPYYTGEHAKAIGNCPIWVKTPLKLANGTKYRPPAKLLK | |
| | ERGFFGAIAGFLEGGWEGMIAGWYGYTSHGAHGVAVAADLKSTQEAINKIT | |
| | KNLNSLSELEVKNLQRLSGAMDELHNEILELDEKVDDLRADTISSQIELAV | |
| | LLSNEGIINSEDEHLLALERKLKKMLGPSAVEIGNGCFETKHKCNQTCLDR | |
| | IAAGTEDAGEFSLPTEDSLNITAASLNDDGLDNHTILLYYSTAASSLAVTL | |
| | MIAIFVVYMVSRDNVSCSICL | |

Table 3 depicts antigen amino acid sequences which may be encoded by nucleic acids of a vaccine composition of the invention. The amino acid sequences comprise a signaling peptide sequence (denoted with single underlining), an extracellular sequence, and a transmembrane domain sequence (denoted with double underlining). In some embodiments, a vaccine composition comprises nucleic acids encoding any one of (or all of) the sequences in Table 3.

TABLE 4
Stabilized amino acid sequences of antigen extracellular domains

| | | SEQ ID |
|---|---|---|
| Strain | Amino Acid Sequence of Antigen | NO: |
| A/New_York/1/191 | ADTICIGYHANNSTDTVDTVLEKNVTVTHSVNLLEDSHNGKLCKLKGIAPL | 108 |
| 8 A_/_H1N1 | QLGKCNIAGWLLGNPECDLLLTASSWSYIVETSNSENGTCYPGDFIDYEEL | |
| | REQLSSVSSFEKFEIFPKTSSWPNHETTKGVTAACSYAGASSFYRNLLWLT | |
| | KKGSSYPKLSKSYVNNKGKEVLVLWGVHHPPTGTDQQSLYQNADAYVSVGS | |
| | SKYNRRFTPEIAARPKVRDQAGRMNYYWTLLEPGDTITFEATGNLIAPWYA | |
| | FALNRGSGSGIITSDAPVHDCNTKCQTPHGAINSSLPFQNIHPVTIGECPK | |
| | YVRSTKLRMATGLRNIPSIQSRGLFGAIAGFIEGGWTGMIDGWYGYHFQNE | |
| | QGSGYAADRKSTQNAIDGITNLVNSVIEKMNTQFTSVGKEENHLEKRIENL | |
| | NRKVDDGFLDVWTYNAELLVLLLNERTLDYHDSNVKNLYEKVRSQLKNNAK | |
| | EIGNGCFEFYHKCDDSCMESVKNGTYDYPKYSEESKLNREEIDGVKLES | |
| A/Wisconsin/588/ | ADTLCIGYHANNSTDTVDTVLEKNVTVTHSVNLLEDKHNGKLCKLRGVAPL | 109 |
| 2019 A_/_H1N1 | HLGKCNIAGWILGNPECESLSTARSWSYIVETSNSDNGTCYPGDFINYEEL | |
| | REQLSSVSSFERFEIFPKTSSWPNHDSDNGVTAACPHAGAKSFYKNLIWLV | |
| | KKGKSYPKINQTYINDKGKEVLVLWGIHHPPTIADQQSLYQNADAYVFVGT | |
| | SRYSKKFKPEIATRPKVRDQEGRMNYYWTLVEPGDKITFEATGNLVAPRYA | |
| | FTMERDAGSGIIISDTPVHDCNTTCQTPEGAINTSLPFQNVHPITIGKCPK | |
| | YVKSTKLRLATGLRNVPSIQSRGLFGAIAGFIEGGWTGMVDGWYGYHFQNE | |
| | QGSGYAADLKSTQNAIDKITNAVNSVIEKMNTQFTAVGKEENHLEKRIENL | |
| | NKKVDDGELDIWTYNAELLVLLANERTLDYHDSNVKNLYEKVRNQLKNNAK | |
| | EIGNGCFEFYHKCDNTCMESVKNGTYDYPKYSEEAKLNREKIDGVKLDS | |
| A/Brisbane/59/20 | ADTICIGYHANNSTDTVDTVLEKNVTVTHSVNLLENSHNGKLCLLKGIAPL | 110 |
| 07 A_/_H1N1 | QLGNCSVAGWILGNPECELLISKESWSYIVEKPNPENGTCYPGHFADYEEL | |
| | REQLSSVSSFERFEIFPKESSWPNHTVTGVSASCSHNGESSFYRNLLWLTG | |
| | KNGLYPNLSKSYANNKEKEVLVLWGVHHPPNIGNQKALYHTENAYVSVVSS | |
| | HYSRKFTPEIAKRPKVRDQEGRINYYWTLLEPGDTIIFEANGNLIAPRYAF | |
| | ALSRGFGSGIINSNAPMDKCDAKCQTPQGAINSSLPFQNVHPVTIGECPKY | |
| | VRSAKLRMVTGLRNIPSIQSRGLFGAIAGFIEGGWTGMVDGWYGYHFQNEQ | |
| | GSGYAADQKSTQNAINGITNAVNSVIEKMNTQFTAVGKEENKLERRMENLN | |
| | KKVDDGFIDIWTYNAELLVLLANERTLDFHDSNVKNLYEKVKSQLKNNAKE | |
| | IGNGCFEFYHKCNDECMESVKNGTYDYPKYSEESKLNREKIDGVKLES | |
| A/Wisconsin/28/2 | ADTLCIGYHANNSTDTVDTVLEKNVTVTHSVNLLENRHNGKLCKLRGVAPL | 111 |
| 011.2011/12 | HLGKCNIAGWLLGNPECESLSTASSWSYIVETSNSDNGTCYPGDFINYEEL | |
| A_/_H1N1 | REQLSSVSSFERFEIFPKTSSWPNHDTNRGVTAACPHDGTNSFYRNLIWLV | |
| | KKGNSYPKINKSYINNKEKEILVLWAIHHPSTSADQQSLYQNADAYVFVGS | |
| | SRYSRKFEPEVATRPKVRDQAGRMNYYWTLVEPGDKITFEATGNLVVPRYA | |
| | FALKRNSGSGIIISDTSVHDCDTNCQTPNGAINTSLPFQNIHPVTIGECPK | |
| | YVKSTKLRMATGLRNIPSIQSRGLFGAIAGFIEGGWTGMIDGWYGYHFQNE | |
| | QGSGYAADLKSTQNAIDGITNLVNSVIEKMNTQFTAVGKEFSHLERRIENL | |
| | NKKVDDGELDIWTYNAELLVLLLNERTLDYHDSNVKNLYEKVRSQLKNNAK | |
| | EIGNGCFEFYHKCDDMCMESVKNGTYDYPKYSEEAKLNREEIDGVKLES | |
| A/Denver/57 | ADTICIGYHANNSTDTVDTVLEKNVTVTHSVNLLEDSHNGKLCRLKGKAPL | 112 |
| A_/_H1N1 | QLGNCNIAGWVLGNPECESLLSNRSWSYIAETPNSENGTCYPGDEADYEEL | |
| | REQLSSVSSFERFEIFPKERSWPNHTTRGVTAACPHARKSSFYKNLVWLTE | |
| | ANGSYPNLSRSYVNNQEKEVLVLWGVHHPSNIEEQRALYRKDNAYVSVVSS | |
| | NYNRRFTPEIAKRPKVRDQSGRMNYYWTLLEPGDTIIFEATGNLIAPWYAF | |
| | ALSRGPGSGIITSNAPLDECDTKCQTPQGAINSSLPFQNIHPVTIGECPKY | |
| | VRSTKLRMVTGLRNIPSVQSRGLFGAIAGFIEGGWTGMMDGWYGYHFQNEQ | |
| | GSGYAADQKSTQNAINGITNAVNSVIEKMNTQFTAVGKEENKLEKRMENLN | |
| | KKVDDGFMDIWTYNAELLVLLANERTLDFHDSNVKNLYEKVKNQLRNNAKE | |
| | LGNGCFEFYHKCDNECMESVKNGTYDYPKYSEESKLNREKIDGVKLES | |
| A/Beijing/262/19 | ADTICIGYHANNSTDTVDTVLEKNVTVTHSVNLLEDSHNGKLCLLKGIAPL | 113 |
| 95 A_/_H1N1 | QLGNCSVAGWILGNPECESLISKESWSYIVETPNPENGTCYPGYFADYEEL | |
| | REQLSSVSSFERFEIFPKESSWPNHTVTGVTASCSHNGKSSFYRNLLWLTE | |
| | KNGLYPNLSNSYVNNKEKEVLVLWGVHHPSNIRDQRAIYHTENAYVSVVSS | |
| | HYSRRFTPEIAKRPKVRGQEGRINYYWTLLEPGDTIIFEANGNLIAPWYAF | |
| | ALSRGFGSGIITSNAPMNECDAKCQTPQGAINSSLPFQNVHPVTIGECPKY | |
| | VRSTKLRMVTGLRNIPSIQSRGLFGAIAGFIEGGWTGMMDGWYGYHFQNEQ | |
| | GSGYAADQKSTQNAINGITNLVNSVIEKMNTQFTAVGKEENKLERRMENLN | |
| | KKVDDGELDIWTYNAELLVLLLNERTLDFHDSNVKNLYEKVKSQLKNNAKE | |
| | IGNGCFEFYHKCNNECMESVKNGTYDYPKYSEESKLNREKIDGVKLES | |
| A/Puerto_Rico/8/ | ADTICIGYHANNSTDTVDTVLEKNVTVTHSVNLLEDSHNGKLCRLKGIAPL | 114 |
| 1934 A_/_H1N1 | QLGKCNIAGWLLGNPECDPLLPVRSWSYIVETPNSENGICYPGDFIDYEEL | |
| | REQLSSVSSFERFEIFPKESSWPNHNTNKGVTAACSHEGKSSFYRNLLWLT | |
| | EKEGSYPKLKNSYVNKKGKEVLVLWGIHHPPNSKEQQNLYQNENAYVSVVT | |
| | SNYNRRFTPEIAERPKVRDQAGRMNYYWTLLKPGDTIIFEANGNLIAPMYA | |
| | FALSRGFGSGIITSNASMHECNTKCQTPLGAINSSLPYQNIHPVTIGECPK | |
| | YVRSAKLRMVTGLRNIPSIQSRGLFGAIAGFIEGGWTGMIDGWYGYHFQNE | |
| | QGSGYAADQKSTQNAINGITNAVNTVIEKMNIQFTAVGKEENKLEKRMENL | |
| | NKKVDDGELDIWTYNAELLVLLANERTLDFHDSNVKNLYEKVKSQLKNNAK | |
| | EIGNGCFEFYHKCDNECMESVRNGTYDYPKYSEESKLNREKVDGVKLES | |
| A/California/07/ | ADTLCIGYHANNSTDTVDTVLEKNVTVTHSVNLLEDKHNGKLCKLRGVAPL | 115 |
| 2009 A_/_H1N1 | HLGKCNIAGWILGNPECESLSTASSWSYIVETPSSDNGTCYPGDFIDYEEL | |
| | REQLSSVSSFERFEIFPKTSSWPNHDSNKGVTAACPHAGAKSFYKNLIWLV | |
| | KKGNSYPKLSKSYINDKGKEVLVLWGIHHPSTSADQQSLYQNADAYVFVGS | |
| | SRYSKKFKPEIAIRPKVRDQEGRMNYYWTLVEPGDKITFEATGNLVVPRYA | |
| | FAMERNAGSGIIISDTPVHDCNTTCQTPKGAINTSLPFQNIHPITIGKCPK | |
| | YVKSTKLRLATGLRNIPSIQSRGLFGAIAGFIEGGWTGMVDGWYGYHFQNE | |
| | QGSGYAADLKSTQNAIDEITNLVNSVIEKMNTQFTAVGKEFNHLEKRIENL | |
| | NKKVDDGELDIWTYNAELLVLLLNERTLDYHDSNVKNLYEKVRSQLKNNAK | |
| | EIGNGCFEFYHKCDNTCMESVKNGTYDYPKYSEEAKLNREEIDGVKLES | |
| A/Indiana/11/201 | TATLCLGHHAVPNGTIVKTITNDRIEVTNATELVQNSSIGEICDSPHQILD | 116 |
| 8 A_/_H3N2 | GENCTLIDALLGDPQCDGFQNKKWDLFVERSKAYSNCYPYDVPDYASLRSL | |
| | VASSGTLEENNESFNWTGVKQNGTSSACIRKSSSSFFSRLNWLTHLNYTYP | |
| | ALNVTMPNNEQFDKLYIWGVHHPGTDKDQIFLYAQSSGRITVSTKRSQQAV | |
| | IPNIGSRPRIRDIPSRISIYWTIVKPGDILLINSTGNLIAPRGYFKIQSGK | |
| | SSIMRSDAPIGKCKSECITPNGSIPNDKPFQNVNRITYGACPRYVKHSTLK | |
| | LATGMRNVPEKQTRGIFGAIAGFIENGWEGMVDGWYGFRFQNSEGRGQAAD | |
| | LKSTQAAIDQINGLLNRLIGKTNEKFHQIEKEFSEVEGRIQDLEKYVEDTK | |
| | IDLWSYNAELLVALLNQHTIDLTDSEMNKLFEKTKKQLRENAEDMGNGCFK | |
| | IYHKCDNACIGSIRNGTYDHNVYRDEALNNRFQIKGVELKS | |
| -A/California/07/ | TATLCLGHHAVPNGTIVKTITNDQIEVTNATELVQSSSTGGICDSPHQILD | 117 |
| 2004 A_/_H3N2 | GENCTLIDALLGDPQCDGFQNKKWDLFVERSKAYSNCYPYDVPDYASLRSL | |
| | VASSGTLEFNNESFNWTGVTQNGTSSSCKRRSNNSFFSRLNWLTHLKFKYP | |
| | ALNVTMPNNEKFDKLYIWGVHHPGTNNDQISLYTQASGRITVSTKRSQQTV | |
| | IPNIGSRPRVRDIPSRISIYWTIVKPGDILLINSTGNLIAPRGYFKIRSGK | |
| | SSIMRSDAPIGKCNSECITPNGSIPNDKPFQNVNRITYGACPRYVKQNTLK | |
| | LATGMRNVPEKQTRGIFGAIAGFIENGWEGMVDGWYGFRFQNSEGIGQAAD | |
| | LKSTQAAINQINGLLNRLIGKTNEKFHQIEKEFSEVEGRIQDLEKYVEDTK | |
| | IDLWSYNAELLVALLNQHTIDLTDSEMNKLFERTKKQLRENAEDMGNGCFK | |
| | IYHKCDNACIGSIRNGTYDHDVYRDEALNNRFQIKGVELKS | |
| A/Alaska/01/2021 | TATLCLGHHAVPNGTIVKTITNDRIEVTNATELVQNSSIGEICNSPHQILD | 118 |
| A_/_H3N2 | GGNCTLIDALLGDPQCDGFQNKEWDLFVERSRANSSCYPYDVPDYASLRSL | |
| | VASSGTLEFKNESFNWTGVKQNGTSSACIRGSSSSFFSRLNWLTSLNNIYP | |
| | AQNVTMPNKEQFDKLYIWGVHHPDTDKNQFSLFAQSSGRITVSTKRSQQAV | |
| | IPNIGSRPRIRDIPSRISIYWTIVKPGDILLINSTGNLIAPRGYFKIRSGK | |
| | SSIMRSDAPIGKCKSECITPNGSIPNDKPFQNVNRITYGACPRYVKQSTLK | |
| | LATGMRNVPEKQTRGIFGAIAGFIENGWEGMVDGWYGFRFQNSEGRGQAAD | |
| | LKSTQAAIDQISGLLNRLIGKTNEKFHQIEKEFSEVEGRVQDLEKYVEDTK | |
| | IDLWSYNAELLVALLNQHTIDLTDSEMNKLFEKTKKQLRENAEDMGNGCFK | |
| | IYHKCDNACIGSIRNETYDHNVYRDEALNNRFQIKGVELKS | |
| A/Cambodia/e0826 | TATLCLGHHAVPNGTIVKTITNDRIEVTNATELVQNSSIGEICDSPHQILD | 119 |
| 360/2020 | GGNCTLIDALLGDPQCDGFQNKEWDLFVERSRANSNCYPYDVPDYASLRSL | |
| A_/_H3N2 | VASSGTLEFKNESFNWTGVKQNGTSSACIRGSSSSFFSRLNWLTHLNYTYP | |
| | ALNVTMPNNEQFDKLYIWGVHHPSTDKDQISLFAQPSGRITVSTKRSQQAV | |
| | IPNIGSRPRIRDIPSRISIYWTIVKPGDILLINSTGNLIAPRGYFKIRSGK | |
| | SSIMRSDAPIGKCKSECITPNGSIPNDKPFQNVNRITYGACPRYVKQSTLK | |
| | LATGMRNVPEKQTRGIFGAIAGFIENGWEGMVDGWYGFRFQNSEGRGQAAD | |
| | LKSTQAAIDQINGLLNRLIGKTNEKFHQIEKEFSEVEGRVQDLEKYVEDTK | |
| | IDLWSYNAELLVALLNQHTIDLTDSEMNKLFEKTKKQLRENAEDMGNGCFK | |
| | IYHKCDNACIGSIRNETYDHNVYRDEALNNRFQIKGVELKS | |
| A/Nanchang/933/1 | TATLCLGHHAVPNGTLVKTITNDQIEVTNATELVQSSSTGRICDSPHRILD | 120 |
| 995 A_/_H3N2 | GKNCTLIDALLGDPHCDGFQNKEWDLEVERSKAYSNCYPYDVPDYASLRSL | |
| | VASSGTLEFTNEGENWTGVAQDGTSYACKRGSVKSFFSRLNWLHKLEYKYP | |
| | ALNVTMPNNDKFDKLYIWGVHHPSTDSDQTSLYVQASGRVTVSTKRSQQTV | |
| | IPNIGSRPWVRGISSRISIYWTIVKPGDILLIKSTGNLIAPRGYFKIRSGK | |
| | SSIMRSDAPIGNCNSECITPNGSIPNDKPFQNVNRITYGACPRYVKQNTLK | |
| | LATGMRNVPEKQTRGIFGAIAGFIENGWEGMVDGWYGFREQNSEGTGQAAD | |
| | LKSTQAAINQINGLLNRLIEKTNEKFHQIEKEFSEVEGRIQDLEKYVEDTK | |
| | IDLWSYNAELLVALLNQHTIDLTDSEMNKLFERTRKQLRENAEDMGNGCFK | |
| | IYHKCDNACIGSIRNGTYDHDVYRDEALNNRFQIKGVELKS | |
| A/Memphis/1/1980 | TATLCLGHHAVPNGTLVKTITNDQIEVTNATELVQSSSTGRICDSPHRILD | 121 |
| A_/_H3N2 | GKNCTLVDALLGDPHCDGFQNEKWDLFVERSKAFSNCYPYDVPDYASLRSL | |
| | VASSGTLEFINESFNWTGVTQSGGSYACKRGSDNSFFSRLNWLYESESKYP | |
| | VLNVTMPNNGNFDKLYIWGVHHPSTDKEQTNLYVRASGRVTVSTKRSQQTI | |
| | IPNIGSRPWVRGLSSRISIYWTIVKPGDILLINSNGNLIAPRGYFKIRTGK | |
| | SSIMRSDAPIGTCSSECITPNGSIPNDKPFQNVNKITYGACPKYVKQNTLK | |
| | LATGMRNVPEKQTRGIFGAIAGFIENGWEGMVDGWYGFREQNSEGTGQAAD | |
| | LKSTQAAIDQINGLLNRVIEKTNEKFHQIEKEFSEVEGRIQDLEKYVEDTK | |
| | IDLWSYNAELLVALLNQHTIDLTDSEMNKLFEKTRRQLRENAEDMGNGCFK | |
| | IYHKCDNACIGSIRNGTYDHDVYRDEALNNRFQIKGVELKS | |
| A/Hong_Kong/1/19 | TATLCLGHHAVPNGTLVKTITDDQIEVTNATELVQSSSTGKICNNPHRILD | 122 |
| 68 A_/_H3N2 | GIDCTLIDALLGDPHCDVFQNETWDLEVERSKAFSNCYPYDVPDYASLRSL | |
| | VASSGTLEFITEGFTWTGVTQNGGSNACKRGPGSGFFSRLNWLTKSGSTYP | |
| | VLNVTMPNNDNFDKLYIWGVHHPSTNQEQTSLYVQASGRVTVSTRRSQQTI | |
| | IPNIGSRPWVRGLSSRISIYWTIVKPGDVLVINSNGNLIAPRGYFKMRTGK | |
| | SSIMRSDAPIDTCISECITPNGSIPNDKPFQNVNKITYGACPKYVKQNTLK | |
| | LATGMRNVPEKQTRGLFGAIAGFIENGWEGMIDGWYGFRFQNSEGTGQAAD | |
| | LKSTQAAIDQINGLLNRVIEKTNEKFHQIEKEFSEVEGRIQDLEKYVEDTK | |
| | IDLWSYNAELLVALLNQHTIDLTDSEMNKLFEKTRRQLRENAEDMGNGCFK | |
| | IYHKCDNACIESIRNGTYDHDVYRDEALNNRFQIKGVELKS | |
| A/Bilthoven/1761 | TATLCLGHHAVPNGTLVKTITNDQIEVTNATELVQSSSTGKICDNPHRILD | 123 |
| /1976 A_/_H3N2 | GINCTLIDALLGDPHCDGFQNEKWDLEVERSKAFSNCYPYDVPDYASLRSL | |
| | VASSGTLEFINEGFNWTGVTQNGGSSACKRGPDNGFFSRLNWLYKSGSTYP | |
| | VQNVTMPNNDNSDKLYIWGVHHPSTDKEQTDLYVQASGKVTVSTKRSQQTV | |
| | IPNVGSRPWVRGLSSRVSIYWTIVKPGDILVINSNGNLIAPRGYFKMRTGK | |
| | SSIMRSDAPIGTCSSECITPNGSIPNDKPFQNVNKITYGACPKYVKQNTLK | |
| | LATGMRNVPEKQTRGIFGAIAGFIENGWEGMIDGWYGFREQNSEGTGQAAD | |
| | LKSTQAAIDQINGLLNRVIEKTNEKFHQIEKEFSEVEGRIQDLEKYVEDTK | |
| | IDLWSYNAELLVALLNQHTIDLTDSEMNKLFEKTRRQLRENAEDMGNGCFK | |
| | IYHKCDNACIGSIRNGTYDHDVYRDEALNNRFQIKGVELKS | |
| A/Roma/1949 | ADTICIGYHANNSTDTVDTVLEKNVTVTHSVNLLEDSHNGKLCRLKGIAPL | 124 |
| | QLGKCNIAGWILGNPECESLESKKSWSYIAETPNSENGTCYPGYFADYEEL | |
| | REQLSSVSSFERFEIFPKERSWPKHNVTRGVTAACSHKGKSSFYRNLLWLT | |
| | EKDGSYPNLSKSYVNNKEKEVLVLWGVHHPSNIEDQKTLYRKENAYVSVVS | |
| | SNYNRRFTPEIAERPKVRGQAGRINYYWTLLEPGDTIIFEANGNLIAPWHA | |
| | FALSRGFGSGIITSNASMDECDTKCQTPQGAINSSLPFQNIHPVTIGECPK | |
| | YVRSTKLRMVTGLRNIPSIQSRGLFGAIAGFIEGGWTGMIDGWYGYHFQNE | |
| | QGSGYAADQKSTQNAINGITNAVNSVIEKMNTQFTAVGKEENKLEKRMENL | |
| | NKKVDDGELDIWTYNAELLVLLANERTLDEHDSNVKNLYEKVKSQLKNNAK | |
| | EIGNGCFEFYHKCNNECMESVKNGTYDYPKYSEESKLNREKIDGVKLES | |
| A/MD/12/1991 | ADTLCIGYHANNSTDTVDTVLEKNVTVTHSVNLLEDRHNGKLCKLRGVAPL | 125 |
| | HLGKCNIAGWLLGNPECELLFTASSWSYIVETSNSDNGTCYPGDFINYEEL | |
| | REQLSSVSSFERFEIFPKASSWPDHETNRGVTAACPYAGANSFYRNLIWLV | |
| | KKGNSYPKLSKSYVNNKEKEVLVLWGIHHPPTSTDQQSLYQNADAYVFVGS | |
| | SKYNKKFKPEIATRPKVRGQAGRMNYYWTLVEPGDTITFEATGNLVVPRYA | |
| | FAMKRGSGSGIIISDTPVHDCNTTCQTPKGAINTSLPFQNIHPVTIGECPK | |
| | YVKSTKLRMATGLRNIPSIQSRGLFGAIAGFIEGGWTGMIDGWYGYHFQNE | |
| | QGSGYAADQKSTQNAIDGITNAVNSVIEKMNTQFTAVGKEFNHLEKRIENL | |
| | NKKVDDGFLDVWTYNAELLVLLANERTLDYHDSNVKNLYEKVRSQLKNNAK | |
| | EIGNGCFEFYHKCDDTCMESVKNGTYDYPKYSEESKLNREEIDGVKLES | |
| A/Auckland/588/ | TATLCLGHHAVPNGTLVKTITNDQIEVTNATELVQSSSTGGICDSPHQILD | 126 |
| 2000 | GENCTLIDALLGDPHCDGFQNKEWDLEVERSKAYSNCYPYDVPDYASLRSL | |
| | VASSGTLEENNESFNWTGVAQNGTSSACKRRSNKSFFSRLNWLHQLKYKYP | |
| | ALNVTMPNNEKEDKLYIWGVHHPSTDSDQISLYAQAPGRVTVSTKRSQQTV | |
| | IPNIGSRPWVRGVSSRISIYWTIVKPGDILLINCTGNLIAPRGYFKIRSGK | |
| | SSIMRSDASIGKCNSECITPNGSIPNDKPFQNVNRITYGACPRYVKQNTLK | |
| | LATGMRNVPEKQTRGIFGAIAGFIENGWEGMVDGWYGFRFQNSEGTGQAAD | |
| | LKSTQAAINQINGLLNRLIEKTNEKFHQIEKEFSEVEGRIQDLEKYVEDTK | |
| | IDLWSYNAELLVALLNQHTIDLTDSEMNKLFERTKKQLRENAEDMGNGCFK | |
| | IYHKCDNACIGSIRNGTYDHDVYRDEALNNRFQIKGVELKS | |
| A/Argentina/ | TATLCLGHHAVPNGTIVKTITNDQIEVTNATELVQSSSTGEICDSPHQILD | 127 |
| 28302/2010 | GKNCTLIDALLGDPQCDGFQNKKWDLFVERSKAYSNCYPYDVPDYASLRSL | |
| | VASSGTLEFNNESFNWTGVTQDGTSSACIRGSKNSFFSRLNWLTHLNFKYP | |
| | ALNVTMPNNEQFDKLYIWGVHHPGTDKDQIFLYAQASGRITVSTKRSQQAA | |
| | IPNIGSRPRVRNIPSRISIYWTIVKPGDILLINSTGNLIAPRGYFKIRSGK | |
| | SSIMRSDAPIGKCNSECITPNGSIPNDKPFQNVNRITYGACPRYVKQNTLK | |
| | LATGMRNVPEKQTRGIFGAIAGFIENGWEGMVDGWYGFRFQNSEGRGQAAD | |
| | LKSTQAAIDQINGLLNRLIGKTNEKFHQIEKEFSEVEGRIQDLEKYVEDTK | |
| | IDLWSYNAELLVALLNQHTIDLTDSEMNKLFEKTKKQLRENAEDMGNGCFK | |
| | IYHKCDNACIGSIRNGTYDHDVYRDEALNKRFQIKGIELKS | |
| B/Austria/ | DRICTGITSSNSPHVVKTATQGEVNVTGVIPLTTTPTKSHFANLKGTETRG | 128 |
| 1359417/2021 | KLCPKCLNCTDLDVALGRPKCTGKIPSARVSILHEVRPVTSGCFPIMHDRT | |
| | KIRQLPNLLRGYEHVRLSTHNVINTEDAPGGPYEIGTSGSCLNITNGKGFF | |
| | ATMAWAVPKNKTATNPLTIEVPYICTEEEDQITVWGFHSDDETQMARLYGD | |
| | SKPQKFTSSANGVTTHYVSQIGGFPNQTEDGGLPQSGRIVVDYMVQKSGKT | |
| | GTITYQRGILLPQKVWCASGKSKVIKGSLPLIGEADCLHEKYGGLNKSKPY | |
| | YTGEHAKAIGNCPIWVKTPLKLANGTKYRPPAKLLKERGFFGAIAGFLEGG | |
| | WEGMIAGWYGYTSHGAHGVAVAADLKSTQEAINKITKNLNSLSELEVKNLQ | |
| | RLSGAMDELHNEILELDEKVDDLRADTISSQIELAVLLSNEGIINSEDEHL | |
| | LALERKLKKMLGPSAVEIGNGCFETKHKCNQTCLDRIAAGTEDAGEFSLPT | |
| | FDSLNITAASLNDDGLDNHT | |

In some embodiments, the plurality of antigens comprises an amino acid sequence corresponding to SEQ ID NO: 1 or a sequence at least 70% identical thereto. In some embodiments, the plurality of antigens comprises an amino acid sequence corresponding to SEQ ID NO: 2 or a sequence at least 70% identical thereto. In some embodiments, the amino acid sequence corresponding to SEQ ID NO: 2 or a sequence at least 70% identical thereto further comprises a transmembrane domain. In some embodiments, the transmembrane domain comprises the sequence of SEQ ID NO: 43, or a sequence at least 70% identical thereto. In some embodiments, the plurality of antigens comprises an amino acid sequence corresponding to SEQ ID NO: 3 or a sequence at least 70% identical thereto. In some embodiments, the amino acid sequence corresponding to SEQ ID NO: 3 or a sequence at least 70% identical thereto further comprises a transmembrane domain. In some embodiments, the transmembrane domain comprises the sequence of SEQ ID NO: 44, or a sequence at least 70% identical thereto. In some embodiments, the plurality of antigens comprises an amino acid sequence corresponding to SEQ ID NO: 4 or a sequence at least 70% identical thereto. In some embodiments, the amino acid sequence corresponding to SEQ ID NO: 4 or a sequence at least 70% identical thereto further comprises a transmembrane domain. In some embodiments, the transmembrane domain comprises the sequence of SEQ ID NO: 45, or a sequence at least 70% identical thereto. In some embodiments, the plurality of antigens comprises an amino acid sequence corresponding to SEQ ID NO: 5 or a sequence at least 70% identical thereto. In some embodiments, the amino acid sequence corresponding to SEQ ID NO: 5 or a sequence at least 70% identical thereto further comprises a transmembrane domain. In some embodiments, the transmembrane domain comprises the sequence of SEQ ID NO: 46, or a sequence at least 70% identical thereto. In some embodiments, the plurality of antigens comprises an amino acid sequence corresponding to SEQ ID NO: 6 or a sequence at least 70% identical thereto. In some embodiments, the amino acid sequence corresponding to SEQ ID NO: 6 or a sequence at least 70% identical thereto further comprises a transmembrane domain. In some embodiments, the transmembrane domain comprises the sequence of SEQ ID NO: 47, or a sequence at least 70% identical thereto. In some embodiments, the plurality of antigens comprises an amino acid sequence corresponding to SEQ ID NO: 7 or a sequence at least 70% identical thereto. In some embodiments, the amino acid sequence corresponding to SEQ ID NO: 7 or a sequence at least 70% identical thereto further comprises a transmembrane domain. In some embodiments, the transmembrane domain comprises the sequence of SEQ ID NO: 48, or a sequence at least 70% identical thereto. In some embodiments, the plurality of antigens comprises an amino acid sequence corresponding to SEQ ID NO: 8 or a sequence at least 70% identical thereto. In some embodiments, the amino acid sequence corresponding to SEQ ID NO: 8 or a sequence at least 70% identical thereto further comprises a transmembrane domain. In some embodiments, the transmembrane domain comprises the sequence of SEQ ID NO: 49, or a sequence at least 70% identical thereto. In some embodiments, the plurality of antigens comprises an amino acid sequence corresponding to SEQ ID NO: 9 or a sequence at least 70% identical thereto. In some embodiments, the amino acid sequence corresponding to SEQ ID NO: 9 or a sequence at least 70% identical thereto further comprises a transmembrane domain. In some embodiments, the transmembrane domain comprises the sequence of SEQ ID NO: 50, or a sequence at least 70% identical thereto. In some embodiments, the plurality of antigens comprises an amino acid sequence corresponding to SEQ ID NO: 10 or a sequence at least 70% identical thereto. In some embodiments, the plurality of antigens comprises an amino acid sequence corresponding to SEQ ID NO: 11 or a sequence at least 70% identical thereto. In some embodiments, the plurality of antigens comprises an amino acid sequence corresponding to SEQ ID NO: 12 or a sequence at least 70% identical thereto. In some embodiments, the plurality of antigens comprises an amino acid sequence corresponding to SEQ ID NO: 13 or a sequence at least 70% identical thereto. In some embodiments, the plurality of antigens comprises an amino acid sequence corresponding to SEQ ID NO: 14 or a sequence at least 70% identical thereto. In some embodiments, the plurality of antigens comprises an amino acid sequence corresponding to SEQ ID NO: 15 or a sequence at least 70% identical thereto. In some embodiments, the plurality of antigens comprises an amino acid sequence corresponding to SEQ ID NO: 16 or a sequence at least 70% identical thereto. In some embodiments, the amino acid sequence corresponding to SEQ ID NO: 16 or a sequence at least 70% identical thereto further comprises a transmembrane domain. In some embodiments, the transmembrane domain comprises the sequence of SEQ ID NO: 51, or a sequence at least 70% identical thereto. In some embodiments, the plurality of antigens comprises an amino acid sequence corresponding to SEQ ID NO: 17 or a sequence at least 70% identical thereto. In some embodiments, the amino acid sequence corresponding to SEQ ID NO: 17 or a sequence at least 70% identical thereto further comprises a transmembrane domain. In some embodiments, the transmembrane domain comprises the sequence of SEQ ID NO: 52, or a sequence at least 70% identical thereto. In some embodiments, the plurality of antigens comprises an amino acid sequence corresponding to SEQ ID NO: 18 or a sequence at least 70% identical thereto. In some embodiments, the amino acid sequence corresponding to SEQ ID NO: 18 or a sequence at least 70% identical thereto further comprises a transmembrane domain. In some embodiments, the transmembrane domain comprises the sequence of SEQ ID NO: 53, or a sequence at least 70% identical thereto. In some embodiments, the plurality of antigens comprises an amino acid sequence corresponding to SEQ ID NO: 19 or a sequence at least 70% identical thereto. In some embodiments, the amino acid sequence corresponding to SEQ ID NO: 19 or a sequence at least 70% identical thereto further comprises a transmembrane domain. In some embodiments, the transmembrane domain comprises the sequence of SEQ ID NO: 54, or a sequence at least 70% identical thereto. In some embodiments, the plurality of antigens comprises an amino acid sequence corresponding to SEQ ID NO: 20 or a sequence at least 70% identical thereto. In some embodiments, the amino acid sequence corresponding to SEQ ID NO: 20 or a sequence at least 70% identical thereto further comprises a transmembrane domain. In some embodiments, the transmembrane domain comprises the sequence of SEQ ID NO: 55, or a sequence at least 70% identical thereto. In some embodiments, the plurality of antigens comprises an amino acid sequence corresponding to SEQ ID NO: 21 or a sequence at least 70% identical thereto. In some embodiments, the amino acid sequence corresponding to SEQ ID NO: 21 or a sequence at least 70% identical thereto further comprises a transmembrane domain. In some embodiments, the transmembrane domain comprises the sequence of SEQ ID NO: 56, or a sequence at least 70% identical thereto. In some embodiments, the plurality of antigens comprises an amino acid sequence corresponding to SEQ ID NO: 22 or a sequence at least 70% identical thereto. In some embodiments, the amino acid sequence corresponding to SEQ ID NO: 22 or a sequence at least 70% identical thereto further comprises a transmembrane domain. In some embodiments, the transmembrane domain comprises the sequence of SEQ ID NO: 57, or a sequence at least 70% identical thereto. In some embodiments, the plurality of antigens comprises an amino acid sequence corresponding to SEQ ID NO: 23 or a sequence at least 70% identical thereto. In some embodiments, the plurality of antigens comprises an amino acid sequence corresponding to SEQ ID NO: 24 or a sequence at least 70% identical thereto. In some embodiments, the amino acid sequence corresponding to SEQ ID NO: 24 or a sequence at least 70% identical thereto further comprises a transmembrane domain. In some embodiments, the transmembrane domain comprises the sequence of SEQ ID NO: 58, or a sequence at least 70% identical thereto. In some embodiments, the plurality of antigens comprises an amino acid sequence corresponding to SEQ ID NO: 25 or a sequence at least 70% identical thereto. In some embodiments, the plurality of antigens comprises an amino acid sequence corresponding to SEQ ID NO: 26 or a sequence at least 70% identical thereto. In some embodiments, the plurality of antigens comprises an amino acid sequence corresponding to SEQ ID NO: 27 or a sequence at least 70% identical thereto. In some embodiments, the plurality of antigens comprises an amino acid sequence corresponding to SEQ ID NO: 28 or a sequence at least 70% identical thereto. In some embodiments, the plurality of antigens comprises an amino acid sequence corresponding to SEQ ID NO: 29 or a sequence at least 70% identical thereto. In some embodiments, the plurality of antigens comprises an amino acid sequence corresponding to SEQ ID NO: 30 or a sequence at least 70% identical thereto. In some embodiments, the plurality of antigens comprises an amino acid sequence corresponding to SEQ ID NO: 31 or a sequence at least 70% identical thereto. In some embodiments, the plurality of antigens comprises an amino acid sequence corresponding to SEQ ID NO: 32 or a sequence at least 70% identical thereto. In some embodiments, the plurality of antigens comprises an amino acid sequence corresponding to SEQ ID NO: 33 or a sequence at least 70% identical thereto. In some embodiments, the plurality of antigens comprises an amino acid sequence corresponding to SEQ ID NO: 34 or a sequence at least 70% identical thereto. In some embodiments, the plurality of antigens comprises an amino acid sequence corresponding to SEQ ID NO: 35 or a sequence at least 70% identical thereto. In some embodiments, the plurality of antigens comprises an amino acid sequence corresponding to SEQ ID NO: 36 or a sequence at least 70% identical thereto. In some embodiments, the plurality of antigens comprises an amino acid sequence corresponding to SEQ ID NO: 37 or a sequence at least 70% identical thereto. In some embodiments, the plurality of antigens comprises an amino acid sequence corresponding to SEQ ID NO: 38 or a sequence at least 70% identical thereto. In some embodiments, the amino acid sequence corresponding to SEQ ID NO: 38 or a sequence at least 70% identical thereto further comprises a transmembrane domain. In some embodiments, the transmembrane domain comprises the sequence of SEQ ID NO: 59, or a sequence at least 70% identical thereto. In some embodiments, the plurality of antigens comprises an amino acid sequence corresponding to SEQ ID NO: 39 or a sequence at least 70% identical thereto. In some embodiments, the amino acid sequence corresponding to SEQ ID NO: 39 or a sequence at least 70% identical thereto further comprises a transmembrane domain. In some embodiments, the transmembrane domain comprises the sequence of SEQ ID NO: 60, or a sequence at least 70% identical thereto. In some embodiments, the plurality of antigens comprises an amino acid sequence corresponding to SEQ ID NO: 40 or a sequence at least 70% identical thereto. In some embodiments, the amino acid sequence corresponding to SEQ ID NO: 40 or a sequence at least 70% identical thereto further comprises a transmembrane domain. In some embodiments, the transmembrane domain comprises the sequence of SEQ ID NO: 61, or a sequence at least 70% identical thereto. In some embodiments, the plurality of antigens comprises an amino acid sequence corresponding to SEQ ID NO: 41 or a sequence at least 70% identical thereto. In some embodiments, the amino acid sequence corresponding to SEQ ID NO: 41 or a sequence at least 70% identical thereto further comprises a transmembrane domain. In some embodiments, the transmembrane domain comprises the sequence of SEQ ID NO: 62, or a sequence at least 70% identical thereto. In some embodiments, the plurality of antigens comprises an amino acid sequence corresponding to SEQ ID NO: 42 or a sequence at least 70% identical thereto. In some embodiments, the amino acid sequence corresponding to SEQ ID NO: 42 or a sequence at least 70% identical thereto further comprises a transmembrane domain. In some embodiments, the transmembrane domain comprises the sequence of SEQ ID NO: 63, or a sequence at least 70% identical thereto.

In some embodiments, the plurality of antigens comprises a polypeptide with amino acid sequence corresponding to SEQ ID NO: 108 or a sequence at least 90% identical thereto. In some embodiments, the plurality of antigens comprises a polypeptide with amino acid sequence corresponding to SEQ ID NO: 109 or a sequence at least 90% identical thereto. In some embodiments, the plurality of antigens comprises a polypeptide with amino acid sequence corresponding to SEQ ID NO: 110 or a sequence at least 90% identical thereto. In some embodiments, the plurality of antigens comprises a polypeptide with amino acid sequence corresponding to SEQ ID NO: 111 or a sequence at least 90% identical thereto. In some embodiments, the plurality of antigens comprises a polypeptide with amino acid sequence corresponding to SEQ ID NO: 112 or a sequence at least 90% identical thereto. In some embodiments, the plurality of antigens comprises a polypeptide with amino acid sequence corresponding to SEQ ID NO: 113 or a sequence at least 90% identical thereto. In some embodiments, the plurality of antigens comprises a polypeptide with amino acid sequence corresponding to SEQ ID NO: 114 or a sequence at least 90% identical thereto. In some embodiments, the plurality of antigens comprises a polypeptide with amino acid sequence corresponding to SEQ ID NO: 115 or a sequence at least 90% identical thereto. In some embodiments, the plurality of antigens comprises a polypeptide with amino acid sequence corresponding to SEQ ID NO: 116 or a sequence at least 90% identical thereto. In some embodiments, the plurality of antigens comprises a polypeptide with amino acid sequence corresponding to SEQ ID NO: 117 or a sequence at least 90% identical thereto. In some embodiments, the plurality of antigens comprises a polypeptide with amino acid sequence corresponding to SEQ ID NO: 118 or a sequence at least 90% identical thereto. In some embodiments, the plurality of antigens comprises a polypeptide with amino acid sequence corresponding to SEQ ID NO: 119 or a sequence at least 90% identical thereto. In some embodiments, the plurality of antigens comprises a polypeptide with amino acid sequence corresponding to SEQ ID NO: 120 or a sequence at least 90% identical thereto. In some embodiments, the plurality of antigens comprises a polypeptide with amino acid sequence corresponding to SEQ ID NO: 121 or a sequence at least 90% identical thereto. In some embodiments, the plurality of antigens comprises a polypeptide with amino acid sequence corresponding to SEQ ID NO: 122 or a sequence at least 90% identical thereto. In some embodiments, the plurality of antigens comprises a polypeptide with amino acid sequence corresponding to SEQ ID NO: 123 or a sequence at least 90% identical thereto. In some embodiments, the plurality of antigens comprises a polypeptide with amino acid sequence corresponding to SEQ ID NO: 124 or a sequence at least 90% identical thereto. In some embodiments, the plurality of antigens comprises a polypeptide with amino acid sequence corresponding to SEQ ID NO: 125 or a sequence at least 90% identical thereto. In some embodiments, the plurality of antigens comprises a polypeptide with amino acid sequence corresponding to SEQ ID NO: 126 or a sequence at least 90% identical thereto. In some embodiments, the plurality of antigens comprises a polypeptide with amino acid sequence corresponding to SEQ ID NO: 127 or a sequence at least 90% identical thereto. In some embodiments, the plurality of antigens comprises a polypeptide with amino acid sequence corresponding to SEQ ID NO: 128 or a sequence at least 90% identical thereto.

In some embodiments, the plurality of antigens comprises a polypeptide with amino acid sequence corresponding to SEQ ID NO: 108 or a sequence at least 95% identical thereto. In some embodiments, the plurality of antigens comprises a polypeptide with amino acid sequence corresponding to SEQ ID NO: 109 or a sequence at least 95% identical thereto. In some embodiments, the plurality of antigens comprises a polypeptide with amino acid sequence corresponding to SEQ ID NO: 110 or a sequence at least 95% identical thereto. In some embodiments, the plurality of antigens comprises a polypeptide with amino acid sequence corresponding to SEQ ID NO: 111 or a sequence at least 95% identical thereto. In some embodiments, the plurality of antigens comprises a polypeptide with amino acid sequence corresponding to SEQ ID NO: 112 or a sequence at least 95% identical thereto. In some embodiments, the plurality of antigens comprises a polypeptide with amino acid sequence corresponding to SEQ ID NO: 113 or a sequence at least 95% identical thereto. In some embodiments, the plurality of antigens comprises a polypeptide with amino acid sequence corresponding to SEQ ID NO: 114 or a sequence at least 95% identical thereto. In some embodiments, the plurality of antigens comprises a polypeptide with amino acid sequence corresponding to SEQ ID NO: 115 or a sequence at least 95% identical thereto. In some embodiments, the plurality of antigens comprises a polypeptide with amino acid sequence corresponding to SEQ ID NO: 116 or a sequence at least 95% identical thereto. In some embodiments, the plurality of antigens comprises a polypeptide with amino acid sequence corresponding to SEQ ID NO: 117 or a sequence at least 95% identical thereto. In some embodiments, the plurality of antigens comprises a polypeptide with amino acid sequence corresponding to SEQ ID NO: 118 or a sequence at least 95% identical thereto. In some embodiments, the plurality of antigens comprises a polypeptide with amino acid sequence corresponding to SEQ ID NO: 119 or a sequence at least 95% identical thereto. In some embodiments, the plurality of antigens comprises a polypeptide with amino acid sequence corresponding to SEQ ID NO: 120 or a sequence at least 95% identical thereto. In some embodiments, the plurality of antigens comprises a polypeptide with amino acid sequence corresponding to SEQ ID NO: 121 or a sequence at least 95% identical thereto. In some embodiments, the plurality of antigens comprises a polypeptide with amino acid sequence corresponding to SEQ ID NO: 122 or a sequence at least 95% identical thereto. In some embodiments, the plurality of antigens comprises a polypeptide with amino acid sequence corresponding to SEQ ID NO: 123 or a sequence at least 95% identical thereto. In some embodiments, the plurality of antigens comprises a polypeptide with amino acid sequence corresponding to SEQ ID NO: 124 or a sequence at least 95% identical thereto. In some embodiments, the plurality of antigens comprises a polypeptide with amino acid sequence corresponding to SEQ ID NO: 125 or a sequence at least 95% identical thereto. In some embodiments, the plurality of antigens comprises a polypeptide with amino acid sequence corresponding to SEQ ID NO: 126 or a sequence at least 95% identical thereto. In some embodiments, the plurality of antigens comprises a polypeptide with amino acid sequence corresponding to SEQ ID NO: 127 or a sequence at least 95% identical thereto. In some embodiments, the plurality of antigens comprises a polypeptide with amino acid sequence corresponding to SEQ ID NO: 128 or a sequence at least 95% identical thereto.

In some embodiments, the plurality of antigens comprises a polypeptide with amino acid sequence corresponding to SEQ ID NO: 108 or a sequence at least 98% identical thereto. In some embodiments, the plurality of antigens comprises a polypeptide with amino acid sequence corresponding to SEQ ID NO: 109 or a sequence at least 98% identical thereto. In some embodiments, the plurality of antigens comprises a polypeptide with amino acid sequence corresponding to SEQ ID NO: 110 or a sequence at least 98% identical thereto. In some embodiments, the plurality of antigens comprises a polypeptide with amino acid sequence corresponding to SEQ ID NO: 111 or a sequence at least 98% identical thereto. In some embodiments, the plurality of antigens comprises a polypeptide with amino acid sequence corresponding to SEQ ID NO: 112 or a sequence at least 98% identical thereto. In some embodiments, the plurality of antigens comprises a polypeptide with amino acid sequence corresponding to SEQ ID NO: 113 or a sequence at least 98% identical thereto. In some embodiments, the plurality of antigens comprises a polypeptide with amino acid sequence corresponding to SEQ ID NO: 114 or a sequence at least 98% identical thereto. In some embodiments, the plurality of antigens comprises a polypeptide with amino acid sequence corresponding to SEQ ID NO: 115 or a sequence at least 98% identical thereto. In some embodiments, the plurality of antigens comprises a polypeptide with amino acid sequence corresponding to SEQ ID NO: 116 or a sequence at least 98% identical thereto. In some embodiments, the plurality of antigens comprises a polypeptide with amino acid sequence corresponding to SEQ ID NO: 117 or a sequence at least 98% identical thereto. In some embodiments, the plurality of antigens comprises a polypeptide with amino acid sequence corresponding to SEQ ID NO: 118 or a sequence at least 98% identical thereto. In some embodiments, the plurality of antigens comprises a polypeptide with amino acid sequence corresponding to SEQ ID NO: 119 or a sequence at least 98% identical thereto. In some embodiments, the plurality of antigens comprises a polypeptide with amino acid sequence corresponding to SEQ ID NO: 120 or a sequence at least 98% identical thereto. In some embodiments, the plurality of antigens comprises a polypeptide with amino acid sequence corresponding to SEQ ID NO: 121 or a sequence at least 98% identical thereto. In some embodiments, the plurality of antigens comprises a polypeptide with amino acid sequence corresponding to SEQ ID NO: 122 or a sequence at least 98% identical thereto. In some embodiments, the plurality of antigens comprises a polypeptide with amino acid sequence corresponding to SEQ ID NO: 123 or a sequence at least 98% identical thereto. In some embodiments, the plurality of antigens comprises a polypeptide with amino acid sequence corresponding to SEQ ID NO: 124 or a sequence at least 98% identical thereto. In some embodiments, the plurality of antigens comprises a polypeptide with amino acid sequence corresponding to SEQ ID NO: 125 or a sequence at least 98% identical thereto. In some embodiments, the plurality of antigens comprises a polypeptide with amino acid sequence corresponding to SEQ ID NO: 126 or a sequence at least 98% identical thereto. In some embodiments, the plurality of antigens comprises a polypeptide with amino acid sequence corresponding to SEQ ID NO: 127 or a sequence at least 98% identical thereto. In some embodiments, the plurality of antigens comprises a polypeptide with amino acid sequence corresponding to SEQ ID NO: 128 or a sequence at least 98% identical thereto.

In some embodiments, the plurality of antigens comprises a polypeptide with amino acid sequence corresponding to SEQ ID NO: 108 or a sequence at least 99% identical thereto. In some embodiments, the plurality of antigens comprises a polypeptide with amino acid sequence corresponding to SEQ ID NO: 109 or a sequence at least 99% identical thereto. In some embodiments, the plurality of antigens comprises a polypeptide with amino acid sequence corresponding to SEQ ID NO: 110 or a sequence at least 99% identical thereto. In some embodiments, the plurality of antigens comprises a polypeptide with amino acid sequence corresponding to SEQ ID NO: 111 or a sequence at least 99% identical thereto. In some embodiments, the plurality of antigens comprises a polypeptide with amino acid sequence corresponding to SEQ ID NO: 112 or a sequence at least 99% identical thereto. In some embodiments, the plurality of antigens comprises a polypeptide with amino acid sequence corresponding to SEQ ID NO: 113 or a sequence at least 99% identical thereto. In some embodiments, the plurality of antigens comprises a polypeptide with amino acid sequence corresponding to SEQ ID NO: 114 or a sequence at least 99% identical thereto. In some embodiments, the plurality of antigens comprises a polypeptide with amino acid sequence corresponding to SEQ ID NO: 115 or a sequence at least 99% identical thereto. In some embodiments, the plurality of antigens comprises a polypeptide with amino acid sequence corresponding to SEQ ID NO: 116 or a sequence at least 99% identical thereto. In some embodiments, the plurality of antigens comprises a polypeptide with amino acid sequence corresponding to SEQ ID NO: 117 or a sequence at least 99% identical thereto. In some embodiments, the plurality of antigens comprises a polypeptide with amino acid sequence corresponding to SEQ ID NO: 118 or a sequence at least 99% identical thereto. In some embodiments, the plurality of antigens comprises a polypeptide with amino acid sequence corresponding to SEQ ID NO: 119 or a sequence at least 99% identical thereto. In some embodiments, the plurality of antigens comprises a polypeptide with amino acid sequence corresponding to SEQ ID NO: 120 or a sequence at least 99% identical thereto. In some embodiments, the plurality of antigens comprises a polypeptide with amino acid sequence corresponding to SEQ ID NO: 121 or a sequence at least 99% identical thereto. In some embodiments, the plurality of antigens comprises a polypeptide with amino acid sequence corresponding to SEQ ID NO: 122 or a sequence at least 99% identical thereto. In some embodiments, the plurality of antigens comprises a polypeptide with amino acid sequence corresponding to SEQ ID NO: 123 or a sequence at least 99% identical thereto. In some embodiments, the plurality of antigens comprises a polypeptide with amino acid sequence corresponding to SEQ ID NO: 124 or a sequence at least 99% identical thereto. In some embodiments, the plurality of antigens comprises a polypeptide with amino acid sequence corresponding to SEQ ID NO: 125 or a sequence at least 99% identical thereto. In some embodiments, the plurality of antigens comprises a polypeptide with amino acid sequence corresponding to SEQ ID NO: 126 or a sequence at least 99% identical thereto. In some embodiments, the plurality of antigens comprises a polypeptide with amino acid sequence corresponding to SEQ ID NO: 127 or a sequence at least 99% identical thereto. In some embodiments, the plurality of antigens comprises a polypeptide with amino acid sequence corresponding to SEQ ID NO: 128 or a sequence at least 99% identical thereto.

In some embodiments, the vaccine composition comprises eight distinct antigens comprising amino acid sequences selected from the group consisting of SEQ ID NOS: 1-8, 16-23, or 29. In some embodiments, the vaccine composition comprises eight distinct antigens comprising amino acid sequences selected from the group consisting of at least 6 distinct antigens comprising amino acid sequences selected from the group consisting of SEQ ID NOS: 1-8, 16-23, or 29, or sequences at least 70% identical to any one of such sequences. In some embodiments, the vaccine composition comprises eight distinct antigens comprising amino acid sequences selected from the group consisting of SEQ ID NOS: 1-8, 16-23, or 29, or sequences at least 70% identical to any one of such sequences. In some embodiments, the vaccine composition comprises at least 10 distinct antigens comprising amino acid sequences selected from the group consisting of SEQ ID NOS: 1-8, 16-23, or 29, or sequences at least 70% identical to any one of such sequences. In some embodiments, the vaccine composition comprises at least 12 distinct antigens comprising amino acid sequences selected from the group consisting of SEQ ID NOS: 1-8, 16-23, or 29, or sequences at least 70% identical to any one of such sequences. In some embodiments, the vaccine composition comprises at least 14 distinct antigens comprising amino acid sequences selected from the group consisting of SEQ ID NOS: 1-8, 16-23, or 29, or sequences at least 70% identical to any one of such sequences. In some embodiments, the vaccine composition comprises at least 16 distinct antigens comprising amino acid sequences selected from the group consisting of SEQ ID NOS: 1-8, 16-23, or 29, or sequences at least 70% identical to any one of such sequences. In some embodiments, the vaccine composition further comprises a pharmaceutically acceptable carrier.

In some embodiments, the vaccine composition comprises eight or more distinct antigens comprising amino acid sequences selected from the group consisting of SEQ ID NOS: 2-9, 16-22, 24, 38-42. In some embodiments, the vaccine composition comprises eight or more distinct antigens comprising amino acid sequences selected from the group consisting of at least 6 distinct antigens comprising amino acid sequences selected from the group consisting of SEQ ID NOS: 2-9, 16-22, 24, 38-42, or sequences at least 70% identical to any one of such sequences. In some embodiments, the vaccine composition comprises eight or more distinct antigens comprising amino acid sequences selected from the group consisting of SEQ ID NOS: 2-9, 16-22, 24, 38-42, or sequences at least 70% identical to any one of such sequences. In some embodiments, the vaccine composition comprises at least 10 distinct antigens comprising amino acid sequences selected from the group consisting of SEQ ID NOS: 2-9, 16-22, 24, 38-42, or sequences at least 70% identical to any one of such sequences. In some embodiments, the vaccine composition comprises at least 12 distinct antigens comprising amino acid sequences selected from the group consisting of SEQ ID NOS: 2-9, 16-22, 24, 38-42, or sequences at least 70% identical to any one of such sequences. In some embodiments, the vaccine composition comprises at least 14 distinct antigens comprising amino acid sequences selected from the group consisting of SEQ ID NOS: 2-9, 16-22, 24, 38-42, or sequences at least 70% identical to any one of such sequences. In some embodiments, the vaccine composition comprises at least 16 distinct antigens comprising amino acid sequences selected from the group consisting of SEQ ID NOS: 2-9, 16-22, 24, 38-42, or sequences at least 70% identical to any one of such sequences. In some embodiments, the vaccine composition further comprises a pharmaceutically acceptable carrier.

In some embodiments, the vaccine composition comprises four or more distinct antigens comprising amino acid sequences selected from the group consisting of SEQ ID NOS: 2-9, 38-39, or sequences at least 90% identical to any one of such sequences. In some embodiments, the vaccine composition comprises six or more distinct antigens comprising amino acid sequences selected from the group consisting of SEQ ID NOS: 2-9, 38-39, or sequences at least 90% identical to any one of such sequences. In some embodiments, the vaccine composition comprises eight or more distinct antigens comprising amino acid sequences selected from the group consisting of SEQ ID NOS: 2-9, 38-39, or sequences at least 90% identical to any one of such sequences. In some embodiments, the vaccine composition comprises antigens comprising SEQ ID NOS: 2-9, 38-39, or sequences at least 90% identical to any one of such sequences. In some embodiments, the vaccine composition comprises four or more distinct antigens comprising amino acid sequences selected from the group consisting of SEQ ID NOS: 16-22, 24, 40-41, or sequences at least 90% identical to any one of such sequences. In some embodiments, the vaccine composition comprises six or more distinct antigens comprising amino acid sequences selected from the group consisting of SEQ ID NOS: 16-22, 24, 40-41, or sequences at least 90% identical to any one of such sequences. In some embodiments, the vaccine composition comprises eight or more distinct antigens comprising amino acid sequences selected from the group consisting of SEQ ID NOS: 16-22, 24, 40-41, or sequences at least 90% identical to any one of such sequences. In some embodiments, the vaccine composition comprises antigens comprising SEQ ID NOS: 16-22, 24, 40-41, or sequences at least 90% identical to any one of such sequences. In some embodiments, the vaccine composition comprises an antigen comprising SEQ ID NO: 42, or a sequence at least 90% identical thereto.

In some embodiments, the vaccine composition comprises four or more distinct antigens comprising amino acid sequences selected from the group consisting of SEQ ID NOS: 108-115, 124-125, or sequences at least 90% identical to any one of such sequences. In some embodiments, the vaccine composition comprises six or more distinct antigens comprising amino acid sequences selected from the group consisting of SEQ ID NOS: 108-115, 124-125, or sequences at least 90% identical to any one of such sequences. In some embodiments, the vaccine composition comprises eight or more distinct antigens comprising amino acid sequences selected from the group consisting of SEQ ID NOS: 108-115, 124-125, or sequences at least 90% identical to any one of such sequences. In some embodiments, the vaccine composition comprises antigens comprising SEQ ID NOS: 108-115, 124-125, or sequences at least 90% identical to any one of such sequences. In some embodiments, the vaccine composition comprises four or more distinct antigens comprising amino acid sequences selected from the group consisting of SEQ ID NOS: 116-123, 126-127, or sequences at least 90% identical to any one of such sequences. In some embodiments, the vaccine composition comprises six or more distinct antigens comprising amino acid sequences selected from the group consisting of SEQ ID NOS: 116-123, 126-127, or sequences at least 90% identical to any one of such sequences. In some embodiments, the vaccine composition comprises eight or more distinct antigens comprising amino acid sequences selected from the group consisting of SEQ ID NOS: 116-123, 126-127, or sequences at least 90% identical to any one of such sequences. In some embodiments, the vaccine composition comprises antigens comprising SEQ ID NOS: 116-123, 126-127, or sequences at least 90% identical to any one of such sequences. In some embodiments, the vaccine composition comprises an antigen comprising SEQ ID NO: 128, or a sequence at least 90% identical thereto.

In some embodiments, each antigen of a plurality of antigens is present in the vaccine composition in an amount of less than about 5 micrograms (μg). In some embodiments, each antigen of a plurality of antigens is present in the vaccine composition in an amount of less than about 5 μg, less than about 4 μg, less than about 3 μg, less than about 2 μg, or less than about 1 pg. In some embodiments, the plurality of antigens comprises at least 6, at least 8, at least 10, at least 12, at least 14, at least 16, or at least 17 distinct antigens. In some embodiments, the plurality of antigens comprises no more than 30 antigens. In some embodiments, the plurality of antigens comprises no more than 30, 29, 28, 27, 26, 25, 24, 23, 22, 21, 20, 19, 18, 17, 16, 15, 14, 13, 12, 11, 10, 9, 8, 7, or 6 antigens. In some embodiments, the plurality of antigens comprises no more than 25 antigens. In some embodiments, the plurality of antigens comprises no more than 20 antigens.

The present disclosure provides a nucleic acid vaccine composition comprising one or more nucleic acids encoding for a plurality of antigens as disclosed herein. In some embodiments, the one or more nucleic acids are a messenger RNA (mRNA). In some embodiments, the mRNA is encapsulated in lipid nanoparticles. In some embodiments, the one or more nucleic acids encode for an antigen comprising or consisting of an amino acid sequence corresponding to SEQ ID NO: 1 or a sequence at least 70% identical thereto. In some embodiments, the one or more nucleic acids encode for an antigen comprising or consisting of an amino acid sequence corresponding to SEQ ID NO: 2 or a sequence at least 70% identical thereto. In some embodiments, the one or more nucleic acids encoding for the antigen further encode for a transmembrane domain. In some embodiments, the transmembrane domain comprises the sequence of SEQ ID NO: 43, or a sequence at least 70% identical thereto. In some embodiments, the one or more nucleic acids encode for an antigen comprising or consisting of an amino acid sequence corresponding to SEQ ID NO: 3 or a sequence at least 70% identical thereto. In some embodiments, the one or more nucleic acids encoding for the antigen further encode for a transmembrane domain. In some embodiments, the transmembrane domain comprises the sequence of SEQ ID NO: 44, or a sequence at least 70% identical thereto. In some embodiments, the one or more nucleic acids encode for an antigen comprising or consisting of an amino acid sequence corresponding to SEQ ID NO: 4 or a sequence at least 70% identical thereto. In some embodiments, the one or more nucleic acids encoding for the antigen further encode for a transmembrane domain. In some embodiments, the transmembrane domain comprises the sequence of SEQ ID NO: 45, or a sequence at least 70% identical thereto. In some embodiments, the one or more nucleic acids encode for an antigen comprising or consisting of an amino acid sequence corresponding to SEQ ID NO: 5 or a sequence at least 70% identical thereto. In some embodiments, the one or more nucleic acids encoding for the antigen further encode for a transmembrane domain. In some embodiments, the transmembrane domain comprises the sequence of SEQ ID NO: 46, or a sequence at least 70% identical thereto. In some embodiments, the one or more nucleic acids encode for an antigen comprising or consisting of an amino acid sequence corresponding to SEQ ID NO: 6 or a sequence at least 70% identical thereto. In some embodiments, the one or more nucleic acids encoding for the antigen further encode for a transmembrane domain. In some embodiments, the transmembrane domain comprises the sequence of SEQ ID NO: 47, or a sequence at least 70% identical thereto. In some embodiments, the one or more nucleic acids encode for an antigen comprising or consisting of an amino acid sequence corresponding to SEQ ID NO: 7 or a sequence at least 70% identical thereto. In some embodiments, the one or more nucleic acids encoding for the antigen further encode for a transmembrane domain. In some embodiments, the transmembrane domain comprises the sequence of SEQ ID NO: 48, or a sequence at least 70% identical thereto. In some embodiments, the one or more nucleic acids encode for an antigen comprising or consisting of an amino acid sequence corresponding to SEQ ID NO: 8 or a sequence at least 70% identical thereto. In some embodiments, the one or more nucleic acids encoding for the antigen further encode for a transmembrane domain. In some embodiments, the transmembrane domain comprises the sequence of SEQ ID NO: 49, or a sequence at least 70% identical thereto. In some embodiments, the one or more nucleic acids encode for an antigen comprising or consisting of an amino acid sequence corresponding to SEQ ID NO: 9 or a sequence at least 70% identical thereto. In some embodiments, the one or more nucleic acids encoding for the antigen further encode for a transmembrane domain. In some embodiments, the transmembrane domain comprises the sequence of SEQ ID NO: 50, or a sequence at least 70% identical thereto. In some embodiments, the one or more nucleic acids encode for an antigen comprising or consisting of an amino acid sequence corresponding to SEQ ID NO: 10 or a sequence at least 70% identical thereto. In some embodiments, the one or more nucleic acids encode for an antigen comprising or consisting of an amino acid sequence corresponding to SEQ ID NO: 11 or a sequence at least 70% identical thereto. In some embodiments, the one or more nucleic acids encode for an antigen comprising or consisting of an amino acid sequence corresponding to SEQ ID NO: 12 or a sequence at least 70% identical thereto. In some embodiments, the one or more nucleic acids encode for an antigen comprising or consisting of an amino acid sequence corresponding to SEQ ID NO: 13 or a sequence at least 70% identical thereto. In some embodiments, the one or more nucleic acids encode for an antigen comprising or consisting of an amino acid sequence corresponding to SEQ ID NO: 14 or a sequence at least 70% identical thereto. In some embodiments, the one or more nucleic acids encode for an antigen comprising or consisting of an amino acid sequence corresponding to SEQ ID NO: 15 or a sequence at least 70% identical thereto. In some embodiments, the one or more nucleic acids encode for an antigen comprising or consisting of an amino acid sequence corresponding to SEQ ID NO: 16 or a sequence at least 70% identical thereto. In some embodiments, the one or more nucleic acids encoding for the antigen further encode for a transmembrane domain. In some embodiments, the transmembrane domain comprises the sequence of SEQ ID NO: 51, or a sequence at least 70% identical thereto. In some embodiments, the one or more nucleic acids encode for an antigen comprising or consisting of an amino acid sequence corresponding to SEQ ID NO: 17 or a sequence at least 70% identical thereto. In some embodiments, the one or more nucleic acids encoding for the antigen further encode for a transmembrane domain. In some embodiments, the transmembrane domain comprises the sequence of SEQ ID NO: 52, or a sequence at least 70% identical thereto. In some embodiments, the one or more nucleic acids encode for an antigen comprising or consisting of an amino acid sequence corresponding to SEQ ID NO: 18 or a sequence at least 70% identical thereto. In some embodiments, the one or more nucleic acids encoding for the antigen further encode for a transmembrane domain. In some embodiments, the transmembrane domain comprises the sequence of SEQ ID NO: 53, or a sequence at least 70% identical thereto. In some embodiments, the one or more nucleic acids encode for an antigen comprising or consisting of an amino acid sequence corresponding to SEQ ID NO: 19 or a sequence at least 70% identical thereto. In some embodiments, the one or more nucleic acids encoding for the antigen further encode for a transmembrane domain. In some embodiments, the transmembrane domain comprises the sequence of SEQ ID NO: 54, or a sequence at least 70% identical thereto. In some embodiments, the one or more nucleic acids encode for an antigen comprising or consisting of an amino acid sequence corresponding to SEQ ID NO: 20 or a sequence at least 70% identical thereto. In some embodiments, the one or more nucleic acids encoding for the antigen further encode for a transmembrane domain. In some embodiments, the transmembrane domain comprises the sequence of SEQ ID NO: 55, or a sequence at least 70% identical thereto. In some embodiments, the one or more nucleic acids encode for an antigen comprising or consisting of an amino acid sequence corresponding to SEQ ID NO: 21 or a sequence at least 70% identical thereto. In some embodiments, the one or more nucleic acids encoding for the antigen further encode for a transmembrane domain. In some embodiments, the transmembrane domain comprises the sequence of SEQ ID NO: 56, or a sequence at least 70% identical thereto. In some embodiments, the one or more nucleic acids encode for an antigen comprising or consisting of an amino acid sequence corresponding to SEQ ID NO: 22 or a sequence at least 70% identical thereto. In some embodiments, the one or more nucleic acids encoding for the antigen further encode for a transmembrane domain. In some embodiments, the transmembrane domain comprises the sequence of SEQ ID NO: 57, or a sequence at least 70% identical thereto. In some embodiments, the one or more nucleic acids encode for an antigen comprising or consisting of an amino acid sequence corresponding to SEQ ID NO: 23 or a sequence at least 70% identical thereto. In some embodiments, the one or more nucleic acids encode for an antigen comprising or consisting of an amino acid sequence corresponding to SEQ ID NO: 24 or a sequence at least 70% identical thereto. In some embodiments, the one or more nucleic acids encoding for the antigen further encode for a transmembrane domain. In some embodiments, the transmembrane domain comprises the sequence of SEQ ID NO: 58, or a sequence at least 70% identical thereto. In some embodiments, the one or more nucleic acids encode for an antigen comprising or consisting of an amino acid sequence corresponding to SEQ ID NO: 25 or a sequence at least 70% identical thereto. In some embodiments, the one or more nucleic acids encode for an antigen comprising or consisting of an amino acid sequence corresponding to SEQ ID NO: 26 or a sequence at least 70% identical thereto. In some embodiments, the one or more nucleic acids encode for an antigen comprising or consisting of an amino acid sequence corresponding to SEQ ID NO: 27 or a sequence at least 70% identical thereto. In some embodiments, the one or more nucleic acids encode for an antigen comprising or consisting of an amino acid sequence corresponding to SEQ ID NO: 28 or a sequence at least 70% identical thereto. In some embodiments, the one or more nucleic acids encode for an antigen comprising or consisting of an amino acid sequence corresponding to SEQ ID NO: 29 or a sequence at least 70% identical thereto. In some embodiments, the one or more nucleic acids encode for an antigen comprising or consisting of an amino acid sequence corresponding to SEQ ID NO: 30 or a sequence at least 70% identical thereto. In some embodiments, the one or more nucleic acids encode for an antigen comprising or consisting of an amino acid sequence corresponding to SEQ ID NO: 31 or a sequence at least 70% identical thereto. In some embodiments, the one or more nucleic acids encode for an antigen comprising or consisting of an amino acid sequence corresponding to SEQ ID NO: 32 or a sequence at least 70% identical thereto. In some embodiments, the one or more nucleic acids encode for an antigen comprising or consisting of an amino acid sequence corresponding to SEQ ID NO: 33 or a sequence at least 70% identical thereto. In some embodiments, the one or more nucleic acids encode for an antigen comprising or consisting of an amino acid sequence corresponding to SEQ ID NO: 34 or a sequence at least 70% identical thereto. In some embodiments, the one or more nucleic acids encode for an antigen comprising or consisting of an amino acid sequence corresponding to SEQ ID NO: 35 or a sequence at least 70% identical thereto. In some embodiments, the one or more nucleic acids encode for an antigen comprising or consisting of an amino acid sequence corresponding to SEQ ID NO: 36 or a sequence at least 70% identical thereto. In some embodiments, the one or more nucleic acids encode for an antigen comprising or consisting of an amino acid sequence corresponding to SEQ ID NO: 37 or a sequence at least 70% identical thereto. In some embodiments, the one or more nucleic acids encode for an antigen comprising or consisting of an amino acid sequence corresponding to SEQ ID NO: 38 or a sequence at least 70% identical thereto. In some embodiments, the one or more nucleic acids encoding for the antigen further encode for a transmembrane domain. In some embodiments, the transmembrane domain comprises the sequence of SEQ ID NO: 59, or a sequence at least 70% identical thereto. In some embodiments, the one or more nucleic acids encode for an antigen comprising or consisting of an amino acid sequence corresponding to SEQ ID NO: 39 or a sequence at least 70% identical thereto. In some embodiments, the one or more nucleic acids encoding for the antigen further encode for a transmembrane domain. In some embodiments, the transmembrane domain comprises the sequence of SEQ ID NO: 60, or a sequence at least 70% identical thereto. In some embodiments, the one or more nucleic acids encode for an antigen comprising or consisting of an amino acid sequence corresponding to SEQ ID NO: 40 or a sequence at least 70% identical thereto. In some embodiments, the one or more nucleic acids encoding for the antigen further encode for a transmembrane domain. In some embodiments, the transmembrane domain comprises the sequence of SEQ ID NO: 61, or a sequence at least 70% identical thereto. In some embodiments, the one or more nucleic acids encode for an antigen comprising or consisting of an amino acid sequence corresponding to SEQ ID NO: 41 or a sequence at least 70% identical thereto. In some embodiments, the one or more nucleic acids encoding for the antigen further encode for a transmembrane domain. In some embodiments, the transmembrane domain comprises the sequence of SEQ ID NO: 62, or a sequence at least 70% identical thereto. In some embodiments, the one or more nucleic acids encode for an antigen comprising or consisting of an amino acid sequence corresponding to SEQ ID NO: 42 or a sequence at least 70% identical thereto. In some embodiments, the one or more nucleic acids encoding for the antigen further encode for a transmembrane domain. In some embodiments, the transmembrane domain comprises the sequence of SEQ ID NO: 63, or a sequence at least 70% identical thereto. In some embodiments, the nucleic acid vaccine composition comprises a nucleic acid having any one of the sequences described in Table 3. In some embodiments, the nucleic acid vaccine composition comprises a nucleic acid having any one of SEQ ID NOS: 64-84. In some embodiments, the nucleic acid encodes a signal peptide sequence. In some embodiments, the nucleic acid encodes a transmembrane domain. In some embodiments, the nucleic acid encodes an extracellular domain.

In some embodiments, the one or more nucleic acids encoding for an antigen encode a signal peptide, extracellular domain, and transmembrane domain. In some embodiments, the one or more nucleic acids encode for an antigen comprising or consisting of an amino acid sequence corresponding to SEQ ID NO: 85 or a sequence at least 90% identical thereto. In some embodiments, the one or more nucleic acids encode for an antigen comprising or consisting of an amino acid sequence corresponding to SEQ ID NO: 86 or a sequence at least 90% identical thereto. In some embodiments, the one or more nucleic acids encode for an antigen comprising or consisting of an amino acid sequence corresponding to SEQ ID NO: 87 or a sequence at least 90% identical thereto. In some embodiments, the one or more nucleic acids encode for an antigen comprising or consisting of an amino acid sequence corresponding to SEQ ID NO: 88 or a sequence at least 90% identical thereto. In some embodiments, the one or more nucleic acids encode for an antigen comprising or consisting of an amino acid sequence corresponding to SEQ ID NO: 89 or a sequence at least 90% identical thereto. In some embodiments, the one or more nucleic acids encode for an antigen comprising or consisting of an amino acid sequence corresponding to SEQ ID NO: 90 or a sequence at least 90% identical thereto. In some embodiments, the one or more nucleic acids encode for an antigen comprising or consisting of an amino acid sequence corresponding to SEQ ID NO: 91 or a sequence at least 90% identical thereto. In some embodiments, the one or more nucleic acids encode for an antigen comprising or consisting of an amino acid sequence corresponding to SEQ ID NO: 92 or a sequence at least 90% identical thereto. In some embodiments, the one or more nucleic acids encode for an antigen comprising or consisting of an amino acid sequence corresponding to SEQ ID NO: 93 or a sequence at least 90% identical thereto. In some embodiments, the one or more nucleic acids encode for an antigen comprising or consisting of an amino acid sequence corresponding to SEQ ID NO: 94 or a sequence at least 90% identical thereto. In some embodiments, the one or more nucleic acids encode for an antigen comprising or consisting of an amino acid sequence corresponding to SEQ ID NO: 95 or a sequence at least 90% identical thereto. In some embodiments, the one or more nucleic acids encode for an antigen comprising or consisting of an amino acid sequence corresponding to SEQ ID NO: 96 or a sequence at least 90% identical thereto. In some embodiments, the one or more nucleic acids encode for an antigen comprising or consisting of an amino acid sequence corresponding to SEQ ID NO: 97 or a sequence at least 90% identical thereto. In some embodiments, the one or more nucleic acids encode for an antigen comprising or consisting of an amino acid sequence corresponding to SEQ ID NO: 98 or a sequence at least 90% identical thereto. In some embodiments, the one or more nucleic acids encode for an antigen comprising or consisting of an amino acid sequence corresponding to SEQ ID NO: 99 or a sequence at least 90% identical thereto. In some embodiments, the one or more nucleic acids encode for an antigen comprising or consisting of an amino acid sequence corresponding to SEQ ID NO: 100 or a sequence at least 90% identical thereto. In some embodiments, the one or more nucleic acids encode for an antigen comprising or consisting of an amino acid sequence corresponding to SEQ ID NO: 101 or a sequence at least 90% identical thereto. In some embodiments, the one or more nucleic acids encode for an antigen comprising or consisting of an amino acid sequence corresponding to SEQ ID NO: 102 or a sequence at least 90% identical thereto. In some embodiments, the one or more nucleic acids encode for an antigen comprising or consisting of an amino acid sequence corresponding to SEQ ID NO: 103 or a sequence at least 90% identical thereto. In some embodiments, the one or more nucleic acids encode for an antigen comprising or consisting of an amino acid sequence corresponding to SEQ ID NO: 104 or a sequence at least 90% identical thereto. In some embodiments, the one or more nucleic acids encode for an antigen comprising or consisting of an amino acid sequence corresponding to SEQ ID NO: 105 or a sequence at least 90% identical thereto.

In some embodiments, the one or more nucleic acids encode for an antigen comprising or consisting of an amino acid sequence corresponding to SEQ ID NO: 108 or a sequence at least 90% identical thereto. In some embodiments, the one or more nucleic acids encode for an antigen comprising or consisting of an amino acid sequence corresponding to SEQ ID NO: 109 or a sequence at least 90% identical thereto. In some embodiments, the one or more nucleic acids encode for an antigen comprising or consisting of an amino acid sequence corresponding to SEQ ID NO: 110 or a sequence at least 90% identical thereto. In some embodiments, the one or more nucleic acids encode for an antigen comprising or consisting of an amino acid sequence corresponding to SEQ ID NO: 111 or a sequence at least 90% identical thereto. In some embodiments, the one or more nucleic acids encode for an antigen comprising or consisting of an amino acid sequence corresponding to SEQ ID NO: 112 or a sequence at least 90% identical thereto. In some embodiments, the one or more nucleic acids encode for an antigen comprising or consisting of an amino acid sequence corresponding to SEQ ID NO: 113 or a sequence at least 90% identical thereto. In some embodiments, the one or more nucleic acids encode for an antigen comprising or consisting of an amino acid sequence corresponding to SEQ ID NO: 114 or a sequence at least 90% identical thereto. In some embodiments, the one or more nucleic acids encode for an antigen comprising or consisting of an amino acid sequence corresponding to SEQ ID NO: 115 or a sequence at least 90% identical thereto. In some embodiments, the one or more nucleic acids encode for an antigen comprising or consisting of an amino acid sequence corresponding to SEQ ID NO: 116 or a sequence at least 90% identical thereto. In some embodiments, the one or more nucleic acids encode for an antigen comprising or consisting of an amino acid sequence corresponding to SEQ ID NO: 117 or a sequence at least 90% identical thereto. In some embodiments, the one or more nucleic acids encode for an antigen comprising or consisting of an amino acid sequence corresponding to SEQ ID NO: 118 or a sequence at least 90% identical thereto. In some embodiments, the one or more nucleic acids encode for an antigen comprising or consisting of an amino acid sequence corresponding to SEQ ID NO: 119 or a sequence at least 90% identical thereto. In some embodiments, the one or more nucleic acids encode for an antigen comprising or consisting of an amino acid sequence corresponding to SEQ ID NO: 120 or a sequence at least 90% identical thereto. In some embodiments, the one or more nucleic acids encode for an antigen comprising or consisting of an amino acid sequence corresponding to SEQ ID NO: 121 or a sequence at least 90% identical thereto. In some embodiments, the one or more nucleic acids encode for an antigen comprising or consisting of an amino acid sequence corresponding to SEQ ID NO: 122 or a sequence at least 90% identical thereto. In some embodiments, the one or more nucleic acids encode for an antigen comprising or consisting of an amino acid sequence corresponding to SEQ ID NO: 123 or a sequence at least 90% identical thereto. In some embodiments, the one or more nucleic acids encode for an antigen comprising or consisting of an amino acid sequence corresponding to SEQ ID NO: 124 or a sequence at least 90% identical thereto. In some embodiments, the one or more nucleic acids encode for an antigen comprising or consisting of an amino acid sequence corresponding to SEQ ID NO: 125 or a sequence at least 90% identical thereto. In some embodiments, the one or more nucleic acids encode for an antigen comprising or consisting of an amino acid sequence corresponding to SEQ ID NO: 126 or a sequence at least 90% identical thereto. In some embodiments, the one or more nucleic acids encode for an antigen comprising or consisting of an amino acid sequence corresponding to SEQ ID NO: 127 or a sequence at least 90% identical thereto. In some embodiments, the one or more nucleic acids encode for an antigen comprising or consisting of an amino acid sequence corresponding to SEQ ID NO: 128 or a sequence at least 90% identical thereto.

In some embodiments, the one or more nucleic acids encode for an antigen comprising or consisting of an amino acid sequence corresponding to SEQ ID NO: 108 or a sequence at least 95% identical thereto. In some embodiments, the one or more nucleic acids encode for an antigen comprising or consisting of an amino acid sequence corresponding to SEQ ID NO: 109 or a sequence at least 95% identical thereto. In some embodiments, the one or more nucleic acids encode for an antigen comprising or consisting of an amino acid sequence corresponding to SEQ ID NO: 110 or a sequence at least 95% identical thereto. In some embodiments, the one or more nucleic acids encode for an antigen comprising or consisting of an amino acid sequence corresponding to SEQ ID NO: 111 or a sequence at least 95% identical thereto. In some embodiments, the one or more nucleic acids encode for an antigen comprising or consisting of an amino acid sequence corresponding to SEQ ID NO: 112 or a sequence at least 95% identical thereto. In some embodiments, the one or more nucleic acids encode for an antigen comprising or consisting of an amino acid sequence corresponding to SEQ ID NO: 113 or a sequence at least 95% identical thereto. In some embodiments, the one or more nucleic acids encode for an antigen comprising or consisting of an amino acid sequence corresponding to SEQ ID NO: 114 or a sequence at least 95% identical thereto. In some embodiments, the one or more nucleic acids encode for an antigen comprising or consisting of an amino acid sequence corresponding to SEQ ID NO: 115 or a sequence at least 95% identical thereto. In some embodiments, the one or more nucleic acids encode for an antigen comprising or consisting of an amino acid sequence corresponding to SEQ ID NO: 116 or a sequence at least 95% identical thereto. In some embodiments, the one or more nucleic acids encode for an antigen comprising or consisting of an amino acid sequence corresponding to SEQ ID NO: 117 or a sequence at least 95% identical thereto. In some embodiments, the one or more nucleic acids encode for an antigen comprising or consisting of an amino acid sequence corresponding to SEQ ID NO: 118 or a sequence at least 95% identical thereto. In some embodiments, the one or more nucleic acids encode for an antigen comprising or consisting of an amino acid sequence corresponding to SEQ ID NO: 119 or a sequence at least 95% identical thereto. In some embodiments, the one or more nucleic acids encode for an antigen comprising or consisting of an amino acid sequence corresponding to SEQ ID NO: 120 or a sequence at least 95% identical thereto. In some embodiments, the one or more nucleic acids encode for an antigen comprising or consisting of an amino acid sequence corresponding to SEQ ID NO: 121 or a sequence at least 95% identical thereto. In some embodiments, the one or more nucleic acids encode for an antigen comprising or consisting of an amino acid sequence corresponding to SEQ ID NO: 122 or a sequence at least 95% identical thereto. In some embodiments, the one or more nucleic acids encode for an antigen comprising or consisting of an amino acid sequence corresponding to SEQ ID NO: 123 or a sequence at least 95% identical thereto. In some embodiments, the one or more nucleic acids encode for an antigen comprising or consisting of an amino acid sequence corresponding to SEQ ID NO: 124 or a sequence at least 95% identical thereto. In some embodiments, the one or more nucleic acids encode for an antigen comprising or consisting of an amino acid sequence corresponding to SEQ ID NO: 125 or a sequence at least 95% identical thereto. In some embodiments, the one or more nucleic acids encode for an antigen comprising or consisting of an amino acid sequence corresponding to SEQ ID NO: 126 or a sequence at least 95% identical thereto. In some embodiments, the one or more nucleic acids encode for an antigen comprising or consisting of an amino acid sequence corresponding to SEQ ID NO: 127 or a sequence at least 95% identical thereto. In some embodiments, the one or more nucleic acids encode for an antigen comprising or consisting of an amino acid sequence corresponding to SEQ ID NO: 128 or a sequence at least 95% identical thereto.

In some embodiments, the one or more nucleic acids encode for an antigen comprising or consisting of an amino acid sequence corresponding to SEQ ID NO: 108 or a sequence at least 98% identical thereto. In some embodiments, the one or more nucleic acids encode for an antigen comprising or consisting of an amino acid sequence corresponding to SEQ ID NO: 109 or a sequence at least 98% identical thereto. In some embodiments, the one or more nucleic acids encode for an antigen comprising or consisting of an amino acid sequence corresponding to SEQ ID NO: 110 or a sequence at least 98% identical thereto. In some embodiments, the one or more nucleic acids encode for an antigen comprising or consisting of an amino acid sequence corresponding to SEQ ID NO: 111 or a sequence at least 98% identical thereto. In some embodiments, the one or more nucleic acids encode for an antigen comprising or consisting of an amino acid sequence corresponding to SEQ ID NO: 112 or a sequence at least 98% identical thereto. In some embodiments, the one or more nucleic acids encode for an antigen comprising or consisting of an amino acid sequence corresponding to SEQ ID NO: 113 or a sequence at least 98% identical thereto. In some embodiments, the one or more nucleic acids encode for an antigen comprising or consisting of an amino acid sequence corresponding to SEQ ID NO: 114 or a sequence at least 98% identical thereto. In some embodiments, the one or more nucleic acids encode for an antigen comprising or consisting of an amino acid sequence corresponding to SEQ ID NO: 115 or a sequence at least 98% identical thereto. In some embodiments, the one or more nucleic acids encode for an antigen comprising or consisting of an amino acid sequence corresponding to SEQ ID NO: 116 or a sequence at least 98% identical thereto. In some embodiments, the one or more nucleic acids encode for an antigen comprising or consisting of an amino acid sequence corresponding to SEQ ID NO: 117 or a sequence at least 98% identical thereto. In some embodiments, the one or more nucleic acids encode for an antigen comprising or consisting of an amino acid sequence corresponding to SEQ ID NO: 118 or a sequence at least 98% identical thereto. In some embodiments, the one or more nucleic acids encode for an antigen comprising or consisting of an amino acid sequence corresponding to SEQ ID NO: 119 or a sequence at least 98% identical thereto. In some embodiments, the one or more nucleic acids encode for an antigen comprising or consisting of an amino acid sequence corresponding to SEQ ID NO: 120 or a sequence at least 98% identical thereto. In some embodiments, the one or more nucleic acids encode for an antigen comprising or consisting of an amino acid sequence corresponding to SEQ ID NO: 121 or a sequence at least 98% identical thereto. In some embodiments, the one or more nucleic acids encode for an antigen comprising or consisting of an amino acid sequence corresponding to SEQ ID NO: 122 or a sequence at least 98% identical thereto. In some embodiments, the one or more nucleic acids encode for an antigen comprising or consisting of an amino acid sequence corresponding to SEQ ID NO: 123 or a sequence at least 98% identical thereto. In some embodiments, the one or more nucleic acids encode for an antigen comprising or consisting of an amino acid sequence corresponding to SEQ ID NO: 124 or a sequence at least 98% identical thereto. In some embodiments, the one or more nucleic acids encode for an antigen comprising or consisting of an amino acid sequence corresponding to SEQ ID NO: 125 or a sequence at least 98% identical thereto. In some embodiments, the one or more nucleic acids encode for an antigen comprising or consisting of an amino acid sequence corresponding to SEQ ID NO: 126 or a sequence at least 98% identical thereto. In some embodiments, the one or more nucleic acids encode for an antigen comprising or consisting of an amino acid sequence corresponding to SEQ ID NO: 127 or a sequence at least 98% identical thereto. In some embodiments, the one or more nucleic acids encode for an antigen comprising or consisting of an amino acid sequence corresponding to SEQ ID NO: 128 or a sequence at least 98% identical thereto.

In some embodiments, the one or more nucleic acids encode for an antigen comprising or consisting of an amino acid sequence corresponding to SEQ ID NO: 108 or a sequence at least 99% identical thereto. In some embodiments, the one or more nucleic acids encode for an antigen comprising or consisting of an amino acid sequence corresponding to SEQ ID NO: 109 or a sequence at least 99% identical thereto. In some embodiments, the one or more nucleic acids encode for an antigen comprising or consisting of an amino acid sequence corresponding to SEQ ID NO: 110 or a sequence at least 99% identical thereto. In some embodiments, the one or more nucleic acids encode for an antigen comprising or consisting of an amino acid sequence corresponding to SEQ ID NO: 111 or a sequence at least 99% identical thereto. In some embodiments, the one or more nucleic acids encode for an antigen comprising or consisting of an amino acid sequence corresponding to SEQ ID NO: 112 or a sequence at least 99% identical thereto. In some embodiments, the one or more nucleic acids encode for an antigen comprising or consisting of an amino acid sequence corresponding to SEQ ID NO: 113 or a sequence at least 99% identical thereto. In some embodiments, the one or more nucleic acids encode for an antigen comprising or consisting of an amino acid sequence corresponding to SEQ ID NO: 114 or a sequence at least 99% identical thereto. In some embodiments, the one or more nucleic acids encode for an antigen comprising or consisting of an amino acid sequence corresponding to SEQ ID NO: 115 or a sequence at least 99% identical thereto. In some embodiments, the one or more nucleic acids encode for an antigen comprising or consisting of an amino acid sequence corresponding to SEQ ID NO: 116 or a sequence at least 99% identical thereto. In some embodiments, the one or more nucleic acids encode for an antigen comprising or consisting of an amino acid sequence corresponding to SEQ ID NO: 117 or a sequence at least 99% identical thereto. In some embodiments, the one or more nucleic acids encode for an antigen comprising or consisting of an amino acid sequence corresponding to SEQ ID NO: 118 or a sequence at least 99% identical thereto. In some embodiments, the one or more nucleic acids encode for an antigen comprising or consisting of an amino acid sequence corresponding to SEQ ID NO: 119 or a sequence at least 99% identical thereto. In some embodiments, the one or more nucleic acids encode for an antigen comprising or consisting of an amino acid sequence corresponding to SEQ ID NO: 120 or a sequence at least 99% identical thereto. In some embodiments, the one or more nucleic acids encode for an antigen comprising or consisting of an amino acid sequence corresponding to SEQ ID NO: 121 or a sequence at least 99% identical thereto. In some embodiments, the one or more nucleic acids encode for an antigen comprising or consisting of an amino acid sequence corresponding to SEQ ID NO: 122 or a sequence at least 99% identical thereto. In some embodiments, the one or more nucleic acids encode for an antigen comprising or consisting of an amino acid sequence corresponding to SEQ ID NO: 123 or a sequence at least 99% identical thereto. In some embodiments, the one or more nucleic acids encode for an antigen comprising or consisting of an amino acid sequence corresponding to SEQ ID NO: 124 or a sequence at least 99% identical thereto. In some embodiments, the one or more nucleic acids encode for an antigen comprising or consisting of an amino acid sequence corresponding to SEQ ID NO: 125 or a sequence at least 99% identical thereto. In some embodiments, the one or more nucleic acids encode for an antigen comprising or consisting of an amino acid sequence corresponding to SEQ ID NO: 126 or a sequence at least 99% identical thereto. In some embodiments, the one or more nucleic acids encode for an antigen comprising or consisting of an amino acid sequence corresponding to SEQ ID NO: 127 or a sequence at least 99% identical thereto. In some embodiments, the one or more nucleic acids encode for an antigen comprising or consisting of an amino acid sequence corresponding to SEQ ID NO: 128 or a sequence at least 99% identical thereto.

In some embodiments, the one or more nucleic acids are RNAs and comprise an RNA sequence selected from SEQ ID NOS: 64-84. In some embodiments, the one or more nucleic acids comprise RNA corresponding to SEQ ID NO: 64, or a sequence at least 90% identical thereto. In some embodiments, the one or more nucleic acids comprise RNA corresponding to SEQ ID NO: 65, or a sequence at least 90% identical thereto. In some embodiments, the one or more nucleic acids comprise RNA corresponding to SEQ ID NO: 66, or a sequence at least 90% identical thereto. In some embodiments, the one or more nucleic acids comprise RNA corresponding to SEQ ID NO: 67, or a sequence at least 90% identical thereto. In some embodiments, the one or more nucleic acids comprise RNA corresponding to SEQ ID NO: 68, or a sequence at least 90% identical thereto. In some embodiments, the one or more nucleic acids comprise RNA corresponding to SEQ ID NO: 69, or a sequence at least 90% identical thereto. In some embodiments, the one or more nucleic acids comprise RNA corresponding to SEQ ID NO: 70, or a sequence at least 90% identical thereto. In some embodiments, the one or more nucleic acids comprise RNA corresponding to SEQ ID NO: 71, or a sequence at least 90% identical thereto. In some embodiments, the one or more nucleic acids comprise RNA corresponding to SEQ ID NO: 72, or a sequence at least 90% identical thereto. In some embodiments, the one or more nucleic acids comprise RNA corresponding to SEQ ID NO: 73, or a sequence at least 90% identical thereto. In some embodiments, the one or more nucleic acids comprise RNA corresponding to SEQ ID NO: 74, or a sequence at least 90% identical thereto. In some embodiments, the one or more nucleic acids comprise RNA corresponding to SEQ ID NO: 75, or a sequence at least 90% identical thereto. In some embodiments, the one or more nucleic acids comprise RNA corresponding to SEQ ID NO: 76, or a sequence at least 90% identical thereto. In some embodiments, the one or more nucleic acids comprise RNA corresponding to SEQ ID NO: 77, or a sequence at least 90% identical thereto. In some embodiments, the one or more nucleic acids comprise RNA corresponding to SEQ ID NO: 78, or a sequence at least 90% identical thereto. In some embodiments, the one or more nucleic acids comprise RNA corresponding to SEQ ID NO: 79, or a sequence at least 90% identical thereto. In some embodiments, the one or more nucleic acids comprise RNA corresponding to SEQ ID NO: 80, or a sequence at least 90% identical thereto. In some embodiments, the one or more nucleic acids comprise RNA corresponding to SEQ ID NO: 81, or a sequence at least 90% identical thereto. In some embodiments, the one or more nucleic acids comprise RNA corresponding to SEQ ID NO: 82, or a sequence at least 90% identical thereto. In some embodiments, the one or more nucleic acids comprise RNA corresponding to SEQ ID NO: 83, or a sequence at least 90% identical thereto. In some embodiments, the one or more nucleic acids comprise RNA corresponding to SEQ ID NO: 84, or a sequence at least 90% identical thereto.

In some embodiments, the one or more nucleic acids encode eight distinct antigens comprising amino acid sequences selected from the group consisting of SEQ ID NOS: 1-8, 16-23, or 29, or sequences at least 70% identical thereto. In some embodiments, the one or more nucleic acids encode six distinct antigens comprising amino acid sequences selected from the group consisting of SEQ ID NOS: 1-8, 16-23, or 29, or sequences at least 70% identical thereto. In some embodiments, the one or more nucleic acids encode eight distinct antigens comprising amino acid sequences selected from the group consisting of SEQ ID NOS: 1-8, 16-23, or 29, or sequences at least 70% identical thereto. In some embodiments, the one or more nucleic acids encode ten distinct antigens comprising amino acid sequences selected from the group consisting of SEQ ID NOS: 1-8, 16-23, or 29, or sequences at least 70% identical thereto. In some embodiments, the one or more nucleic acids encode twelve distinct antigens comprising amino acid sequences selected from the group consisting of SEQ ID NOS: 1-8, 16-23, or 29, or sequences at least 70% identical thereto. In some embodiments, the one or more nucleic acids encode fourteen distinct antigens comprising amino acid sequences selected from the group consisting of SEQ ID NOS: 1-8, 16-23, or 29, or sequences at least 70% identical thereto. In some embodiments, the one or more nucleic acids encode sixteen distinct antigens comprising amino acid sequences selected from the group consisting of SEQ ID NOS: 1-8, 16-23, or 29, or sequences at least 70% identical to any one of such sequences.

In some embodiments, the one or more nucleic acids encode eight distinct antigens comprising amino acid sequences selected from the group consisting of SEQ ID NOS: 2-9, 16-22, 24, 38-42, or sequences at least 70% identical thereto. In some embodiments, the one or more nucleic acids encode six distinct antigens comprising amino acid sequences selected from the group consisting of SEQ ID NOS: 2-9, 16-22, 24, 38-42, or sequences at least 70% identical thereto. In some embodiments, the one or more nucleic acids encode eight distinct antigens comprising amino acid sequences selected from the group consisting of SEQ ID NOS: 2-9, 16-22, 24, 38-42, or sequences at least 70% identical thereto. In some embodiments, the one or more nucleic acids encode ten distinct antigens comprising amino acid sequences selected from the group consisting of SEQ ID NOS: 2-9, 16-22, 24, 38-42, or sequences at least 70% identical thereto. In some embodiments, the one or more nucleic acids encode twelve distinct antigens comprising amino acid sequences selected from the group consisting of SEQ ID NOS: 2-9, 16-22, 24, 38-42, or sequences at least 70% identical thereto. In some embodiments, the one or more nucleic acids encode fourteen distinct antigens comprising amino acid sequences selected from the group consisting of SEQ ID NOS: 2-9, 16-22, 24, 38-42, or sequences at least 70% identical thereto. In some embodiments, the one or more nucleic acids encode sixteen distinct antigens comprising amino acid sequences selected from the group consisting of SEQ ID NOS: 2-9, 16-22, 24, 38-42, or sequences at least 70% identical to any one of such sequences.

In some embodiments, the one or more nucleic acids encode four or more distinct antigens comprising amino acid sequences selected from the group consisting of SEQ ID NOS: 2-9, 38-39, or sequences at least 90% identical to any one of such sequences. In some embodiments, the one or more nucleic acids encode six or more distinct antigens comprising amino acid sequences selected from the group consisting of SEQ ID NOS: 2-9, 38-39, or sequences at least 90% identical to any one of such sequences. In some embodiments, the one or more nucleic acids encode eight or more distinct antigens comprising amino acid sequences selected from the group consisting of SEQ ID NOS: 2-9, 38-39, or sequences at least 90% identical to any one of such sequences. In some embodiments, the one or more nucleic acids encode antigens comprising SEQ ID NOS: 2-9, 38-39, or sequences at least 90% identical to any one of such sequences. In some embodiments, the one or more nucleic acids encode four or more distinct antigens comprising amino acid sequences selected from the group consisting of SEQ ID NOS: 16-22, 24, 40-41, or sequences at least 90% identical to any one of such sequences. In some embodiments, the one or more nucleic acids encode six or more distinct antigens comprising amino acid sequences selected from the group consisting of SEQ ID NOS: 16-22, 24, 40-41, or sequences at least 90% identical to any one of such sequences. In some embodiments, the one or more nucleic acids encode eight or more distinct antigens comprising amino acid sequences selected from the group consisting of SEQ ID NOS: 16-22, 24, 40-41, or sequences at least 90% identical to any one of such sequences. In some embodiments, the one or more nucleic acids encode antigens comprising amino acid sequences corresponding to SEQ ID NOS: 16-22, 24, 40-41, or sequences at least 90% identical to any one of such sequences. In some embodiments, the one or more nucleic acids encode an antigen comprising SEQ ID NO: 42, or a sequence at least 90% identical thereto.

In some embodiments, the one or more nucleic acids encode four or more distinct antigens comprising amino acid sequences selected from the group consisting of SEQ ID NOS: 108-115, 124-125, or sequences at least 90% identical to any one of such sequences. In some embodiments, the one or more nucleic acids encode six or more distinct antigens comprising amino acid sequences selected from the group consisting of SEQ ID NOS: 108-115, 124-125, or sequences at least 90% identical to any one of such sequences. In some embodiments, the one or more nucleic acids encode eight or more distinct antigens comprising amino acid sequences selected from the group consisting of SEQ ID NOS: 108-115, 124-125, or sequences at least 90% identical to any one of such sequences. In some embodiments, the one or more nucleic acids encode antigens comprising SEQ ID NOS: 108-115, 124-125, or sequences at least 90% identical to any one of such sequences. In some embodiments, the one or more nucleic acids encode four or more distinct antigens comprising amino acid sequences selected from the group consisting of SEQ ID NOS: 116-123, 126-1271, or sequences at least 90% identical to any one of such sequences. In some embodiments, the one or more nucleic acids encode six or more distinct antigens comprising amino acid sequences selected from the group consisting of SEQ ID NOS: 116-123, 126-127, or sequences at least 90% identical to any one of such sequences. In some embodiments, the one or more nucleic acids encode eight or more distinct antigens comprising amino acid sequences selected from the group consisting of SEQ ID NOS: 116-123, 126-127, or sequences at least 90% identical to any one of such sequences. In some embodiments, the one or more nucleic acids encode antigens comprising SEQ ID NOS: 116-123, 126-127, or sequences at least 90% identical to any one of such sequences. In some embodiments, the one or more nucleic acids encode an antigen comprising SEQ ID NO: 128, or a sequence at least 90% identical thereto.

In some embodiments, the one or more nucleic acids encode for an antigen comprising or consisting of an amino acid sequence corresponding to SEQ ID NO: 1. In some embodiments, the one or more nucleic acids encode for an antigen comprising or consisting of an amino acid sequence corresponding to SEQ ID NO: 2. In some embodiments, the one or more nucleic acids encode for an antigen comprising or consisting of an amino acid sequence corresponding to SEQ ID NO: 3. In some embodiments, the one or more nucleic acids encode for an antigen comprising or consisting of an amino acid sequence corresponding to SEQ ID NO: 4. In some embodiments, the one or more nucleic acids encode for an antigen comprising or consisting of an amino acid sequence corresponding to SEQ ID NO: 5. In some embodiments, the one or more nucleic acids encode for an antigen comprising or consisting of an amino acid sequence corresponding to SEQ ID NO: 6. In some embodiments, the one or more nucleic acids encode for an antigen comprising or consisting of an amino acid sequence corresponding to SEQ ID NO: 7. In some embodiments, the one or more nucleic acids encode for an antigen comprising or consisting of an amino acid sequence corresponding to SEQ ID NO: 8. In some embodiments, the one or more nucleic acids encode for an antigen comprising or consisting of an amino acid sequence corresponding to SEQ ID NO: 9. In some embodiments, the one or more nucleic acids encode for an antigen comprising or consisting of an amino acid sequence corresponding to SEQ ID NO: 10. In some embodiments, the one or more nucleic acids encode for an antigen comprising or consisting of an amino acid sequence corresponding to SEQ ID NO: 11. In some embodiments, the one or more nucleic acids encode for an antigen comprising or consisting of an amino acid sequence corresponding to SEQ ID NO: 12. In some embodiments, the one or more nucleic acids encode for an antigen comprising or consisting of an amino acid sequence corresponding to SEQ ID NO: 13. In some embodiments, the one or more nucleic acids encode for an antigen comprising or consisting of an amino acid sequence corresponding to SEQ ID NO: 14. In some embodiments, the one or more nucleic acids encode for an antigen comprising or consisting of an amino acid sequence corresponding to SEQ ID NO: 15. In some embodiments, the one or more nucleic acids encode for an antigen comprising or consisting of an amino acid sequence corresponding to SEQ ID NO: 16. In some embodiments, the one or more nucleic acids encode for an antigen comprising or consisting of an amino acid sequence corresponding to SEQ ID NO: 17. In some embodiments, the one or more nucleic acids encode for an antigen comprising or consisting of an amino acid sequence corresponding to SEQ ID NO: 18. In some embodiments, the one or more nucleic acids encode for an antigen comprising or consisting of an amino acid sequence corresponding to SEQ ID NO: 19. In some embodiments, the one or more nucleic acids encode for an antigen comprising or consisting of an amino acid sequence corresponding to SEQ ID NO: 20. In some embodiments, the one or more nucleic acids encode for an antigen comprising or consisting of an amino acid sequence corresponding to SEQ ID NO: 21. In some embodiments, the one or more nucleic acids encode for an antigen comprising or consisting of an amino acid sequence corresponding to SEQ ID NO: 22. In some embodiments, the one or more nucleic acids encode for an antigen comprising or consisting of an amino acid sequence corresponding to SEQ ID NO: 23. In some embodiments, the one or more nucleic acids encode for an antigen comprising or consisting of an amino acid sequence corresponding to SEQ ID NO: 24. In some embodiments, the one or more nucleic acids encode for an antigen comprising or consisting of an amino acid sequence corresponding to SEQ ID NO: 25. In some embodiments, the one or more nucleic acids encode for an antigen comprising or consisting of an amino acid sequence corresponding to SEQ ID NO: 26. In some embodiments, the one or more nucleic acids encode for an antigen comprising or consisting of an amino acid sequence corresponding to SEQ ID NO: 27. In some embodiments, the one or more nucleic acids encode for an antigen comprising or consisting of an amino acid sequence corresponding to SEQ ID NO: 28. In some embodiments, the one or more nucleic acids encode for an antigen comprising or consisting of an amino acid sequence corresponding to SEQ ID NO: 29. In some embodiments, the one or more nucleic acids encode for an antigen comprising or consisting of an amino acid sequence corresponding to SEQ ID NO: 30. In some embodiments, the one or more nucleic acids encode for an antigen comprising or consisting of an amino acid sequence corresponding to SEQ ID NO: 31. In some embodiments, the one or more nucleic acids encode for an antigen comprising or consisting of an amino acid sequence corresponding to SEQ ID NO: 32. In some embodiments, the one or more nucleic acids encode for an antigen comprising or consisting of an amino acid sequence corresponding to SEQ ID NO: 33. In some embodiments, the one or more nucleic acids encode for an antigen comprising or consisting of an amino acid sequence corresponding to SEQ ID NO: 34. In some embodiments, the one or more nucleic acids encode for an antigen comprising or consisting of an amino acid sequence corresponding to SEQ ID NO: 35. In some embodiments, the one or more nucleic acids encode for an antigen comprising or consisting of an amino acid sequence corresponding to SEQ ID NO: 36. In some embodiments, the one or more nucleic acids encode for an antigen comprising or consisting of an amino acid sequence corresponding to SEQ ID NO: 37. In some embodiments, the one or more nucleic acids encode for an antigen comprising or consisting of an amino acid sequence corresponding to SEQ ID NO: 38. In some embodiments, the one or more nucleic acids encode for an antigen comprising or consisting of an amino acid sequence corresponding to SEQ ID NO: 39. In some embodiments, the one or more nucleic acids encode for an antigen comprising or consisting of an amino acid sequence corresponding to SEQ ID NO: 40. In some embodiments, the one or more nucleic acids encode for an antigen comprising or consisting of an amino acid sequence corresponding to SEQ ID NO: 41. In some embodiments, the one or more nucleic acids encode for an antigen comprising or consisting of an amino acid sequence corresponding to SEQ ID NO: 42.

In some embodiments, the one or more nucleic acids encode for an antigen comprising or consisting of an amino acid sequence corresponding to SEQ ID NO: 85. In some embodiments, the one or more nucleic acids encode for an antigen comprising or consisting of an amino acid sequence corresponding to SEQ ID NO: 86. In some embodiments, the one or more nucleic acids encode for an antigen comprising or consisting of an amino acid sequence corresponding to SEQ ID NO: 87. In some embodiments, the one or more nucleic acids encode for an antigen comprising or consisting of an amino acid sequence corresponding to SEQ ID NO: 88. In some embodiments, the one or more nucleic acids encode for an antigen comprising or consisting of an amino acid sequence corresponding to SEQ ID NO: 89. In some embodiments, the one or more nucleic acids encode for an antigen comprising or consisting of an amino acid sequence corresponding to SEQ ID NO: 90. In some embodiments, the one or more nucleic acids encode for an antigen comprising or consisting of an amino acid sequence corresponding to SEQ ID NO: 91. In some embodiments, the one or more nucleic acids encode for an antigen comprising or consisting of an amino acid sequence corresponding to SEQ ID NO: 92. In some embodiments, the one or more nucleic acids encode for an antigen comprising or consisting of an amino acid sequence corresponding to SEQ ID NO: 93. In some embodiments, the one or more nucleic acids encode for an antigen comprising or consisting of an amino acid sequence corresponding to SEQ ID NO: 94. In some embodiments, the one or more nucleic acids encode for an antigen comprising or consisting of an amino acid sequence corresponding to SEQ ID NO: 95. In some embodiments, the one or more nucleic acids encode for an antigen comprising or consisting of an amino acid sequence corresponding to SEQ ID NO: 96. In some embodiments, the one or more nucleic acids encode for an antigen comprising or consisting of an amino acid sequence corresponding to SEQ ID NO: 97. In some embodiments, the one or more nucleic acids encode for an antigen comprising or consisting of an amino acid sequence corresponding to SEQ ID NO: 98. In some embodiments, the one or more nucleic acids encode for an antigen comprising or consisting of an amino acid sequence corresponding to SEQ ID NO: 99. In some embodiments, the one or more nucleic acids encode for an antigen comprising or consisting of an amino acid sequence corresponding to SEQ ID NO: 100. In some embodiments, the one or more nucleic acids encode for an antigen comprising or consisting of an amino acid sequence corresponding to SEQ ID NO: 101. In some embodiments, the one or more nucleic acids encode for an antigen comprising or consisting of an amino acid sequence corresponding to SEQ ID NO: 102. In some embodiments, the one or more nucleic acids encode for an antigen comprising or consisting of an amino acid sequence corresponding to SEQ ID NO: 103. In some embodiments, the one or more nucleic acids encode for an antigen comprising or consisting of an amino acid sequence corresponding to SEQ ID NO: 104. In some embodiments, the one or more nucleic acids encode for an antigen comprising or consisting of an amino acid sequence corresponding to SEQ ID NO: 105.

In some embodiments, the one or more nucleic acids encode eight distinct antigens comprising amino acid sequences selected from the group consisting of SEQ ID NOS: 1-8, 16-23, or 29. In some embodiments, the one or more nucleic acids encode six distinct antigens comprising amino acid sequences selected from the group consisting of SEQ ID NOS: 1-8, 16-23, or 29. In some embodiments, the one or more nucleic acids encode eight distinct antigens comprising amino acid sequences selected from the group consisting of SEQ ID NOS: 1-8, 16-23, or 29. In some embodiments, the one or more nucleic acids encode ten distinct antigens comprising amino acid sequences selected from the group consisting of SEQ ID NOS: 1-8, 16-23, or 29. In some embodiments, the one or more nucleic acids encode twelve distinct antigens comprising amino acid sequences selected from the group consisting of SEQ ID NOS: 1-8, 16-23, or 29. In some embodiments, the one or more nucleic acids encode fourteen distinct antigens comprising amino acid sequences selected from the group consisting of SEQ ID NOS: 1-8, 16-23, or 29. In some embodiments, the one or more nucleic acids encode sixteen distinct antigens comprising amino acid sequences selected from the group consisting of SEQ ID NOS: 1-8, 16-23, or 29. In some embodiments, the one or more nucleic acids encode eight distinct antigens comprising amino acid sequences selected from the group consisting of SEQ ID NOS: 2-9, 16-22, 24, 38-42. In some embodiments, the one or more nucleic acids encode six distinct antigens comprising amino acid sequences selected from the group consisting of SEQ ID NOS: 2-9, 16-22, 24, 38-42. In some embodiments, the one or more nucleic acids encode eight distinct antigens comprising amino acid sequences selected from the group consisting of SEQ ID NOS: 2-9, 16-22, 24, 38-42. In some embodiments, the one or more nucleic acids encode ten distinct antigens comprising amino acid sequences selected from the group consisting of SEQ ID NOS: 2-9, 16-22, 24, 38-42. In some embodiments, the one or more nucleic acids encode twelve distinct antigens comprising amino acid sequences selected from the group consisting of SEQ ID NOS: 2-9, 16-22, 24, 38-42. In some embodiments, the one or more nucleic acids encode fourteen distinct antigens comprising amino acid sequences selected from the group consisting of SEQ ID NOS: 2-9, 16-22, 24, 38-42. In some embodiments, the one or more nucleic acids encode sixteen distinct antigens comprising amino acid sequences selected from the group consisting of SEQ ID NOS: 2-9, 16-22, 24, 38-42.

In some embodiments, the nucleic acid vaccine composition further comprises a pharmaceutically acceptable carrier. In some embodiments, the one or more nucleic acids that encode a plurality of antigens are present in the nucleic acid vaccine composition in an amount of less than about 5 micrograms (μg) such as, for example, from about 1 ng to about 5 μg. In some embodiments, the one or more nucleic acids that encode a plurality of antigens are present in the nucleic acid vaccine composition in an amount of less than about 3 micrograms (μg) such as, for example, from about 1 ng to about 3 μg. In some embodiments, the one or more nucleic acids that encode a plurality of antigens are present in the nucleic acid vaccine composition in an amount of less than about 5 μg, less than about 4 μg, less than about 3 μg, less than about 2 μg, or less than about 1 μg. In some embodiments, the one or more nucleic acids that encode a plurality of antigens are present in the nucleic acid vaccine composition in an amount of more than 1 ng. In some embodiments, the plurality of antigens comprises at least 6, at least 7, at least 8, at least 9, at least 10, at least 11, at least 12, at least 13, at least 14, at least 15, at least 16, or at least 17 distinct antigens. In some embodiments, the plurality of antigens comprises no more than 30 antigens. In some embodiments, the plurality of antigens comprises no more than 30, 29, 28, 27, 26, 25, 24, 23, 22, 21, 20, 19, 18, 17, 16, 15, 14, 13, 12, 11, 10, 9, 8, 7, or 6 antigens. In some embodiments, the plurality of antigens comprises no more than 25 antigens. In some embodiments, the plurality of antigens comprises no more than 20 antigens.

The present disclosure provides a method for preventing an influenza infection in a subject. In some embodiments, the method comprises administering to the subject a vaccine composition as described herein. In another aspect, the present disclosure provides a method of inducing an immune response (e.g., a protective immune response) in a subject by administering to the subject a vaccine composition as disclosed herein. Generation of an immune response via administration of a vaccine composition as described herein can do one or more of: (1) protect a human against influenza virus infection, (2) decrease the likelihood of infection (or decrease the severity of one or more symptoms associated with infection) by an influenza virus, or (3) enhance the immune system of the subject to resist subsequent infection by an influenza virus. The vaccine composition may be administered intramuscularly, subcutaneously, intramuscularly, intranasally, or orally. The nucleic acid vaccine composition can be formulated for delivery as a mRNA/LNP, or DNA. An infection rate in subjects receiving the vaccine may be reduced by 2% (or more) relative or more relative to a placebo control or relative to a subject who did not receive the vaccine composition.

The term “about” as used herein, refers to a range that is 10% greater than or less than (±) a stated numerical value within the context of the particular usage.

As used in this specification and the appended claims, the singular forms “a,” “an,” and “the” include plural references unless the context clearly dictates otherwise. Thus, for example, references to “a method” include one or more methods, and/or steps of the type described herein and/or which will become apparent to those persons skilled in the art upon reading this disclosure.

As used in this specification and the appended claims, reference to a sequence at least 70% identical to a described sequence refers to at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identical to the identified sequence.

### EXAMPLES

### Example 1: Serum Activity to mRNA Antigens in Mice

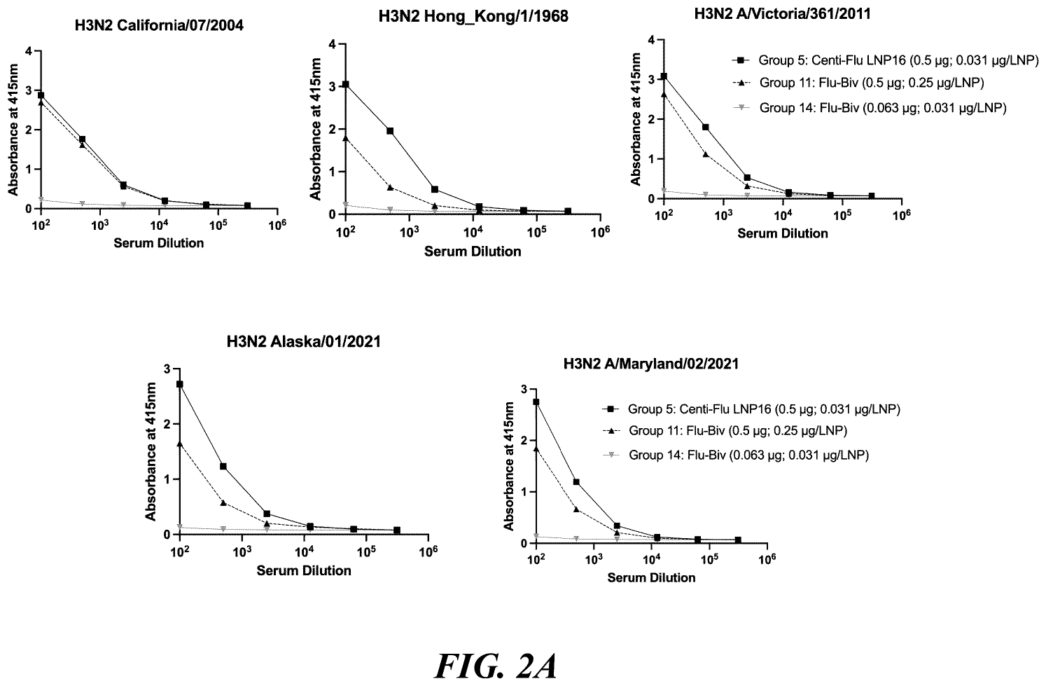

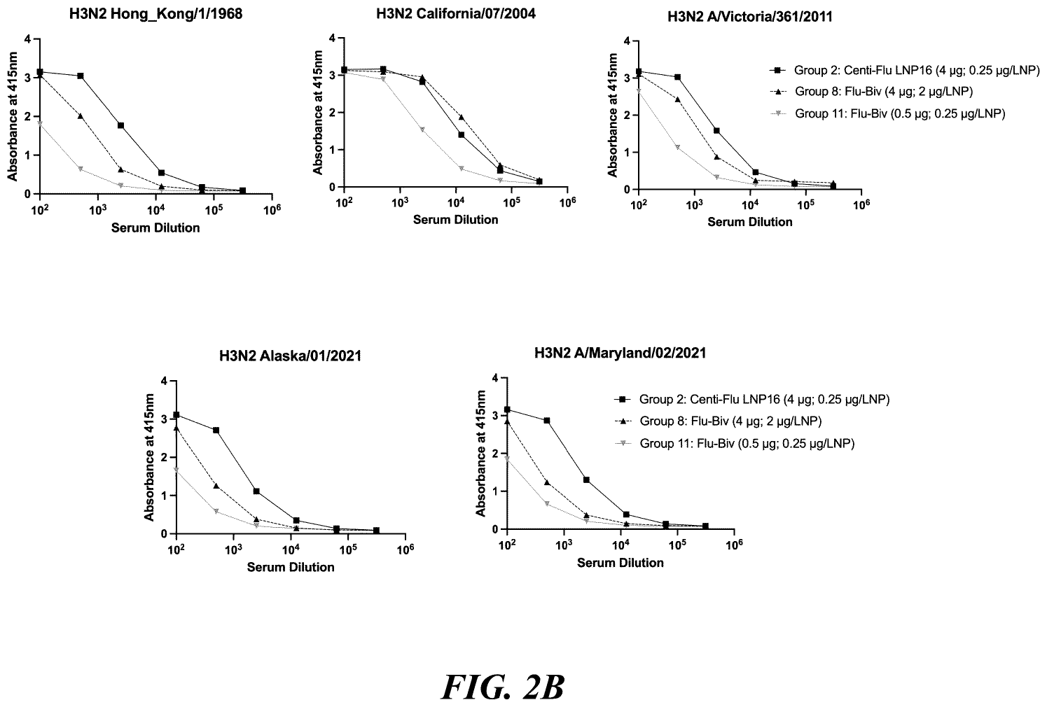

Mice were injected with either Centi-Flu (mRNA encoding a mixture of 16 hemagglutinin antigens, including eight H1, corresponding to SEQ ID NOs. 2-9; and eight H3, corresponding to SEQ ID NOs. 16-22 and 24) or Flu-Biv (mRNA encoding a mixture of 2 hemagglutinin antigens, including one H1 (SEQ ID NO. 4) and one H3 (SEQ ID NO. 17). All mRNA vaccine compositions were complexed in lipid nanoparticles (LNPs). Specifically, the same LNP composition as BNT162b2 was used. The serum was collected 16 days after immunization and measured by ELISA for antibodies that bind to recombinant hemagglutinin proteins of H3N2 Hong Kong/1/1968 (present in Centi-Flu), H3N2 California/07/2004 (present in Centi-Flu and Flu-Biv), H3N2 Alaska/01/2021 (present in Centi-Flu), H3N2 A/Victoria/361/2011 (heterologous to both Centi-Flu and Flu-Biv), or H3N2 A/Maryland/02/2021 (heterologous to both Centi-Flu and Flu-Biv). FIG. 2A suggests that at a lower matched per-antigen dose, Flu-Biv induced no response while Centi-Flu induced a robust response. For example, when the mice were injected with Centi-Flu LNP at 0.031 μg per antigen (=0.5 ug total mRNA) or Flu-Biv at 0.031 μg per antigen (=0.063 ug total mRNA), Flu-Biv induced no response, while Centi-Flu induced a robust response. Furthermore, this difference cannot be explained by the total antigen dose, as Flu-Biv at 0.25 ug per antigen (=0.5 ug total mRNA) still exhibits a weaker response in all unmatched antigens than Centi-Flu at 0.031 ug/antigen (=0.5 ug total mRNA). FIG. 2B suggests that at a higher per-antigen dose, Flu-Biv induced a weak response, while Centi-Flu induced a more potent response. For example, when the mice were injected with Centi-Flu LNP at 0.25 μg per antigen (=4 ug total mRNA) or Flu-Biv at 0.25 μg per antigen (=0.5 ug total mRNA), Flu-Biv induced a weak response, while Centi-Flu induced a robust response. Furthermore, this difference cannot be explained by the total antigen dose, as Flu-Biv at 2 μg per antigen (=4 ug total mRNA) still exhibits a weaker response in all unmatched antigens than Centi-Flu at 0.25 ug/antigen (=4 ug total mRNA). Therefore, it is the unique combination of (1) using six or more homologous distinct antigen components and (2) mRNA encoding each component at a low dose, that achieves the desired serological effect.

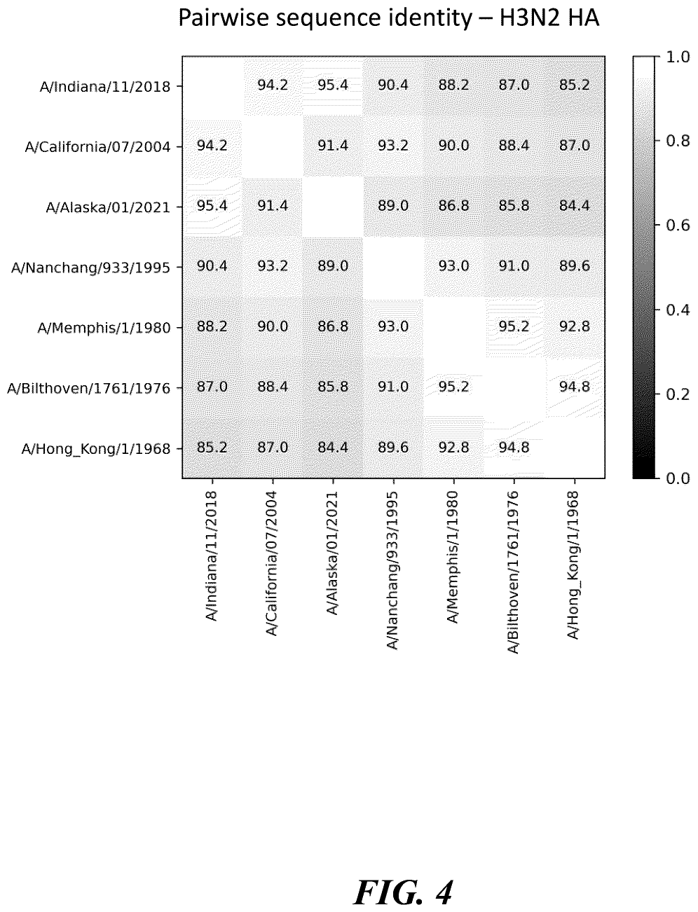

One example set of eight homologous distinct antigen components encoded by mRNA in the Centi-Flu vaccine composition are A/Hong Kong/1/1968 (SEQ ID NO. 22), A/Nanchang/933/1995 (SEQ ID NO. 20), A/California/07/2004 (SEQ ID NO. 17), A/Memphis/1/1980 (SEQ ID NO. 21), A/Alaska/01/2021 (SEQ ID NO. 18), A/Indiana/11/2018 (SEQ ID NO. 16), A/Cambodia/e0826360/2020 (SEQ ID NO. 19), and A/Bilthoven/1971/1976 (SEQ ID NO. 24). FIG. 4 shows that any two of these eight H3N2 homologous distinct antigen components have pairwise sequence identity less than 96%, and greater than 80%. Any subset of six of these eight constitute sets of six homologous distinct antigen components with pairwise sequence identity less than 96% and greater than 80%.

mRNA generation from DNA plasmids and subsequent encapsulation in lipid nanoparticles (LNPs) was performed using methods well-known in the art, summarized here. DNA plasmids were constructed that comprise, under the SP6 promoter, the same 5′ UTR as used in the clinically approved BNT162b2 vaccine (SEQ ID NO: 106), DNA encoding an antigen component, the same 3′ UTR as used in BNT162b2 (SEQ ID NO: 107), and a poly-adenosine(120) tail (SEQ ID NO: 151). An alternative 3′ UTR from human HBA1 is shown as SEQ ID NO: 129. The plasmids were linearized using a NotI restriction site and utilized as templates for in vitro transcription (IVT) using SP6 RNA polymerase and a mix of nucleoside triphosphates. N1-methyl-pseudouridine-5′-triphosphate was used instead of uridine-5′-triphosphate for IVT. The Vaccinia enzymatic capping system (New England BioLabs) was used to generate capped mRNA. Purified mRNA was further encapsulated in the same LNP formulation as BNT162b2 (46.3% ionizable lipid ALC-0315, 9.4% phospholipid DSPC, 1.6% ALC-0159 PEG-lipid, and 42.7% cholesterol) to obtain the LNP-encapsulated RNA.

Codon optimization of nucleic acid sequences may be performed using various methods well-known in the art, including LinearDesign.

TABLE 5
Table of UTR Sequences

| SEQ | | |
|---|---|---|
| ID | | |
| NO: | Sequence | Description |
| 106 | GAGAATAAACTAGTATTCTT | 5′ UTR sequence |
| | CTGGTCCCCACAGACTCAGA | |
| | GAGAACCCGCCACC | |
| 107 | TGATGACTCGAGCTGGTACT | 3′ UTR sequence |
| | GCATGCACGCAATGCTAGCT | |
| | GCCCCTTTCCCGTCCTGGGT | |
| | ACCCCGAGTCTCCCCCGACC | |
| | TCGGGTCCCAGGTATGCTCC | |
| | CACCTCCACCTGCCCCACTC | |
| | ACCACCTCTGCTAGTTCCAG | |
| | ACACCTCCCAAGCACGCAGC | |
| | AATGCAGCTCAAAACGCTTA | |
| | GCCTAGCCACACCCCCACGG | |
| | GAAACAGCAGTGATTAACCT | |
| | TTAGCAATAAACGAAAGTTT | |
| | AACTAAGCTATACTAACCCC | |
| | AGGGTTGGTCAATTTCGTGC | |
| | CAGCCACACCCTGGAGCTAG | |
| | CA | |
| 129 | GCTGGAGCCTCGGTGGCCAT | Alternative 3′ UTR |
| | GCTTCTTGCCCCTTGGGCCT | sequence |
| | CCCCCCAGCCCCTCCTCCCC | |
| | TTCCTGCACCCGTACCCCCG | |
| | TGGTCTTTGAATAAAGTCTG | |
| | AGTGGGCGGCA | |

Serum ELISA assays in all Examples were performed as follows: The indicated proteins (5 ug/mL) were added to microtiter plates (CoStar), in coating buffer (0.1 M sodium bicarbonate, pH 8.6). After incubation at 4° C. overnight and blocking with 3% bovine serum albumin (BSA) in PBS, for 1 hour at 37° C., serially diluted serum/plasma in blocking buffer was added to individual wells and incubated for 1 hour at 37° C. Then, plates were washed three times with 0.05% PBST. Horseradish peroxidase (HRP)-conjugated anti-IgG secondary antibody was added to wells and incubated for 1 hour at 37° C. After washing three times with PBST, 2,2′-azino-bis-3-ethylbenzothiazoline-6-sulfonic (Thermo Fisher Scientific Inc., Waltham, MA, USA) or 3,3′,5,5′-tetramethylbenzidine liquid substrate system (Thermo Fisher Scientific Inc.) was added to the wells. Absorbance was measured at 405 or 650 nm, respectively, using a microplate spectrophotometer (Multiskan GO, Thermo Fisher Scientific Inc.).

### Example 2: Serum Activity to mRNA Antigens in Ferret

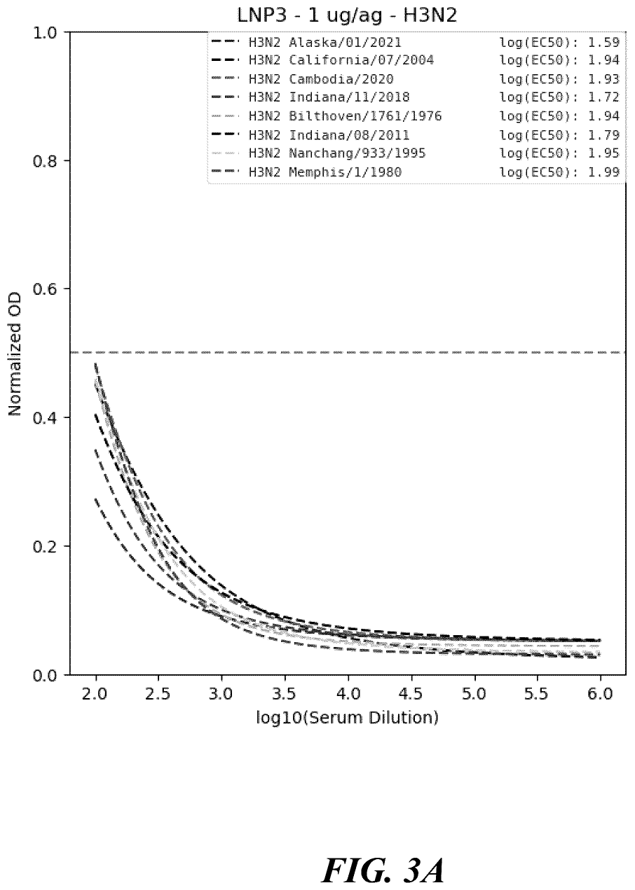

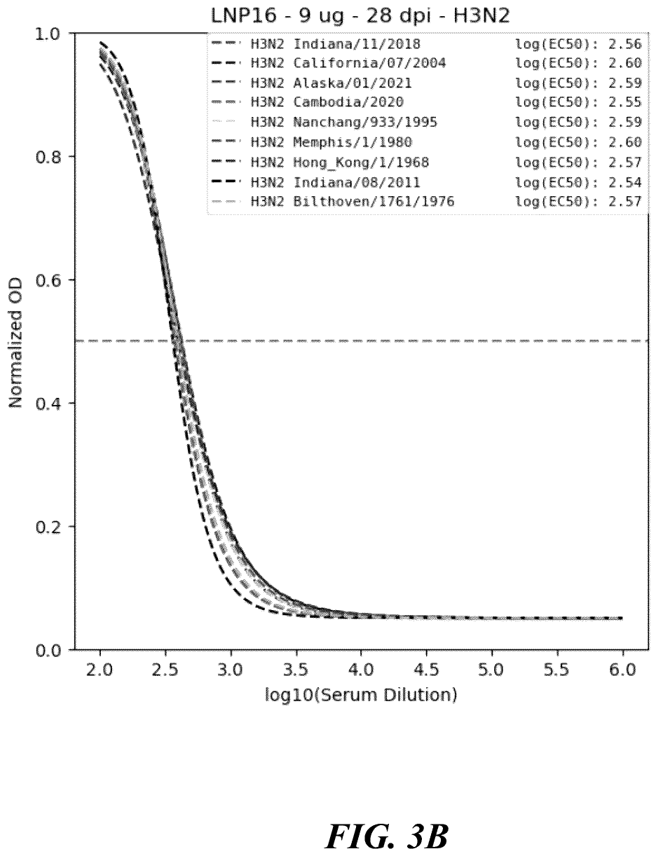

Ferrets were immunized once with mRNA encoding any one of eight distinct antigens complexed with a lipid nanoparticle (LNP). Specifically, ferrets were immunized with 1 μg of LNP-encapsulated mRNA encoding the hemagglutinin corresponding to H3N2 Alaska/01/2021 (SEQ ID NO: 18), H3N2 California/07/2004 (SEQ ID NO: 17), H3N2 Cambodia/2020 (SEQ ID NO: 19), H3N2 Indiana/11/2018 (SEQ ID NO: 16), H3N2 Bilthoven/1761/1976 (SEQ ID NO: 24), H3N2 Nanchang/933/1995 (SEQ ID NO: 20), or H3N2 Memphis/1/1980 (SEQ ID NO: 21). Serum was obtained 28 days after immunization. Serum reactivity to the recombinant protein of each antigen with which the ferrets were immunized was measured, as shown in FIG. 3A. The antigen-matched serum reactivity (i.e., serum from mice immunized with a given antigen tested against that same antigen) was very weak in all cases (EC50 not attained in any group even at the highest tested serum dilution of 1:100).

Ferrets were immunized with LNP-encapsulated mRNAs encoding the hemagglutinin corresponding to 8 H3N2 antigens (SEQ ID NOs: 18, 24, 9, 19, 16, 22, 20, 21), each at about 0.5 pg per antigen (total of 4 μg of H3N2 antigens). Serum reactivity to the recombinant protein of each antigen with which the ferrets were immunized was measured, as shown in FIG. 3B. The Centi-Flu immunized ferrets exhibited potent responses against all immunized antigens, as shown in FIG. 3B. It suggests that the single antigen at a low per-antigen dose (1 ug per antigen) is insufficient, but Centi-Flu at an even lower per-antigen dose (0.5 ug per antigen) induces potent and broad reactivity. Therefore, it is the unique combination of (1) using six or more homologous distinct antigen components and (2) mRNA encoding each component at a low dose, that achieves the desired serological effect.

All LNP-encapsulated RNA constructs were generated as described in Example 1. Serum ELISA was performed as described in Example 1.

One exemplary set of eight homologous distinct antigen components encoded by mRNA in the Centi-Flu vaccine composition are A/Hong Kong/1/1968 (SEQ ID NO. 22), A/Nanchang/933/1995 (SEQ ID NO. 20), A/California/07/2004 (SEQ ID NO. 17), A/Memphis/1/1980 (SEQ ID NO. 21), A/Alaska/01/2021 (SEQ ID NO. 18), A/Indiana/11/2018 (SEQ ID NO. 16), A/Cambodia/e0826360/2020 (SEQ ID NO. 19), and A/Bilthoven/1971/1976 (SEQ ID NO. 24). FIG. 4 shows that any two of these eight H3N2 homologous distinct antigen components have pairwise sequence identity less than 96%, and greater than 80%. Any subset of six of these eight constitute sets of six homologous distinct antigen components with pairwise sequence identity less than 96% and greater than 80%.

### Example 3: Hemagglutination Inhibition Tests on Immunized Mice

Mice were immunized twice (d0, d21) with an LNP-encapsulated mixture of 20 mRNAs (“LNP20”). Specifically, the 20 mRNAs encode antigens comprising SEQ ID NOS: 85-104, provided in Table 3; for example, using mRNA sequences provided in Table 2 (SEQ ID NOS: 64-83). Each mRNA of the mixture encoded for one influenza hemagglutinin (10 H1 antigens, 10 H3 antigens). Mice were immunized with a total mRNA dose of 18 μg, 6 μg, 2 μg, or 0.7 μg. Hemagglutination inhibition (HAI) was tested using serum drawn at 35 days post-immunization (dpi). HAI was tested against strains included in the LNP20 mixture (H1N1 A/Puerto Rico/8/1934, H1N1 A/California/04/2009, and H3N2 A/Alaska 01/2021); as well as against heterologous strains not included in the LNP20 mixture (H1N1 A/Connecticut/01/2021, H1N1 A/New Caledonia/20/99, H3N2 A/Fujian/411/2002, H3N2 A/Darwin/9/2021, and B/Michigan/01/2021). HAI titers >40 were observed for all strains except B/Michigan/01/2021, which is expected since LNP20 did not include mRNA encoding any influenza B hemagglutinin antigens.

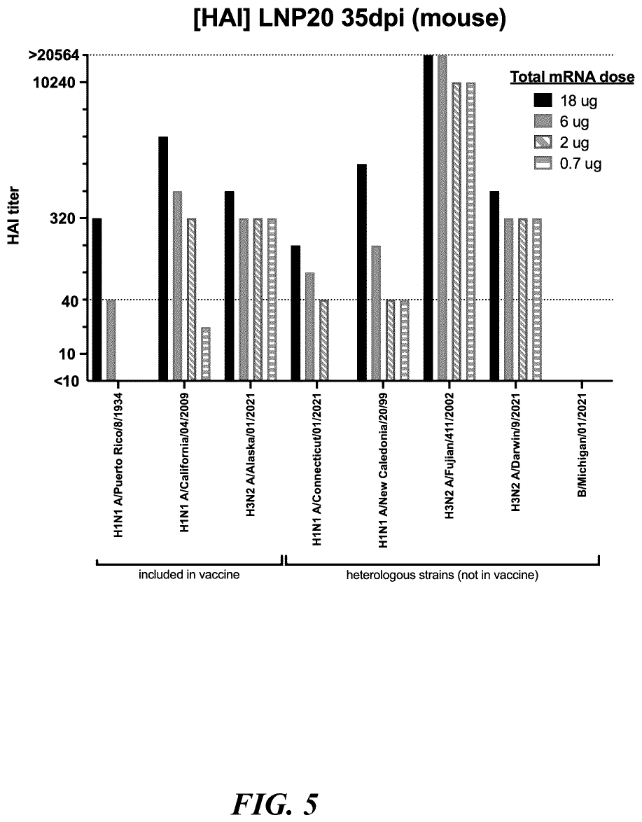

The results are depicted in FIG. 5. This demonstrates the potent and broad humoral response induced by LNP20.

### Example 4: Hemagglutination Inhibition Tests on Immunized Mice

Mice were immunized twice (d0, d21) with an LNP-encapsulated mixture of 18 mRNAs (“LNP18”). Each mRNA encoded for one influenza hemagglutinin (8×H1, 8×H3, 2×HAB). Specifically, the mRNAs of LNP18 encode antigens comprising SEQ ID NOS: 85-100 provided in Table 3; for example, using mRNA sequences provided in Table 2 (SEQ ID NOS: 64, 67-79, and 82-83). LNP18 further comprises mRNAs encoding two HAB strains: B/Guangdong-Maonan/316/2021 and B/Utah/02/2012. Mice were immunized with a total mRNA dose of 12 pg. Hemagglutination inhibition (HAI) was tested using serum drawn at 63 days post-immunization (dpi). HAI was tested against strains included in the LNP20 mixture (H1N1 A/Brisbane/59/2007, H1N1 A/California/04/2009, and H3N2 A/Alaska/01/2021); as well as against heterologous strains not included in the LNP20 mixture (all others). HAI titers >40 were observed for 13/16 strains, and ≥32 for 15/16 strains.

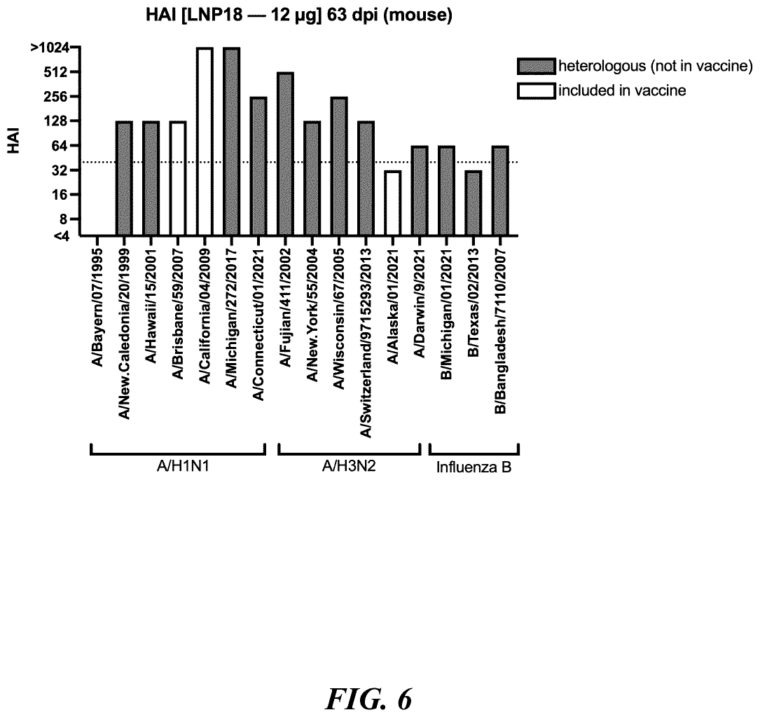

The results are depicted in FIG. 6. This demonstrates the potent and broad humoral response induced by LNP18.

### Example 5: Hemagglutination Inhibition Tests on Immunized Rats

Rats were immunized twice (d0, d21) with an LNP-encapsulated mixture of 18 mRNAs (“LNP18”). Each mRNA encoded for one influenza hemagglutinin (8×H1, 8×H3, 2×HAB). Specifically, the mRNAs of LNP18 encode antigens comprising SEQ ID NOS: 85-100 provided in Table 3; for example, using mRNA sequences provided in Table 2 (SEQ ID NOS: 64, 67-79, and 82-83. Rats were immunized with a total mRNA dose of 12 μg. Hemagglutination inhibition (HAI) was tested using serum drawn at 63 days post-immunization (dpi). HAI was tested against strains included in the LNP20 mixture (H1N1 A/Brisbane/59/2007, H1N1 A/California/04/2009, and H3N2 A/Alaska/01/2021); as well as against heterologous strains not included in the LNP20 mixture (all others). HAI titers >40 were observed for 12/16 strains, and >32 for 14/16 strains.

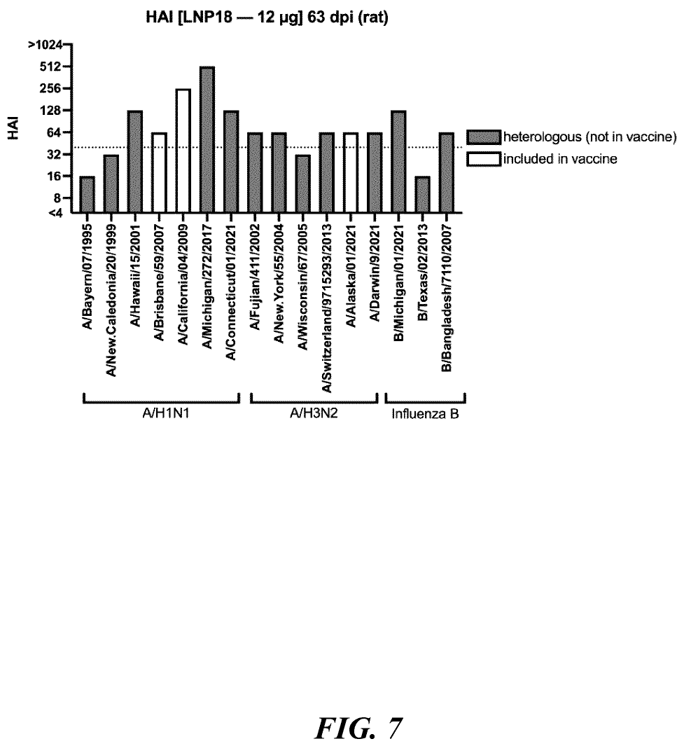

The results are depicted in FIG. 7. This demonstrates the potent and broad humoral response induced by LNP18.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The invention claimed is:

1.-219. (canceled)

220. A vaccine composition comprising a plurality of distinct antigens, the vaccine composition comprising:
(a) an antigen comprising an amino acid sequence corresponding to SEQ ID NO: 3 or a sequence at least 90% identical to SEQ ID NO: 3;
(b) an antigen comprising an amino acid sequence corresponding to SEQ ID NO: 16 or a sequence at least 90% identical to SEQ ID NO: 16;
(c) an antigen comprising an amino acid sequence corresponding to SEQ ID NO: 18 or a sequence at least 90% identical to SEQ ID NO: 18;
(d) an antigen comprising an amino acid sequence corresponding to SEQ ID NO: 19 or a sequence at least 90% identical to SEQ ID NO: 19;
(e) an antigen comprising an amino acid sequence corresponding to SEQ ID NO: 21 or a sequence at least 90% identical to SEQ ID NO: 21;
(f) an antigen comprising an amino acid sequence corresponding to SEQ ID NO: 24 or a sequence at least 90% identical to SEQ ID NO: 24;
(g) an antigen comprising an amino acid sequence corresponding to SEQ ID NO: 39 or a sequence at least 90% identical to SEQ ID NO: 39;
(h) an antigen comprising an amino acid sequence corresponding to SEQ ID NO: 40 or a sequence at least 90% identical to SEQ ID NO: 40;
(i) an antigen comprising an amino acid sequence corresponding to SEQ ID NO: 41 or a sequence at least 90% identical to SEQ ID NO: 41; and
(j) an antigen comprising an amino acid sequence corresponding to SEQ ID NO: 42 or a sequence at least 90% identical to SEQ ID NO: 42.

221. The vaccine composition of claim 220, wherein the vaccine composition comprises:
(a) an antigen comprising an amino acid sequence corresponding to SEQ ID NO: 3 or a sequence at least 99% identical to SEQ ID NO: 3;
(b) an antigen comprising an amino acid sequence corresponding to SEQ ID NO: 16 or a sequence at least 99% identical to SEQ ID NO: 16;
(c) an antigen comprising an amino acid sequence corresponding to SEQ ID NO: 18 or a sequence at least 99% identical to SEQ ID NO: 18;
(d) an antigen comprising an amino acid sequence corresponding to SEQ ID NO: 19 or a sequence at least 99% identical to SEQ ID NO: 19;
(e) an antigen comprising an amino acid sequence corresponding to SEQ ID NO: 21 or a sequence at least 99% identical to SEQ ID NO: 21;
(f) an antigen comprising an amino acid sequence corresponding to SEQ ID NO: 24 or a sequence at least 99% identical to SEQ ID NO: 24;
(g) an antigen comprising an amino acid sequence corresponding to SEQ ID NO: 39 or a sequence at least 99% identical to SEQ ID NO: 39;
(h) an antigen comprising an amino acid sequence corresponding to SEQ ID NO: 40 or a sequence at least 99% identical to SEQ ID NO: 40;
(i) an antigen comprising an amino acid sequence corresponding to SEQ ID NO: 41 or a sequence at least 99% identical to SEQ ID NO: 41; and
(j) an antigen comprising an amino acid sequence corresponding to SEQ ID NO: 42 or a sequence at least 99% identical to SEQ ID NO: 42.

222. The vaccine composition of claim 220, further comprising a pharmaceutically acceptable carrier.

223. The vaccine composition of claim 220, wherein each distinct antigen of the plurality of distinct antigens is present in the vaccine composition in an amount of less than about 5 micrograms (μg).

224. The vaccine composition of claim 223, wherein each distinct antigen of the plurality of distinct antigens is present in the vaccine composition in an amount of less than about 3 micrograms (μg).

225. The vaccine composition of claim 220, wherein the plurality of distinct antigens comprises at least 12 distinct antigens.

226. The vaccine composition of claim 220, wherein the plurality of distinct antigens comprises no more than 30 distinct antigens.

227. The vaccine composition of claim 220, wherein the plurality of distinct antigens is sufficient to elicit an immune response in a subject yet each individual distinct antigen of the plurality of distinct antigens is present in the composition in an amount that is insufficient to elicit in an immune response in a subject.

228. The vaccine composition of claim 220, wherein the vaccine composition comprises nineteen different sequences selected from the group consisting of SEQ ID NOS: 108-121, 123, and 125-128, or sequences at least 90% identical thereto.

229. The vaccine composition of claim 228, wherein the vaccine composition comprises nineteen different sequences selected from the group consisting of SEQ ID NOS: 108-121, 123, and 125-128.

230. A nucleic acid vaccine composition comprising one or more nucleic acids encoding for a plurality of distinct antigens, wherein the plurality of distinct antigens comprises:
(a) an antigen comprising an amino acid sequence corresponding to SEQ ID NO: 3 or a sequence at least 90% identical to SEQ ID NO: 3;
(b) an antigen comprising an amino acid sequence corresponding to SEQ ID NO: 16 or a sequence at least 90% identical to SEQ ID NO: 16;
(c) an antigen comprising an amino acid sequence corresponding to SEQ ID NO: 18 or a sequence at least 90% identical to SEQ ID NO: 18;
(d) an antigen comprising an amino acid sequence corresponding to SEQ ID NO: 19 or a sequence at least 90% identical to SEQ ID NO: 19;
(e) an antigen comprising an amino acid sequence corresponding to SEQ ID NO: 21 or a sequence at least 90% identical to SEQ ID NO: 21;
(f) an antigen comprising an amino acid sequence corresponding to SEQ ID NO: 24 or a sequence at least 90% identical to SEQ ID NO: 24;
(g) an antigen comprising an amino acid sequence corresponding to SEQ ID NO: 39 or a sequence at least 90% identical to SEQ ID NO: 39;
(h) an antigen comprising an amino acid sequence corresponding to SEQ ID NO: 40 or a sequence at least 90% identical to SEQ ID NO: 40;
(i) an antigen comprising an amino acid sequence corresponding to SEQ ID NO: 41 or a sequence at least 90% identical to SEQ ID NO: 41; and
(j) an antigen comprising an amino acid sequence corresponding to SEQ ID NO: 42 or a sequence at least 90% identical to SEQ ID NO: 42.

231. The vaccine composition of claim 230, wherein the plurality of distinct antigens comprises:
(a) an antigen comprising an amino acid sequence corresponding to SEQ ID NO: 3 or a sequence at least 99% identical to SEQ ID NO: 3;
(b) an antigen comprising an amino acid sequence corresponding to SEQ ID NO: 16 or a sequence at least 99% identical to SEQ ID NO: 16;
(c) an antigen comprising an amino acid sequence corresponding to SEQ ID NO: 18 or a sequence at least 99% identical to SEQ ID NO: 18;
(d) an antigen comprising an amino acid sequence corresponding to SEQ ID NO: 19 or a sequence at least 99% identical to SEQ ID NO: 19;
(e) an antigen comprising an amino acid sequence corresponding to SEQ ID NO: 21 or a sequence at least 99% identical to SEQ ID NO: 21;
(f) an antigen comprising an amino acid sequence corresponding to SEQ ID NO: 24 or a sequence at least 99% identical to SEQ ID NO: 24;
(g) an antigen comprising an amino acid sequence corresponding to SEQ ID NO: 39 or a sequence at least 99% identical to SEQ ID NO: 39;
(h) an antigen comprising an amino acid sequence corresponding to SEQ ID NO: 40 or a sequence at least 99% identical to SEQ ID NO: 40;
(i) an antigen comprising an amino acid sequence corresponding to SEQ ID NO: 41 or a sequence at least 99% identical to SEQ ID NO: 41; and
(j) an antigen comprising an amino acid sequence corresponding to SEQ ID NO: 42 or a sequence at least 99% identical to SEQ ID NO: 42.

232. The nucleic acid vaccine composition of claim 230, wherein the one or more nucleic acids comprise messenger RNA (mRNA).

233. The nucleic acid vaccine composition of claim 232, wherein the mRNA is encapsulated in lipid nanoparticles (LNPs).

234. The nucleic acid vaccine composition of claim 230, wherein each nucleic acid encoding a distinct antigen is present in the nucleic acid vaccine composition in an amount of less than about 5 micrograms (μg).

235. The nucleic acid vaccine composition of claim 234, wherein each nucleic acid encoding a distinct antigen is present in the nucleic acid vaccine composition in an amount of less than about 3 micrograms (μg).

236. The nucleic acid vaccine composition of claim 230, wherein the plurality of distinct antigens comprises at least 12 distinct antigens.

237. The nucleic vaccine composition of claim 230, wherein the plurality of distinct antigens comprises no more than 30 distinct antigens.

238. The nucleic acid vaccine composition of claim 230, wherein the vaccine composition comprises nineteen nucleic acid sequences encoding SEQ ID NOS: 108-121, 123, and 125-128, or sequences at least 90% identical thereto.

239. The nucleic acid vaccine composition of claim 238, wherein the vaccine composition comprises nineteen nucleic acid sequences encoding SEQ ID NOS: 108-121, 123, and 125-128.
